(19) United States
(12) Patent Application Publication
(10) Pub. No.: US 2026/0258040 A1
(43) Pub. Date: Sep. 3, 2026

(54) PYRAN AND CYCLOHEXYL ARYL ETHER ANALOGS AS INHIBITORS OF THE MENIN-MLL INTERACTION

(71) Applicant: Syndax Pharmaceuticals, Inc., New York, NY (US)

(72) Inventors: Gerard M. MCGEEHAN, Newtown Square, PA (US); William H. MILLER, Collegeville, PA (US); Swapan K. SAMANTA, Hyderabad (IN); Virsinha REDDY, Hyderabad (IN)

(21) Appl. No.: 19/553,056

(22) Filed: Feb. 27, 2026

Related U.S. Application Data
(60) Provisional application No. 63/764,041, filed on Feb. 27, 2025.

(51) Int. Cl. C07D 471/10; A61K 31/506; A61K 31/53; A61P 35/02

(52) U.S. Cl. CPC C07D 471/10; A61K 31/506; A61K 31/53; A61P 35/02

(57) ABSTRACT

The present disclosure is directed to pyran and cyclohexyl aryl ether analogs of Formula I, or a stereoisomer thereof, or a pharmaceutically acceptable salt thereof, as inhibitors of the interaction of menin with MLL and MLL fusion proteins, pharmaceutical compositions containing the same, and their use in the treatment of cancer and other diseases mediated by the menin-MLL interaction.

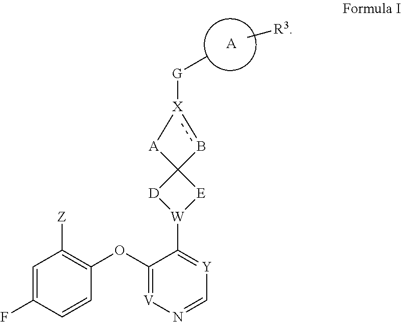

### RELATED APPLICATIONS

This application claims priority to, and the benefit of, U.S. Provisional Application No. 63/764,041, filed Feb. 27, 2025, the entire contents of which are incorporated herein by reference.

### BACKGROUND

The mixed-lineage leukemia (MLL), also known as KMT2A, gene encodes a protein that is a histone methyltransferase that is mutated in clinically and biologically distinctive subsets of acute leukemia. Rearranged mixed lineage leukemia (MLL-r) involves recurrent translocations of the 11q23 chromosome locus which lead to an aggressive form of acute leukemia with limited therapeutic options. These translocations target the MLL gene creating an oncogenic fusion protein comprising the amino-terminus of MLL fused in frame with more than 60 different fusion protein partners. Menin, a ubiquitously expressed, nuclear protein encoded by the multiple endocrine neoplasia type 1 (MEN1) tumor suppressor gene, has a high affinity binding interaction with MLL fusion proteins and is an essential co-factor of oncogenic MLL-r fusion proteins. Disruption of this interaction leads to selective growth inhibition and apoptosis of MLL-r leukemia cells both in vitro and in vivo.

The interaction between menin and MLL or MLL fusion proteins is an attractive target for therapeutic intervention, and there is a need for novel agents that inhibit the menin-MLL interaction for the treatment of various diseases and conditions, including leukemia, other cancers, and diabetes.

### SUMMARY

In one aspect, the present disclosure is directed to a compound of Formula I:

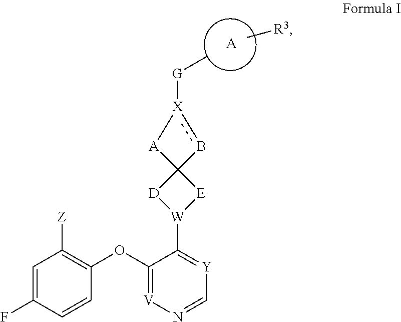

a stereoisomer, or a pharmaceutically acceptable salt thereof, wherein:
[symbol] denotes a single bond or a double bond, as valency permits;
A, B, D, and E are each independently selected from —C(R^{A1})(R^{A2})—, C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂)—;
V is N or CH;
Y is N or CH;
G is O, CH₂, or NH;
Ring A is cyclohexyl or 6-membered heterocycloalkyl which has one heteroatom ring member being an oxygen;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{x}, wherein R^{x} is absent, H, halo, CN, OH, C₁₋₆ alkyl, C₁₋₆ alkoxy, amino, C₁₋₆ alkyl amino, or C₂₋₈ dialkylamino;
Z is halo, C₁₋₆ alkyl, C₁₋₆ haloalkyl, C₁₋₄ cyanoalkyl, C₂₋₆ alkenyl, C₂₋₆ alkynyl, CN, NO₂, SR^{a3}, C(O)R^{b3}, C(O)NR¹R², C(O)OR^{a3}, OC(O)R^{b3}, OC(O)NR³R^{d3}, C(═NR^{e3})NR^{c3}R^{d3} NR^{c3}C(═NR^{e3})NR^{c3}R^{d3}, NR^{c3}R^{d3}, NR^{c3}C(O)OR^{a3}, NR^{c3}C(O)NR^{c3}R^{d3}, NR^{c3}S(O)R^{b3}, NR^{c3}S(O)₂NR^{c3}R^{d3}, S(O)R^{b3}, S(O)NR³R^{d3}, S(O)₂R^{b3}, and P(O)R^{c3}R^{d3}, wherein said C₁₋₆ alkyl, C₁₋₄ haloalkyl, C₁₋₄ cyanoalkyl, C₂₋₆ alkenyl, and C₂₋₆ alkynyl are each optionally substituted with 1, 2, 3, or 4 substituents independently selected from Cy², halo, CN, NO₂, CN, NO₂, OR^{a3}, SR^{a3}, C(O)R^{b3}, C(O)NR³R^{d3}, C(O)OR^{a3}, OC(O)R^{b3}, OC(O)NR^{c3}R^{d3}, C(═NR^{e3})NR³R^{d3} NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{A1} is independently selected from absent, H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A2} is independently selected from H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A3} is independently selected from H, C₁₋₄ alkyl, C₁₋₄ alkoxy, C₁₋₄ haloalkyl, C(O)R^{z}, and C(O)OR^{z}, wherein said C₁₋₆ alkyl is optionally substituted with phenyl, C₁₋₄ alkoxy, C₁₋₄ haloalkoxy, CN, NO₂, or OH;
R^{z} is H, C₁₋₆ alkyl, or phenyl;
each Cy² is independently selected from C₆₋₁₄ aryl, C₃₋₁₈ cycloalkyl, 5-16 membered heteroaryl, and 4-18 membered heterocycloalkyl, each of which is optionally substituted with 1, 2, 3, or 4 substituents independently selected from R^{Cy2};
each R^{Cy2} is independently selected from halo, C₁₋₆ alkyl, C₁₋₄ haloalkyl, C₁₋₄ cyanoalkyl, C₂₋₆ alkenyl, C₂₋₆ alkynyl, phenyl, C₃₋₇ cycloalkyl, 5-6 membered heteroaryl, 4-7 membered heterocycloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{e5}R^{d5}, NR^{c5}R^{d5}, NR^{e5}C(O)R^{b5}, NR^{c5}C(O)OR^{a5}, NR^{c5}C(O)NR⁵R⁴⁵, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein said C₁₋₆ alkyl, C₁₋₄ haloalkyl, C₁₋₄ cyanoalkyl, C₂₋₆ alkenyl, C₂₋₆ alkynyl, phenyl, C₃₋₇ cycloalkyl, 5-6 membered heteroaryl, and 4-7 membered heterocycloalkyl are each optionally substituted with 1, 2, 3, or 4 substituents independently selected from CN, NO₂, OR^{a5}, SR^{a5}, C(O)R^{b5}, C(O)NR^{c5}R⁴⁵, C(O)OR^{a5}, OC(O)R^{b5}, OC(O)NR⁵R^{d5}, C(═NR^{e5})NR⁵R^{d5}, NR^{c5}C(═NR^{e5})NR^{c5}R^{d5}, NR^{c5}R^{d5}, NR⁵C(O)R^{b5}, NR^{c5}C(O)OR^{a5}, NR^{c5}C(O)NR⁵R^{d5}, NR^{c5}S(O)R^{b5}, NR^{c5}S(O)₂R^{b5}, NR^{c5}S(O)₂NR⁶⁵R^{d5}, S(O)R^{b5}, S(O)NR^{c5}R^{d5}, S(O)₂R^{b5}, and S(O)₂NR^{c5}R^{d5},
R¹ is
   (a) H, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a},
   (c) -C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as},
      each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs}, each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es};
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1} NR^{c1}C(═NR^{c1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{e1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{e1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1} NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, each R^{a1}, R^{b1}, R^{e1}, R^{d1}, R^{a3}, R^{b3}, R^{e3}, R^{d3}, R^{a5}, R^{b5}, R^{e5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1}, R^{e3}, and R^{e5} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a} is independently H, CN, or C₁₋₆ alkyl optionally substituted with one or more halo, C₆₋₁₀ aryl, or N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In one aspect, the present disclosure is directed to a compound of Formula II:

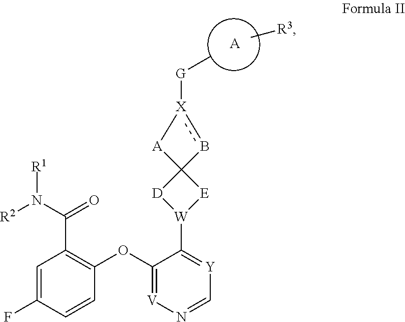

a stereoisomer, or a pharmaceutically acceptable salt thereof, wherein:
[symbol] denotes a single bond or a double bond, as valency permits;
A, B, D, and E are each independently selected from —C(R^{A1})(R^{A2})—, —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂);
V is N or CH;
Y is N or CH;
G is O, CH₂, or NH;
Ring A is cyclohexyl or 6-membered heterocycloalkyl which has one heteroatom ring member being an oxygen;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{x}, wherein R^{x} is absent, H, halo, CN, OH, C₁₋₆ alkyl, C₁₋₆ alkoxy, amino, C₁₋₆ alkyl amino, or C₂₋₈ dialkylamino;
each R^{A1} is independently selected from absent, H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A2} is independently selected from H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A3} is independently selected from H, C₁₋₄ alkyl, C₁₋₄ alkoxy, C₁₋₄ haloalkyl, C(O)R^{z}, and C(O)OR², wherein said C₁₋₆ alkyl is optionally substituted with phenyl, C₁₋₄ alkoxy, C₁₋₄ haloalkoxy, CN, NO₂, or OH;
R^{z} is H, C₁₋₆ alkyl, or phenyl;
R¹ is
   (a) H, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) -C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
      each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es},
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{c1})NR^{c1}R^{d1}, NR^{c1}R^{d1}, NR^{c1}C(O)R^{b1}, NR^{c1}C(O)OR^{a1}, NR^{e1}C(O)NR^{c1}R^{d1} NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1} NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, and R^{d1} is independently selected from H, C₁₋₆ alkyl, C₁₋₆ haloalkyl, C₂-6 alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a} is independently H, CN, or C₁₋₆ alkyl optionally substituted with one or more halo, C₆₋₁₀ aryl, or N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In one aspect, the present disclosure is directed to a compound of Formula III:

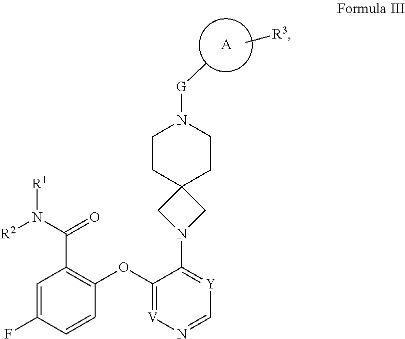

a stereoisomer, or a pharmaceutically acceptable salt thereof, wherein:
V is N or CH;
Y is N or CH;
G is O, CH₂, or NH;
Ring A is cyclohexyl or 6-membered heterocycloalkyl which has one heteroatom ring member being an oxygen;
R¹ is
   (a) -C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as}; each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
   (b) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es},
      each R^{1es} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1} NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{e1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{e1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, and R^{d1} is independently selected from H, C₁₋₆ alkyl, C₁₋₆ haloalkyl, C₂-6 alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In one aspect, the present disclosure is directed to a compound of Formula III-a:

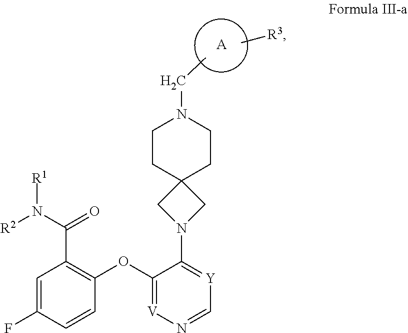

a stereoisomer, or a pharmaceutically acceptable salt thereof, wherein:
V is N or CH;
Y is N or CH;
Ring A is cyclohexyl or 6-membered heterocycloalkyl which has one heteroatom ring member being an oxygen;
R¹ is
   (a) -C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
      each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′,
   (b) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es},
      each R^{1es} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{e1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1} NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{e1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and
each R^{a1}, R^{b1}, R^{e1}, and R^{d1} is independently selected from H, C₁₋₆ alkyl, C₁₋₆ haloalkyl, C₂-6 alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In one aspect, the present disclosure is directed to a compound of Formula III-b:

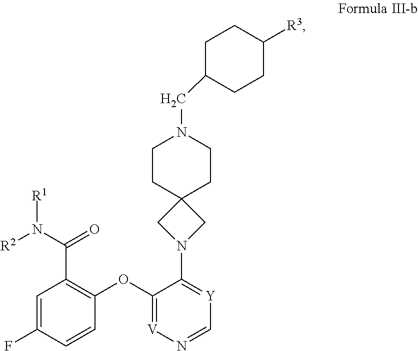

a stereoisomer, or a pharmaceutically acceptable salt thereof, wherein:
V is N or CH;
Y is N or CH;
R¹ is
   (a) -C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as},
      each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
   (b) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1} NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{e1}C(O)R^{b1}, NR^{c1}C(O)OR^{a1}, NR^{c1}C(O)NR^{c1}R^{d1}, NR^{e1}S(O)R^{b1}, NR^{c1}S(O)₂R^{b1}, NR^{e1}S(O)₂NR^{c1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, and R^{d1} is independently selected from H, C₁₋₆ alkyl, C₁₋₆ haloalkyl, C₂-6 alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In one aspect, the present disclosure is directed to a compound of Formula III-c:

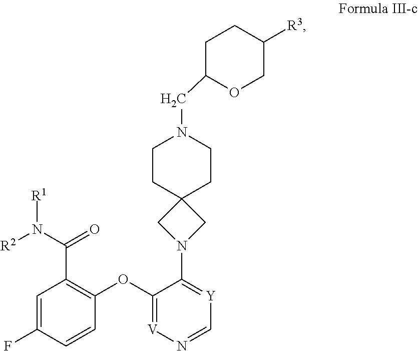

a stereoisomer, or a pharmaceutically acceptable salt thereof, wherein:
V is N or CH;
Y is N or CH;
R¹ is
   (a) -C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
      each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
   (b) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs}.
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{e1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{e1}S(O)₂NR^{c1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, and R^{d1} is independently selected from H, C₁₋₆ alkyl, C₁₋₆ haloalkyl, C₂-6 alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered 5 heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In one aspect, the present application relates to a pharmaceutical composition comprising a compound of the application, or a pharmaceutically acceptable salt, and a pharmaceutically acceptable carrier.

10 In one aspect, the present application relates to a pharmaceutical composition comprising a therapeutically effective amount of a compound of the application, or a pharmaceutically acceptable salt, and a pharmaceutically acceptable carrier.

In one aspect, the present application relates to a pharmaceutical composition comprising a compound of the application, and a pharmaceutically acceptable carrier.

In one aspect, the present application relates to a pharmaceutical composition comprising a therapeutically effective amount of a compound of the application, and a pharmaceutically acceptable carrier.

The present disclosure further provides a use of a compound of Formula I, II, III, III-a, III-b, III-c, III-b-1, III-b-2, III-b-3, III-c-1, III-c-2, or III-c-3, or a stereoisomer, or a pharmaceutically acceptable salt thereof in a method of treating cancer in a patient in need thereof.

The present disclosure further provides a compound of Formula I, II, III, III-a, III-b, III-c, III-b-1, III-b-2, III-b-3, III-c-1, III-c-2, or III-c-3, or a stereoisomer, or a pharmaceutically acceptable salt thereof for use in a method of treating cancer in a patient in need thereof.

The present disclosure further provides a compound of Formula I, II, III, III-a, III-b, III-c, III-b-1, III-b-2, III-b-3, III-c-1, III-c-2, or III-c-3, or a stereoisomer, or a pharmaceutically acceptable salt thereof for use in the manufacture of a medicament for the treatment of cancer in a patient in need thereof.

The present disclosure further provides a method of inhibiting the interaction between menin and MLL comprising contacting the menin and MLL with a compound of Formula I, II, III, III-a, III-b, III-c, III-b-1, III-b-2, III-b-3, III-c-1, III-c-2, or III-c-3, or a stereoisomer, or a pharmaceutically acceptable salt thereof.

The present disclosure further provides a method of inhibiting the interaction between menin and MLL comprising contacting the menin and MLL with an effective amount of a compound of Formula I, II, III, III-a, III-b, III-c, III-b-1, III-b-2, III-b-3, III-c-1, III-c-2, or III-c-3, or a stereoisomer, or a pharmaceutically acceptable salt thereof.

The present disclosure further provides a method of inhibiting the interaction between menin and MLL comprising contacting the menin and MLL with a pharmaceutical composition comprising a compound of Formula I, II, III, III-a, III-b, III-c, III-b-1, III-b-2, III-b-3, III-c-1, III-c-2, or III-c-3, or a pharmaceutically acceptable salt thereof, and at least one pharmaceutically acceptable carrier.

The present disclosure further provides a method of treating cancer in a patient comprising administering to the patient a therapeutically effective amount of a compound of Formula I, II, III, III-a, III-b, III-c, III-b-1, III-b-2, III-b-3, III-c-1, III-c-2, or III-c-3, a stereoisomer, or a pharmaceutically acceptable salt thereof.

The present disclosure further provides a method of inhibiting the interaction between menin and MLL comprising contacting the menin and MLL with a compound of Formula I, II, III, III-a, III-b, III-c, III-b-1, III-b-2, III-b-3, III-c-1, III-c-2, or III-c-3, a stereoisomer, or a pharmaceutically acceptable salt thereof.

The present disclosure further provides a method of treating cancer in a patient comprising administering to the patient an effective amount of a compound of Formula I, II, III, III-a, III-b, III-c, III-b-1, III-b-2, III-b-3, III-c-1, III-c-2, or III-c-3, a stereoisomer, or a pharmaceutically acceptable salt thereof.

The present disclosure further provides a method of treating cancer in a patient comprising administering to the patient a compound of Formula I, II, III, III-a, III-b, III-c, III-b-1, III-b-2, III-b-3, III-c-1, III-c-2, or III-c-3, a stereoisomer, or a pharmaceutically acceptable salt thereof.

The present disclosure further provides a method of treating cancer in a patient comprising administering to the patient a pharmaceutical composition comprising a compound of Formula I, II, III, III-a, III-b, III-c, III-b-1, III-b-2, III-b-3, III-c-1, III-c-2, or III-c-3, a stereoisomer, or a pharmaceutically acceptable salt thereof, and at least one pharmaceutically acceptable carrier.

The present disclosure further provides a pharmaceutical composition comprising a compound of Formula I, II, III, III-a, III-b, III-c, III-b-1, III-b-2, III-b-3, III-c-1, III-c-2, or III-c-3, a stereoisomer, or a pharmaceutically acceptable salt thereof, and at least one pharmaceutically acceptable carrier.

The present disclosure further provides a pharmaceutical composition comprising a salt or crystalline form of a compound of Formula I, II, III, III-a, III-b, III-c, III-b-1, III-b-2, III-b-3, III-c-1, III-c-2, or III-c-3, and at least one pharmaceutically acceptable carrier.

The present disclosure further provides a compound of Formula I, II, III, III-a, III-b, III-c, III-b-1, III-b-2, III-b-3, III-c-1, III-c-2, or III-c-3, wherein the compound is useful for the treatment of cancer and wherein the compound minimizes hERG binding.

The details of the disclosure are set forth in the accompanying description below. Although methods and materials similar or equivalent to those described herein can be used in the practice or testing of the present application, illustrative methods and materials are now described. In the case of conflict, the present specification, including definitions, will control. In addition, the materials, methods, and examples are illustrative only and are not intended to be limiting. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### DETAILED DESCRIPTION

In some embodiments, the present disclosure is directed to a compound of Formula I:

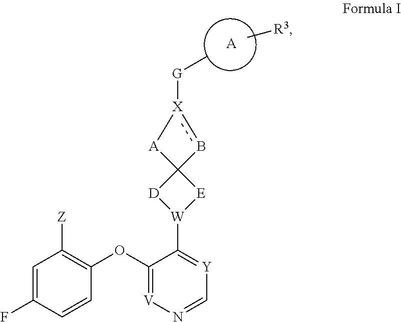

a stereoisomer, or a pharmaceutically acceptable salt thereof, wherein:
[symbol] denotes a single bond or a double bond, as valency permits;
A, B, D, and E are each independently selected from —C(R^{A1})(R^{A2})—, —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂);
V is N or CH;
Y is N or CH;
G is O, CH₂, or NH;
Ring A is cyclohexyl or 6-membered heterocycloalkyl which has one heteroatom ring member being an oxygen;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{x}, wherein R^{x} is absent, H, halo, CN, OH, C₁₋₆ alkyl, C₁₋₆ alkoxy, amino, C₁₋₆ alkyl amino, or C₂₋₈ dialkylamino;
Z is halo, C₁₋₆ alkyl, C₁₋₄ haloalkyl, C₁₋₄ cyanoalkyl, C₂₋₆ alkenyl, C₂₋₆ alkynyl, CN, NO₂, SR^{a3}, C(O)R^{b3}, C(O)NR¹R², C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)OR^{a3}, NR^{c3}C(O)NR^{c3}R^{d3}, NR^{c3}S(O)R^{b3}, NR^{c3}S(O)₂NR^{c3}R^{d3}, S(O)R^{b3}, S(O)NR^{c3}R^{d3}, S(O)₂R^{b3}, and P(O)R^{e3}R^{d3}, wherein said C₁₋₆ alkyl, C₁₋₄ haloalkyl, C₁₋₄ cyanoalkyl, C₂₋₆ alkenyl, and C₂₋₆ alkynyl are each optionally substituted with 1, 2, 3, or 4 substituents independently selected from Cy², halo, CN, NO₂, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR³R^{d3}, S(O)R^{b3}, S(O)NR^{c3}R^{d3}, S(O)₂R^{b3}, and S(O)₂NR^{c3}R^{d3};
each R^{A1} is independently selected from absent, H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A2} is independently selected from H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A3} is independently selected from H, C₁₋₄ alkyl, C₁₋₄ alkoxy, C₁₋₄ haloalkyl, C(O)R^{z}, and C(O)OR², wherein said C₁₋₆ alkyl is optionally substituted with phenyl, C₁₋₄ alkoxy, C₁₋₄ haloalkoxy, CN, NO₂, or OH;
R^{z} is H, C₁₋₆ alkyl, or phenyl;
each Cy² is independently selected from C₆₋₁₄ aryl, C₃₋₁₈ cycloalkyl, 5-16 membered heteroaryl, and 4-18 membered heterocycloalkyl, each of which is optionally substituted with 1, 2, 3, or 4 substituents independently selected from R^{Cy2};
each R^{Cy2} is independently selected from halo, C₁₋₆ alkyl, C₁₋₄ haloalkyl, C₁₋₄ cyanoalkyl, C₂₋₆ alkenyl, C₂₋₆ alkynyl, phenyl, C₃₋₇ cycloalkyl, 5-6 membered heteroaryl, 4-7 membered heterocycloalkyl, CN, NO₂, OR^{a5}, SR^{a5}, C(O)R^{b5}, C(O)NR^{c5}R^{d5}, C(O)OR^{a5}, OC(O)R^{b5} OC(O)NR^{c5}R^{d5}, C(═NR^{e5})NR^{e5}R^{d5}, NR⁵C(═NR^{e5})NR⁵R^{d5}, NR⁶⁵R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR⁵R^{d5}, S(O)R^{b5}, S(O)NR⁵R^{d5}, S(O)₂R^{b5}, and S(O)₂NR^{c5}R^{d5}, wherein said C₁₋₆ alkyl, C₁₋₄ haloalkyl, C₁₋₄ cyanoalkyl, C₂₋₆ alkenyl, C₂₋₆ alkynyl, phenyl, C₃₋₇ cycloalkyl, 5-6 membered heteroaryl, and 4-7 membered heterocycloalkyl are each optionally substituted with 1, 2, 3, or 4 substituents independently selected from CN, NO₂, OR^{a5}, SR^{a5}, C(O)R^{b5}, C(O)NR^{e5}R^{d5}, C(O)OR^{a5}, OC(O)R^{b5}, OC(O)NR^{c5}R^{d5}, C(═NR^{e5})NR⁵R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
R¹ is
   (a) H, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R¹s,
   each R^{1s} is independently oxo or ═NR^{4a},
   (c) -C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as}; each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es},
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{e1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{c1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1} NR^{c1}S(O)R^{b1}, NR^{e1}S(O)₂R^{b1}, NR^{e1}S(O)₂NR^{e1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{e1}R^{d1}, NR^{e1}S(O)₂NR^{c1}R^{d1}, S(O)R^{b1}, S(O)NR^{c1}R^{d1}, S(O)₂R^{b1}, and S(O)₂NR^{c1}R^{d1}, each R^{a1}, R^{b1}, R^{e1}, R^{d1}, R^{a3}, R^{b3}, R^{e3}, R^{d3}, R^{a5}, R^{b5}, R^{e5}, and R⁴⁵ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1}, R^{e3}, and R^{e5} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a} is independently H, CN, or C₁₋₆ alkyl optionally substituted with one or more halo, C₆₋₁₀ aryl, or N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula I, a stereoisomer, or a pharmaceutically acceptable salt thereof, wherein:
[symbol] denotes a single bond or a double bond, as valency permits;
A, B, D, and E are each independently selected from —C(R^{A1})(R^{A2})—, —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂);
V is N or CH;
Y is N or CH;
G is CH₂;
Ring A is cyclohexyl or 6-membered heterocycloalkyl which has one heteroatom ring member being an oxygen;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{x}, wherein R^{x} is absent, H, halo, CN, OH, C₁₋₆ alkyl, C₁₋₆ alkoxy, amino, C₁₋₆ alkyl amino, or C₂₋₈ dialkylamino;
Z is halo, C₁₋₆ alkyl, C₁₋₄ haloalkyl, C₁₋₄ cyanoalkyl, C₂₋₆ alkenyl, C₂₋₆ alkynyl, CN, NO₂, SR^{a3}, C(O)R^{b3}, C(O)NR¹R², C(O)OR^{a3}, OC(O)R^{b3}, OC(O)NR³R^{d3}, C(═NR^{e3})NR^{c3}R^{d3} NR^{c3}C(═NR^{e3})NR^{c3}R^{d3}, NR^{c3}R^{d3}, NR^{c3}C(O)OR^{a3}, NR^{c3}C(O)NR^{c3}R^{d3}, NR^{c3}S(O)R^{b3}, NR^{c3}S(O)₂NR^{c3}R^{d3}, S(O)R^{b3}, S(O)NR^{c3}R^{d3}, S(O)₂R^{b3}, and P(O)R^{c3}R^{d3}, wherein said C₁₋₆ alkyl, C₁₋₄ haloalkyl, C₁₋₄ cyanoalkyl, C₂₋₆ alkenyl, and C₂₋₆ alkynyl are each optionally substituted with 1, 2, 3, or 4 substituents independently selected from Cy², halo, CN, NO₂, CN, NO₂, OR^{a3}, SR^{a3}, C(O)R^{b3}, C(O)NR³R^{d3}, C(O)OR^{a3}, OC(O)R^{b3}, OC(O)NR³R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR³R^{d3}, S(O)R^{b3}, S(O)NR^{c3}R^{d3}, S(O)₂R^{b3}, and S(O)₂NR^{c3}R^{d3};
each R^{A1} is independently selected from absent, H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A2} is independently selected from H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A3} is independently selected from H, C₁₋₄ alkyl, C₁₋₄ alkoxy, C₁₋₄ haloalkyl, C(O)R^{z}, and C(O)OR², wherein said C₁₋₆ alkyl is optionally substituted with phenyl, C₁₋₄ alkoxy, C₁₋₄ haloalkoxy, CN, NO₂, or OH;
R^{z} is H, C₁₋₆ alkyl, or phenyl;
each Cy² is independently selected from C₆₋₁₄ aryl, C₃₋₁₈ cycloalkyl, 5-16 membered heteroaryl, and 4-18 membered heterocycloalkyl, each of which is optionally substituted with 1, 2, 3, or 4 substituents independently selected from R^{Cy2};
each R^{Cy2} is independently selected from halo, C₁₋₆ alkyl, C₁₋₄ haloalkyl, C₁₋₄ cyanoalkyl, C₂₋₆ alkenyl, C₂₋₆ alkynyl, phenyl, C₃₋₇ cycloalkyl, 5-6 membered heteroaryl, 4-7 membered heterocycloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R⁴⁵, S(O)R^{b5}, S(O)NR⁵R^{d5}, S(O)₂R^{b5}, and S(O)₂NR^{c5}R^{d5}, wherein said C₁₋₆ alkyl, C₁₋₄ haloalkyl, C₁₋₄ cyanoalkyl, C₂₋₆ alkenyl, C₂₋₆ alkynyl, phenyl, C₃₋₇ cycloalkyl, 5-6 membered heteroaryl, and 4-7 membered heterocycloalkyl are each optionally substituted with 1, 2, 3, or 4 substituents independently selected from CN, NO₂, OR^{a5}, SR^{a5}, C(O)R^{b5}, C(O)NR^{c5}R^{d5}, C(O)OR^{a5}, OC(O)R^{b5}, OC(O)NR⁵R^{d5}, C(═NR^{e5})NR^{e5}R^{d5}, NR^{c5}C(═NR^{e5})NR⁵R^{d5}, NR⁶⁵R^{d5}, NR⁵C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es},
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{e1}R^{d1}, C(═NR^{e1})NR^{c1}R^{d1} NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1} NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, R^{d1}, R^{a3}, R^{b3}, R^{e3}, R^{d3}, R^{a5}, R^{b5}, R^{e5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1}, R^{e3}, and R^{e5} is independently selected from H, C₁₋₆ alkyl, and CN;
each R₅ is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
   wherein any cycloalkyl or heterocycloalkyl group is optionally further substituted with 1 or 2 oxo groups.

In some embodiments, the present disclosure is directed to a compound of Formula I, a stereoisomer, or a pharmaceutically acceptable salt thereof, wherein:
a stereoisomer, or a pharmaceutically acceptable salt thereof, wherein:
[symbol] denotes a single bond;
A, B, D, and E are each independently selected from —C(R^{A1})(R^{A2})— and —C(R^{A1})(R^{A2})—C(R^{A1})(R^{A2})—;
V is N or CH;
Y is N or CH;
G is CH₂;
Ring A is cyclohexyl or 6-membered heterocycloalkyl which has one heteroatom ring 10 member being an oxygen;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{x}, wherein R^{x} is absent, H, halo, CN, OH, C₁₋₆ alkyl, C₁₋₆ alkoxy, amino, C₁₋₆ alkyl amino, or C₂₋₈ dialkylamino;
Z is halo, C₁₋₆ alkyl, C₁₋₄ haloalkyl, C₁₋₄ cyanoalkyl, C₂₋₆ alkenyl, C₂₋₆ alkynyl, CN, NO₂, SR^{a3}, C(O)R^{b3}, C(O)NR¹R², C(O)OR^{a3}, OC(O)R^{b3}, OC(O)NR³R^{d3}, C(═NR^{e3})NR^{c3}R^{d3}, NR^{c3}C(═NR^{e3})NR^{c3}R^{d3}, NR^{c3}R^{d3}, NR^{c3}C(O)OR^{a3}, NR^{c3}C(O)NR^{c3}R^{d3}, NR^{c3}S(O)R^{b3}, NR^{c3}S(O)₂NR^{c3}R^{d3}, S(O)R^{b3}, S(O)NR^{c3}R^{d3}, S(O)₂R^{b3}, and P(O)R^{c3}R^{d3}, wherein said C₁₋₆ alkyl, C₁₋₄ haloalkyl, C₁₋₄ cyanoalkyl, C₂₋₆ alkenyl, and C₂₋₆ alkynyl are each optionally substituted with 1, 2, 3, or 4 substituents independently selected from Cy², halo, CN, NO₂, CN, NO₂, OR^{a3}, SR^{a3}, C(O)R^{b3}, C(O)NR³R^{d3}, C(O)OR^{a3}, OC(O)R^{b3}, OC(O)NR³R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR³C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{A1} is independently selected from absent, H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A2} is independently selected from H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each Cy² is independently selected from C₆₋₁₄ aryl, C₃₋₁₈ cycloalkyl, 5-16 membered heteroaryl, and 4-18 membered heterocycloalkyl, each of which is optionally substituted with 1, 2, 3, or 4 substituents independently selected from R^{Cy2};
each R^{Cy2} is independently selected from halo, C₁₋₆ alkyl, C₁₋₄ haloalkyl, C₁₋₄ cyanoalkyl, C₂₋₆ alkenyl, C₂₋₆ alkynyl, phenyl, C₃₋₇ cycloalkyl, 5-6 membered heteroaryl, 4-7 membered heterocycloalkyl, CN, NO₂, OR^{a5}, SR^{a5}, C(O)R^{b5}, C(O)NR⁵R^{d5}, C(O)OR^{a5}, OC(O)R^{b5}, OC(O)NR^{c5}R^{d5}, C(═NR^{e5})NR^{e5}R^{d5}, NR^{e5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R⁴⁵, S(O)₂R^{b5}, and S(O)₂NR^{c5}R^{d5}, wherein said C₁₋₆ alkyl, C₁₋₄ haloalkyl, C₁₋₄ cyanoalkyl, C₂₋₆ alkenyl, C₂₋₆ alkynyl, phenyl, C₃₋₇ cycloalkyl, 5-6 membered heteroaryl, and 4-7 membered heterocycloalkyl are each optionally substituted with 1, 2, 3, or 4 substituents independently selected from CN, NO₂, OR^{a5}, SR^{a5}, C(O)R^{b5}, C(O)NR^{c5}R^{d5}, C(O)OR^{a5}, OC(O)R^{b5}, OC(O)NR⁵R^{d5}, C(═NR^{e5})NR^{c5}R^{d5}, NR^{c5}C(═NR^{e5})NR⁶⁵R^{d5}, NR⁶⁵R^{d5}, NR^{c5}C(O)R^{b5} NR^{c5}C(O)OR^{a5}, NR^{c5}C(O)NR⁵R^{d5}, NR^{c5}S(O)R^{b5}, NR⁶⁵S(O)₂R^{b5}, NR^{c5}S(O)₂NR⁶⁵R^{d5}, S(O)R^{b5} S(O)NR^{c5}R^{d5}, S(O)₂R^{b5}, and S(O)₂NR^{c5}R^{d5},
R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs},
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1} NR^{c1}C(═NR^{e1})NR^{c1}R^{d1}, NR^{c1}R^{d1}, NR^{e1}C(O)R^{b1}, NR^{c1}C(O)OR^{a1}, NR^{e1}C(O)NR^{c1}R^{d1} NR^{e1}S(O)R^{b1}, NR^{c1}S(O)₂R^{b1}, NR^{c1}S(O)₂NR^{c1}R^{d1}, S(O)R^{b1}, S(O)NR^{e1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{e1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{e1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1},
each R^{a1}, R^{b1}, R^{e1}, R^{d1}, R^{a3}, R^{b3}, R^{e3}, R^{d3}, R^{a5}, R^{b5}, R^{e5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1}, R^{e3}, and R^{e5} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl; wherein any cycloalkyl or heterocycloalkyl group is optionally further substituted with 1 or 2 oxo groups.

In some embodiments, the present disclosure is directed to a compound of Formula I, a stereoisomer, or a pharmaceutically acceptable salt thereof, wherein:
a stereoisomer, or a pharmaceutically acceptable salt thereof, wherein:
[symbol] denotes a single bond;
A, B, D, and E are each independently selected from —C(R^{A1})(R^{A2})—and —C(R^{A1})(R^{A2})—C(R^{A1})(R^{A2})—;
V is N or CH;
Y is N or CH;
G is CH₂;
Ring A is cyclohexyl or 6-membered heterocycloalkyl which has one heteroatom ring member being an oxygen;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{x}, wherein R^{x} is absent, H, halo, CN, OH, C₁₋₆ alkyl, C₁₋₆ alkoxy, amino, C₁₋₆ alkyl amino, or C₂₋₈ dialkylamino;
Z is halo, C₁₋₆ alkyl, C₁₋₄ haloalkyl, C₁₋₄ cyanoalkyl, C₂₋₆ alkenyl, C₂₋₆ alkynyl, CN, NO₂, SR^{a3}, C(O)R^{b3}, C(O)NR¹R², C(O)OR^{a3}, OC(O)R^{b3}, OC(O)NR³R^{d3}, C(═NR^{e3})NR^{c3}R^{d3}, NR^{c3}C(═NR^{e3})NR^{c3}R^{d3}, NR^{c3}R^{d3}, NR^{c3}C(O)OR^{a3}, NR^{c3}C(O)NR^{c3}R^{d3}, NR^{c3}S(O)R^{b3}, NR^{c3}S(O)₂NR^{c3}R^{d3}, S(O)R^{b3}, S(O)NR^{c3}R^{d3}, S(O)₂R^{b3}, and P(O)R^{c3}R^{d3}, wherein said C₁₋₆ alkyl, C₁₋₄ haloalkyl, C₁₋₄ cyanoalkyl, C₂₋₆ alkenyl, and C₂₋₆ alkynyl are each optionally substituted with 1, 2, 3, or 4 substituents independently selected from Cy², halo, CN, NO₂, CN, NO₂, OR^{a3}, SR^{a3}, C(O)R^{b3}, C(O)NR³R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3} NR^{c3}S(O)R^{b3}, NR^{c3}S(O)₂R^{b3}, NR^{c3}S(O)₂NR³R^{d3}, S(O)R^{b3}, S(O)NR^{c3}R^{d3}, S(O)₂R^{b3}, and S(O)₂NR^{c3}R^{d3};
each R^{A1} is independently selected from absent, H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A2} is independently selected from H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each Cy² is independently selected from C₆₋₁₄ aryl, C₃₋₁₈ cycloalkyl, 5-16 membered heteroaryl, and 4-18 membered heterocycloalkyl, each of which is optionally substituted with 1, 2, 3, or 4 substituents independently selected from R^{Cy2};
each R^{Cy2} is independently selected from halo, C₁₋₆ alkyl, C₁₋₄ haloalkyl, C₁₋₄ cyanoalkyl, C₂₋₆ alkenyl, C₂₋₆ alkynyl, phenyl, C₃₋₇ cycloalkyl, 5-6 membered heteroaryl, 4-7 membered heterocycloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R⁴⁵, S(O)R^{b5}, S(O)NR⁵R^{d5}, S(O)₂R^{b5}, and S(O)₂NR^{c5}R^{d5}, wherein said C₁₋₆ alkyl, C₁₋₄ haloalkyl, C₁₋₄ cyanoalkyl, C₂₋₆ alkenyl, C₂₋₆ alkynyl, phenyl, C₃₋₇ cycloalkyl, 5-6 membered heteroaryl, and 4-7 membered heterocycloalkyl are each optionally substituted with 1, 2, 3, or 4 substituents independently selected from CN, NO₂, OR^{a5}, SR^{a5}, C(O)R^{b5}, C(O)NR^{c5}R^{d5}, C(O)OR^{a5}, OC(O)R^{b5}, OC(O)NR⁵R^{d5}, C(═NR^{e5})NR^{e5}R^{d5}, NR^{c5}C(═NR^{e5})NR⁵R^{d5}, NR⁶⁵R^{d5}, NR⁵C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es},
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is C₁₋₆ alkyl optionally substituted with 1, 2, 3, 4, or 5 substituents independently selected from R^{g};
R³ is NR^{e1}S(O)₂R^{b1} or NR^{c1}S(O)₂NR^{c1}R^{d1};
each R^{b1}, R^{e1}, R^{d1}, R^{a3}, R^{b3}, R^{c3}, R^{d3}, R^{a5}, R^{b5}, R^{e5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted with 1, 2, 3, 4, or 5 substituents independently selected from R^{g};
each R^{e3} and R^{e5} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, and C₁₋₆ haloalkoxy;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula I, a stereoisomer, or a pharmaceutically acceptable salt thereof, wherein:
[symbol] denotes a single bond;
A, B, D, and E are each independently selected from —C(R^{A1})(R^{A2})—, —C(R^{A1})(R^{A2})—C(R^{A1})(R^{A2})—, —C(R^{A1})(R^{A2})—O—, and —C(R^{A1})(R^{A2})—NR^{A3}_, wherein no more than one of A, B, D, and E is —C(R^{A1})(R^{A2})—O—or —C(R^{A1})(R^{A2})—NR^{A3}—;
V is N or CH;
10 Y is N or CH;
G is CH₂;
Ring A is cyclohexyl or 6-membered heterocycloalkyl which has one heteroatom ring member being an oxygen;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{x}, wherein R^{x} is absent, H, halo, CN, OH, C₁₋₆ alkyl, C₁₋₆ alkoxy, amino, C₁₋₆ alkyl amino, or C₂₋₈ dialkylamino;
Z is halo, C₁₋₆ alkyl, C₁₋₄ haloalkyl, C₁₋₄ cyanoalkyl, C₂₋₆ alkenyl, C₂₋₆ alkynyl, CN, NO₂, SR^{a3}, C(O)R^{b3}, C(O)NR¹R², C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)OR^{a3}, NR^{c3}C(O)NR^{c3}R^{d3}, NR^{c3}S(O)R^{b3}, NR^{c3}S(O)₂NR^{c3}R^{d3}, S(O)R^{b3}, S(O)NR³R^{d3}, S(O)₂R^{b3}, and P(O)R^{c3}R^{d3}, each R^{A1} is independently selected from absent, H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A2} is independently selected from H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A3} is independently selected from H, C₁₋₄ alkyl, C₁₋₄ alkoxy, C₁₋₄ haloalkyl, C(O)R^{z}, and C(O)OR², wherein said C₁₋₆ alkyl is optionally substituted with phenyl, C₁₋₄ alkoxy, C₁₋₄ haloalkoxy, CN, NO₂, or OH;
R^{z} is H, C₁₋₆ alkyl, or phenyl;
   R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs}.
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, and 3-12 membered heterocycloalkyl, are each optionally substituted with 1, 2, 3, 4, or 5 substituents independently selected from R^{g};
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{e1}C(O)OR^{a1}, NR^{c1}C(O)NR^{c1}R^{d1} NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, R^{d1}, R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, are each optionally substituted with 1, 2, 3, 4, or 5 substituents independently selected from R⁸; each R^{e1} and R^{e3} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, and C₁₋₆ haloalkoxy;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula I, a stereoisomer, or a pharmaceutically acceptable salt thereof, wherein:
[symbol] denotes a single or double bond, as valency permits;
A, B, D, and E are each independently selected from —C(R^{A1})(R^{A2})—, —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂)—;
V is N or CH;
Y is N;
G is CH₂;
Ring A is cyclohexyl or 6-membered heterocycloalkyl which has one heteroatom ring member being an oxygen;
W is N;
X is N;
Z is halo, C₁₋₆ alkyl, C₁₋₄ haloalkyl, C₁₋₄ cyanoalkyl, C₂₋₆ alkenyl, C₂₋₆ alkynyl, CN, NO₂, SR^{a3}, C(O)R^{b3}, C(O)NR¹R², C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)OR^{a3}, NR^{c3}C(O)NR^{c3}R^{d3}, NR^{c3}S(O)R^{b3}, NR^{c3}S(O)₂NR^{c3}R^{d3}, S(O)R^{b3}, S(O)NR^{c3}R^{d3}, S(O)₂R^{b3}, and P(O)R^{c3}R^{d3}, wherein said C₁₋₆ alkyl, C₁₋₄ haloalkyl, C₁₋₄ cyanoalkyl, C₂₋₆ alkenyl, and C₂₋₆ alkynyl are each optionally substituted with 1, 2, 3, or 4 substituents independently selected from Cy², halo, CN, NO₂, CN, NO₂, OR^{a3}, SR^{a3}, C(O)R^{b3}, C(O)NR³R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{A1} is independently selected from absent, H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A2} is independently selected from H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A3} is independently selected from H, C₁₋₄ alkyl, C₁₋₄ alkoxy, C₁₋₄ haloalkyl, C(O)R^{z}, and C(O)OR², wherein said C₁₋₆ alkyl is optionally substituted with phenyl, C₁₋₄ alkoxy, C₁₋₄ haloalkoxy, CN, NO₂, or OH;
R^{z} is H, C₁₋₆ alkyl, or phenyl;
each Cy² is independently selected from C₆₋₁₄ aryl, C₃₋₁₈ cycloalkyl, 5-16 membered heteroaryl, and 4-18 membered heterocycloalkyl, each of which is optionally substituted with 1, 2, 3, or 4 substituents independently selected from R^{Cy2};
each R^{Cy2} is independently selected from halo, C₁₋₆ alkyl, C₁₋₄ haloalkyl, C₁₋₄ cyanoalkyl, C₂₋₆ alkenyl, C₂₋₆ alkynyl, phenyl, C₃₋₇ cycloalkyl, 5-6 membered heteroaryl, 4-7 membered heterocycloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{e5}C(═NR^{e5})NR⁵R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR⁵S(O)₂NR^{e5}R^{d5}, S(O)R^{b5}, S(O)NR^{c5}R^{d5}, S(O)₂R^{b5}, and S(O)₂NR^{c5}R^{d5}, wherein said C₁₋₆ alkyl, C₁₋₄ haloalkyl, C₁₋₄ cyanoalkyl, C₂₋₆ alkenyl, C₂₋₆ alkynyl, phenyl, C₃₋₇ cycloalkyl, 5-6 membered heteroaryl, and 4-7 membered heterocycloalkyl are each optionally substituted with 1, 2, 3, or 4 substituents independently selected from CN, NO₂, OR^{a5}, SR^{a5}, C(O)R^{b5}, C(O)NR^{c5}R⁴⁵, C(O)OR^{a5}, OC(O)R^{b5}, OC(O)NR⁶⁵R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{e5}R^{d5}, NR^{c5}S(O)R^{b5}, NR^{c5}S(O)₂R^{b5}, NR⁵S(O)₂NR^{c5}R^{d5}, S(O)R^{b5}, S(O)NR^{c5}R^{d5}, S(O)₂R^{b5}, and S(O)₂NR⁵R^{d5};
R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently C₁₋₆ alkyl; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs},
      each R^{1es} is independently C₁₋₆ alkyl;
R² is C₁₋₆ alkyl;
R³ is NR^{c1}S(O)₂R^{b1} or NR^{e1}S(O)₂NR^{c1}R^{d1}, and
each R^{b1}, R^{e1}, R^{d1}, R^{a3}, R^{b3}, R^{e3}, R^{d3}, R^{a5}, R^{b5}, R^{e5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e3} and R^{e5} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl.

In some embodiments, the present disclosure is directed to a compound of Formula II:

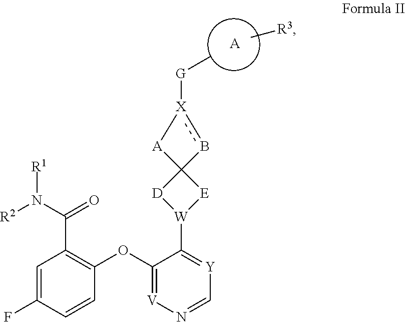

a stereoisomer, or a pharmaceutically acceptable salt thereof, wherein:
[symbol] denotes a single bond or a double bond, as valency permits;
A, B, D, and E are each independently selected from —C(R^{A1})(R^{A2})—, —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂)—;
V is N or CH;
Y is N or CH;
G is O, CH₂, or NH;
Ring A is cyclohexyl or 6-membered heterocycloalkyl which has one heteroatom ring member being an oxygen;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{x}, wherein R^{x} is absent, H, halo, CN, OH, C₁₋₆ alkyl, C₁₋₆ alkoxy, amino, C₁₋₆ alkyl amino, or C₂₋₈ dialkylamino;
each R^{A1} is independently selected from absent, H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A2} is independently selected from H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A3} is independently selected from H, C₁₋₄ alkyl, C₁₋₄ alkoxy, C₁₋₄ haloalkyl, C(O)R^{z}, and C(O)OR², wherein said C₁₋₆ alkyl is optionally substituted with phenyl, C₁₋₄ alkoxy, C₁₋₄ haloalkoxy, CN, NO₂, or OH;
R^{z} is H, C₁₋₆ alkyl, or phenyl;
R¹ is
   (a) H, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R¹s,
   each R^{1s} is independently oxo or ═NR^{4a};
   (c) -C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as},
      each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es},
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1} SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{c1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1} NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{e1}C(O)OR^{a1}, NR^{c1}C(O)NR^{c1}R^{d1}, NR^{c1}S(O)R^{b1}, NR^{e1}S(O)₂R^{b1}, NR^{c1}S(O)₂NR^{c1}R^{d1}, S(O)R^{b1}, S(O)NR¹R^{d1}, S(O)₂R^{b1}, and S(O)₂NR^{c1}R^{d1};
each R^{a1}, R^{b1}, R^{e1}, and R^{d1} is independently selected from H, C₁₋₆ alkyl, C₁₋₆ haloalkyl, C₂-6 alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a} is independently H, CN, or C₁₋₆ alkyl optionally substituted with one or more halo, C₆₋₁₀ aryl, or N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II, a stereoisomer, or a pharmaceutically acceptable salt thereof, wherein:
[symbol] denotes a single bond;
A, B, D, and E are each independently selected from —C(R^{A1})(R^{A2})—, —C(R^{A1})(R^{A2})—C(R^{A1})(R^{A2})—, —C(R^{A1})(R^{A2})—O—, and —C(R^{A1})(R^{A2})—NR^{A3}—, wherein no more than one of A, B, D, and E is-C(R^{A1})(R^{A2})—O—or —C(R^{A1})(R^{A2})—NR^{A3}—;
V is N or CH;
Y is N or CH;
G is CH₂;
Ring A is cyclohexyl or 6-membered heterocycloalkyl which has one heteroatom ring member being an oxygen;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{x}, wherein R^{x} is absent, H, halo, CN, OH, C₁₋₆ alkyl, C₁₋₆ alkoxy, amino, C₁₋₆ alkyl amino, or C₂₋₈ dialkylamino;
each R^{A1} is independently selected from absent, H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A2} is independently selected from H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A3} is independently selected from H, C₁₋₄ alkyl, C₁₋₄ alkoxy, C₁₋₄ haloalkyl, C(O)R^{z}, and C(O)OR², wherein said C₁₋₆ alkyl is optionally substituted with phenyl, C₁₋₄ alkoxy, C₁₋₄ haloalkoxy, CN, NO₂, or OH;
R^{z} is H, C₁₋₆ alkyl, or phenyl;
   R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, and 3-12 membered heterocycloalkyl are each optionally substituted with 1, 2, 3, 4, or 5 substituents independently selected from R^{g};
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{e1}C(O)OR^{a1}, NR^{c1}C(O)NR^{c1}R^{d1}, NR^{c1}S(O)R^{b1}, NR^{e1}S(O)₂R^{b1}, NR^{c1}S(O)₂NR^{c1}R^{d1}, S(O)R^{b1}, S(O)NR^{e1}R^{d1}, S(O)₂R^{b1}, and S(O)₂NR^{c1}R^{d1};
each R^{a1}, R^{b1}, R^{e1}, and R^{d1} is independently selected from H, C₁₋₆ alkyl, C₁₋₆ haloalkyl, C₂-6 alkenyl, C₂₋₆ alkynyl, C₆₋₁₀ aryl, C₃₋₁₀ cycloalkyl, 5-10 membered heteroaryl, and 4-10 membered heterocycloalkyl, wherein said C₁₋₆ alkyl, C₁₋₆ haloalkyl, C₂₋₆ alkenyl, C₂₋₆ alkynyl, C₆-aryl, C₃₋₁₀ cycloalkyl, 5-10 membered heteroaryl, and 4-10 membered heterocycloalkyl are each optionally substituted with 1, 2, 3, 4, or 5 substituents independently selected from R^{g};
each R^{e1} is independently selected from H, C₁₋₆ alkyl, and CN;
each R₅ is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, and C₁₋₆ haloalkoxy;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II, a stereoisomer, or a pharmaceutically acceptable salt thereof, wherein:
[symbol] denotes a single or double bond, as valency permits;
A, B, D, and E are each independently selected from —C(R^{A1})(R^{A2})—, —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)—, wherein no more than one of A, B, D, and E is C(R^{A1})(R^{A2})—O—, —C(R^{A1})(R^{A2})—NR^{A3}—, —C(R^{A1})(R^{A2})—C(═O)—, —C(═O)—, or —N═C(NH₂)—;
V is N or CH;
Y is N;
G is CH₂;
Ring A is cyclohexyl or 6-membered heterocycloalkyl which has one heteroatom ring member being an oxygen;
W is N;
X is N;
each R^{A1} is independently selected from absent, H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A2} is independently selected from H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A3} is independently selected from H, C₁₋₄ alkyl, C₁₋₄ alkoxy, C₁₋₄ haloalkyl, C(O)R^{z}, and C(O)OR², wherein said C₁₋₆ alkyl is optionally substituted with phenyl, C₁₋₄ alkoxy, C₁₋₄ haloalkoxy, CN, NO₂, or OH;
R^{z} is H, C₁₋₆ alkyl, or phenyl;
   R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently C₁₋₆ alkyl; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
      each R les is independently C₁₋₆ alkyl;
R² is C₁₋₆ alkyl;
R³ is NR^{c1}S(O)₂R^{b1} or NR^{e1}S(O)₂NR^{c1}R^{d1}, and
each R^{b1}, R^{e1}, and R^{d1} is independently selected from H or C₁₋₆ alkyl.

In some embodiments, the present disclosure is directed to a compound of Formula III:

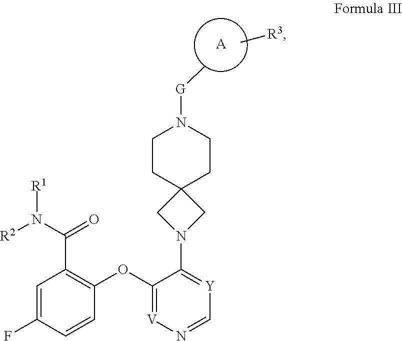

a stereoisomer, or a pharmaceutically acceptable salt thereof, wherein:
V is N or CH;
Y is N or CH;
G is O, CH₂, or NH;
Ring A is cyclohexyl or 6-membered heterocycloalkyl which has one heteroatom ring member being an oxygen;
R¹ is
   (a) -C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as},
      each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
   (b) 3- to 12-membered heterocycloalkyl which has at least one nitrogen and optionally one or more other heteroatom ring members, wherein the 3- to 12-membered heterocycloalkyl is optionally substituted with one or more R^{1bs}.
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es},
      each R^{1es} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R³²′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{e1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{e1}C(O)NR^{c1}R^{d1} NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, and R^{d1} is independently selected from H, C₁₋₆ alkyl, C₁₋₆ haloalkyl, C₂-6 alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R₅ is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula III, a stereoisomer, or a pharmaceutically acceptable salt thereof, wherein:
V is N or CH;
Y is N or CH;
G is CH₂;
Ring A is cyclohexyl or 6-membered heterocycloalkyl which has one heteroatom ring member being an oxygen;
   R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs},
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R³²′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, are each optionally substituted with 1, 2, 3, 4, or 5 substituents independently selected from R^{g};
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1} NR^{c1}C(═NR^{e1})NR^{c1}R^{d1}, NR^{c1}R^{d1}, NR^{c1}C(O)R^{b1}, NR^{c1}C(O)OR^{a1}, NR^{e1}C(O)NR^{c1}R^{d1} NR^{c1}S(O)R^{b1}, NR^{c1}S(O)₂R^{b1}, NR^{e1}S(O)₂NR^{e1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, and R^{d1} is independently selected from H, C₁₋₆ alkyl, C₁₋₆ haloalkyl, C₂-6 alkenyl, C₂₋₆ alkynyl, C₆₋₁₀ aryl, C₃₋₁₀ cycloalkyl, 5-10 membered heteroaryl, 4-10 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃-cycloalkyl, 5-10 membered heteroaryl, and 4-10 membered heterocycloalkyl are each optionally substituted with 1, 2, 3, 4, or 5 substituents independently selected from R⁸;
each R^{e1} and R^{e3} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, and C₁₋₆ haloalkoxy;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula III, a stereoisomer, or a pharmaceutically acceptable salt thereof, wherein:
V is N or CH;
Y is N;
G is O, CH₂, or NH;
Ring A is cyclohexyl or 6-membered heterocycloalkyl which has one heteroatom ring member being an oxygen;
W is N;
X is N;
   R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently C₁₋₆ alkyl; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs},
      each R^{1cs} is independently C₁₋₆ alkyl;
R² is C₁₋₆ alkyl;
R³ is NR^{e1}S(O)₂R^{b1} or NR^{e1}S(O)₂NR^{c1}R^{d1}, and
each R^{b1}, R^{e1}, and R^{d1} is independently selected from H and C₁₋₆ alkyl.

In some embodiments, the present disclosure is directed to a compound of Formula III-a:

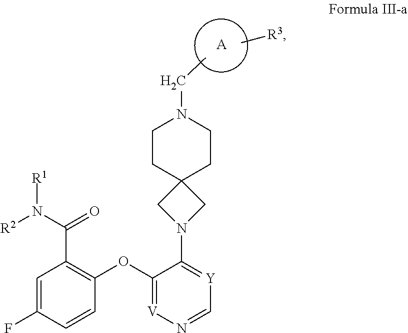

a stereoisomer, or a pharmaceutically acceptable salt thereof, wherein:
V is N or CH;
Y is N or CH;
Ring A is cyclohexyl or 6-membered heterocycloalkyl which has one heteroatom ring member being an oxygen;
R¹ is
   (a) -C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as}
      each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
   (b) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es},
      each R^{1es} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{e1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1} NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, and R^{d1} is independently selected from H, C₁₋₆ alkyl, C₁₋₆ haloalkyl, C₂-6 alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula III-a, a stereoisomer, or a pharmaceutically acceptable salt thereof, wherein:
V is N or CH;
Y is N or CH;
Ring A is cyclohexyl or 6-membered heterocycloalkyl which has one heteroatom ring member being an oxygen;
   R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es}, each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
      each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, are each optionally substituted with 1, 2, 3, 4, or 5 substituents independently selected from R^{g};
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1} SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(—NR^{c1})NR^{c1}R^{d1}, NR^{c1}R^{d1}, NR^{c1}C(O)R^{b1}, NR^{e1}C(O)OR^{a1}, NR^{c1}C(O)NR^{c1}R^{d1} NR^{e1}S(O)R^{b1}, NR^{c1}S(O)₂R^{b1}, NR^{e1}S(O)₂NR^{c1}R^{d1}, S(O)R^{b1}, S(O)NR^{c1}R^{d1}, S(O)₂R^{b1}, and S(O)₂NR^{c1}R^{d1;}
each R^{a1}, R^{b1}, R^{e1}, and R^{d1} is independently selected from H, C₁₋₆ alkyl, C₁₋₆ haloalkyl, C₂-6 alkenyl, C₂₋₆ alkynyl, C₆₋₁₀ aryl, C₃₋₁₀ cycloalkyl, 5-10 membered heteroaryl, 4-10 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃-cycloalkyl, 5-10 membered heteroaryl, and 4-10 membered heterocycloalkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, and C₁₋₆ haloalkoxy; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula III-a, a stereoisomer, or a pharmaceutically acceptable salt thereof, wherein:
V is N or CH;
Y is N;
Ring A is cyclohexyl or 6-membered heterocycloalkyl which has one heteroatom ring member being an oxygen;
   R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs}.
      each R^{1bs} is independently C₁₋₆ alkyl; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs},
      each R^{1es} is independently C₁₋₆ alkyl;
R² is C₁₋₆ alkyl;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{e1}S(O)₂R^{b1}, NR^{e1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR¹R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1;}
each R^{a1}, R^{b1}, R^{e1}, and R^{d1} is independently selected from H, C₁₋₆ alkyl, C₁₋₆ haloalkyl, C₂-6 alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, the present disclosure is directed to a compound of Formula III-a, a stereoisomer, or a pharmaceutically acceptable salt thereof, wherein:
V is N or CH;
Y is N;
Ring A is cyclohexyl or 6-membered heterocycloalkyl which has one heteroatom ring member being an oxygen;
   R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es},
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is C₁₋₆ alkyl;
R³ is NR^{c1}S(O)₂R^{b1} or NR^{c1}S(O)₂NR^{c1}R^{d1}; and
each R^{b1}, R^{e1}, and R^{d1} is independently selected from H or C₁₋₆ alkyl.

In some embodiments, the present disclosure is directed to a compound of Formula III-a, a stereoisomer, or a pharmaceutically acceptable salt thereof, wherein:
V is N or CH;
Y is N;
Ring A is cyclohexyl or 6-membered heterocycloalkyl which has one heteroatom ring member being an oxygen;
   R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently C₁₋₆ alkyl; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es},
      each R^{1es} is independently C₁₋₆ alkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g};
R³ is NR^{e1}S(O)₂R^{b1} or NR^{e1}S(O)₂NR^{c1}R^{d1},
each R^{b1}, R^{e1}, and R^{d1} is independently selected from H or C₁₋₆ alkyl;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, the present disclosure is directed to a compound of Formula III-b:

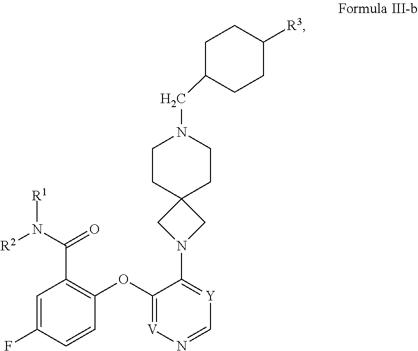

a stereoisomer, or a pharmaceutically acceptable salt thereof, wherein:
V is N or CH;
Y is N or CH;
R¹ is
   (a) -C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as},
      each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
   (b) 3- to 12-membered heterocycloalkyl which has at least one nitrogen and optionally one or more other heteroatom ring members, wherein the 3- to 12-membered heterocycloalkyl is optionally substituted with one or more R^{1bs};
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs},
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{e1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1} NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{e1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1;}
each R^{a1}, R^{b1}, R^{e1}, and R^{d1} is independently selected from H, C₁₋₆ alkyl, C₁₋₆ haloalkyl, C₂-heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula III-b, a stereoisomer, or a pharmaceutically acceptable salt thereof, wherein:
V is N or CH;
Y is N or CH;
   R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs}.
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es},
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl are each optionally substituted with 1, 2, 3, 4, or 5 substituents independently selected from R^{g};
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{c1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1} NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1;}
each R^{a1}, R^{b1}, R^{e1}, and R^{d1} is independently selected from H, C₁₋₆ alkyl, C₁₋₆ haloalkyl, C₂-6 alkenyl, C₂₋₆ alkynyl, C₆₋₁₀ aryl, C₃₋₁₀ cycloalkyl, 5-10 membered heteroaryl, 4-10 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃-cycloalkyl, 5-10 membered heteroaryl, and 4-10 membered heterocycloalkyl are each optionally substituted with 1, 2, 3, 4, or 5 substituents independently selected from R^{g};
each R^{e1} is independently selected from H, C₁₋₆ alkyl, and CN;
each R₅ is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula III-b, a stereoisomer, or a pharmaceutically acceptable salt thereof, wherein:
V is N or CH;
Y is N;
W is N;
X is N;
   R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently C₁₋₆ alkyl; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es},
      each R^{1cs} is independently C₁₋₆ alkyl;
R² is C₁₋₆ alkyl;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{e1}S(O)R^{b1}, NR^{e1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{e1}R^{d1}, NR^{c1}C(O)R^{b1}, NR^{c1}C(O)OR^{a1}, NR^{c1}C(O)NR^{c1}R^{d1} NR^{c1}S(O)R^{b1}, NR^{c1}S(O)₂R^{b1}, NR^{c1}S(O)₂NR^{e1}R^{d1}, S(O)R^{b1}, S(O)NR^{c1}R^{d1}, S(O)₂R^{b1}, and S(O)₂NR^{c1}R^{d1;}
each R^{a1}, R^{b1}, R^{e1}, and R^{d1} is independently selected from H, C₁₋₆ alkyl, C₁₋₆ haloalkyl, C₂-6 alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R₅ is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, the present disclosure is directed to a compound of Formula III-b, a stereoisomer, or a pharmaceutically acceptable salt thereof, wherein:

V is N or CH;
Y is N;
W is N;
X is N;
   R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es}, each R^{1es} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
      each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is C₁₋₆ alkyl;
R³ is NR^{c1}S(O)₂R^{b1} or NR^{e1}S(O)₂NR^{c1}R^{d1}, and each R^{b1}, R^{e1}, and R^{d1} is independently selected from H or C₁₋₆ alkyl.

In some embodiments, the present disclosure is directed to a compound of Formula III-b, a stereoisomer, or a pharmaceutically acceptable salt thereof, wherein:
V is N or CH;
Y is N;
   R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs};
      each R^{1bs} is independently C₁₋₆ alkyl; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es};
      each R^{1cs} is independently C₁₋₆ alkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g};
R³ is NR^{e1}S(O)₂R^{b1} or NR^{e1}S(O)₂NR^{c1}R^{d1;}
each R^{b1}, R^{e1}, and R^{d1} is independently selected from H or C₁₋₆ alkyl;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, the present disclosure is directed to a compound of Formula III-c:

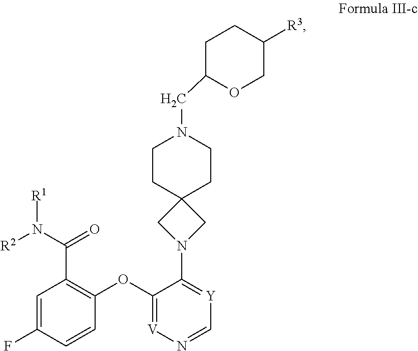

a stereoisomer, or a pharmaceutically acceptable salt thereof, wherein:
V is N or CH;
Y is N or CH;
R¹ is
   (a) -C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as},
      each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
   (b) 3- to 12-membered heterocycloalkyl which has at least one nitrogen and optionally one or more other heteroatom ring members, wherein the 3- to 12-membered heterocycloalkyl is optionally substituted with one or more R^{1bs};
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs},
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{e1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1} NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{e1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1;}
each R^{a1}, R^{b1}, R^{e1}, and R^{d1} is independently selected from H, C₁₋₆ alkyl, C₁₋₆ haloalkyl, C₂-heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula III-c, a stereoisomer, or a pharmaceutically acceptable salt thereof, wherein:
V is N or CH;
Y is N or CH;
   R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs}.
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es},
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, are each optionally substituted with 1, 2, 3, 4, or 5 substituents independently selected from R^{g};
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1} NR^{c1}C(═NR^{c1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1} NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1;}
each R^{a1}, R^{b1}, R^{e1}, and R^{d1} is independently selected from H, C₁₋₆ alkyl, C₁₋₆ haloalkyl, C₂-6 alkenyl, C₂₋₆ alkynyl, C₆₋₁₀ aryl, C₃₋₁₀ cycloalkyl, 5-10 membered heteroaryl, 4-10 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃-cycloalkyl, 5-10 membered heteroaryl, 4-10 membered heterocycloalkyl, are each optionally substituted with 1, 2, 3, 4, or 5 substituents independently selected from R^{g};
each R^{e1} is independently selected from H, C₁₋₆ alkyl, and CN;
each R₅ is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, and C₁₋₆ haloalkoxy; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula III-c, a stereoisomer, or a pharmaceutically acceptable salt thereof, wherein:
V is N or CH;
Y is N;
   R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs};
      each R^{1bs} is independently C₁₋₆ alkyl; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es}.
      each R^{1es} is independently C₁₋₆ alkyl;
R² is C₁₋₆ alkyl;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{e1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1;}
each R^{a1}, R^{b1}, R^{e1}, and R^{d1} is independently selected from H, C₁₋₆ alkyl, C₁₋₆ haloalkyl, C₂-6 alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, the present disclosure is directed to a compound of Formula III-c, a stereoisomer, or a pharmaceutically acceptable salt thereof, wherein:
V is N or CH;
Y is N;
   R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs},
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is C₁₋₆ alkyl;
R³ is NR^{c1}S(O)₂R^{b1} or NR^{e1}S(O)₂NR^{c1}R^{d1}, and
each R^{b1}, R^{e1}, and R^{d1} is independently selected from H or C₁₋₆ alkyl.

In some embodiments, the present disclosure is directed to a compound of Formula III-c, a stereoisomer, or a pharmaceutically acceptable salt thereof, wherein:
V is N or CH;
Y is N;
W is N;
X is N;
   R¹ is
   (a) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently C₁₋₆ alkyl; or
   (b) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
      each R^{1es} is independently C₁₋₆ alkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g};
R³ is NR^{c1}S(O)₂R^{b1} or NR^{c1}S(O)₂NR^{c1}R^{d1},
each R^{b1}, R^{e1}, and Rd1, is independently selected from H or C₁₋₆ alkyl;
each R₅ is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, the present disclosure is directed to a compound of any one of the following Formulae III-b-1, III-b-2, III-b-3, III-c-1, III-c-2, or III-c-3:

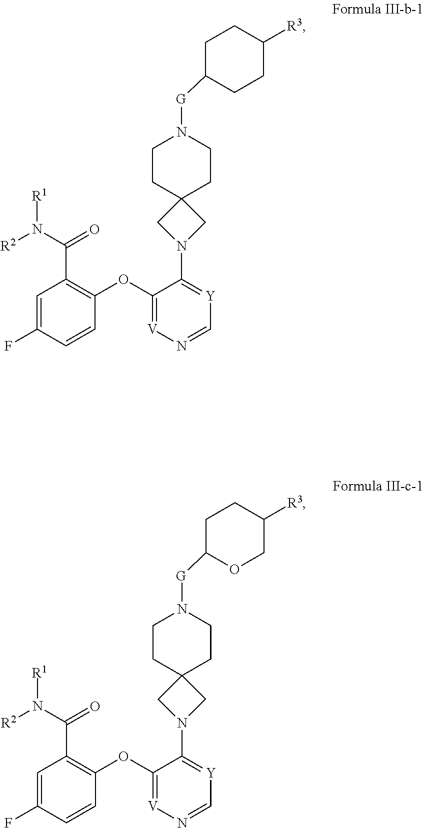

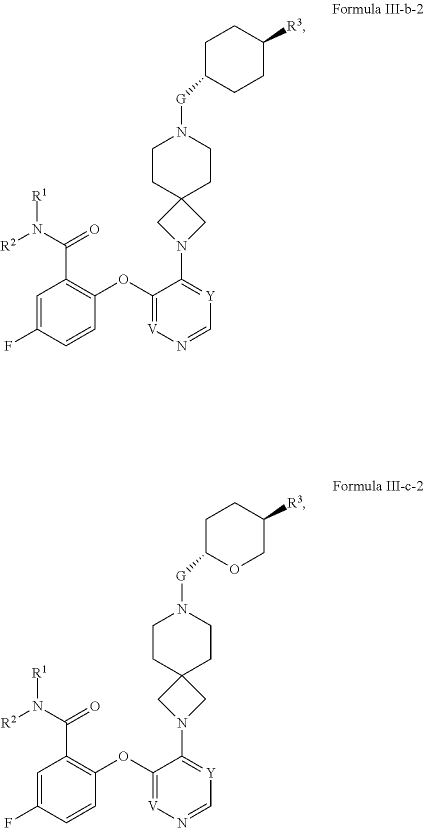

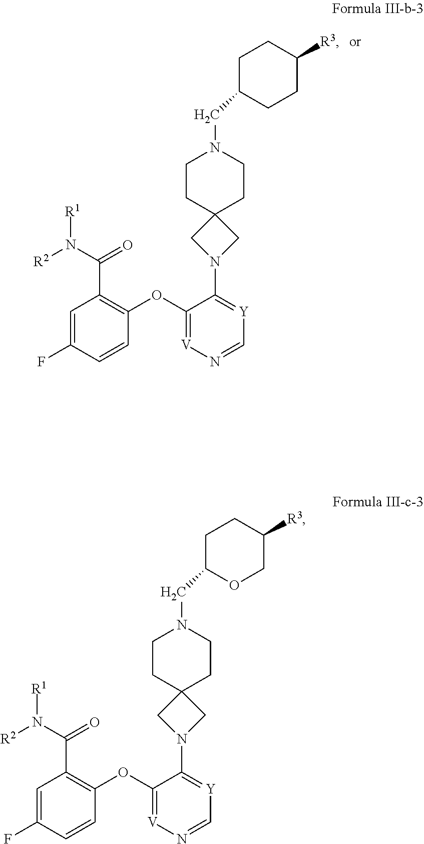

or a pharmaceutically acceptable salt thereof, wherein the variables are as defined herein.

In some embodiments, the present disclosure is directed to a compound of Formula III-b-1, or a pharmaceutically acceptable salt thereof.

In some embodiments, the present disclosure is directed to a compound of Formula III-b-2, or a pharmaceutically acceptable salt thereof.

In some embodiments, the present disclosure is directed to a compound of Formula III-b-3, or a pharmaceutically acceptable salt thereof.

In some embodiments, the present disclosure is directed to a compound of Formula III-c-1, or a pharmaceutically acceptable salt thereof.

In some embodiments, the present disclosure is directed to a compound of Formula III-c-2, or a pharmaceutically acceptable salt thereof.

In some embodiments, the present disclosure is directed to a compound of Formula III-c-3, or a pharmaceutically acceptable salt thereof.

### EMBODIMENTS

For any of Formulae I, II, III, III-a, III-b, III-c, III-b-1, III-b-2, III-b-3, III-c-1, III-c-2, and III-c-3, where applicable, the following embodiments are considered both alone and in conjunction with another where a stable compound is formed.

### Embodiments Covering a, B, D, and E

In some embodiments, A, B, D, and E are each independently selected from —C(R^{A1})(R^{A2})—, —C(R^{A1})(R^{A2})—C(R^{A1})(R⁴²)—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)—, wherein no more than one of A, B, D, and E is C(R^{A1})(R^{A2})—O—, —C(R^{A1})(R^{A2})—NR^{A3}—, —C(R^{A1})(R^{A2})—C(═O)—, —C(═O)—, or —N═C(NH₂)—.

In some embodiments, A, B, D, and E are each independently selected from —C(R^{A1})(R^{A2})—and —C(R^{A1})(R^{A2})—C(R^{A1})(R^{A2})—.

In some embodiments, B is-C(R^{A1})(R^{A2})—.

In some embodiments, B is-C(R^{A1})(R^{A2})—C(R^{A1})(R^{A2})—.

In some embodiments, B is-C(R^{A1})(R^{A2})—, wherein R^{A1} is absent.

In some embodiments, B is-C(R^{A1})(R^{A2})—C(R^{A1})(R^{A2})—, wherein one R^{A1} is absent.

In some embodiments, B is-C(R^{A2})—.

In some embodiments, Bis-C(R^{A2})—C(R^{A1})(R^{A2})—.

### Embodiments Covering R^{a1}

In some embodiments, each R^{A1} is independently selected from absent, H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH.

In some embodiments, each R^{A1} is independently selected from absent, H, and C₁₋₄ alkyl.

In some embodiments, R^{A1} is absent.

In some embodiments, R^{A1} is H.

In some embodiments, R^{A1} is C₁₋₄ alkyl.

### Embodiments Covering R^{a2}

In some embodiments, each R^{A2} is independently selected from H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH.

In some embodiments, each R^{A2} is independently selected from H and C₁₋₄ alkyl.

### Embodiments Covering R^{a3}

In some embodiments, each R^{A3} is independently selected from H, C₁₋₄ alkyl, C₁₋₄ alkoxy, C₁₋₄ haloalkyl, C(O)R^{z}, and C(O)OR², wherein said C₁₋₆ alkyl is optionally substituted with phenyl, C₁₋₄ alkoxy, C₁₋₄ haloalkoxy, CN, NO₂, or OH; and wherein R^{z} is H, C₁₋₆ alkyl, or phenyl.

In some embodiments, each R^{A3} is independently selected from H and C₁₋₄ alkyl.

### Embodiments Covering V

In some embodiments, V is N or CH.

In some embodiments, V is N.

In some embodiments, V is CH.

### Embodiments Covering Y

In some embodiments, Y is N or CH.

In some embodiments, Y is N.

In some embodiments, Y is CH.

### Embodiments Covering G

In some embodiments, G is O, CH₂, or NH.

In some embodiments, G is O.

In some embodiments, G is CH₂.

In some embodiments, G is NH.

### Embodiments Covering Ring a (Substituted with R³)

In some embodiments, Ring A is cyclohexyl or 6-membered heterocycloalkyl which has one heteroatom ring member being an oxygen.

In some embodiments, Ring A is cyclohexyl.

In some embodiments, Ring A is 6-membered heterocycloalkyl which has one heteroatom ring member being an oxygen.

In some embodiments, Ring A is

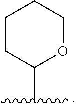 [SMILES: *C1CCCCO1]

### Embodiments Covering W

In some embodiments, W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino.

In some embodiments, W is N.

In some embodiments, W is CH.

Embodiments covering X

In some embodiments, X is N or CR^{X}, wherein R^{X} is absent, H, halo, CN, OH, C₁₋₆ alkyl;

C₁₋₆ alkoxy, amino, C₁₋₆ alkyl amino, or C₂₋₈ dialkylamino.

In some embodiments, X is N.

In some embodiments, X is CR^{X}, wherein R^{X} is absent, H, halo, CN, OH, C₁₋₆ alkyl, C₁₋₆ alkoxy, amino, C₁₋₆ alkyl amino, or C₂₋₈ dialkylamino.

In some embodiments, X is CH.

In some embodiments, X is C.

In some embodiments, when [symbol] is a double bond, X is C.

### Embodiments covering the Spiro Moiety

In some embodiments, the spiro moiety represented by the below formula:

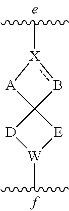

wherein e and f indicate points of attachment to the remainder of the molecule, is selected from:

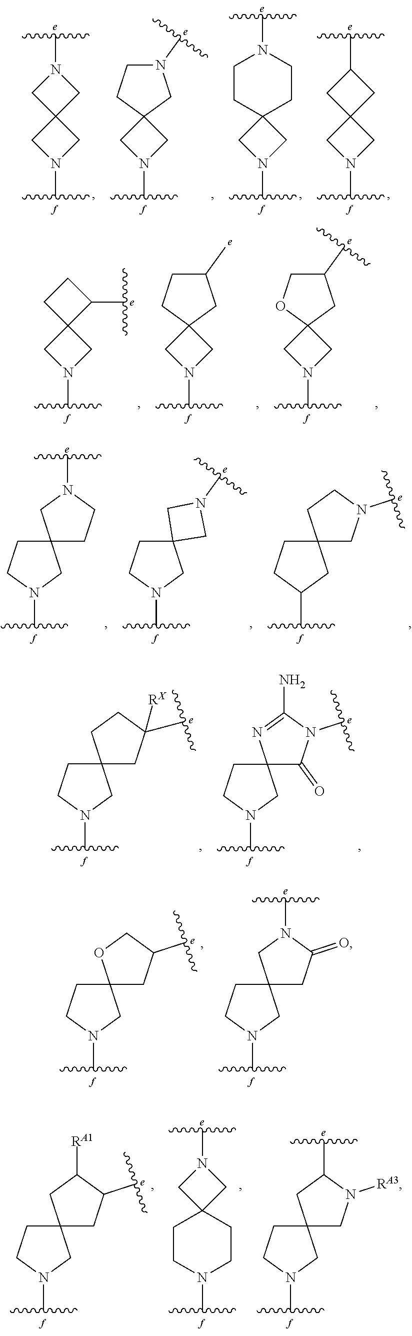

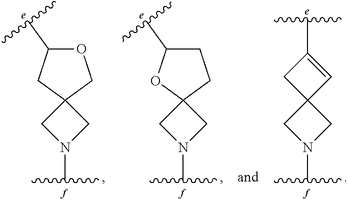

In some embodiments, the spiro moiety represented by the below formula:

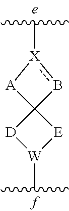

wherein e and f indicate points of attachment to the remainder of the molecule, is,

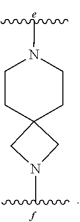 [SMILES: *N1CCC2(CC1)CN(*)C2]

### Embodiments Covering Z

In some embodiments, Z is halo, C₁₋₆ alkyl, C₁₋₄ haloalkyl, C₁₋₄ cyanoalkyl, C₂₋₆ alkenyl, C₂₋₆ alkynyl, CN, NO₂, SR^{a3}, C(O)R^{b3}, C(O)NR¹R², C(O)OR^{a3}, OC(O)R^{b3}, OC(O)NR³R^{d3}, C(═NR^{e3})NR^{c3}R^{d3}, NR^{c3}C(═NR^{c3})NR^{c3}R^{d3}, NR^{c3}R^{d3}, NR^{c3}C(O)OR^{a3}, NR^{c3}C(O)NR^{c3}R^{d3}, NR^{c3}S(O)R^{b3}, NR^{c3}S(O)₂NR^{c3}R^{d3}, S(O)R^{b3}, S(O)NR^{c3}R^{d3}, S(O)₂R^{b3}, and P(O)Rc3R^{d3}, wherein said C₁₋₆ alkyl, C₁₋₄ haloalkyl, C₁₋₄ cyanoalkyl, C₂₋₆ alkenyl, and C₂₋₆ alkynyl are each optionally substituted with 1, 2, 3, or 4 substituents independently selected from Cy2, halo, CN, NO₂, CN, NO2, OR^{a3}, SR^{a3}, C(O)R^{b3}, C(O)NR^{c3}R^{d3}, C(O)OR^{a3}, OC(O)R^{b3}, OC(O)NR⁶³R^{d3}, C(═NR^{c3})NR^{c3}R^{d3}, NR^{c3}C(═NR^{c3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR³R^{d3}, S(O)R^{b3}, S(O)NR^{c3}R^{d3}, S(O)₂R^{b3}, and S(O)₂NR^{c3}R^{d3}, wherein:

R^{a3}, R^{b3}, Rc3, R^{d3}, R^{e3}, R¹, R², and Cy2 are as defined above.

In some embodiments, Z is C(O)NR¹R², wherein R¹ and R² are as defined herein.

In some embodiments, Z is C(O)NR¹R², wherein:
R¹ is H, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R⁸;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸;
each R^{g} is independently selected from OH, NO2, CN, halo, oxo, OBn, N(RN)₂, C₁₋₆ alkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, Z is C(O)NR¹R², wherein:
R¹ is 3- to 12-membered heterocycloalkyl comprising a sulfur atom, wherein the sulfur atom of the 3- to 12-membered heterocycloalkyl is optionally substituted with one or two R^{1s}; each R^{1s} is independently oxo or ═NR^{4a};
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸;
each R^{g} is independently selected from OH, NO2, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a} is independently H, CN, or C₁₋₆ alkyl optionally substituted with one or more halo, C₆₋₁₀ aryl, or N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, Z is C(O)NR¹R², wherein:
R¹ is —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
   each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸;
each R^{g} is independently selected from OH, NO2, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, Z is C(O)NR¹R², wherein:
R¹ is 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸;
each R^{g} is independently selected from OH, NO2, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, Z is C(O)NR¹R², wherein:
R¹ is C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸;
each R^{g} is independently selected from OH, NO2, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, Z is C(O)NR¹R², wherein R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy.

### Embodiments Covering R¹

In some embodiments, R¹ is
(a) H, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
(b)₃- to 12-membered heterocycloalkyl comprising a sulfur atom, wherein the sulfur atom of the 3- to 12-membered heterocycloalkyl is optionally substituted with one or two Rls, each R^{1s} is independently oxo or ═NR^{4a},
(c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as}; each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
(d)₃- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
(e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R³²′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a} is independently H, CN, or C₁₋₆ alkyl optionally substituted with one or more halo, C₆₋₁₀ aryl, or N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, R¹ is H, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R⁸;
each R⁸ is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, R¹ is 3- to 12-membered heterocycloalkyl comprising a sulfur atom, wherein the sulfur atom of the 3- to 12-membered heterocycloalkyl is optionally substituted with one or two R^{1s};
each R^{1s} is independently oxo or ═NR^{4a};
each R^{4a} is independently H, CN, or C₁₋₆ alkyl optionally substituted with one or more halo, C₆₋₁₀ aryl, or N(R^{4a}′)(R^{4b}′); and each R^{4a}′ and R^{4b}′ is independently H, C₁₋₆ alkyl, C₃₋₁₂ cycloalkyl, or 3- to 12-membered heterocycloalkyl.

In some embodiments, R¹ is —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as} ;
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₁₋₆ alkylene-N(R³²′)(R^{3b}′), wherein the C₁₋₆ alkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₁₋₅ alkylene-N(R^{3a}′)(R^{3b}′), wherein the C₁₋₅ alkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₁₋₄ alkylene-N(R^{3a}′)(R^{3b}′), wherein the C₁₋₄ alkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₁₋₃ alkylene-N(R^{3a}′)(R^{3b}′), wherein the C₁₋₃ alkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₁₋₂ alkylene-N(R^{3a}′)(R^{3b}′), wherein the C₁₋₂ alkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3e}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —CH₂—N(R^{3a}′)(R^{3b}′), wherein the —CH₂— is optionally substituted with one or more R^{1as} ;
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —(CH₂)₂—N(R^{3a}′)(R^{3b}′), wherein the —(CH₂)₂— is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —(CH₂)₃—N(R^{3a}′)(R^{3b}′), wherein the —(CH₂)₃— is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —(CH₂)₄—N(R^{3a}′)(R^{3b}′), wherein the —(CH₂)₄— is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —(CH₂)₅—N(R^{3a}′)(R^{3b}′), wherein the —(CH₂)₅— is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —(CH₂)₆—N(R^{3a}′)(R^{3b}′), wherein the —(CH₂)₆— is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₃₋₁₂ cycloalkylene-N(R^{3a}′)(R^{3b}′), wherein the C₃₋₁₂ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3e}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₃₋₁₁ cycloalkylene-N(R^{3a}′)(R^{3b}′), wherein the C₃₋₁₁ cycloalkylene is optionally substituted with one or more R^{1as} ;
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₃₋₁₀ cycloalkylene-N(R^{3a}′)(R^{3b}′), wherein the C₃₋₁₀ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₃₋₉ cycloalkylene-N(R^{3a}′)(R^{3b}′), wherein the C₃₋₉ cycloalkylene is optionally substituted with one or more R^{1as} ;
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R³²′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₃₋₈ cycloalkylene-N(R^{3a}′)(R^{3b}′), wherein the C₃₋₈ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₃₋₇ cycloalkylene-N(R^{3a}′)(R^{3b}′), wherein the C₃₋₇ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₃₋₆ cycloalkylene-N(R^{3a}′)(R^{3b}′), wherein the C₃₋₆ cycloalkylene is optionally substituted with one or more R^{1as} ;
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3e}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₃₋₅ cycloalkylene-N(R^{3a}′)(R^{3b}′), wherein the C₃₋₅ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₃₋₄ cycloalkylene-N(R^{3a}′)(R^{3b}′), wherein the C₃₋₄ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₃ cycloalkylene-N(R^{3a}′)(R^{3b}′), wherein the C₃ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₄ cycloalkylene-N(R^{3a}′)(R^{3b}′), wherein the C₄ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₅ cycloalkylene-N(R^{3a}′)(R^{3b}′), wherein the C₅ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₆ cycloalkylene-N(R^{3a}′)(R^{3b}′), wherein the C₆ cycloalkylene is optionally substituted with one or more R^{1as} ;
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₇ cycloalkylene-N(R^{3a}′)(R^{3b}′), wherein the C₇ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₈ cycloalkylene-N(R^{3a}′)(R^{3b}′), wherein the Ca cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₉ cycloalkylene-N(R^{3a}′)(R^{3b}′), wherein the C₉ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₁₀ cycloalkylene-N(R^{3a}′)(R^{3b}′), wherein the C₁₀ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₁₁ cycloalkylene-N(R^{3a}′)(R^{3b}′), wherein the C₁₁ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R³²′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is —C₁₂ cycloalkylene-N(R^{3a}′)(R^{3b}′), wherein the C₁₂ cycloalkylene is optionally substituted with one or more R^{1as} ;
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R³²′ is independently H, C₁₋₆ alkyl, C₁₋₆ haloalkyl, C(O)R^{3e}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 3- to 12-membered heterocycloalkyl which has at least one nitrogen and optionally one or more other heteroatom ring members, wherein the 3- to 12-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 3- to 11-membered heterocycloalkyl which has at least one nitrogen and optionally one or more other heteroatom ring members, wherein the 3- to 11-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 3- to 10-membered heterocycloalkyl which has at least one nitrogen and optionally one or more other heteroatom ring members, wherein the 3- to 10-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R³²′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 3- to 9-membered heterocycloalkyl which has at least one nitrogen and optionally one or more other heteroatom ring members, wherein the 3- to 9-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R³²′), N(R^{3a}′)(R^{3b}′), or S(═O)₂R^{3ª′;}
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 3- to 8-membered heterocycloalkyl which has at least one nitrogen and optionally one or more other heteroatom ring members, wherein the 3- to 8-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 3- to 7-membered heterocycloalkyl which has at least one nitrogen and optionally one or more other heteroatom ring members, wherein the 3- to 7-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 3- to 6-membered heterocycloalkyl which has at least one nitrogen and optionally one or more other heteroatom ring members, wherein the 3- to 6-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 3- to 5-membered heterocycloalkyl which has at least one nitrogen and optionally one or more other heteroatom ring members, wherein the 3- to 5-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3ª′;}
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 3- to 4-membered heterocycloalkyl which has at least one nitrogen and optionally one or more other heteroatom ring members, wherein the 3- to 4-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3ª′;}
each R^{3a}′ is independently H, C₁₋₆ alkyl, C₁₋₆ haloalkyl, C(O)R^{3e}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 12-membered heterocycloalkyl which has at least one nitrogen and optionally one or more other heteroatom ring members, wherein the 12-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 11-membered heterocycloalkyl which has at least one nitrogen and optionally one or more other heteroatom ring members, wherein the 11-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 10-membered heterocycloalkyl which has at least one nitrogen and optionally one or more other heteroatom ring members, wherein the 10-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R³²′), N(R³²′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 9-membered heterocycloalkyl which has at least one nitrogen and optionally one or more other heteroatom ring members, wherein the 9-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 8-membered heterocycloalkyl which has at least one nitrogen and optionally one or more other heteroatom ring members, wherein the 8-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 7-membered heterocycloalkyl which has at least one nitrogen and optionally one or more other heteroatom ring members, wherein the 7-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 6-membered heterocycloalkyl which has at least one nitrogen and optionally one or more other heteroatom ring members, wherein the 6-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 5-membered heterocycloalkyl which has at least one nitrogen and optionally one or more other heteroatom ring members, wherein the 5-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3ª′;}
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 4-membered heterocycloalkyl which has at least one nitrogen and optionally one or more other heteroatom ring members, wherein the 4-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 3-membered heterocycloalkyl which has at least one nitrogen and optionally one or more other heteroatom ring members, wherein the 3-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₁₁ cycloalkyl substituted with one or more 3- to 12-membered heterocycloalkyl which has at least one nitrogen and optionally one or more other heteroatom ring members; and wherein each of the C₃₋₁₁ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₁₀ cycloalkyl substituted with one or more 3- to 12-membered heterocycloalkyl which has at least one nitrogen and optionally one or more other heteroatom ring members; and wherein each of the C₃₋₁₀ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₉ cycloalkyl substituted with one or more 3- to 12-membered heterocycloalkyl which has at least one nitrogen and optionally one or more other heteroatom ring members; and wherein each of the C₃₋₉ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₈ cycloalkyl substituted with one or more 3- to 12-membered heterocycloalkyl which has at least one nitrogen and optionally one or more other heteroatom ring members; and wherein each of the C₃₋₈ cycloalkyl and the 3- to 12-membered heterocycloalkyl is optionally substituted with one or more R^{1cs};

each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₇ cycloalkyl substituted with one or more 3- to 12-membered heterocycloalkyl which has at least one nitrogen and optionally one or more other heteroatom ring members; and wherein each of the C₃₋₇ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₆ cycloalkyl substituted with one or more 3- to 12-membered heterocycloalkyl which has at least one nitrogen and optionally one or more other heteroatom ring members; and wherein each of the C₃₋₆ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′); N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₅ cycloalkyl substituted with one or more 3- to 12-membered heterocycloalkyl which has at least one nitrogen and optionally one or more other heteroatom ring members; and wherein each of the C₃₋₅ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R³²′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₄ cycloalkyl substituted with one or more 3- to 12-membered heterocycloalkyl which has at least one nitrogen and optionally one or more other heteroatom ring members; and wherein each of the C₃₋₄ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is cyclopropyl substituted with one or more 3- to 12-membered heterocycloalkyl which has at least one nitrogen and optionally one or more other heteroatom ring members; and wherein each of the cyclopropyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R³²′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is cyclobutyl substituted with one or more 3- to 12-membered heterocycloalkyl which has at least one nitrogen and optionally one or more other heteroatom ring members; and wherein each of the cyclobutyl and the 3- to 12-membered heterocycloalkyl is optionally substituted with one or more R^{1cs}, each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is cyclopentyl substituted with one or more 3- to 12-membered heterocycloalkyl which has at least one nitrogen and optionally one or more other heteroatom ring members; and wherein each of the cyclopentyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is cyclohexyl substituted with one or more 3- to 12-membered heterocycloalkyl which has at least one nitrogen and optionally one or more other heteroatom ring members; and wherein each of the cyclohexyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is cycloheptyl substituted with one or more 3- to 12-membered heterocycloalkyl which has at least one nitrogen and optionally one or more other heteroatom ring members; and wherein each of the cycloheptyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is cyclooctyl substituted with one or more 3- to 12-membered heterocycloalkyl which has at least one nitrogen and optionally one or more other heteroatom ring members; and wherein each of the cyclooctyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is cyclononyl substituted with one or more 3- to 12-membered heterocycloalkyl which has at least one nitrogen and optionally one or more other heteroatom ring members; and wherein each of the cyclononyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is cyclodecyl substituted with one or more 3- to 12-membered heterocycloalkyl which has at least one nitrogen and optionally one or more other heteroatom ring members; and wherein each of the cyclodecyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R³²′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is:
a)

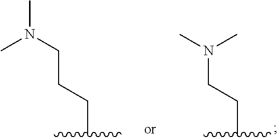

b)

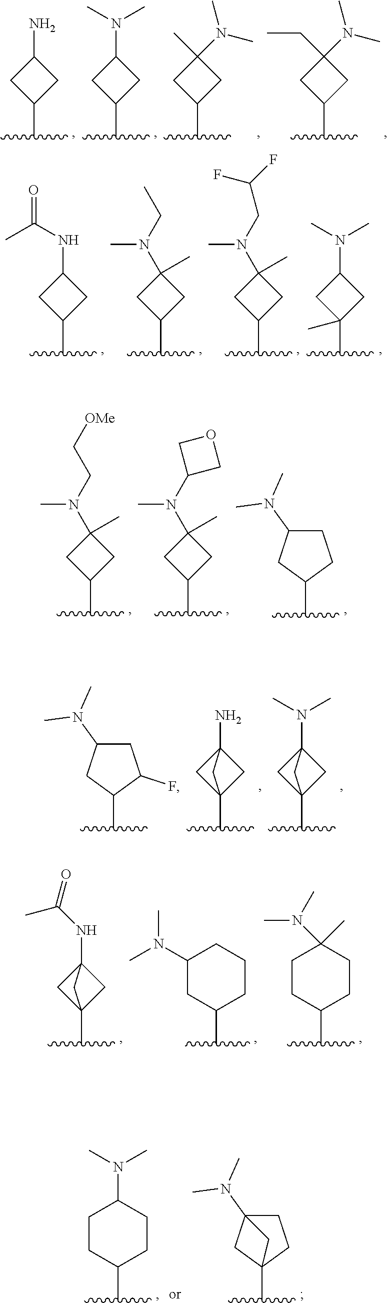

c)

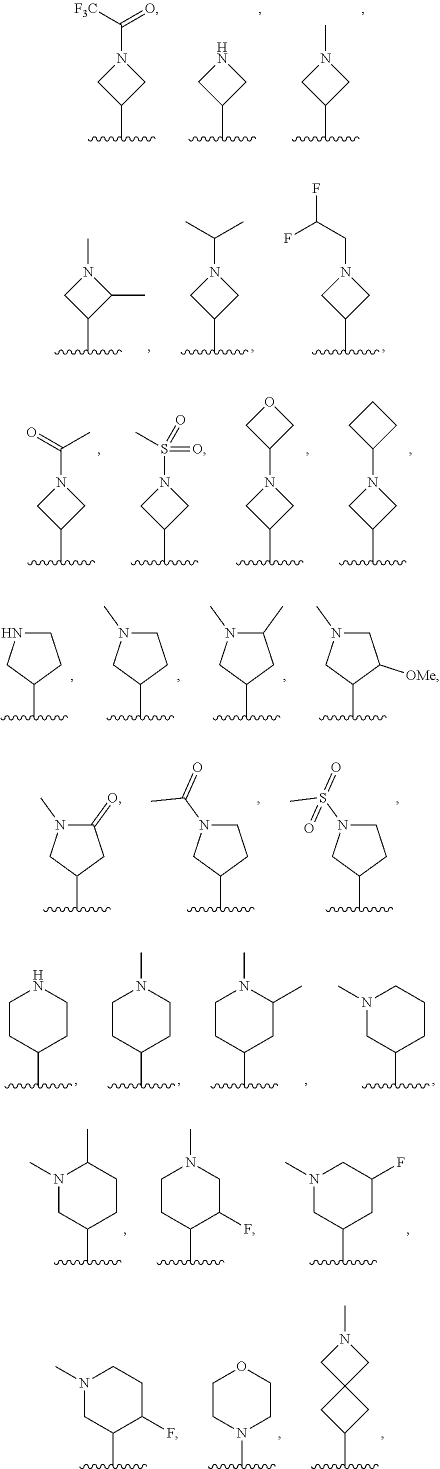

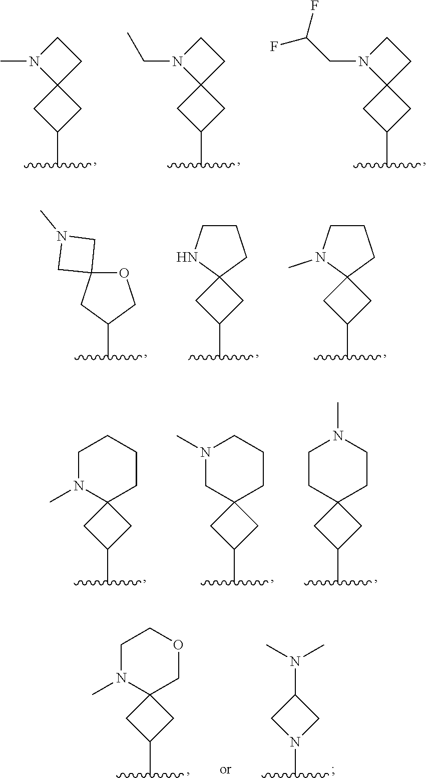

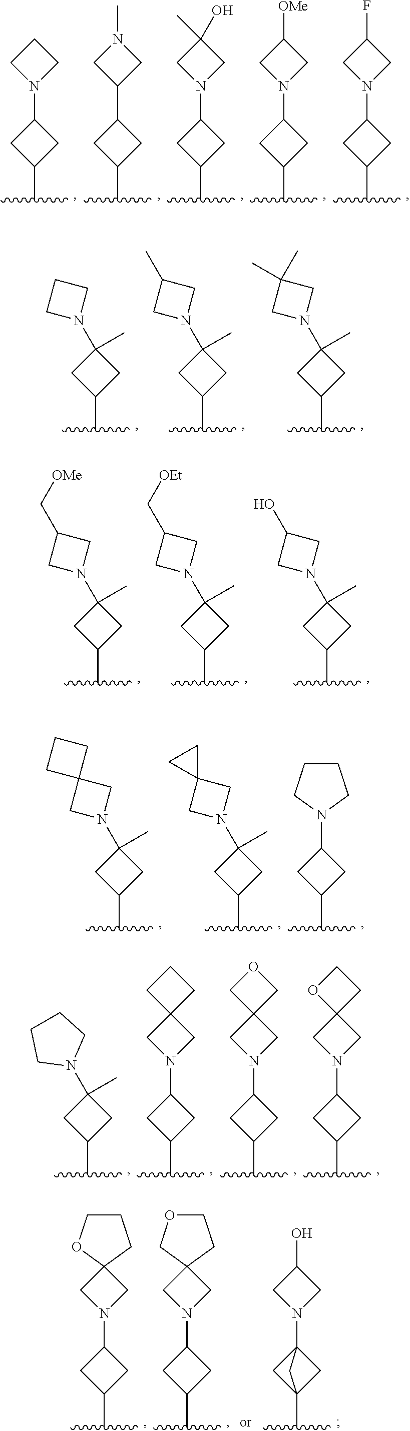

or
d)

In some embodiments, R¹ is

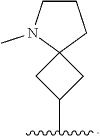 [SMILES: *C1CC2(CCCN2C)C1]

In some embodiments, R¹ is

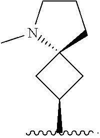 [SMILES: *[C@H]1C[C@@]2(CCCN2C)C1]

In some embodiments, R¹ is

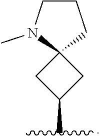 [SMILES: *[C@H]1C[C@]2(CCCN2C)C1]

In some embodiments, R¹ is

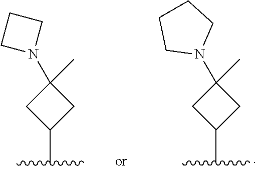

In some embodiments, R¹ is

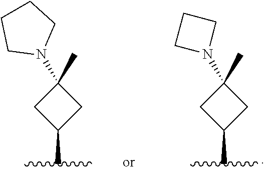

In some embodiments, R¹ is

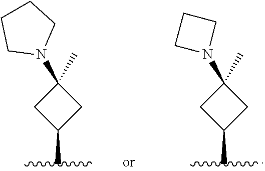

In some embodiments, R¹ is

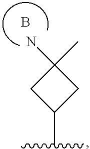

wherein Ring B is a 3- to 12-membered heterocycloalkyl which has at least one nitrogen and optionally one or more other heteroatom ring members, wherein the heterocycloalkyl is optionally substituted.

In some embodiments, R¹ is

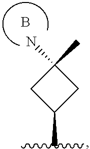

wherein Ring B is a 3- to 12-membered heterocycloalkyl which has at least one nitrogen and optionally one or more other heteroatom ring members, wherein the heterocycloalkyl is optionally substituted.

In some embodiments, R¹ is

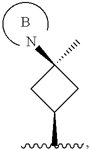

wherein Ring B is a 3- to 12-membered heterocycloalkyl which has at least one nitrogen and optionally one or more other heteroatom ring members, wherein the heterocycloalkyl is optionally substituted.

In some embodiments, R¹ is

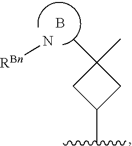

wherein Ring B is a 3- to 12-membered heterocycloalkyl which has at least one nitrogen and optionally one or more other heteroatom ring members, wherein the heterocycloalkyl is optionally substituted, wherein R^{Bn} is an optionally substituted C₁₋₆ alkyl.

In some embodiments, R¹ is

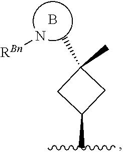

wherein Ring B is a 3- to 12-membered heterocycloalkyl which has at least one nitrogen and optionally one or more other heteroatom ring members, wherein the heterocycloalkyl is optionally substituted, wherein R^{Bn} is an optionally substituted C₁₋₆ alkyl.

In some embodiments, R¹ is

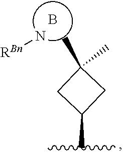

wherein Ring B is a 3- to 12-membered heterocycloalkyl which has at least one nitrogen and optionally one or more other heteroatom ring members, wherein the heterocycloalkyl is optionally substituted, wherein R^{Bn} is an optionally substituted C₁₋₆ alkyl.

In some embodiments, R^{Bn} is an optionally substituted C₁ alkyl.

In some embodiments, R^{Bn} is an optionally substituted C₂ alkyl.

In some embodiments, R^{Bn} is an optionally substituted C₃ alkyl.

In some embodiments, R^{Bn} is a C₁ alkyl.

In some embodiments, R^{Bn} is a C₂ alkyl.

In some embodiments, R^{Bn} is a C₃ alkyl.

### Embodiments Covering R²

In some embodiments, R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R⁸;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, R² is C₁₋₆ alkyl optionally substituted with 1, 2, 3, 4, or 5 substituents independently selected from R⁸, wherein:
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, R² is H.

In some embodiments, R² is C₁₋₆ alkyl.

In some embodiments, R² is C₁₋₅ alkyl.

In some embodiments, R² is C₁₋₄ alkyl.

In some embodiments, R² is C₁₋₃ alkyl.

In some embodiments, R² is C₁₋₂ alkyl.

In some embodiments, R² is methyl.

In some embodiments, R² is ethyl.

In some embodiments, R² is propyl.

In some embodiments, R² is isopropyl.

In some embodiments, R² is butyl.

In some embodiments, R² is tert-butyl.

In some embodiments, R² is pentyl.

In some embodiments, R² is hexyl.

### Embodiments Covering R¹ and R²

In some embodiments, R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy.

### Embodiments Covering R³

In some embodiments, R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{e1}C(═NR^{c1})NR^{c1}R^{d1}, NR^{c1}R^{d1}, NR^{e1}C(O)R^{b1}, NR^{c1}C(O)OR^{a1}, NR^{c1}C(O)NR^{c1}R^{d1}, NR^{c1}S(O)R^{b1}, NR^{c1}S(0)₂R^{b1}, NR^{c1}S(0)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆-aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{e1}C(O)OR^{a1}, NR^{c1}C(O)NR^{c1}R^{d1} NR^{c1}S(O)R^{b1}, NR^{e1}S(O)₂R^{b1}, NR^{e1}S(O)₂NR^{e1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R⁸;
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, R³ is halo.

In some embodiments, R³ is F.

In some embodiments, R³ is C₁.

In some embodiments, R³ is I.

In some embodiments, R³ is Br.

In some embodiments, R³ is C₁₋₆ alkyl.

In some embodiments, R³ is C₁₋₅ alkyl.

In some embodiments, R³ is C₁₋₄ alkyl.

In some embodiments, R³ is C₁₋₃ alkyl.

In some embodiments, R³ is C₁₋₂ alkyl.

In some embodiments, R³ is methyl.

In some embodiments, R³ is ethyl.

In some embodiments, R³ is propyl.

In some embodiments, R³ is isopropyl.

In some embodiments, R³ is butyl.

In some embodiments, R³ is tert-butyl.

In some embodiments, R³ is C₁₋₄ haloalkyl.

In some embodiments, R³ is C₁₋₄ cyanoalkyl.

In some embodiments, R³ is C₂₋₆ alkenyl.

In some embodiments, R³ is C₂₋₆ alkynyl.

In some embodiments, R³ is C₆₋₁₀ aryl.

In some embodiments, R³ is C₃₋₁₀ cycloalkyl.

In some embodiments, R³ is 5-10 membered heteroaryl.

In some embodiments, R³ is 4-10 membered heterocycloalkyl.

In some embodiments, R³ is CN.

In some embodiments, R³ is NO₂.

In some embodiments, R³ is OR^{a1}, wherein R^{a1} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl.

In some embodiments, R³ is SR^{a1}, wherein R^{a1} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl.

In some embodiments, R³ is C(O)R^{b1}, wherein R^{b1} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl.

In some embodiments, R³ is C(O)NR^{c1}R^{d1}, wherein each R^{e1} and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl.

In some embodiments, R³ is C(O)OR^{a1}, wherein R^{a1} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl.

In some embodiments, R³ is OC(O)R^{b1}, wherein R^{b1} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl.

In some embodiments, R³ is OC(O)NR^{c1}R^{d1}, wherein each R^{e1} and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl.

In some embodiments, R³ is C(═NR^{e1})NR^{c1}R^{d1}, wherein each R^{e1} and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, and wherein R^{e1} is selected from H, C₁₋₆ alkyl, and CN.

In some embodiments, R³ is NR^{c1}C(═NR^{e1})NR^{c1}R^{d1}, wherein each R^{e1} and R^{d1} is independently selected from H, C₁₋₆ alkyl, C₁₋₆ haloalkyl, C₂₋₆ alkenyl, C₂₋₆ alkynyl, C₆₋₁₀ aryl, C₃-10 cycloalkyl, 5-10 membered heteroaryl, 4-10 membered heterocycloalkyl, -C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, and wherein R^{e1} is selected from H, C₁₋₆ alkyl, and CN.

In some embodiments, R³ is NR^{c1}R^{d1}, wherein each R^{e1} and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl.

In some embodiments, R³ is NR^{c1}C(O)R^{b1}, wherein each R^{b1} and R^{e1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl.

In some embodiments, R³ is NR^{e1}C(O)OR^{a1}, wherein each R^{a1} and R^{e1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl.

In some embodiments, R³ is NR^{c1}C(O)NR^{c1}R^{d1}, wherein each R^{e1} and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl.

In some embodiments, R³ is NR^{c1}S(O)R^{b1}, wherein each Rb1 and R^{e1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl.

In some embodiments, R³ is NR^{c1}S(O)₂R^{b1}, wherein each R^{b1} and R^{e1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl,—C₆₋₁₀ aryl-C₁₋₆ alkyl,—C₃₋₁₀ cycloalkyl-C₁₋₆ alkyl,-(5-10 membered heteroaryl)-C₁₋₆ alkyl, and -(4-10 membered heterocycloalkyl)-C₁₋₆ alkyl.

In some embodiments, R³ is NR^{c1}S(O)₂R^{b1}, wherein each Rb1 and R^{e1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl.

In some embodiments, R³ is NR^{e1}s (O)₂R^{b1}, and each Rb1 and R^{e1} is independently selected from H and C₁₋₆ alkyl.

In some embodiments, R³ is NHS(O) (C₂H₅).

In some embodiments, R³ is

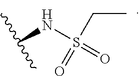

In some embodiments, R³ is NR^{c1}S(O)₂NR^{c1}R^{d1}, wherein each R^{e1} and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl,—C₆₋₁₀ aryl-C₁₋₆ alkyl,—C₃₋₁₀ cycloalkyl-C₁₋₆ alkyl,-(5-10 membered heteroaryl)-C₁₋₆ alkyl, and -(4-10 membered heterocycloalkyl)-C₁₋₆ alkyl.

In some embodiments, R³ is NR^{c1}S(O)₂NR^{e1}R^{d1}, and wherein each R^{e1} and R^{d1} is independently selected from H, C₁₋₆ alkyl, C₁₋₆ haloalkyl, C₂₋₆ alkenyl, C₂₋₆ alkynyl, C₆₋₁₀ aryl, C₃-10 cycloalkyl, 5-10 membered heteroaryl, and 4-10 membered heterocycloalkyl.

In some embodiments, R³ is NR^{c1}S(O)₂NR^{c1}R^{d1}, and each R^{e1} and R^{d1} is independently selected from H and C₁₋₆ alkyl.

In some embodiments, R³ is NHS(O)₂NH(C₂H₅).

In some embodiments, R³ is

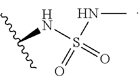

In some embodiments, R³ is S(O)R^{b1}, wherein Rb1 is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl,—C₆₋₁₀ aryl-C₁₋₆ alkyl,—C₃₋₁₀ cycloalkyl-C₁₋₆ alkyl,-(5-10 membered heteroaryl)-C₁₋₆ alkyl, and -(4-10 membered heterocycloalkyl)-C₁₋₆ alkyl.

In some embodiments, R³ is S(O)NR^{c1}R^{d1}, wherein each R^{e1} and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl,—C₆₋₁₀ aryl-C₁₋₆ alkyl,—C₃₋₁₀ cycloalkyl-C₁₋₆ alkyl,-(5-10 membered heteroaryl)-C₁₋₆ alkyl, and -(4-10 membered heterocycloalkyl)-C₁₋₆ alkyl.

In some embodiments, R³ is S(O)₂R^{b1}, wherein Rb1 is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl,—C₆₋₁₀ aryl-C₁₋₆ alkyl,—C₃₋₁₀ cycloalkyl-C₁₋₆ alkyl,-(5-10 membered heteroaryl)-C₁₋₆ alkyl, and -(4-10 membered heterocycloalkyl)-C₁₋₆ alkyl.

In some embodiments, R³ is S(O)₂NR^{c1}R^{d1}, wherein each R^{e1} and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl,—C₆₋₁₀ aryl-C₁₋₆ alkyl,—C₃₋₁₀ cycloalkyl-C₁₋₆ alkyl,-(5-10 membered heteroaryl)-C₁₋₆ alkyl, and -(4-10 membered heterocycloalkyl)-C₁₋₆ alkyl.

In some embodiments, R³ is propyl optionally substituted with one ORal. In some embodiments, R^{a1} is methyl.

In some embodiments, R³ is isopropyl optionally substituted with one ORal. In some embodiments, R^{a1} is methyl.

In some embodiments, R³ is butyl optionally substituted with one ORal. In some embodiments, R^{a1} is methyl.

In some embodiments, R³ is tert-butyl optionally substituted with one ORal. In some embodiments, R^{a1} is methyl.

In some embodiments, R³ is pentyl optionally substituted with one ORal. In some embodiments, R^{a1} is methyl.

In some embodiments, R³ is hexyl optionally substituted with one ORal. In some embodiments, R^{a1} is methyl.

In some embodiments, R³ is C₂₋₆ alkenyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{e1}S(O)R^{b1}, NR^{c1}S(O)₂R^{b1} NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, R³ is C₂₋₅ alkenyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1} NR^{c1}S(O)₂NR^{c1}R^{d1}, S(O)R^{b1}, S(O)NR^{c1}R^{d1}, S(O)₂R^{b1}, and S(O)₂NR^{c1}Rd1

In some embodiments, R³ is C₂₋₄ alkenyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1} NR^{c1}S(O)₂NR^{c1}R^{d1}, S(O)R^{b1}, S(O)NR^{c1}R^{d1}, S(O)₂R^{b1}, and S(O)₂NR^{c1}R^{d1}

In some embodiments, R³ is C₂₋₃ alkenyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{e1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}Rd1

In some embodiments, R³ is C₂₋₆ alkynyl optionally substituted with 1, 2, 3, or 4 substituents independently selected from CN, NO₂, OR^{a1}, SR^{a1}, C(O)R^{b1}, C(O)NR^{c1}R^{d1} C(O)OR^{a1}, OC(O)R^{b1}, OC(O)NR^{c1}R^{d1}, C(═NR^{e1})NR^{c1}R^{d1}, NR^{e1}C(═NR^{e1})NR^{c1}R^{d1}, NR^{c1}R^{d1} NR^{c1}C(O)R^{b1}, NR^{c1}C(O)OR^{a1}, NR^{c1}C(O)NR^{c1}R^{d1}, NR^{c1}S(O)R^{b1} NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}Rd1

In some embodiments, R³ is C₂₋₅ alkynyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{e1}R^{d1}, NR^{c1}C(═NR^{e1})NR^{c1}R^{d1}, NR^{c1}Rd1 NR^{c1}C(O)R^{b1}, NR^{c1}C(O)OR^{a1}, NR^{c1}C(O)NR^{c1}R^{d1}, NR^{e1}S(O)R^{b1}, NR^{e1}S(O)₂R^{b1}, NR^{c1}S(0)₂NR^{c1}R^{d1}, S(O)R^{b1}, S(O)NR^{c1}R^{d1}, S(O)₂R^{b1}, and S(O)₂NR^{c1}Rd1

In some embodiments, R³ is C₂₋₄ alkynyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{e1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}Rd1

In some embodiments, R³ is C₂₋₃ alkynyl optionally substituted with 1, 2, 3, or 4 substituents independently selected from CN, NO₂, OR^{a1}, SR^{a1}, C(O)R^{b1}, C(O)NR^{c1}R^{d1}; C(O)OR^{a1}, OC(O)R^{b1}, OC(O)NR^{c1}R^{d1}, C(═NR^{e1})NR^{c1}R^{d1}, NR^{e1}C(═NR^{e1})NR^{e1}R^{d1}, NR^{e1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1} NR^{c1}S(O)₂NR^{c1}R^{d1}, S(O)R^{b1}, S(O)NR^{c1}R^{d1}, S(O)₂R^{b1}, and S(O)₂NR^{c1}R^{d1}

In some embodiments, R³ is C₁₋₆ alkoxy optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{e1}C(O)R^{b1}, NR^{c1}C(O)OR^{a1}, NR^{c1}C(O)NR^{c1}R^{d1}, NR^{c1}S(O)R^{b1}, NR^{c1}S(0)₂R^{b1}, NR^{c1}S(0)₂NR^{c1}R^{d1}, S(O)R^{b1}, S(O)NR^{c1}R^{d1}, S(O)₂R^{b1}, and S(O)₂NR^{c1}R^{d1}

In some embodiments, R³ is C₁₋₅ alkoxy optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{e1}R^{d1}, NR^{c1}S(O)R^{b1}, NR^{e1}S(0)₂R^{b1}, NR^{e1}S(0)₂NR^{c1}R^{d1}, S(O)R^{b1}, S(O)NR^{c1}R^{d1}, S(O)₂R^{b1}, and S(O)₂NR^{c1}R^{d1}

In some embodiments, R³ is C₁₋₄ alkoxy optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{e1}S(O)R^{b1}, NR^{e1}S(0)₂R^{b1}, NR^{c1}S(0)₂NR^{c1}R^{d1}, S(O)R^{b1}, S(O)NR^{c1}R^{d1}, S(O)₂R^{b1}, and S(O)₂NR^{c1}Rd1

In some embodiments, R³ is C₁₋₃ alkoxy optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{e1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}Rd1

In some embodiments, R³ is C₁₋₂ alkoxy optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{e1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{e1}S(O)₂R^{b1}, NR^{e1}S(O)₂NR^{c1}R^{d1}, S(O)R^{b1} S(O)NR^{c1}R^{d1}, S(O)₂R^{b1}, and S(O)₂NR^{c1}Rd1

In some embodiments, R³ is a C₃₋₁₀ cycloalkyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1} NR^{c1}S(O)₂NR^{c1}R^{d1}, S(O)R^{b1}, S(O)NR^{c1}R^{d1}, S(O)₂R^{b1}, and S(O)₂NR^{c1}Rd1

In some embodiments, R³ is a C₃₋₉ cycloalkyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{e1}R^{d1}, NR^{e1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(0)₂R^{b1} NR^{c1}S(O)₂NR^{c1}R^{d1}, S(O)R^{b1}, S(O)NR^{c1}R^{d1}, S(O)₂R^{b1}, and S(O)₂NR^{c1}Rd1

In some embodiments, R³ is a C₃₋₈ cycloalkyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}

In some embodiments, R³ is a C₃₋₇ cycloalkyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{e1}R^{d1}, NR^{c1}C(═NR^{e1})NR^{c1}R^{d1}, NR^{c1}R^{d1}, NR^{c1}C(O)R^{b1}, NR^{e1}C(O)OR^{a1}, NR^{c1}C(O)NR^{c1}R^{d1}, NR^{c1}S(O)R^{b1} NR^{c1}S(0)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}Rd1

In some embodiments, R³ is a C₃₋₆ cycloalkyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{e1}R^{d1}, NR^{c1}C(═NR^{e1})NR^{c1}R^{d1}, NR^{e1}R^{d1} NR^{c1}C(O)R^{b1}, NR^{c1}C(O)OR^{a1}, NR^{c1}C(O)NR^{c1}R^{d1}, NR^{c1}S(O)R^{b1} NR^{c1}S(O)₂R^{b1}, NR^{c1}S(0)₂NR^{c1}R^{d1}, S(O)R^{b1}, S(O)NR^{c1}R^{d1}, S(O)₂R^{b1}, and S(O)₂NR^{c1}R^{d1}

In some embodiments, R³ is a C₃₋₅ cycloalkyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{e1}R^{d1}, NR^{c1}C(═NR^{e1})NR^{c1}R^{d1}, NR^{e1}R^{d1}, NR^{c1}C(O)R^{b1}, NR^{c1}C(O)OR^{a1}, NR^{e1}C(O)NR^{c1}R^{d1}, NR^{c1}S(O)R^{b1}, NR^{c1}S(0)₂R^{b1} NR^{c1}S(O)₂NR^{c1}R^{d1}, S(O)R^{b1}, S(O)NR^{c1}R^{d1}, S(O)₂R^{b1}, and S(O)₂NR^{c1}Rd1

In some embodiments, R³ is a C₃₋₄ cycloalkyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1} NR^{c1}S(O)₂NR^{c1}R^{d1}, S(O)R^{b1}, S(O)NR^{c1}R^{d1}, S(O)₂R^{b1}, and S(O)₂NR^{c1}R^{d1}

In some embodiments, R³ is a C₆₋₁₀ aryl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(0)₂NR^{c1}R^{d1}, S(O)R^{b1}, S(O)NR^{c1}R^{d1}, S(O)₂R^{b1}, and S(O)₂NR^{c1}R^{d1}

In some embodiments, R³ is a C₆₋₉ aryl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{e1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}Rd1

In some embodiments, R³ is a C₆₋₈ aryl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{e1}S(O)R^{b1}, NR^{e1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}Rd1

In some embodiments, R³ is a C₆₋₇ aryl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{e1}C(═NR^{e1})NR^{c1}R^{d1}, NR^{e1}R^{d1}, NR^{e1}C(O)R^{b1}, NR^{c1}C(O)OR^{a1}, NR^{c1}C(O)NR^{c1}R^{d1}, NR^{e1}S(O)R^{b1}, NR^{c1}S(0)₂R^{b1}, NR^{c1}S(O)₂NR^{c1}R^{d1}, S(O)R^{b1} S(O)NR^{c1}R^{d1}, S(O)₂R^{b1}, and S(O)₂NR^{c1}Rd1

In some embodiments, R³ is a 5-10 membered heteroaryl optionally substituted with 1, 2, 3, or 4 substituents independently selected from CN, NO₂, OR^{a1}, SR^{a1}, C(O)R^{b1}, C(O)NR^{c1}Rd1;
C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1} NR^{c1}S(O)₂NR^{c1}R^{d1}, S(O)R^{b1}, S(O)NR^{c1}R^{d1}, S(O)₂R^{b1}, and S(O)₂NR^{c1}Rd1

In some embodiments, R³ is a 5-9 membered heteroaryl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{e1}R^{d1}, NR^{e1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1} NR^{c1}S(O)₂NR^{c1}R^{d1}, S(O)R^{b1}, S(O)NR^{c1}R^{d1}, S(O)₂R^{b1}, and S(O)₂NR^{c1}Rd1

In some embodiments, R³ is a 5-8 membered heteroaryl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{e1}R^{d1}, NR^{e1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(0)₂R^{b1} NR^{c1}S(O)₂NR^{c1}R^{d1}, S(O)R^{b1}, S(O)NR^{c1}R^{d1}, S(O)₂R^{b1}, and S(O)₂NR^{c1}R^{d1}

In some embodiments, R³ is a 5-7 membered heteroaryl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═aNR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}Rd1

In some embodiments, R³ is a 5-6 membered heteroaryl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{e1}C(═NR^{e1})NR^{e1}R^{d1}, NR^{c1}Rd1 NR^{c1}C(O)R^{b1}, NR^{c1}C(O)OR^{a1}, NR^{c1}C(O)NR^{c1}R^{d1}, NR^{c1}S(0)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}Rd1

In some embodiments, R³ is a 4-10 membered heterocycloalkyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{e1}S(O)R^{b1}, NR^{c1}S(O)₂R^{b1}, NR^{c1}S(0)₂NR^{c1}R^{d1}, S(O)R^{b1}, S(O)NR^{c1}R^{d1}, S(O)₂R^{b1}, and S(O)₂NR^{c1}Rd1

In some embodiments, R³ is a 4-9 membered heterocycloalkyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1} NR^{c1}R^{d1}, NR^{c1}C(O)R^{b1}, NR^{c1}C(O)OR^{a1}, NR^{c1}C(O)NR^{c1}R^{d1}, NR^{e1}s (O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}Rd1

In some embodiments, R³ is a 4-8 membered heterocycloalkyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{e1}C(O)R^{b1}, NR^{c1}C(O)OR^{a1}, NR^{c1}C(O)NR^{e1}R^{d1}, NR^{e1}S(O)R^{b1}, NR^{c1}S(O)₂R^{b1} NR^{c1}S(O)₂NR^{c1}R^{d1}, S(O)R^{b1}, S(O)NR^{c1}R^{d1}, S(O)₂R^{b1}, and S(O)₂NR^{c1}R^{d1}

In some embodiments, R³ is a 4-7 membered heterocycloalkyl optionally substituted with 1, 2, 3, or 4 substituents independently selected from CN, NO₂, OR^{a1}, SR^{a1}, C(O)R^{b1}, C(O)NR^{e1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1} NR^{c1}R^{d1}, NR^{c1}C(O)R^{b1}, NR^{c1}C(O)OR^{a1}, NR^{c1}C(O)NR^{c1}R^{d1}, NR^{e1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}

In some embodiments, R³ is a 4-6 membered heterocycloalkyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1} NR^{c1}R^{d1}, NR^{c1}C(O)R^{b1}, NR^{e1}C(O)OR^{a1}, NR^{e1}C(O)NR^{c1}R^{d1}, NR^{e1}S(O)R^{b1}, NR^{e1}S(O)₂R^{b1} NR^{c1}S(O)₂NR^{c1}R^{d1}, S(O)R^{b1}, S(O)NR^{c1}R^{d1}, S(O)₂R^{b1}, and S(O)₂NR^{c1}Rd1

In some embodiments, R³ is a 4-5 membered heterocycloalkyl optionally substituted with 1, 2, 3, or 4 substituents independently selected from CN, NO₂, OR^{a1}, SR^{a1}, C(O)R^{b1}, C(O)NR^{c1}R^{d1}, C(O)OR^{a1}, QC(O)R^{b1}, OC(O)NR^{c1}R^{d1}, C(═NR^{e1})NR^{c1}R^{d1}, NR^{c1}C(═NR^{e1})NR^{c1}R^{d1} NR^{c1}R^{d1}, NR^{c1}C(O)R^{b1}, NR^{c1}C(O)OR^{a1}, NR^{c1}C(O)NR^{c1}R^{d1}, NR^{e1}s (O)R^{b1}, NR^{e1}S(0)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}

In some embodiments, R³ is

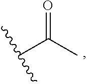 [SMILES: *C(C)=O]

In some embodiments, R³ is

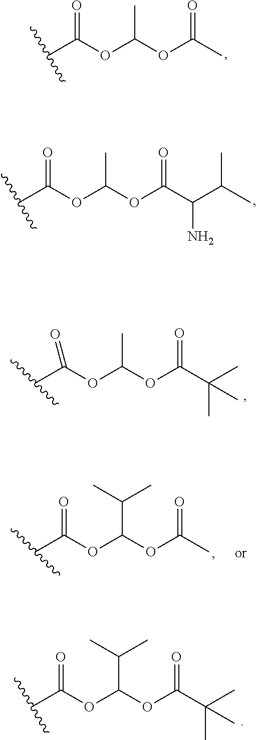

In some embodiments, R³ is

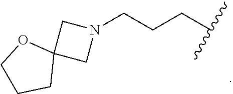 [SMILES: *CCCN1CC2(CCCO2)C1]

### Exemplary Combinations

In some embodiments, V is CH, Y is N, and G is CH₂.

In some embodiments, V is CH, Y is N, G is CH₂, and Ring A is cyclohexyl.

In some embodiments, V is CH, Y is N, G is CH₂, and Ring A is 6-membered heterocycloalkyl which has one heteroatom ring member being an oxygen.

In some embodiments, V is CH, Y is N, G is CH₂, Ring A is cyclohexyl, and R³ is NHS(O)₂ (C₂H₅).

In some embodiments, V is CH, Y is N, G is CH₂, Ring A is 6-membered heterocycloalkyl which has one heteroatom ring member being an oxygen, and R³ is NHS(O)₂ (C₂H₅).

In some embodiments, V is CH, Y is N, G is CH₂, Ring A is cyclohexyl, and R³ is NHS(O)₂NH(C₂H₅).

In some embodiments, V is CH, Y is N, G is CH₂, Ring A is 6-membered heterocycloalkyl which has one heteroatom ring member being an oxygen, and R³ is NHS(O)₂NH(C₂H₅). In some embodiments, V is CH, Y is N, and G is CH₂, and R² is isopropyl.

In some embodiments, V is CH, Y is N, G is CH₂, Ring A is cyclohexyl, and R² is isopropyl.

In some embodiments, V is CH, Y is N, G is CH₂, Ring A is 6-membered heterocycloalkyl which has one heteroatom ring member being an oxygen, and R² is isopropyl.

In some embodiments, V is CH, Y is N, G is CH₂, Ring A is cyclohexyl, R³ is NHS(O)₂ (C₂H₅), and R² is isopropyl.

In some embodiments, V is CH, Y is N, G is CH₂, Ring A is 6-membered heterocycloalkyl which has one heteroatom ring member being an oxygen, R³ is NHS(O)₂ (C₂H₅), and R² is isopropyl.

In some embodiments, V is CH, Y is N, G is CH₂, Ring A is cyclohexyl, R³ is NHS(O)₂NH(C₂H₅), and R² is isopropyl.

In some embodiments, V is CH, Y is N, G is CH₂, Ring A is 6-membered heterocycloalkyl which has one heteroatom ring member being an oxygen, R³ is NHS(O)₂NH(C₂H₅), and R² is isopropyl.

In some embodiments, V is CH, Y is N, G is CH₂, and R¹ is

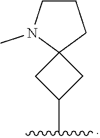 [SMILES: *C1CC2(CCCN2C)C1]

In some embodiments, V is CH, Y is N, G is CH₂, Ring A is cyclohexyl, and R¹ is

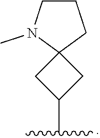 [SMILES: *C1CC2(CCCN2C)C1]

In some embodiments, V is CH, Y is N, G is CH₂, Ring A is 6-membered heterocycloalkyl which has one heteroatom ring member being an oxygen, and R¹ is

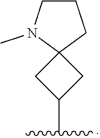 [SMILES: *C1CC2(CCCN2C)C1]

In some embodiments, V is CH, Y is N, G is CH₂, Ring A is cyclohexyl, R³ is NHS(O)₂ (C₂H₅), and R¹ is

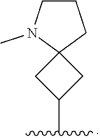 [SMILES: *C1CC2(CCCN2C)C1]

In some embodiments, V is CH, Y is N, G is CH₂, Ring A is 6-membered heterocycloalkyl which has one heteroatom ring member being an oxygen, R³ is NHS(O)₂ (C₂H₅), and R¹ is

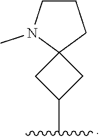 [SMILES: *C1CC2(CCCN2C)C1]

In some embodiments, V is CH, Y is N, G is CH₂, Ring A is cyclohexyl, R³ is NHS(O)₂NH(C₂H₅), and R¹ is

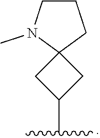 [SMILES: *C1CC2(CCCN2C)C1]

In some embodiments, V is CH, Y is N, G is CH₂, Ring A is 6-membered heterocycloalkyl which has one heteroatom ring member being an oxygen, R³ is NHS(O)₂NH(C₂H₅), and R¹ is

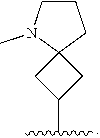 [SMILES: *C1CC2(CCCN2C)C1]

In some embodiments, V is CH, Y is N, G is CH₂, R² is isopropyl, and R¹ is

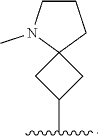 [SMILES: *C1CC2(CCCN2C)C1]

In some embodiments, V is CH, Y is N, G is CH₂, Ring A is cyclohexyl, R² is isopropyl, and R¹ is

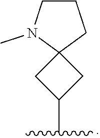 [SMILES: *C1CC2(CCCN2C)C1]

In some embodiments, V is CH, Y is N, G is CH₂, Ring A is 6-membered heterocycloalkyl which has one heteroatom ring member being an oxygen, R² is isopropyl, and R¹ is

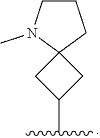 [SMILES: *C1CC2(CCCN2C)C1]

In some embodiments, V is CH, Y is N, G is CH₂, Ring A is cyclohexyl, R³ is NHS(O)₂ (C₂H₅), R² is isopropyl, and R¹ is

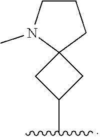 [SMILES: *C1CC2(CCCN2C)C1]

In some embodiments, V is CH, Y is N, G is CH₂, Ring A is 6-membered heterocycloalkyl which has one heteroatom ring member being an oxygen, R³ is NHS(O)₂ (C₂H₅), R² is isopropyl, and R¹ is

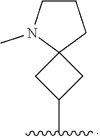 [SMILES: *C1CC2(CCCN2C)C1]

In some embodiments, V is CH, Y is N, G is CH₂, Ring A is cyclohexyl, R³ is NHS(O)₂NH(C₂H₅), R² is isopropyl, and R¹ is

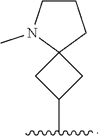 [SMILES: *C1CC2(CCCN2C)C1]

In some embodiments, V is CH, Y is N, G is CH₂, Ring A is 6-membered heterocycloalkyl which has one heteroatom ring member being an oxygen, R³ is NHS(0)₂NH(C₂H₅), R² is isopropyl, and R¹ is

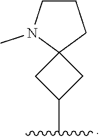 [SMILES: *C1CC2(CCCN2C)C1]

In some embodiments, V is CH, Y is N, G is CH₂, and R¹ is

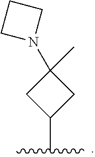 [SMILES: *C1CC(C)(N2CCC2)C1]

In some embodiments, V is CH, Y is N, G is CH₂, Ring A is cyclohexyl, and R¹ is

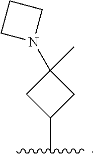 [SMILES: *C1CC(C)(N2CCC2)C1]

In some embodiments, V is CH, Y is N, G is CH₂, Ring A is 6-membered heterocycloalkyl which has one heteroatom ring member being an oxygen, and R¹ is

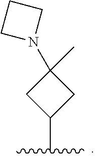 [SMILES: *C1CC(C)(N2CCC2)C1]

In some embodiments, V is CH, Y is N, G is CH₂, Ring A is cyclohexyl, R³ is NHS(O)₂ (C₂H₅), and R¹ is

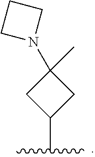 [SMILES: *C1CC(C)(N2CCC2)C1]

In some embodiments, V is CH, Y is N, G is CH₂, Ring A is 6-membered heterocycloalkyl which has one heteroatom ring member being an oxygen, R³ is NHS(O)₂ (C₂H₅), and R¹ is

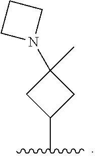 [SMILES: *C1CC(C)(N2CCC2)C1]

In some embodiments, V is CH, Y is N, G is CH₂, Ring A is cyclohexyl, R³ is NHS(O)₂NH(C₂H₅), and R¹ is

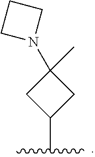 [SMILES: *C1CC(C)(N2CCC2)C1]

In some embodiments, V is CH, Y is N, G is CH₂, Ring A is 6-membered heterocycloalkyl which has one heteroatom ring member being an oxygen, R³ is NHS(O)₂NH(C₂H₅), and R¹ is

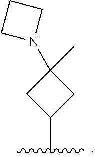 [SMILES: *C1CC(C)(N2CCC2)C1]

In some embodiments, V is CH, Y is N, G is CH₂, R² is isopropyl, and R¹ is

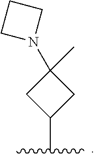 [SMILES: *C1CC(C)(N2CCC2)C1]

In some embodiments, V is CH, Y is N, G is CH₂, Ring A is cyclohexyl, R² is isopropyl, and R¹ is

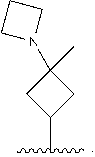 [SMILES: *C1CC(C)(N2CCC2)C1]

In some embodiments, V is CH, Y is N, G is CH₂, Ring A is 6-membered heterocycloalkyl which has one heteroatom ring member being an oxygen, R² is isopropyl, and R¹ is

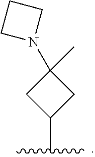 [SMILES: *C1CC(C)(N2CCC2)C1]

In some embodiments, V is CH, Y is N, G is CH₂, Ring A is cyclohexyl, R³ is NHS(O)₂ (C₂H₅), R² is isopropyl, and R¹ is

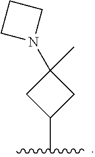 [SMILES: *C1CC(C)(N2CCC2)C1]

In some embodiments, V is CH, Y is N, G is CH₂, Ring A is 6-membered heterocycloalkyl which has one heteroatom ring member being an oxygen, R³ is NHS(O)₂ (C₂H₅), R² is isopropyl, and R¹ is

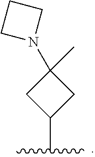 [SMILES: *C1CC(C)(N2CCC2)C1]

In some embodiments, V is CH, Y is N, G is CH₂, Ring A is cyclohexyl, R³ is NHS(O)₂NH(C₂H₅), R² is isopropyl, and R¹ is

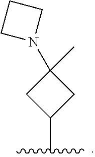 [SMILES: *C1CC(C)(N2CCC2)C1]

In some embodiments, V is CH, Y is N, G is CH₂, Ring A is 6-membered heterocycloalkyl which has one heteroatom ring member being an oxygen, R³ is NHS(O)₂NH(C₂H₅), R² is isopropyl, and R¹ is

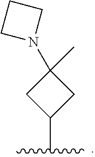 [SMILES: *C1CC(C)(N2CCC2)C1]

In some embodiments, V is CH, Y is N, G is CH₂, and R¹ is

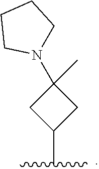 [SMILES: *C1CC(C)(N2CCCC2)C1]

In some embodiments, V is CH, Y is N, G is CH₂, Ring A is cyclohexyl, and R¹ is

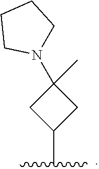 [SMILES: *C1CC(C)(N2CCCC2)C1]

In some embodiments, V is CH, Y is N, G is CH₂, Ring A is 6-membered heterocycloalkyl which has one heteroatom ring member being an oxygen, and R¹ is

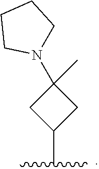 [SMILES: *C1CC(C)(N2CCCC2)C1]

In some embodiments, V is CH, Y is N, G is CH₂, Ring A is cyclohexyl, R³ is NHS(O)₂ (C₂H₅), and R¹ is

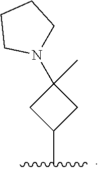 [SMILES: *C1CC(C)(N2CCCC2)C1]

In some embodiments, V is CH, Y is N, G is CH₂, Ring A is 6-membered heterocycloalkyl which has one heteroatom ring member being an oxygen, R³ is NHS(O)₂ (C₂H₅), and R¹ is

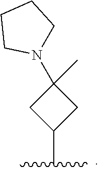 [SMILES: *C1CC(C)(N2CCCC2)C1]

In some embodiments, V is CH, Y is N, G is CH₂, Ring A is cyclohexyl, R³ is NHS(O)₂NH(C₂H₅), and R¹ is

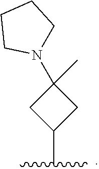 [SMILES: *C1CC(C)(N2CCCC2)C1]

In some embodiments, V is CH, Y is N, G is CH₂, Ring A is 6-membered heterocycloalkyl which has one heteroatom ring member being an oxygen, R³ is NHS(O)₂NH(C₂H₅), and R¹ is

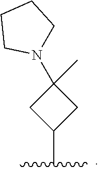 [SMILES: *C1CC(C)(N2CCCC2)C1]

In some embodiments, V is CH, Y is N, G is CH₂, R² is isopropyl, and R¹ is

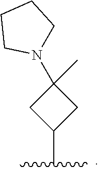 [SMILES: *C1CC(C)(N2CCCC2)C1]

In some embodiments, V is CH, Y is N, G is CH₂, Ring A is cyclohexyl, R² is isopropyl, and R¹ is

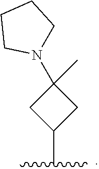 [SMILES: *C1CC(C)(N2CCCC2)C1]

In some embodiments, V is CH, Y is N, G is CH₂, Ring A is 6-membered heterocycloalkyl which has one heteroatom ring member being an oxygen, R² is isopropyl, and R¹ is

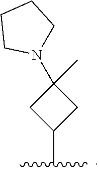 [SMILES: *C1CC(C)(N2CCCC2)C1]

In some embodiments, V is CH, Y is N, G is CH₂, Ring A is cyclohexyl, R³ is NHS(O)₂ (C₂H₅), R² is isopropyl, and R¹ is

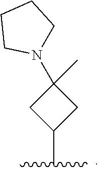 [SMILES: *C1CC(C)(N2CCCC2)C1]

In some embodiments, V is CH, Y is N, G is CH₂, Ring A is 6-membered heterocycloalkyl which has one heteroatom ring member being an oxygen, R³ is NHS(O)₂ (C₂H₅), R² is isopropyl, and R¹ is

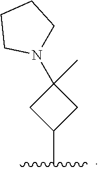 [SMILES: *C1CC(C)(N2CCCC2)C1]

In some embodiments, V is CH, Y is N, G is CH₂, Ring A is cyclohexyl, R³ is NHS(0)₂NH(C₂H₅), R² is isopropyl, and R¹ is

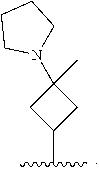 [SMILES: *C1CC(C)(N2CCCC2)C1]

In some embodiments, V is CH, Y is N, G is CH₂, Ring A is 6-membered heterocycloalkyl which has one heteroatom ring member being an oxygen, R³ is NHS(O)₂NH(C₂H₅), R² is isopropyl, and R¹ is

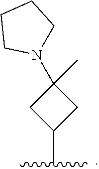 [SMILES: *C1CC(C)(N2CCCC2)C1]

In some embodiments, V is N, Y is N, and G is CH₂.

In some embodiments, V is N, Y is N, G is CH₂, and Ring A is cyclohexyl.

In some embodiments, V is N, Y is N, G is CH₂, and Ring A is 6-membered heterocycloalkyl which has one heteroatom ring member being an oxygen.

In some embodiments, V is N, Y is N, G is CH₂, Ring A is cyclohexyl, and R³ is NHS(O)₂ (C₂H₅).

In some embodiments, V is N, Y is N, G is CH₂, Ring A is 6-membered heterocycloalkyl which has one heteroatom ring member being an oxygen, and R³ is NHS(O)₂ (C₂H₅).

In some embodiments, V is N, Y is N, G is CH₂, Ring A is cyclohexyl, and R³ is NHS(0)₂NH(C₂H₅).

In some embodiments, V is N, Y is N, G is CH₂, Ring A is 6-membered heterocycloalkyl which has one heteroatom ring member being an oxygen, and R³ is NHS(O)₂NH(C₂H₅). In some embodiments, V is N, Y is N, and G is CH₂, and R² is isopropyl.

In some embodiments, V is N, Y is N, G is CH₂, Ring A is cyclohexyl, and R² is isopropyl. In some embodiments, V is N, Y is N, G is CH₂, Ring A is 6-membered heterocycloalkyl which has one heteroatom ring member being an oxygen, and R² is isopropyl.

In some embodiments, V is N, Y is N, G is CH₂, Ring A is cyclohexyl, R³ is NHS(O)₂ (C₂H₅), and R² is isopropyl.

In some embodiments, V is N, Y is N, G is CH₂, Ring A is 6-membered heterocycloalkyl which has one heteroatom ring member being an oxygen, R³ is NHS(O)₂ (C₂H₅), and R² is isopropyl.

In some embodiments, V is N, Y is N, G is CH₂, Ring A is cyclohexyl, R³ is NHS(O)₂NH(C₂H₅), and R² is isopropyl.

In some embodiments, V is N, Y is N, G is CH₂, Ring A is 6-membered heterocycloalkyl which has one heteroatom ring member being an oxygen, R³ is NHS(O)₂NH(C₂H₅), and R² is isopropyl.

In some embodiments, V is N, Y is N, G is CH₂, and R¹ is

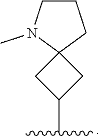 [SMILES: *C1CC2(CCCN2C)C1]

In some embodiments, V is N, Y is N, G is CH₂, Ring A is cyclohexyl, and R¹ is

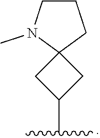 [SMILES: *C1CC2(CCCN2C)C1]

In some embodiments, V is N, Y is N, G is CH₂, Ring A is 6-membered heterocycloalkyl which has one heteroatom ring member being an oxygen, and R¹ is

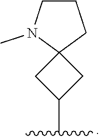 [SMILES: *C1CC2(CCCN2C)C1]

In some embodiments, V is N, Y is N, G is CH₂, Ring A is cyclohexyl, R³ is NHS(O)₂ (C₂H₅), and R¹ is

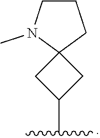 [SMILES: *C1CC2(CCCN2C)C1]

In some embodiments, V is N, Y is N, G is CH₂, Ring A is 6-membered heterocycloalkyl which has one heteroatom ring member being an oxygen, R³ is NHS(O)₂ (C₂H₅), and R¹ is

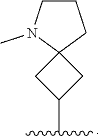 [SMILES: *C1CC2(CCCN2C)C1]

In some embodiments, V is N, Y is N, G is CH₂, Ring A is cyclohexyl, R³ is NHS(O)₂NH(C₂H₅), and R¹ is

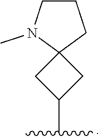 [SMILES: *C1CC2(CCCN2C)C1]

In some embodiments, V is N, Y is N, G is CH₂, Ring A is 6-membered heterocycloalkyl which has one heteroatom ring member being an oxygen, R³ is NHS(O)₂NH(C₂H₅), and R¹ is

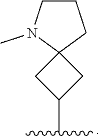 [SMILES: *C1CC2(CCCN2C)C1]

In some embodiments, V is N, Y is N, G is CH₂, R² is isopropyl, and R¹ is

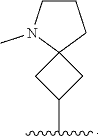 [SMILES: *C1CC2(CCCN2C)C1]

In some embodiments, V is N, Y is N, G is CH₂, Ring A is cyclohexyl, R² is isopropyl, and R¹ is

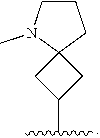 [SMILES: *C1CC2(CCCN2C)C1]

In some embodiments, V is N, Y is N, G is CH₂, Ring A is 6-membered heterocycloalkyl which has one heteroatom ring member being an oxygen, R² is isopropyl, and R¹ is

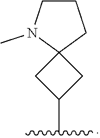 [SMILES: *C1CC2(CCCN2C)C1]

In some embodiments, V is N, Y is N, G is CH₂, Ring A is cyclohexyl, R³ is NHS(O)₂ (C₂H₅), R² is isopropyl, and R¹ is

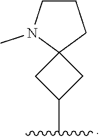 [SMILES: *C1CC2(CCCN2C)C1]

In some embodiments, V is N, Y is N, G is CH₂, Ring A is 6-membered heterocycloalkyl which has one heteroatom ring member being an oxygen, R³ is NHS(O)₂ (C₂H₅), R² is isopropyl, and R¹ is

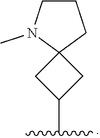 [SMILES: *C1CC2(CCCN2C)C1]

In some embodiments, V is N, Y is N, G is CH₂, Ring A is cyclohexyl, R³ is NHS(O)₂NH(C₂H₅), R² is isopropyl, and R¹ is

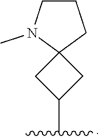 [SMILES: *C1CC2(CCCN2C)C1]

In some embodiments, V is N, Y is N, G is CH₂, Ring A is 6-membered heterocycloalkyl which has one heteroatom ring member being an oxygen, R³ is NHS(O)₂NH(C₂H₅), R² is isopropyl, and R¹ is

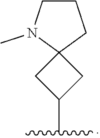 [SMILES: *C1CC2(CCCN2C)C1]

In some embodiments, V is N, Y is N, G is CH₂, and R¹ is

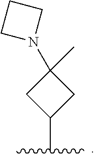 [SMILES: *C1CC(C)(N2CCC2)C1]

In some embodiments, V is N, Y is N, G is CH₂, Ring A is cyclohexyl, and R¹ is

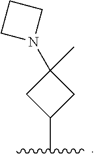 [SMILES: *C1CC(C)(N2CCC2)C1]

In some embodiments, V is N, Y is N, G is CH₂, Ring A is 6-membered heterocycloalkyl which has one heteroatom ring member being an oxygen, and R¹ is

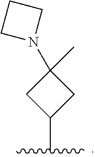 [SMILES: *C1CC(C)(N2CCC2)C1]

In some embodiments, V is N, Y is N, G is CH₂, Ring A is cyclohexyl, R³ is NHS(O)₂ (C₂H₅), and R¹ is

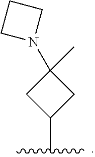 [SMILES: *C1CC(C)(N2CCC2)C1]

In some embodiments, V is N, Y is N, G is CH₂, Ring A is 6-membered heterocycloalkyl which has one heteroatom ring member being an oxygen, R³ is NHS(O)₂ (C₂H₅), and R¹ is

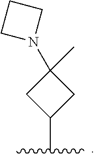 [SMILES: *C1CC(C)(N2CCC2)C1]

In some embodiments, V is N, Y is N, G is CH₂, Ring A is cyclohexyl, R³ is NHS(O)₂NH(C₂H₅), and R¹ is

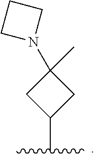 [SMILES: *C1CC(C)(N2CCC2)C1]

In some embodiments, V is N, Y is N, G is CH₂, Ring A is 6-membered heterocycloalkyl which has one heteroatom ring member being an oxygen, R³ is NHS(O)₂NH(C₂H₅), and R¹ is

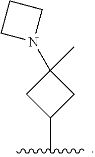 [SMILES: *C1CC(C)(N2CCC2)C1]

In some embodiments, V is N, Y is N, G is CH₂, R² is isopropyl, and R¹ is

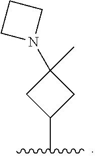 [SMILES: *C1CC(C)(N2CCC2)C1]

In some embodiments, V is N, Y is N, G is CH₂, Ring A is cyclohexyl, R² is isopropyl, and R¹ is

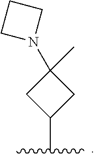 [SMILES: *C1CC(C)(N2CCC2)C1]

In some embodiments, V is N, Y is N, G is CH₂, Ring A is 6-membered heterocycloalkyl which has one heteroatom ring member being an oxygen, R² is isopropyl, and R¹ is

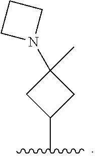 [SMILES: *C1CC(C)(N2CCC2)C1]

In some embodiments, V is N, Y is N, G is CH₂, Ring A is cyclohexyl, R³ is NHS(O)₂ (C₂H₅), R² is isopropyl, and R¹ is

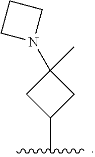 [SMILES: *C1CC(C)(N2CCC2)C1]

In some embodiments, V is N, Y is N, G is CH₂, Ring A is 6-membered heterocycloalkyl which has one heteroatom ring member being an oxygen, R³ is NHS(O)₂ (C₂H₅), R² is isopropyl, and R¹ is

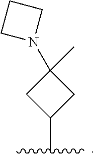 [SMILES: *C1CC(C)(N2CCC2)C1]

In some embodiments, V is N, Y is N, G is CH₂, Ring A is cyclohexyl, R³ is NHS(O)₂NH(C₂H₅), R² is isopropyl, and R¹ is

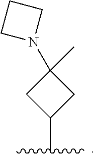 [SMILES: *C1CC(C)(N2CCC2)C1]

In some embodiments, V is N, Y is N, G is CH₂, Ring A is 6-membered heterocycloalkyl which has one heteroatom ring member being an oxygen, R³ is NHS(O)₂NH(C₂H₅), R² is isopropyl, and R¹ is

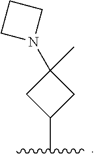 [SMILES: *C1CC(C)(N2CCC2)C1]

In some embodiments, V is N, Y is N, G is CH₂, and R¹ is

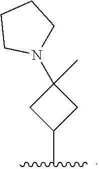 [SMILES: *C1CC(C)(N2CCCC2)C1]

In some embodiments, V is N, Y is N, G is CH₂, Ring A is cyclohexyl, and R¹ is

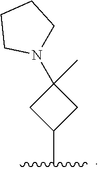 [SMILES: *C1CC(C)(N2CCCC2)C1]

In some embodiments, V is N, Y is N, G is CH₂, Ring A is 6-membered heterocycloalkyl which has one heteroatom ring member being an oxygen, and R¹ is

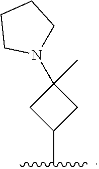 [SMILES: *C1CC(C)(N2CCCC2)C1]

In some embodiments, V is N, Y is N, G is CH₂, Ring A is cyclohexyl, R³ is NHS(O)₂ (C₂H₅), and R¹ is

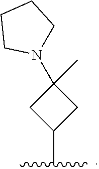 [SMILES: *C1CC(C)(N2CCCC2)C1]

In some embodiments, V is N, Y is N, G is CH₂, Ring A is 6-membered heterocycloalkyl which has one heteroatom ring member being an oxygen, R³ is NHS(O)₂ (C₂H₅), and R¹ is

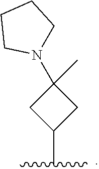 [SMILES: *C1CC(C)(N2CCCC2)C1]

In some embodiments, V is N, Y is N, G is CH₂, Ring A is cyclohexyl, R³ is NHS(0)₂NH(C₂H₅), and R¹ is

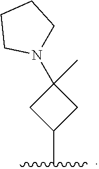 [SMILES: *C1CC(C)(N2CCCC2)C1]

In some embodiments, V is N, Y is N, G is CH₂, Ring A is 6-membered heterocycloalkyl which has one heteroatom ring member being an oxygen, R³ is NHS(O)₂NH(C₂H₅), and R¹ is

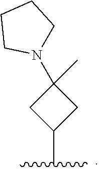 [SMILES: *C1CC(C)(N2CCCC2)C1]

In some embodiments, V is N, Y is N, G is CH₂, R² is isopropyl, and R¹ is

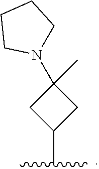 [SMILES: *C1CC(C)(N2CCCC2)C1]

In some embodiments, V is N, Y is N, G is CH₂, Ring A is cyclohexyl, R² is isopropyl, and R¹ is

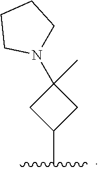 [SMILES: *C1CC(C)(N2CCCC2)C1]

In some embodiments, V is N, Y is N, G is CH₂, Ring A is 6-membered heterocycloalkyl which has one heteroatom ring member being an oxygen, R² is isopropyl, and R¹ is

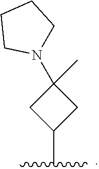 [SMILES: *C1CC(C)(N2CCCC2)C1]

In some embodiments, V is N, Y is N, G is CH₂, Ring A is cyclohexyl, R³ is NHS(O)₂ (C₂H₅), R² is isopropyl, and R¹ is

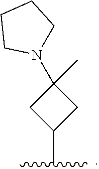 [SMILES: *C1CC(C)(N2CCCC2)C1]

In some embodiments, V is N, Y is N, G is CH₂, Ring A is 6-membered heterocycloalkyl which has one heteroatom ring member being an oxygen, R³ is NHS(O)₂ (C₂H₅), R² is isopropyl, and R¹ is

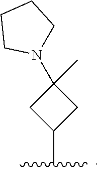 [SMILES: *C1CC(C)(N2CCCC2)C1]

In some embodiments, V is N, Y is N, G is CH₂, Ring A is cyclohexyl, R³ is NHS(O)₂NH(C₂H₅), R² is isopropyl, and R¹ is

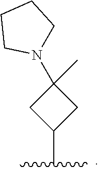 [SMILES: *C1CC(C)(N2CCCC2)C1]

In some embodiments, V is N, Y is N, G is CH₂, Ring A is 6-membered heterocycloalkyl which has one heteroatom ring member being an oxygen, R³ is NHS(O)₂NH(C₂H₅), R² is isopropyl, and R¹ is

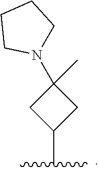 [SMILES: *C1CC(C)(N2CCCC2)C1]

In some embodiments, the spiro moiety represented by the below formula:

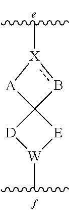

wherein e and f indicate points of attachment to the remainder of the molecule, is

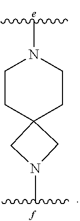 [SMILES: *N1CCC2(CC1)CN(*)C2]

In some embodiments, the compound is of Formula I, II, III, III-a, III-b, III-c, III-b-1, III-b-2, III-b-3, III-c-1, III-c-2, or III-c-3 combined with any of the embodiments described herein.

Any of the groups described above for any variable can be combined with any of the other groups described above, where applicable, for any of the Formulae described herein.

Representative compounds of the present disclosure are shown in the table below.

TABLE 1
Representative Compounds of the Present Disclosure

| Compound | | |
|---|---|---|
| No. | Structure | IUPAC Name |
| 1 | 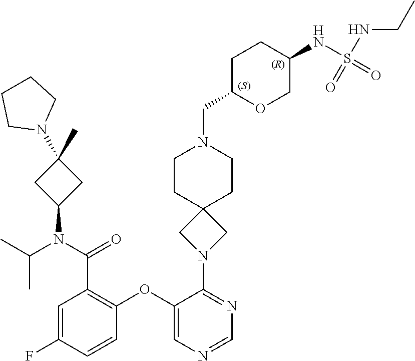 | 2-((4-(7-(((2S,5R)-5-((N- Ethylsulfamoyl)amino)tetrahydro-2H- pyran-2-yl)methyl)-2,7- diazaspiro[3.5]nonan-2-yl)pyrimidin-5- yl)oxy)-5-fluoro-N-isopropyl-N-((1r,3r)-3- methyl-3-(pyrrolidin-1- yl)cyclobutyl)benzamide |
| 2 | 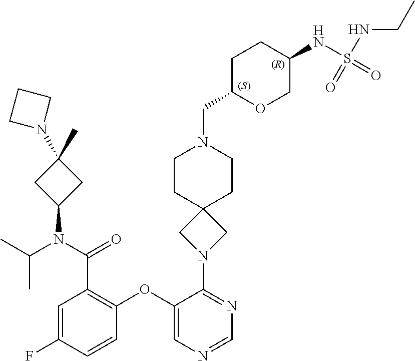 | N-((1r,3r)-3-(Azetidin-1-yl)-3- methylcyclobutyl)-2-((4-(7-(((25,5R)-5- ((N-ethylsulfamoyl)amino)tetrahydro-2H- pyran-2-yl)methyl)-2,7- diazaspiro[3.5]nonan-2-yl)pyrimidin-5- yl)oxy)-5-fluoro-N-isopropylbenzamide |
| 3 | 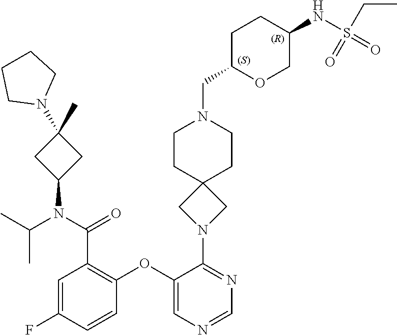 | 2-((4-(7-(((25,5R)-5- (Ethylsulfonamido)tetrahydro-2H-pyran-2- yl)methyl)-2,7-diazaspiro[3.5]nonan-2- yl)pyrimidin-5-yl)oxy)-5-fluoro-N- isopropyl-N-((1r,3r)-3-methyl-3- (pyrrolidin-1-yl)cyclobutyl)benzamide |
| 4 | 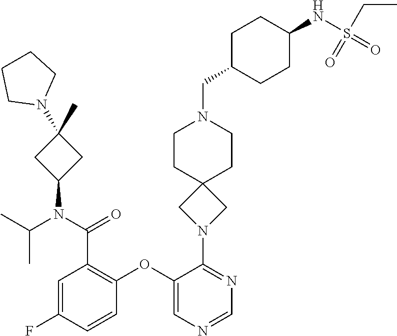 [SMILES: CCS(=O)(=O)N[C@H]1CC[C@H](CN2CCC3(CC2)CN(c2ncncc2Oc2ccc(F)cc2C(=O)N(C(C)C)[C@H]2C[C@](C)(N4CCCC4)C2)C3)CC1] | 2-((4-(7-(((1r,4r)-4- (Ethylsulfonamido)cyclohexyl)methyl)- 2,7-diazaspiro[3.5]nonan-2-yl)pyrimidin-5- yl)oxy)-5-fluoro-N-isopropyl-N-((1r,3r)-3- methyl-3-(pyrrolidin-1- yl)cyclobutyl)benzamide |
| 5 | 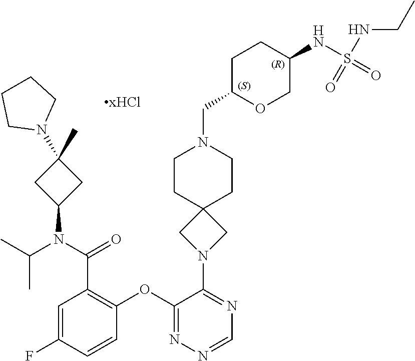 | 2-((5-(7-(((25,5R)-5-((N- Ethylsulfamoyl)amino)tetrahydro-2H- pyran-2-yl)methyl)-2,7- diazaspiro[3.5]nonan-2-y1)-1,2,4-triazin-6- yl)oxy)-5-fluoro-N-isopropyl-N-((1r,3r)-3- methyl-3-(pyrrolidin-1- yl)cyclobutyl)benzamide xHCl |
| 6 | 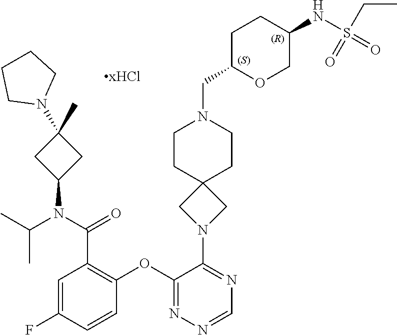 | 2-((5-(7-(((2S,5R)-5- (Ethylsulfonamido)tetrahydro-2H-pyran-2- yl)methyl)-2,7-diazaspiro[3.5]nonan-2-yl)- 1,2,4-triazin-6-yl)oxy)-5-fluoro-N- isopropyl-N-((1r,3r)-3-methyl-3- (pyrrolidin-1-yl)cyclobutyl)benzamide xHCl |
| 7 | 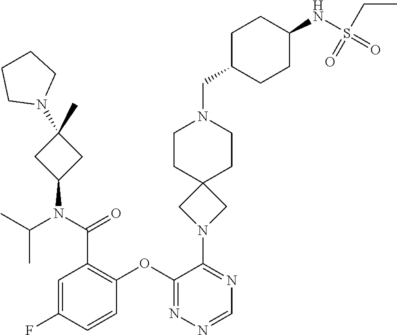 [SMILES: CCS(=O)(=O)N[C@H]1CC[C@H](CN2CCC3(CC2)CN(c2ncnnc2Oc2ccc(F)cc2C(=O)N(C(C)C)[C@H]2C[C@](C)(N4CCCC4)C2)C3)CC1] | 2-((5-(7-(((1r,4r)-4- (Ethylsulfonamido)cyclohexyl)methyl)- 2,7-diazaspiro[3.5]nonan-2-yl)-1,2,4- triazin-6-yl)oxy)-5-fluoro-N-isopropyl-N- ((1r,3r)-3-methyl-3-(pyrrolidin-1- yl)cyclobutyl)benzamide |
| 8 | 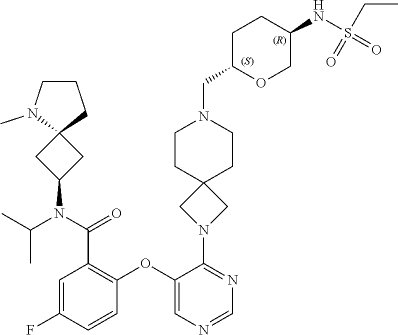 | 2-((4-(7-(((2S,5R)-5- (Ethylsulfonamido)tetrahydro-2H-pyran-2- yl)methyl)-2,7-diazaspiro[3.5]nonan-2- y1)pyrimidin-5-yl)oxy)-5-fluoro-N- isopropyl-N-((2s,4r)-5-methyl-5- azaspiro[3.4]octan-2-yl)benzamide |
| 9 | 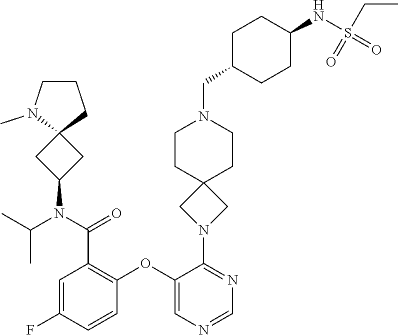 [SMILES: CCS(=O)(=O)N[C@H]1CC[C@H](CN2CCC3(CC2)CN(c2ncncc2Oc2ccc(F)cc2C(=O)N(C(C)C)[C@H]2C[C@@]4(CCCN4C)C2)C3)CC1] | 2-((4-(7-(((1r,4r)-4- (Ethylsulfonamido)cyclohexyl)methyl)- 2,7-diazaspiro[3.5]nonan-2-yl)pyrimidin-5- yl)oxy)-5-fluoro-N-isopropyl-N-((2s,4r)-5- methyl-5-azaspiro[3.4]octan-2- yl)benzamide |

In some embodiments, the present disclosure is directed to a compound as shown in Table 1 or a stereoisomer thereof, or a pharmaceutically acceptable salt thereof.

In some embodiments, the present disclosure is directed to a compound as shown in Table 1 or a pharmaceutically acceptable salt thereof.

In some embodiments, the present disclosure is directed to a compound as shown in Table 1.

In some embodiments, the present disclosure is directed to a compound as shown in Table 1 or a pharmaceutically acceptable salt thereof, wherein the salt is hydrochloride.

In some embodiments, a compound according to any embodiments herein (e.g., Formulae I, II, III, III-a, III-b, III-c, III-b-1, III-b-2, III-b-3, III-c-1, III-c-2, and III-c-3, and Table 1) exhibits an inhibition activity against the binding of menin and MLL. In some embodiments, a compound according to any embodiments herein (e.g., Formulae I, II, III, III-a, III-b, III-c, III-b-1, III-b-2, III-b-3, III-c-1, III-c-2, and III-c-3, and Table 1) exhibits an inhibition activity against the binding of menin and MLL. In some embodiments, a compound according to any embodiments herein (e.g., Formulae I, II, III, III-a, III-b, III-c, III-b-1, III-b-2, III-b-3, III-c-1, III-c-2, and III-c-3, and Table 1) exhibits an inhibition activity against the binding of menin and MLL which is useful in the treatment and/or prevention of one or more diseases in which menin and MLL play a role.

In some embodiments, a compound according to any embodiments herein (e.g., Formulae I, II, III, III-a, III-b, III-c, III-b-1, III-b-2, III-b-3, III-c-1, III-c-2, and III-c-3, and Table 1) exhibits low hERG binding. In some embodiments, a compound according to any embodiments herein (e.g., Formulae I, II, III, III-a, III-b, III-c, III-b-1, III-b-2, III-b-3, III-c-1, III-c-2, and III-c-3, and Table 1) minimizes hERG binding and is useful for the treatment of one or more diseases in which menin and MLL play a role. Without being bound to any theory, one of the primary causes of QT prolongation is thought to be blockage of the hERG potassium channel in cardiac myocytes. In some embodiments, the compounds of the present disclosure (e.g., Formulae I, II, III, III-a, III-b;
III-c, III-b-1, III-b-2, III-b-3, III-c-1, III-c-2, and III-c-3, and Table 1) do not significantly block the hERG potassium channel.

In some embodiments, the compounds of the present disclosure (e.g., Formulae I, II, III, III-a, III-b, III-c, III-b-1, III-b-2, III-b-3, III-c-1, III-c-2, and III-c-3, and Table 1) do not significantly block the hERG potassium channel (e.g., an IC₅₀ greater than 1 μM, 5 μM, 10 μM, 15 μM, 20 μM, 25 μM, 30 μM, 35 μM, 40 μM, or 50 μM) as measured by a standard patch clamp hERG assay.

In some embodiments, the compounds of the present disclosure (e.g., Formulae I, II, III, III-a, III-b, III-c, III-b-1, III-b-2, III-b-3, III-c-1, III-c-2, and III-c-3, and Table 1) do not significantly block the hERG potassium channel (e.g., a compound of the invention has an IC₅₀ greater than 1 μM, 5 μM, 10 μM, 15 μM, 20 μM, 25 μM, 30 μM, 35 μM, 40 μM, or 50 μM for the hERG potassium channel).

In some embodiments and without wishing to be bound to any theory, the present disclosure is directed to inhibitors of the menin-MLL interaction comprising a pyrrolidine moiety (e.g., Formulae I, II, III, III-a, III-b, III-c, III-b-1, III-b-2, III-b-3, III-c-1, III-c-2, and III-c-3, and Table 1) where the pyrrolidine moiety has been found to reduce hERG inhibition.

In some embodiments and without wishing to be bound to any theory, the present disclosure is directed to inhibitors of the menin-MLL interaction comprising a piperidine moiety (e.g., Formulae I, II, III, III-a, III-b, III-c, III-b-1, III-b-2, III-b-3, III-c-1, III-c-2, and III-c-3, and Table 1) where the piperidine moiety has been found to reduce hERG inhibition.

In some embodiments and without wishing to be bound to any theory, the present disclosure is directed to inhibitors of the menin-MLL interaction (e.g., Formulae I, II, III, III-a, III-b, III-c, III-b-1, III-b-2, III-b-3, III-c-1, III-c-2, and III-c-3, and Table 1) which are resistant to metabolism. In some embodiments and without wishing to be bound to any theory, the present disclosure is directed to inhibitors of the menin-MLL interaction (e.g., Formulae I, II, III, III-a, III-b, III-c, III-b-1, III-b-2, III-b-3, III-c-1, III-c-2, and III-c-3, and Table 1) which are resistant to metabolism, where the metabolites are inhibitors of the hERG potassium channel. In some embodiments and without wishing to be bound to any theory, the present disclosure is directed to inhibitors of the menin-MLL interaction (e.g., Formulae I, II, III, III-a, III-b, III-c, III-b-1, III-b-2, III-b-3, III-c-1, III-c-2, and III-c-3, and Table 1) which are resistant to metabolism, where the metabolism decreases the bioavailability of the inhibitor. In some embodiments and without wishing to be bound to any theory, the present disclosure is directed to inhibitors of the menin-MLL interaction (e.g., Formulae I, II, III, III-a, III-b, III-c, III-b-1, III-b-2, III-b-3, III-c-1, III-c-2, and III-c-3, and Table 1) which are resistant to metabolism, where the metabolism decreases the bioavailability of the inhibitor and the corresponding metabolites are more effective (e.g., by IC₅₀, etc.) at binding the hERG potassium channel.

In some embodiments, one or more hydrogen atoms in any of the compounds of Formula I, II, III, III-a, III-b, III-c, III-b-1, III-b-2, III-b-3, III-c-1, III-c-2, or III-c-3 may be replaced with one or more deuterium atoms.

Another aspect is an isotopically labeled compound of any of the formulae delineated herein. Such compounds have one or more isotopic atoms (e.g., ³H, ²H, ¹⁴C, ¹³C, ¹⁸F, ³⁵S, ³²P, ¹²⁵I, and ¹³¹I) introduced into the compound. Such compounds are useful for drug metabolism studies and diagnostics; as well as therapeutic applications. In some embodiments, the compound is an isotopic derivative of any one of the compounds described in Table 1, or a pharmaceutically acceptable salt thereof.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent. A compound of the present disclosure or a pharmaceutically acceptable salt or solvate thereof that contains the aforementioned deuterium atom(s) is within the scope of the disclosure. Further, substitution with deuterium (i.e., 2H) may afford certain therapeutic advantages resulting from greater metabolic stability, e.g., increased in vivo half-life or reduced dosage requirements. For the avoidance of doubt, it is to be understood that, where in this specification a group is qualified by “described herein”, the said group encompasses the first occurring and broadest definition as well as each and all of the particular definitions for that group.

Potency can also be determined by IC₅₀ value. A compound with a lower IC₅₀ value, as determined under substantially similar conditions, is more potent relative to a compound with a higher IC₅₀ value.

Potency can also be determined by EC₅₀ value. A compound with a lower EC₅₀ value, as determined under substantially similar conditions, is more potent relative to a compound with a higher EC₅₀ value.

In some embodiments, a pair of enantiomers have similar potency.

In some embodiments, a pair of enantiomers have different potency. In specific embodiments, the enantiomer with (R) configuration is more potent than the enantiomer with (S) configuration. In other specific embodiments, the enantiomer with(S) configuration is more potent than the enantiomer with (R) configuration.

In some embodiments, a pair of isomers have similar potency.

In some embodiments, a pair of isomers have different potency. In specific embodiments, the trans isomer is more potent than the cis isomer. In specific embodiments, the cis isomer is more potent than the trans isomer.

In some embodiments, a group of diastereomers have similar potency.

In some embodiments, a group of diastereomers have different potency.

In specific embodiments, the diastereomer with (R)(R) configuration is the most potent among the group of diastereomers.

In specific embodiments, the diastereomer with(S) (S) configuration is the most potent among the group of diastereomers.

In specific embodiments, the diastereomer with(S)(R) configuration is the most potent among the group of diastereomers.

In specific embodiments, the diastereomer with (R) (S) configuration is the most potent among the group of diastereomers.

In specific embodiments, the diastereomer with (R)(R) configuration is more potent than the diastereomer with(S) (S) configuration.

In specific embodiments, the diastereomer with(S) (S) configuration is more potent than the diastereomer with (R)(R) configuration.

In specific embodiments, the diastereomer with (R) (S) configuration is more potent than the diastereomer with(S)(R) configuration.

In specific embodiments, the diastereomer with(S)(R) configuration is more potent than the diastereomer with (R) (S) configuration.

The compounds of the application are defined herein by their chemical structures and/or chemical names. Where a compound is referred to by both a chemical structure and a chemical name, and the chemical structure and chemical name conflict, the chemical structure is determinative of the compound's identity.

In another aspect, the application provides a method of synthesizing a compound disclosed herein. The synthesis of the compounds of the application can be found herein and in the Examples below. Other embodiments are a method of making a compound of any of the formulae herein using any one, or combination of, reactions delineated herein. The method can include the use of one or more intermediates or chemical reagents delineated herein.

In some embodiments, each of the intermediates prepared in the Schemes herein are considered embodiments of the present disclosure. In some embodiments, each of the intermediates prepared in the Examples herein are considered embodiments of the present disclosure. In some embodiments, each synthetic step as disclosed in the Schemes herein is separately considered as part of the present disclosure. In some embodiments, each synthetic step as disclosed in the Examples herein is separately considered as part of the present disclosure.

It is appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various cycloalkyl, and heterocyclyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

For compounds of the disclosure in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the term “amino,” employed alone or in combination with other terms, refers to a group of formula-NH₂. In some embodiments, an amine can be substituted by one or more groups, e.g., —N—(C₁-C₆ alkyl)₂. In some embodiments, when two groups are attached to an amine they can be the same or different. Each group is selected independently of each other and can be each independently optionally substituted.

As used herein, the term “halogen” or “halo,” employed alone or in combination with other terms, refers to a halogen atom selected from F, C₁, I or Br. In some embodiments, “halo” refers to a halogen atom selected from F, C₁, or Br. In some embodiments, the halo substituent is F The term “haloalkyl” or “haloalkoxyl” refers to an alkyl or alkoxyl substituted with one or more halogen atoms. In some embodiments, the haloalkyl group is fluorinated. In some embodiments, the haloalkyl group is fluorinated only. In some embodiments, the haloalkyl group is fluoromethyl, difluoromethyl, or trifluoromethyl. In some embodiments, the haloalkyl group is trifluoromethyl. In some embodiments, the haloalkyl group is 2,2,2-trifluoroethyl. In some embodiments, the haloalkyl group is 2,2-difluoroethyl. In some embodiments, the haloalkyl group has 1 to 6 or 1 to 4 carbon atoms.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic. In some embodiments, cycloalkyl may optionally contain one or more alkenylene groups as part of the ring structure. Cycloalkyl groups can include mono- or polycyclic ring systems. Polycyclic ring systems can include fused ring systems and spirocycles. Also included in the definition of cycloalkyl are moieties that have one or more aromatic rings fused (i.e., having a bond in common with) to the cycloalkyl ring, for example, benzo or pyrido derivatives of cyclopentane, cyclopentene, cyclohexane, and the like. A heterocyclyl group that includes a fused aromatic (e.g., aryl or heteroaryl) moiety can be attached to the molecule through an atom from either the aromatic or non-aromatic portion. One or more ring-forming carbon atoms of a cycloalkyl group can be oxidized to form carbonyl linkages. In some embodiments, cycloalkyl is C₃₋₁₀ cycloalkyl, C₃-7 cycloalkyl, or C₅₋₆ cycloalkyl. Exemplary cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, and the like. Further exemplary cycloalkyl groups include cyclopropyl, cyclobutyl, cyclopentyl, and cyclohexyl. Additional example cycloalkyl groups, where the cycloalkyl group has a fused aryl or heteroaryl moiety, include tetrahydronaphthalen-2-yl, 2,3-dihydro-1H-inden-2-yl; 2,3,4,9-tetrahydro-1H-carbazol-7-yl; 2,6,7,8-tetrahydrobenzo[cd]indazol-4-yl; and 5,6,7,8,9,10-hexahydrocyclohepta [b]indol-3-yl. Further exemplary cycloalkyl groups include

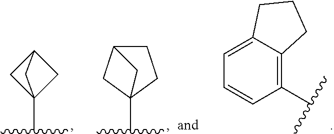

As used herein, the term “aryl,” employed alone or in combination with other terms, refers to a monocyclic or polycyclic (e.g., having 2, 3 or 4 fused rings) aromatic hydrocarbon, such as, but not limited to, phenyl, 1-naphthyl, 2-naphthyl, anthracenyl, phenanthrenyl, and the like. In some embodiments, aryl is C₆₋₁₀ aryl. In some embodiments, aryl is C₆₋₁₄ aryl. In some embodiments, the aryl group is a naphthalene ring or phenyl ring. In some embodiments, the aryl group is phenyl.

As used herein, the term “heteroaryl,” employed alone or in combination with other terms, refers to a monocyclic or polycyclic (e.g., having 2, 3 or 4 fused rings) aromatic heterocylic moiety, having one or more heteroatom ring members selected from nitrogen, sulfur and oxygen. In some embodiments, the heteroaryl group has 1, 2, 3, or 4 heteroatom ring members. In some embodiments, the heteroaryl group has 1, 2, or 3 heteroatom ring members. In some embodiments, the heteroaryl group has 1 or 2 heteroatom ring members. In some embodiments, the heteroaryl group has 1 heteroatom ring member. In some embodiments, the heteroaryl group is 5- to 10-membered or 5- to 6-membered. In some embodiments, the heteroaryl group is 5-membered. In some embodiments, the heteroaryl group is 6-membered. In some embodiments, the heteroaryl group is 9- or 10-membered bicyclic. In some embodiments, the heteroaryl is 9-member bicyclic. When the heteroaryl group contains more than one heteroatom ring member, the heteroatoms may be the same or different. The nitrogen atoms in the ring(s) of the heteroaryl group can be oxidized to form N-oxides. Example heteroaryl groups include, but are not limited to, pyridinyl, pyrimidinyl, pyrazinyl, pyridazinyl, pyrrolyl, pyrazolyl, azolyl, oxazolyl, isoxazolyl, thiazolyl, isothiazolyl, imidazolyl, furanyl, thiophenyl, triazolyl, tetrazolyl, thiadiazolyl, quinolinyl, isoquinolinyl, indolyl, benzothiopheneyl, benzofuranyl, benzisoxazolyl, benzoimidazolyl, imidazo[1,2-b]thiazolyl, purinyl, triazinyl, and the like. In some embodiments, the heteroaryl group is 9H-carbazol-2-yl; 1H-benzo[d]imidazol-6-yl; 1H-indol-6-yl; 1H-indazol-6-yl; 2H-indazol-4-yl; 1H-benzo[d][1,2,3]triazol-6-yl; benzo[d]oxazol-2-yl; quinolin-6-yl; or benzo[d]thiazol-2-yl.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthyridine, indole, benzofuran, purine, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the phrase “optionally substituted” means unsubstituted or substituted. As used herein, the term “substituted” or “substituted with one or more”, means that any one or more hydrogen atoms on the designated atom is replaced with a selection from the indicated groups, provided that the designated atom's normal valency is not exceeded, and that the substitution results in a stable compound. When a substituent is oxo or keto (i.e., —O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “heterocyclyl” or “heterocycloalkyl” employed alone or in combination with other terms, refers to a non-aromatic heterocyclic ring system, which may optionally contain one or more unsaturations as part of the ring structure, and which has at least one heteroatom ring member independently selected from nitrogen, sulfur and oxygen. In some embodiments, the heterocyclyl group has 1, 2, 3, or 4 heteroatom ring members. In some embodiments, the heterocyclyl group has 1, 2, or 3 heteroatom ring members. In some embodiments, the heterocyclyl group has 1 or 2 heteroatom ring members. In some embodiments, the heterocyclyl group has 1 heteroatom ring member. When the heterocyclyl group contains more than one heteroatom in the ring, the heteroatoms may be the same or different. Example ring-forming members include CH, CH₂, C(O), N, NH, O, S, S(O), and S(═O)₂. Heterocyclyl groups can include mono- or polycyclic (e.g., having 2, 3 or 4 fused rings) ring systems. Polycyclic rings can include both fused systems and spirocycles. Also included in the definition of heterocyclyl are moieties that have one or more aromatic rings fused (i.e., having a bond in common with) to the non-aromatic ring, for example, 1, 2, 3, 4-tetrahydro-quinoline, dihydrobenzofuran and the like. A heterocyclyl group that includes a fused aromatic moiety can be attached to the molecule through an atom from either the aromatic or non-aromatic portion. The carbon atoms or heteroatoms in the ring(s) of the heterocyclyl group can be oxidized to form a carbonyl, sulfinyl, or sulfonyl group (or other oxidized linkage) or a nitrogen atom can be quaternized. In some embodiments, heterocyclyl is 5- to 10-membered, 4- to 10-membered, 4- to 7-membered, 5-membered, or 6-membered. Examples of heterocyclyl groups include 1, 2, 3, 4-tetrahydro-quinolinyl, dihydrobenzofuranyl, azetidinyl, azepanyl, pyrrolidinyl, piperidinyl, piperazinyl, morpholinyl, thiomorpholinyl, and pyranyl. Examples of heterocyclyl groups that include one or more fused aromatic groups (e.g., aryl or heteroaryl) include N-(2′-oxospiro[cyclohexane-1,3′-indolin]-6′-yl; 1,2,3,4-tetrahydroisoquinolin-6-yl; 2,3-dihydro-1H-benzo[d]imidazol-5-yl; 1,3-dihydrospiro[indene-2,3′-indolin]-6′-yl; 2,3-dihydrobenzo[d]oxazol-5-yl; 1,2-dihydroquinolin-7-yl; indolin-6-yl; spiro[cyclopentane-1,3′-indolin]-6′-yl; spiro[cyclohexane-1,3′-indolin]-6′-yl; chroman-6-yl; 3,4-dihydro-2H-benzo[b][1,4]oxazin-6-yl; and benzo[d][1,3]dioxol-5-yl.

Examples of “heterocyclyl” or “heterocycloalkyl” groups include

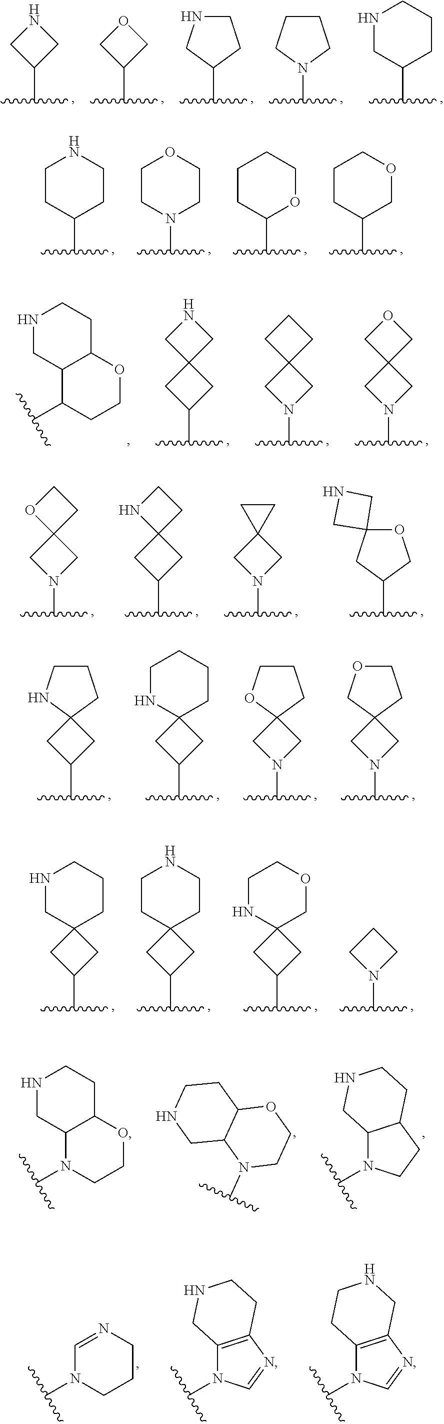

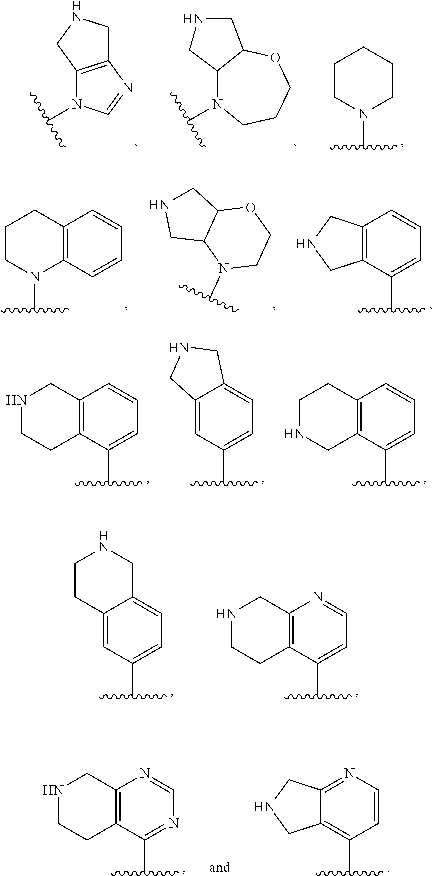

As used herein, the term “alkylene” refers to a refers to a divalent radical derived from an alkane, as exemplified, by (—CH₂-)ₙ, wherein n may be 1 to about 24. By way of example only, such groups include, but are not limited to, groups having 10 or fewer carbon atoms such as the structures-CH₂CH₂— and —CH₂CH₂CH₂CH₂—. As used herein, the terms “Cn-Cm” and “Cn-m”, where each of n and m is an integer, can be used interchangeably.

As used herein, the term “cycloalkylene” refers to a cycloalkyl group wherein two hydrogens are removed to provide a divalent radical. It is understood that the two hydrogens, prior to removal, can be attached to same or different atoms in the cycloalkyl group. Examples of cycloalkylene groups include

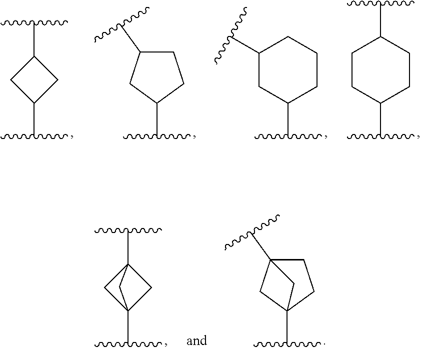

As used herein, “ [symbol] ” represents a bond to the position indicated by the corresponding variable, for example Ring A, R¹, R², R³, G, X, A, B, D, E, Z, W, V, Y, R^{g}, R^{1s}, R^{1as}, R^{1bs}, R^{1cs}, R^{3a}, R^{4a}, R^{4b}, R^{3a}′, R^{3b}′, R^{3c}′, R^{4a}′, or R^{4b}′.

As used in the embodiments describing Ring A, when Ring A is

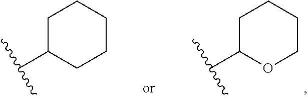 [SMILES: *C1CCCCC1 ; *C1CCCCO1]

is intended that the position of the substituents can be at any position allowed by the available valences according to general formulae I, II, III, III-a, III-b, and III-c, or a stereoisomer, or a pharmaceutically acceptable salt thereof, e.g.

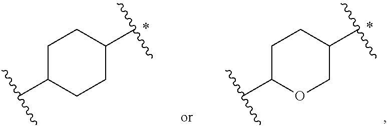

wherein * indicates attachment to R^{3.}

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R and S sequencing rules of Cahn and Prelog, or by the manner in which the molecule rotates the plane of polarised light and designated as dextrorotatory or levorotatory (i.e., as (+) or (−) isomers respectively). A chiral compound can exist as either individual enantiomer or as a mixture thereof. A mixture containing equal proportions of the enantiomers is called a “racemic mixture”. The compounds described herein can be asymmetric (e.g., having one or more stereocenters). All stereoisomers, such as enantiomers and diastereoisomers, are intended unless otherwise indicated. Where a compound name or structure is silent with respect to the stereochemistry of a stereocenter, all possible configurations at the stereocenter are intended. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

When the compounds of the disclosure contain a chiral center, the compounds can be any of the possible stereoisomers. In compounds with a single chiral center, the stereochemistry of the chiral center can be (R) or(S). In compounds with two chiral centers, the stereochemistry of the chiral centers can each be independently (R) or(S) so the configuration of the chiral centers can be (R) and (R), (R) and(S); (S) and (R), or(S) and(S). In compounds with three chiral centers, the stereochemistry each of the three chiral centers can each be independently (R) or(S) so the configuration of the chiral centers can be (R), (R) and (R); (R), (R) and(S); (R), (S) and (R); (R), (S) and(S); (S), (R) and (R); (S), (R) and(S); (S), (S) and (R); or(S), (S) and(S).

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereoisomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

As used herein, the terms “E1” and “E2” correspond to first and second enantiomers to elute from a chiral chromatography column as discussed in the Examples.

As used herein, the terms “D1” and “D2” correspond to first and second diastereomers to elute from a chiral chromatography column as discussed in the Examples.

When a disclosed compound is named or depicted without indicating the stereochemistry of one or more stereocenters, each of the stereoisomers resulting from the possible stereochemistries at the undefined stereocenter(s) are intended to be encompassed. For example, if a stereocenter is not designated as R or S, then either or both are intended.

Compounds of the disclosure also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H—, 2H— and 4H-1, 2, 4-triazole, 1H- and 2H-isoindole, and 1H- and 2H-pyrazole. Tautomeric forms can be in equilibrium or sterically locked into one form by appropriate substitution.

Compounds of the disclosure can also include all isotopes of atoms occurring in the intermediates or final compounds. Isotopes include those atoms having the same atomic number but different mass numbers. Isotopes of constituent atoms of the compounds of the disclosure can be present in natural or non-natural abundance. Examples of isotopes of hydrogen include deuterium and tritium. In some embodiments, the compounds of the disclosure are deuterated, meaning at least one deuterium atom is present in the place of a hydrogen atom. In some embodiments, 1, 2, 3, 4, 5, 6, 7, or 8 hydrogens in a compound of the disclosure are replaced by deuterium. Methods for replacing hydrogen with deuterium in a molecule are known in the art.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified (e.g., in the case of purine rings, unless otherwise indicated, when the compound name or structure has the 9H tautomer, it is understood that the 7H tautomer is also encompassed).

All compounds, and pharmaceutically acceptable salts thereof, can be found together with other substances such as water and solvents (e.g., hydrates and solvates) or can be isolated.

It will be understood that the compounds of the present disclosure and any pharmaceutically acceptable salts thereof, comprise stereoisomers, mixtures of stereoisomers, polymorphs of all isomeric forms of said compounds.

In some embodiments, the compounds of the disclosure, or salts thereof, or crystalline forms of any of the aforementioned, are purified or substantially isolated. By “substantially isolated” is meant that the compound is at least partially or substantially separated from the environment in which it was formed or detected. Partial separation can include, for example, a composition enriched in a compound of the disclosure. Substantial separation can include compositions containing at least about 50%, at least about 60%, at least about 70%, at least about 80%, at least about 90%, at least about 95%, at least about 97%, or at least about 99% by weight of the compounds of the disclosure, or salt thereof. In some embodiments, the compounds of the disclosure, or salts thereof, or crystalline forms of any of the aforementioned, can be prepared with a purity of about 75% or more, 80% or more, 85% or more, 90% or more, 95% or more, 98% or more, or 99% or more.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g., a reaction temperature, that is about the temperature of the room in which the reaction is carried out, for example, a temperature from about 20° C. to about 30° C.

The present disclosure also includes pharmaceutically acceptable salts of the compounds described herein. As used herein, “pharmaceutically acceptable salts” or “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (MeCN) are preferred.

The compounds disclosed herein include the compounds themselves, as well as their salts, their solvates, and their prodrugs, if applicable. A salt, for example, can be formed between an anion and a positively charged group (e.g., protonated amino) on a compound of this disclosure. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluroacetate). The term “pharmaceutically acceptable anion” refers to an anion suitable for forming a pharmaceutically acceptable salt. Likewise, a salt can also be formed between a cation and a negatively charged group (e.g., carboxylate) on a compound of this disclosure. Suitable cations include sodium ion, potassium ion, magnesium ion, calcium ion, and an ammonium cation such as tetramethylammonium ion. The compounds of this disclosure also include those salts containing quaternary nitrogen atoms. Examples of prodrugs include esters and other pharmaceutically acceptable derivatives, which, upon administration to a subject, are capable of providing active compounds of this disclosure.

Additionally, physiologically acceptable, i.e., pharmaceutically compatible, salts can be salts of the compounds disclosed herein with inorganic or organic acids. Preference is given to salts with inorganic acids, such as, for example, hydrochloric acid, hydrobromic acid, phosphoric acid or sulphuric acid, or to salts with organic carboxylic or sulphonic acids, such as, for example, acetic acid, trifluoroacetic acid, propionic acid, maleic acid, fumaric acid, malic acid, citric acid, tartaric acid, lactic acid, benzoic acid, or methanesulphonic acid, ethanesulphonic acid, benzenesulphonic acid, toluenesulphonic acid or naphthalenedisulphonic acid.

Other pharmaceutically compatible salts which may be mentioned are salts with customary bases, such as, for example, alkali metal salts (for example sodium or potassium salts), alkaline earth metal salts (for example calcium or magnesium salts) or ammonium salts, derived from ammonia or organic amines, such as, for example, diethylamine, triethylamine, ethyldiisopropylamine, procaine, dibenzylamine, N-methylmorpholine, dihydroabietylamine or methylpiperidine.

As used herein, “pharmaceutically acceptable salts” can refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts can include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, or an alkaline earth metal ion, e.g., an aluminum ion; or coordinates with an organic base such as ethanolamine, diethanolamine, triethanolamine, tromethamine, N-methylglucamine, diethylamine, diethylaminoethanol, ethylenediamine, imidazole, lysine, arginine, morpholine, 2-hydroxyethylmorpholine, dibenzylethylenediamine, trimethylamine, piperidine, pyrrolidine, benzylamine, tetramethylammonium hydroxide and the like.

It should be understood that all references to pharmaceutically acceptable salts include solvent addition forms (solvates) or crystal forms (polymorphs) as defined herein, of the same salt.

It is to be understood that, unless otherwise stated, any description of a method of treatment or prevention includes use of the compounds to provide such treatment or prevention as is described herein. It is to be further understood, unless otherwise stated, any description of a method of treatment or prevention includes use of the compounds to prepare a medicament to treat or prevent such condition. The treatment or prevention includes treatment or prevention of human or non-human animals including rodents and other disease models.

It is to be understood that, unless otherwise stated, any description of a method of treatment includes use of the compounds to provide such treatment as is described herein. It is to be further understood, unless otherwise stated, any description of a method of treatment includes use of the compounds to prepare a medicament to treat such condition. The treatment includes treatment of human or non-human animals including rodents and other disease models.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3rd edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18th edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

The compounds of the present disclosure may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at a sulfonylurea group.

Accordingly, the present disclosure includes those compounds of the present disclosure as defined hereinbefore when made available by organic synthesis and when made available within the human or animal body by way of cleavage of a prodrug thereof. Accordingly, the present disclosure includes those compounds of the present disclosure that are produced by organic synthetic means and also such compounds that are produced in the human or animal body by way of metabolism of a precursor compound, that is a compound of the present disclosure may be a synthetically-produced compound or a metabolically-produced compound.

A suitable pharmaceutically acceptable prodrug of a compound of the present disclosure is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of the present disclosure that possesses a hydroxy group is, for example, an in vivo cleavable ester or ether thereof. An in vivo cleavable ester or ether of a compound of the present disclosure containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl) piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of the present disclosure that possesses a carboxy group is, for example, an in vivo cleavable amide thereof, for example an amide formed with an amine such as ammonia, a C₁-C₄ alkylamine such as methylamine, a (C₁-C₄ alkyl)₂amine such as dimethylamine, N-ethyl N-methylamine or diethylamine, a C₁-C₄ alkoxy C₂-C₄ alkylamine such as 2 methoxyethylamine, a phenyl C₁-C₄ alkylamine such as benzylamine and amino acids such as glycine or an ester thereof.

A suitable pharmaceutically acceptable prodrug of a compound of the present disclosure that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl, and 4-(C₁-C₄ alkyl) piperazin-1-ylmethyl.

The in vivo effects of a compound of the present disclosure may be exerted in part by one or more metabolites that are formed within the human or animal body after administration of a compound of the present disclosure. As stated hereinbefore, the in vivo effects of a compound of the present disclosure may also be exerted by way of metabolism of a precursor compound (a prodrug).

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### Synthesis

In some aspects, the present disclosure provides a method of preparing a compound disclosed herein.

In some aspects, the present disclosure provides a method of preparing a compound, comprising one or more steps as described herein.

In some aspects, the present disclosure provides a compound obtainable by, or obtained by, or directly obtained by a method for preparing a compound described herein.

In some aspects, the present disclosure provides an intermediate being suitable for use in a method for preparing a compound described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl (Bn). The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively, an acyl group such as a tert butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris (trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively, an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of the present disclosure has been synthesized by any one of the processes defined herein, the processes may then further comprise the additional steps of: (i) removing any protecting groups present; (ii) converting the compound of the present disclosure into another compound of the present disclosure; (iii) forming a pharmaceutically acceptable salt, hydrate or solvate thereof; and/or (iv) forming a prodrug thereof.

The resultant compounds of the present disclosure can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognize which kind of reagents and reactions conditions are to be used and how they are to be applied and adapted in any particular instance-wherever necessary or useful—in order to obtain the compounds of the present disclosure. Furthermore, some of the compounds of the present disclosure can readily be synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply-whenever necessary or useful-synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons). The compounds of the disclosure can be synthesized by the methods described in Schemes 1-5 below. The synthesis of various hydroxyl-substituted heterocycles is well documented in the literature and can be synthesized by known literature methods. The depicted intermediates may also be available as commercial reagents from numerous vendors.

The compounds of the present disclosure can be prepared according to the following general procedures illustrated in the following Schemes. It will be appreciated that, although the general methods may depict the synthesis of certain compounds of the present disclosure, the following general methods, and other methods known to one of ordinary skill in the art, can be applied to all compounds and subclasses and species of each of these compounds, as described herein.

The variables, where used in the following General Schemes, can be replaced to match the variable or moieties of any of the Formulae and Compounds disclosed herein as appropriate.

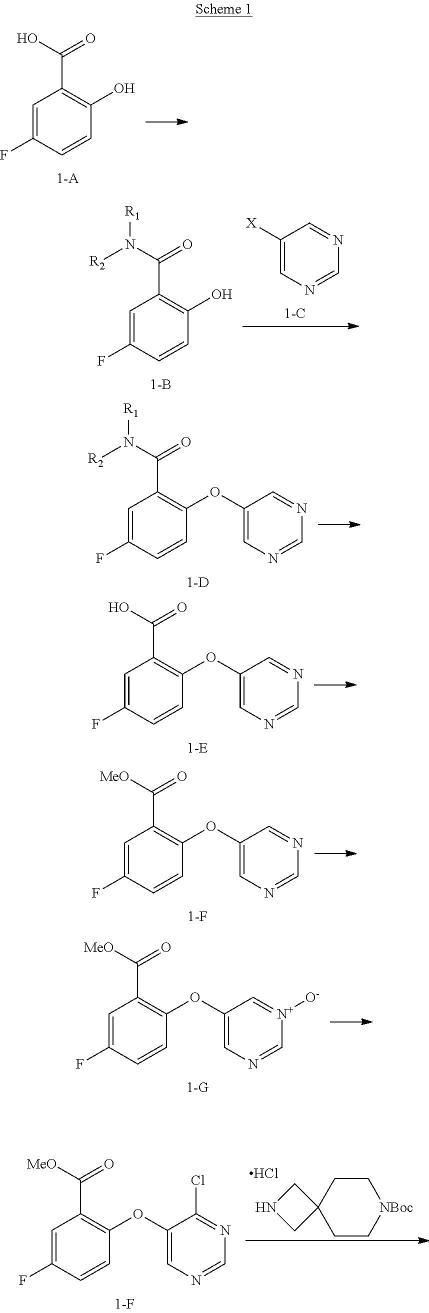

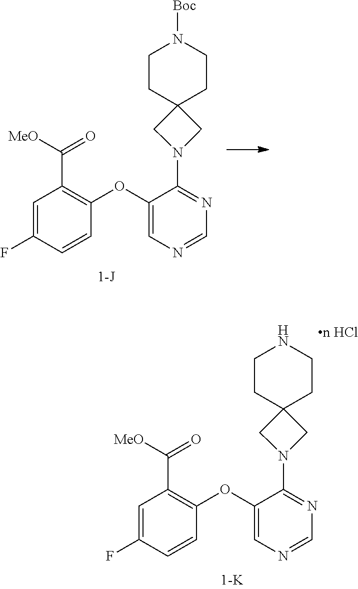 [SMILES: COC(=O)c1cc(F)ccc1Oc1cncnc1N1CC2(CCN(C(=O)OC(C)(C)C)CC2)C1 ; COC(=O)c1cc(F)ccc1Oc1cncnc1N1CC2(CCNCC2)C1]

Scheme 1 illustrates the general method for the preparation of key intermediates like compound 1-K. A suitable salicylic acid derivative, for instance 5-fluoro-2-hydroxybenzoic acid (1-A), is converted initially to the corresponding amide 1-B(Ri and R²═CH₃). Many different conditions have been developed to achieve this type of transformation, and will be generally familiar to those of skill in the art. For instance, the carboxylic acid can be reacted with thionyl chloride or oxalyl chloride, which forms the corresponding acid chloride. This reaction can be conducted in a suitable, neutral solvent like CH₂Cl₂ or THF, or if thionyl chloride is used, can be run with thionyl chloride as the reactant and solvent, if desired. The acid chloride is subsequently reacted with a suitable amine, R₁R₂NH, in the presence of an appropriate acid scavenger, for example Et₃N, (iPr)₂NEt, or pyridine, in a neutral solvent like CH₂Cl₂ or THF. In some instances, pyridine can be used as the acid scavenger and the solvent for the reaction. Sometimes, a strong base like sodium hydride, in a neutral solvent like THF, DMF, or toluene, is used. Alternatively, the carboxylic acid and amine R₁R₂NH can be combined together and coupled using a reagent such as DCC(dicyclohexyl carbodiimide), EDC(1-ethyl-3-(4-dimethylaminopropyl) carbodiimide), HATU (1-[bis(dimethylamino)methylene]-1H-1,2,3-triazolo[4,5-b]pyridinium 3-oxide hexafluorophosphate), HBTU (2-(1H-benzotriazol-1-yl)-1,1,3,3-tetramethyluronium hexafluorophosphate), T3P, or POCl₃, in the presence of an appropriate acid scavenger, for example Et₃N, (iPr)₂NEt, or pyridine, in a neutral solvent, oftentimes DMF, NMP, CH₂Cl₂ or THF. In some instances, when DCC or EDC is used as the coupling reagent, HOBt (hydroxybenzotriazole) might be added to the reaction to facilitate the desired coupling reaction. In the present case, a preferred method is to convert carboxylic acid 1-A to the corresponding acid chloride (not shown in the scheme) using SOCl₂ in THE as solvent. The acid chloride is subsequently reacted with a suitable amine, for example N,N-dimethylamine, in the presence of an acid scavenger, typically Et₃N or (iPr)₂NEt, to afford benzamide 1-B(R₁ and R₂═CH₃). The phenol of 1-B is then reacted with a 5-halopyrimidine (1-C), for example 5-bromopyrimidine or 5-iodopyrimidine to afford the corresponding diaryl ether 1-D. This reaction is typically conducted in the presence of an acid scavenger like Na₂CO₃, K₂CO₃, Cs₂CO₃, K₃PO₄, Et₃N (triethylamine) or (iPr)₂NEt (diisopropylethylamine, also known as Hunig's base), in a suitable, neutral solvent, such as DMA (dimethyl acetamide) or NMP (N-methylpyrrolidinone). In some instances, this transformation can be facilitated by addition of an appropriate catalyst, typically Cu₂O or CuI, in the presence of a suitable ligand, for example rel-(1R,2R)—N¹,N²-bis(2-pyridinylmethylene)-1,2-cyclohexanediamine or 3,4,7,8-tetramethyl-1,10-phenanthroline. The amide group of compound 1-D is subsequently saponified to the corresponding carboxylic acid 1-E. Typically, this transformation is accomplished using LiOH, NaOH or KOH, in an aqueous solvent system, such as methanol/water or THF/water. The resulting carboxylic acid is then converted to ester 1-F. The transformation of a carboxylic acid to an ester is usually achieved by reacting the carboxylic acid with an alcohol, such as methanol of ethanol, in the presence of an acid catalyst like HCl or H₂SO₄. Typically, the alcohol is used as the solvent, and heat can be used to increase the reaction rate. Alternatively, 1-E can be O-alkylated using methyl iodide in the presence of an acid scavenger, oftentimes K₂CO₃, to afford 1-F. Reaction of 1-F with a suitable oxidizing agent, such as urea hydroperoxide in the presence of trifluoroacetic anhydride, or meta-chloroperoxybenzoic acid (mCPBA), in a suitable solvent, typically CH₂Cl₂ or THF, affords the pyrimidine N-oxide 1-G. This then can be chlorinated to afford compound 1-H using an appropriate chlorinating agent, typically POCl₃ or oxalyl chloride, in the presence of an acid scavenger, usually Et₃N, (i-Pr)₂NEt, in a neutral solvent like CH₂Cl₂, ethyl acetate (EtOAc), or isopropyl acetate. The chloride of compound 1-H can be readily displaced with an amine, or a suitably protected spiro-diamine, for instance tert-butyl 2,7-diazaspiro[3.5]nonane-7-carboxylate (1-I), to afford compounds like 1-J. This reaction is conducted in the presence of an acid scavenger, usually Et₃N or (i-Pr)₂NEt, in a neutral solvent like CH₂Cl₂, THE, DMF, IPA, or isopropyl acetate. When a protected diamine is used, the protecting group can be removed subsequently according to the nature of the protecting group. The use of protecting groups in organic synthesis is well-known to those of skill in the art, and conditions for adding and removing protecting groups are described in established reference volumes, for instance Greene's Protective Groups in Organic Synthesis, 4th Edition (ISBN: 9780470053485). For example, the Boc (tert-butyloxycarbonyl) protecting group in compound 1-J can be removed under acidic conditions, using acids like HCl, TFA (trifluoracetic acid), or p-TsOH (para-toluenesulfonic acid), in a suitable solvent, oftentimes CH₂Cl₂ or THF. The Boc group can also be removed using TMSCl in 2,2,2-trifluoroethanol. The desired, deprotected amine can be isolated as the salt, for instance the hydrochloride salt 1-K, or as the free base, following work-up under standard basic conditions. It will be appreciated by those of skill in the art that compound 1-K is a flexible intermediate that can be converted into the compounds of the present invention (4-C and 4-D, see below) in a variety of ways.

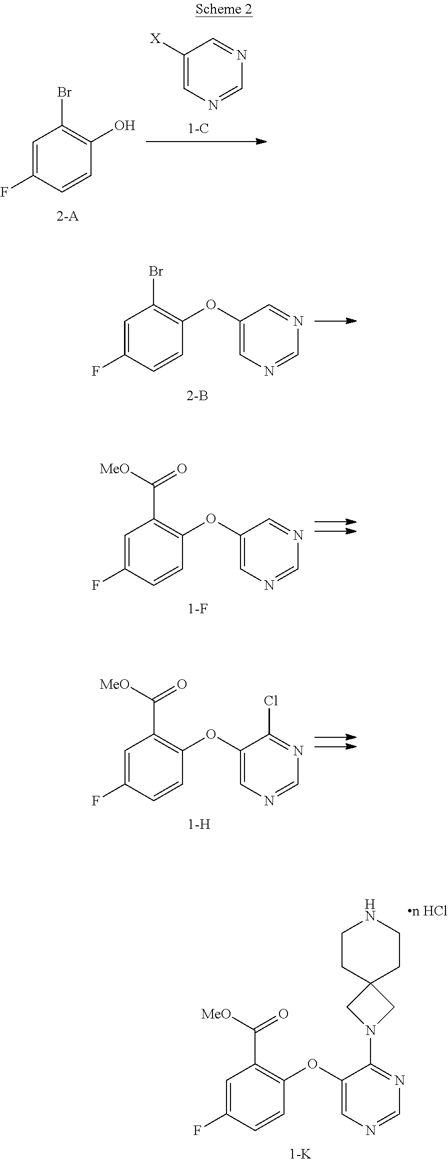

Scheme 2 illustrates an alternative method for the preparation of key intermediates like compound 1-H. A suitably substituted 2-halophenol, for instance 4-fluoro-2-bromophenol (2-A), is reacted with a 5-halopyrimidine (1-C), for example 5-bromopyrimidine or 5-iodopyrimidine, to afford the corresponding diaryl ether (2-B). This reaction is typically conducted in the presence of an acid scavenger like Na₂CO₃, K₂CO₃, Cs₂CO₃, K₃PO₄, Et₃N (triethylamine) or (iPr)₂NEt (diisopropylethylamine, also known as Hunig's base), in a suitable, neutral solvent, such as DMA (dimethyl acetamide) or NMP (N-methylpyrrolidinone). In some instances, this transformation can be facilitated by addition of an appropriate catalyst, typically Cu2O or CuI, in the presence of a suitable ligand, for example rel-(1R,2R)—N¹,N²-bis(2-pyridinylmethylene)-1,2-cyclohexanediamine or 3,4,7,8-tetramethyl-1,10-phenanthroline. Conversion of compound 2-B to compound 1-F can be readily achieved by carbonylation under Pd catalysis, in the presence of an acid scavenger and suitable alcohol (typically used as the solvent in the reaction). Oftentimes, Pd(dppf)Cl₂ (dppf=1,1-bis(diphenylphosphino) ferrocene) is used as the palladium catalyst, but other catalyst systems, such as Pd(Ph₃P)₂Cl₂ (Ph₃P=triphenylphosphine) or Pd(OAc)₂ with Ph₃P, can also be used. Commonly, Et₃N or (i-Pr)₂NEt are used as the acid scavengers, and methanol is used as the solvent. These reactions are most typically run under an atmosphere of CO, but other CO sources, for example oxalic acid, can be used in some instances. Compound 1-F can then be transformed into the key intermediate 1-H and 1-K as described in detail in Scheme 1 (1-F to 1-K).

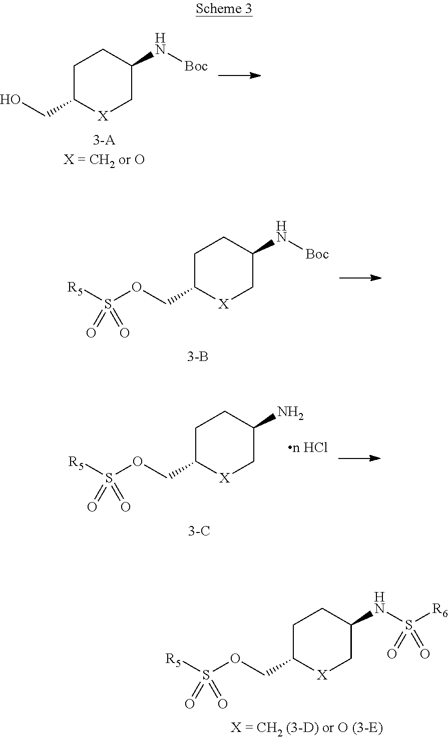

Scheme 3 illustrates the general methods for preparing compounds like 3-D and 3-E, which can be used to alkylate compounds like 1-K. Commercially available tert-butyl ((1r,4r)-4-(hydroxymethyl)cyclohexyl) carbamate (3-A, X═CH₂) or 1,5-anhydro-2,3,4-trideoxy-2-[(1,1-dimethylethoxy) carbonyl]amino]-D-erythro-hexitol (3-A, X═O) is reacted with a suitable sulfonyl halide, preferably p-toluenesulfonyl chloride (TsCl), to afford the sulfonate derivative 3—B(R₅=p-MePhe). This reaction is typically conducted in a neutral solvent like CH₂Cl₂ or THF, in the presence of an acid scavenger, typically Et₃N, Hunig's base, or pyridine. Sometimes, pyridine is used as both the solvent and acid scavenger in this reaction. The Boc group of 3-B is then removed according to the general conditions for Boc deprotection described in Scheme 1, to afford the amino compound 3-C. Reaction of 3-C with a suitable sufonyl halide affords the corresponding sulfonamide derivatives 3-D (R₆=an alkyl or aryl group). This reaction is generally conducted in the same way as described for the transformation of 3-A to 3-B. Other compounds of the present invention can be prepared similarly, via reaction of 3-C with appropriate acylating agents like sulfamoyl halides (to afford sulfamides), acid chlorides (to afford amides), chloroformates (to afford carbamates), or carbamoyl chlorides or isocyanates (to afford ureas).

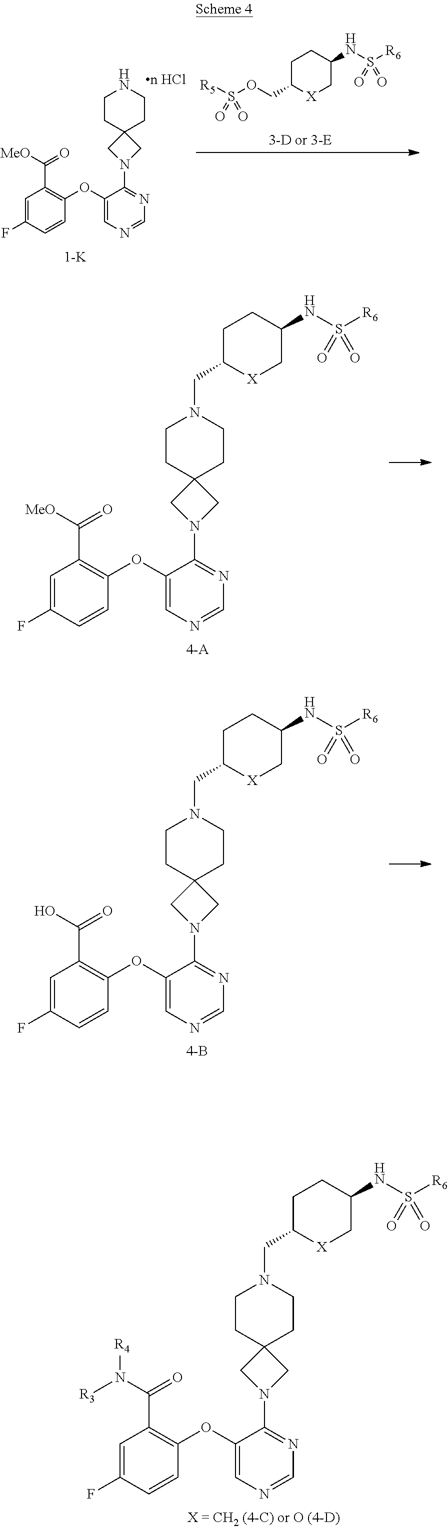

Scheme 4 illustrates methods for converting the key intermediate 1-K into the compounds of the present invention (4-C or 4-D). Reaction of 1-K with an appropriate alkylating agent, for instance compounds 3-D or 3-E (Scheme 3), in the presence of an acid scavenger like Et₃N or (iPr)₂NEt, in a neutral solvent like ACN, THF, DMF or NMP, affords compound 4-A. Sometimes, KI or tetrabutylammonium iodide (TBAI) can be added to facilitate the reaction. Saponification of the ester of 4-A, as generally described previously, provides the carboxylic acid derivative 4-B, which is subsequently converted to the target amide derivative 4-C and 4-D according to the general methods for amide formation described previously.

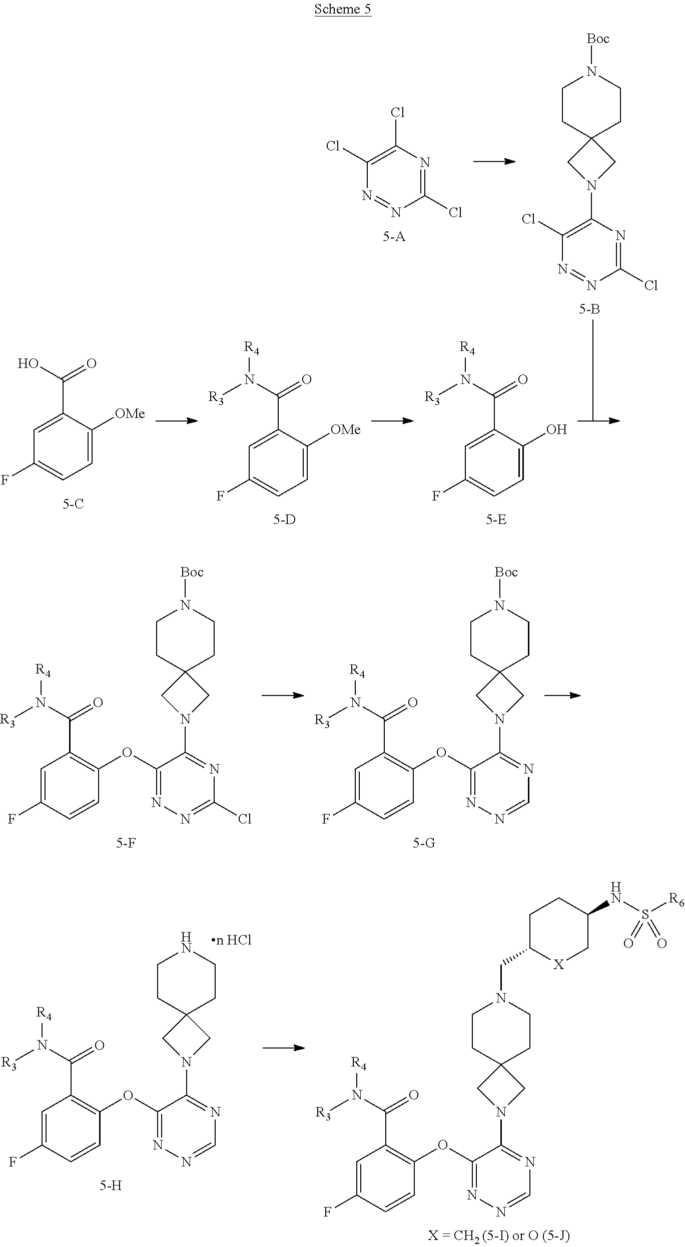

Scheme 5 illustrates the general methods for the preparation of triazine analogs like 5-I and 5-J. Under suitable conditions, 3,5,6-trichloro-1,2,4-triazine (5-A) can be reacted with various amines to afford the product resulting from displacement of the chloro-group at the 5-position. For example, Compound 5-A can be reacted with tert-butyl 2,7-diazaspiro[3.5]nonane-7-10 carboxylate to afford Compound 5-B. Typically, this reaction is conducted in the presence of a suitable base, such as Et₃N or (i-Pr)₂NEt, and in a neutral solvent like CH₂Cl₂, THF or NMP. Separately, a suitable benzoic acid derivative, for example 5-fluoro-2-methoxybenzoic acid (5-C), is converted to an amide (5-D) according to the general conditions for amide formation described previously. The methoxy group of 5-D can then be converted to the corresponding phenol under a variety of conditions, as described in established reference volumes, such as Greene's Protective Groups in Organic Synthesis, 4^{th} Edition (ISBN: 9780470053485). For example, reaction of 5-D with boron tribromide (BBr₃), in a neutral solvent, typically CH₂Cl₂, affords phenol 5-E. Those of skill in the art, who will be familiar with the use of protecting groups in organic synthesis, will recognize that a range of protecting groups might be used to protect the phenol during the previous amide-forming reaction (i.e., 5-C to 5-D), and that selection of the proper protecting group will depend on the nature of the amide-forming reaction and the substituents on the amide nitrogen. Under suitable conditions, phenol 5-E can react with Compound 5-B to afford Compound 5-F, via displacement of the chloro-group at the 6-position of 5-E. This reaction is conducted in the presence of a suitable base, such as DBU (1,8-diazabicyclo[5.4.0]undec-7-ene) or 1,1,3,3-tetramethylguanidine, in a neutral solvent, such as THF or CPME (cyclopentyl methyl ether). Those of skill in the art will recognize that competing displacement of the chloro-group at the 3-position might also occur, to varying degrees, during this reaction. As desired, the chloro-group at the 3-position of Compound 5-F can be retained, displaced by a nucleophile, or removed to afford Compound 5-G. Removal of the chloro-group from Compound 5-F is oftentimes achieved by reaction with hydrogen gas in the presence of a catalyst like Pd—C, Pd(OH)₂/C, Raney Nickel, Ru/C or Ir/C, in a suitable solvent, usually methanol, ethanol, 2,2,2-trifluoroethanol, ethyl acetate, or a combination thereof. The dechlorination reaction might also be accomplished using a metal hydride like NaBH₄, LiBH₄ or 9-BBN, generally in the presence of a palladium catalyst, like Pd(dppf) Cl₂ or Pd(OAc)₂. As described in detail in Scheme 1, the Boc protecting group in 5-G can be removed under a variety of acidic conditions, to afford Compound 5-H. Of course, if a different nitrogen protecting group is used, it is removed according to the nature of the protecting group. Also as described in Scheme 1, the deprotected amine can be isolated as the salt or as the free base. Compound 5-H can then be converted to Compounds 5-I and 5-J according to the general alkylation conditions described in Scheme 4 above.

### General Biological Methods

### Menin-MLL Competition and MV4; 11 Cell Proliferation Assays

Menin-MLL is a competition assay between human Menin and N-terminal portion of human MLL representing amino acids 4-43 of the protein. The interaction between Menin and MLL peptide was monitored by HTRF employing Terbium labeled anti-His6 antibody directed to the N-terminal His6-tag on recombinant Menin and FITC group covalently attached to the MLL peptide. The N-terminal fragment of MLL, retained in all MLL fusion proteins, is involved in the interactions with Menin, and this protein-protein interaction is critical for the MLL fusion proteins mediated leukemogenic transformations.

For IC₅₀ determination test compounds may be prepared as stock solutions. Lower sub-stocks of 50 μM may be prepared from the 10 mM stock solution. To test the compounds in assay, 3.16-fold serial dilutions are made in 100% DMSO. Mid-stock of 50× compounds (50 μM) were serially diluted (3.16 fold) in 100% DMSO in Polypropylene plate. In assay plate 1 micro-litre of the previously prepared compound dilution was stamped. H-FL-Menin diluted to 4 nM in assay buffer (50 mM Tris-HCl, pH 7.4, 50 mM NaCl, freshly prepared 1 mM DTT, 0.01% BSA, 0.005% Triton X-100) was pre-incubated with 8 nM anti-His6-Tb for 30 min at room temperature. FITC-MLL-4-43 was diluted to 2 nM in assay buffer and 25 μL was dispensed into each well of the assay plate followed by addition of 25 μl of pre-incubated H-FL-Menin and anti-His6-Tb mixture. Final concentration H-FL-Menin diluted to 1 nM in assay plate with 2 nM anti-His6-Tb and 1 nM FITC-MLL-4-43. After 1 hr incubation at room temperature, the HTRF signal was measured on the Spark multi-label plate reader. Resulting data were captured as a ratio of RFU520/RFU485×1000. The max values were obtained from 0% inhibition in presence of 2% DMSO and the min. values were 100% inhibition in presence of 1 μM reference compound.

### Cell Proliferation Assay-MV4;11

Compounds were evaluated for its capacity to inhibit the proliferation of the MLLr leukemia cell line MV4-11 that harbors an MLL1-AF4 fusion protein. MV4-11 cells were cultured for 72 hours with limiting dilutions of compounds and viability was measured using CellTiter-Glo.

Compounds were dissolved to obtain as 10 mM solution in DMSO. The stock was diluted 1:5 to the top concentration of 2 mM in 100% DMSO. For IC₅₀ determination, serial 1:3.16 dilutions were prepared in 100% DMSO by diluting 20 μL into 43.5 μL of DMSO for 8 concentrations. Each prepared DMSO solution were further diluted 1:500 in the cell culture media to obtain the 2× dosing solutions. The final concentrations of tested compounds in the cell culture media ranged from 0.632 nM to 2000 nM.

MV4-11 cells were cultured in IMDM with 10% FBS and 1× Penicillin-Streptomycin at 5% CO₂ and 37° C. A cell suspension was prepared containing 15,000cells/ml in the culture medium and 100 μL of this suspension was added per well to a 96-well cell culture plate. Then, 100 μL of 2× dosing media containing test compounds were added bringing the total volume to 200 μL. These cells were cultured for 72 hours at 37° C. and 5% CO₂ in a humidified incubator.

After 72 hours, the cultured cells were mixed and 100 μL was transferred to a 96-well black plate. Cell Titer Glo (100 μl) was then added to this plate. The plate was mixed with shaking for 15 mins at RT, the luminescence was then measured using Tecan Spark 20M spectrophotometer. Cell Viability (%) was determined by RLU of test/RLU average vehicle control*100 and % max inhibition was determined by 100-(% cell viability remaining at the top concentration of compound).

hERG Patch Clamp Assay

The hERG inhibition assay uses a high throughput single cell planar patch clamp approach. Chinese hamster ovary cells transfected with the hERG gene (CHO-hERG) are dispensed into the PatchPlate. Amphotericin is used as a perforating agent to gain electrical access to the cells. The hERG tail current is measured prior to the addition of the test compound by perforated patch clamping. Following addition of the test compound at a defined concentration or range of concentrations a second recording of the hERG current is performed. The degree of inhibition (%) is obtained by measuring the tail current amplitude, which is induced by a one second test pulse to −40 mV after a two second pulse to +20 mV, before and after drug incubation (the difference current is normalized to control and multiplied by 100 to obtain the percent of inhibition). The patch clamp assay can be used according to the knowledge of a person of ordinary skill in the art to accordingly assess the compounds of the present disclosure. In any one of the embodiments described herein, the compound has an IC₅₀ of more than 10, 15, 20, 25, or 30 μM in a standard human ether-a-go-go related gene (hERG) patch clamp assay.

A number of drugs have been withdrawn from late-stage clinical trials due to cardiotoxic effects, therefore it is important to identify and avoid compounds with potential for cardiotoxic effects early in drug discovery. The cardiovascular toxicity of a compound can be measured using a standard human ether-a-go-go related gene (hERG) assay. The human ether-a-go-go related gene (hERG) encodes the inward rectifying voltage gated potassium channel in the heart (IKr), which is involved in cardiac repolarization. Inhibition of the hERG current causes QT interval prolongation resulting in potentially fatal ventricular tachyarrhythmia called Torsade de Pointes. A compound having an IC₅₀ of more than about 10 μM or more than about 15 μM, in the hERG assay may be considered as free from any cardiovascular toxicity. In some embodiments, the compounds of Formulae I, II, III, III-a, III-b, III-c, III-b-1, III-b-2, III-b-3, III-c-1, III-c-2, and III-c-3, and/or Table 1 have reduced hERG binding compared to structural analogs. In some embodiments, the compounds of Formulae I, II, III, III-a, III-b, III-c, III-b-1, III-b-2, III-b-3, III-c-1, III-c-2, and III-c-3, and/or Table 1 have IC₅₀ of more than 10 μM, 15 μM, 20 μM, 25 μM, or 30 μM in the standard patch clamp hERG assay.

In some embodiments, the compounds of the present disclosure (e.g., Formulae I, II, III, III-a, III-b, III-c, III-b-1, III-b-2, III-b-3, III-c-1, III-c-2, and III-c-3, and Table 1) do not significantly block the hERG potassium channel (e.g., an IC₅₀ greater than 1 μM, 5 M, 10 μM, 15 μM, 20 μM, 25 μM, 30 μM, 35 μM, 40 μM, or 50 μM) in the standard patch clamp hERG assay.

### Methods of Use

The compounds of the invention are inhibitors of the interaction of menin with MLL and MLL fusion proteins. In some embodiments, the present disclosure is directed to a method of inhibiting the interaction between menin and MLL or an MLL fusion protein by contacting menin and MLL or the MLL fusion protein with a compound of the disclosure. The contacting can be carried out in vitro or in vivo. In some embodiments, the compounds of the disclosure can bind to menin, thereby interfering with the binding of MLL to menin. In some embodiments, the present disclosure provides a method of inhibiting the activity of menin by contacting menin with a compound of the disclosure in the presence of MLL or an MLL fusion protein. In further embodiments, the present disclosure provides a method of inhibiting the binding of MLL or an MLL fusion protein to menin, comprising contacting menin with a compound of the disclosure in the presence of the MLL or MLL fusion protein.

In some embodiments, compounds of the present disclosure minimize hERG interactions. In some embodiments, the present disclosure is directed to a method of inhibiting the interaction between menin and MLL or an MLL fusion protein by contacting menin and MLL or the MLL fusion protein with a compound of the disclosure while the compounds of the disclosure minimize hERG activity. In some embodiments, the present disclosure is directed to a method of inhibiting the interaction between menin and MLL or an MLL fusion protein by contacting menin and MLL or the MLL fusion protein with a compound of the disclosure while the compound of the disclosure avoids drug-induced blockade of hERG.

Evaluating the hERG activity can be accomplished by many methods known in the art. Including, such methods for the assessment of hERG liability is the patch-clamp electrophysiological assay on hERG transfected cells. Various other strategies including radiolabeled binding assays, functional assays, and rubidium efflux assays also quantify hERG potency.

The compounds of the disclosure are also useful in treating diseases associated with the menin-MLL interaction or menin-MLL fusion protein interaction. For example, diseases and conditions treatable according to the methods of the disclosure include cancer, such as leukemia, and other diseases or disorders mediated by the menin-MLL interaction or menin-MLL fusion protein interaction such as diabetes.

Accordingly, the compounds of the disclosure are believed to be effective against a broad range of cancers, including, but not limited to, hematological cancer (e.g., leukemia and lymphoma), bladder cancer, brain cancer (e.g., glioma, diffuse intrinsic pontine glioma (DIPG)), breast cancer (e.g., triple-negative breast cancer, estrogen-receptor -positive breast cancer (i.e., ER+ breast cancer)), colorectal cancer, cervical cancer, gastrointestinal cancer (e.g., colorectal carcinoma, gastric cancer), genitourinary cancer, head and neck cancer, liver cancer, lung cancer, melanoma, ovarian cancer, pancreatic cancer, prostate cancer (e.g., castration resistant prostate cancer), renal cancer (e.g., renal cell carcinoma), skin cancer, thyroid cancer (e.g., papillary thyroid carcinoma), testicular cancer, sarcoma (e.g., Ewing's sarcoma), and AIDS-related cancers. In some embodiments, the cancer is associated with a rearranged MLL gene. In some embodiments, the pathophysiology of the cancer is dependent on the MLL gene. In some embodiments, the cancer is associated with mutant p53 gain-of-function.

In some embodiments, the specific cancers that may be treated by the compounds, compositions and methods described herein include cardiac cancers, such as for example, sarcoma (e.g., angiosarcoma, fibrosarcoma, rhabdomyosarcoma, and liposarcoma), myxoma, rhabdomyoma, fibroma, lipoma and teratoma; lung cancers, including, for example, bronchogenic carcinoma (e.g., squamous cell, undifferentiated small cell, undifferentiated large cell, and adenocarcinoma), alveolar and bronchiolar carcinoma, bronchial adenoma, sarcoma, lymphoma, chondromatous hamartoma, mesothelioma, non-small cell lung cancer, small cell lung cancer, bronchial adenomas/carcinoids, and pleuropulmonary blastoma; gastrointestinal cancer, including, for example, cancers of the esophagus (e.g., squamous cell carcinoma, adenocarcinoma, leiomyosarcoma, and lymphoma), cancers of the stomach (e.g., carcinoma, lymphoma, and leiomyosarcoma), cancers of the pancreas (e.g., ductal adenocarcinoma, insulinoma, glucagonoma, gastrinoma, carcinoid tumors, and vipoma), cancers of the small bowel (e.g., adenocarcinoma, lymphoma, carcinoid tumors, Kaposi's sarcoma, leiomyoma, hemangioma, lipoma, neurofibroma, and fibroma), cancers of the large bowel or colon, (e.g., adenocarcinoma, tubular adenoma, villous adenoma, hamartoma, and leiomyoma), and other cancers of the digestive tract (e.g., anal cancer, anorectal cancer, appendix cancer, cancer of the anal canal, cancer of the tongue, gallbladder cancer, gastrointestinal stromal tumor (GIST), colon cancer, colorectal cancer, extrahepatic bile duct cancer, intrahepatic bile duct cancer, rectal cancer, and small intestine cancer); genitourinary tract cancers, including, for example, cancers of the kidney (e.g., adenocarcinoma, Wilm's tumor (nephroblastoma), lymphoma, and leukemia), cancers of the bladder and urethra (e.g., squamous cell carcinoma, transitional cell carcinoma, and adenocarcinoma), cancers of the prostate (e.g., adenocarcinoma and sarcoma), cancers of the testis, (e.g., seminoma, teratoma, embryonal carcinoma, teratocarcinoma, choriocarcinoma, sarcoma, interstitial cell carcinoma, fibroma, fibroadenoma, adenomatoid tumors, and lipoma), as well as transitional cell cancer, transitional cell cancer of the renal pelvis and ureter and other urinary organs, urethral cancer, and urinary bladder cancer; liver cancers, including, for example, hepatoma (e.g., hepatocellular carcinoma), cholangiocarcinoma, hepatoblastoma, angiosarcoma, hepatocellular adenoma, and hemangioma; bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell chordoma, osteochrondroma (osteocartilaginous exostoses), benign chondroma, tumor chondroblastoma, chondromyxofibroma, osteoid osteoma and giant cell tumors; nervous system cancers, including, for example, cancers of the skull (e.g., osteoma, hemangioma, granuloma, xanthoma, and osteitis deformans); cancers of the meninges (e.g., meningioma, meningiosarcoma, and gliomatosis); cancers of the brain (e.g., astrocytoma, medulloblastoma, glioma, ependymoma, germinoma (pinealoma), glioblastoma multiforme, oligodendroglioma, schwannoma, retinoblastoma, and congenital tumors); cancers of the spinal cord (e.g., neurofibroma, meningioma, glioma, and sarcoma), and other nervous system cancers (e.g., brain stem glioma, diffuse intrinsic pontine glioma (DIPG), brain tumor, central nervous system cancer, cerebellar astrocytoma, cerebral astrocytoma/malignant glioma, childhood cerebellar astrocytoma, childhood cerebral astrocytoma, primary central nervous system lymphoma, visual pathway and hypothalamic glioma, nervous system lymphoma, supratentorial primitive neuroectodeimal tumors, pineoblastoma and supratentorial primitive neuroectodermal tumors); gynecological cancers, including, for example, cancers of the uterus (e.g., endometrial carcinoma), cancers of the cervix (e.g., cervical carcinoma, and pre tumor cervical dysplasia), cancers of the ovaries (e.g., ovarian carcinoma, including serous cystadenocarcinoma, mucinous cystadenocarcinoma, unclassified carcinoma, granulosa thecal cell tumors, Sertoli Leydig cell tumors, dysgerminoma, and malignant teratoma), cancers of the vulva (e.g., squamous cell carcinoma, intraepithelial carcinoma, adenocarcinoma, fibrosarcoma, and melanoma), cancers of the vagina (e.g., clear cell carcinoma, squamous cell carcinoma, botryoid sarcoma, and embryonal rhabdomyosarcoma), and cancers of the fallopian tubes (e.g., carcinoma); other reproductive tract cancers, including, for example, endometrial cancer, endometrial uterine cancer, germ cell tumor, gestational trophoblastic tumor, gestational trophoblastic tumor glioma, ovarian epithelial cancer, ovarian germ cell tumor, ovarian low malignant potential tumor, penile cancer, vaginal cancer, vulvar cancer, extracranial germ cell tumor, extragonadal germ cell tumor, uterine cancer, uterine corpus cancer, uterine sarcoma; lymphatic and hematologic cancers, including, for example, cancers of the blood (e.g., acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, and myelodysplastic syndrome, Hodgkin's lymphoma, non Hodgkin's lymphoma (malignant lymphoma) and Waldenstrom's macroglobulinemia), and other lymphatic or hematologic cancers including, for example, childhood leukemia, myeloproliferative disorders (e.g., primary myelofibrosis), plasma cell neoplasm/multiple myeloma, myelodysplasia, myelodysplastic syndrome, cutaneous T-cell lymphoma, lymphoid neoplasm, AIDS-related lymphoma, thymoma, thymoma and thymic carcinoma, mycosis fungoides, and Sézary Syndrome; skin cancers, including, for example, malignant melanoma, basal cell carcinoma, squamous cell carcinoma, Kaposi's sarcoma, moles dysplastic nevi, lipoma, angioma, dermatofibroma, keloids, psoriasis, merkel cell carcinoma, merkel cell skin carcinoma, melanoma, and carcinoid tumor; adrenal gland cancers, including, for example, neuroblastoma; other cancers associated with the endocrine system including, for example, adrenocortical carcinoma, multiple endocrine neoplasia (e.g., multiple endocrine neoplasia type I), multiple endocrine neoplasia syndrome, parathyroid cancer, pituitary tumor, pheochromocytoma, islet cell pancreatic cancer, and islet cell tumors); connective tissue cancer (e.g., bone cancer, bone and joint cancer, osteosarcoma and malignant fibrous histiocytoma); cancer associated with the head, neck, and mouth (e.g., head and neck cancer, paranasal sinus and nasal cavity cancer, metastatic squamous neck cancer, mouth cancer, throat cancer, esophageal cancer, laryngeal cancer, pharyngeal cancer, hypopharyngeal cancer, lip and oral cavity cancer, nasopharyngeal cancer, oral cancer, oropharyngeal cancer, and salivary gland cancer); and cancer associated with the eye (e.g., ocular cancer, intraocular melanoma). In some embodiments, the cancer is Ewing's sarcoma.

In some embodiments, the cancer is a hematological cancer such as leukemia or lymphoma. Example leukemia and lymphomas treatable by the compounds of the disclosure include mixed lineage leukemia (MLL), MLL-related leukemia, MLL-associated leukemia, MLL-positive leukemia, MLL-induced leukemia, rearranged mixed lineage leukemia (MLL-r), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL) (also referred to as acute lymphoblastic leukemia or acute lymphoid leukemia), acute myeloid leukemia (AML) (also referred to as acute myelogenous leukemia or acute myeloblastic leukemia), acute granulocytic leukemia, acute nonlymphocytic leukemia, chronic lymphocytic leukemia (CLL) (also referred to as chronic lymphoblastic leukemia), chronic myelogenous leukemia (CML) (also referred to as chronic myeloid leukemia), therapy related leukemia, myelodysplastic syndrome (MDS), myeloproliferative disease (MPD) (such as primary myelofibrosis (PMF)), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sézary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non-Hodgkin's lymphoma (malignant lymphoma), and Waldenstrom's macroglobulinemia. In some embodiments, the acute myeloid leukemia (AML) is abstract nucleophosmin (NPM1)-mutated acute myeloid leukemia (i.e., NPM1mut acute myloid leukemia).

In particular embodiments, compounds of the disclosure are used to treat a rearranged mixed lineage (KMT2A-rearranged) leukemia (MLL-r).

In particular embodiments, compounds of the disclosure are used to treat leukemia associated with a MLL rearrangement, acute lymphocytic leukemia associated with a MLL rearrangement, acute lymphoblastic leukemia associated with a MLL rearrangement, acute lymphoid leukemia associated with a MLL rearrangement, acute myeloid leukemia associated with a MLL rearrangement, acute myelogenous leukemia associated with a MLL rearrangement, or acute myeloblastic leukemia associated with a MLL rearrangement. As used herein, “MLL rearrangement” means a rearrangement of the MLL gene.

In some embodiments, diseases and conditions treatable with compounds of the disclosure include insulin resistance, pre-diabetes, diabetes (e.g., Type 2 diabetes or Type 1 diabetes), and risk of diabetes. In some embodiments, diseases and conditions treatable with compounds of the disclosure include hyperglycemia. In some embodiments, the hyperglycemia is associated with diabetes, such as Type 2 diabetes. In some embodiments, compounds of the disclosure are used to treat loss of response to other anti-diabetic agents and/or reduced beta cell function in a patient or subject. In some embodiments, compounds of the disclosure are used to restore response to other anti-diabetic agents and/or to restore beta cell function and/or to reduce the need for insulin in a patient or subject. In some embodiments, compounds of the disclosure are used to reduce insulin resistance, reduce the risk of diabetes, or reduce increases in blood glucose caused by a statin in a subject taking a statin. In some embodiments, compounds of the disclosure are used to treat diabetes in a subject taking a statin or to prevent diabetes in a subject taking a statin. Methods of the disclosure include decreasing, reducing, inhibiting, suppressing, limiting or controlling in the patient elevated blood glucose levels. In further aspects, methods of the disclosure include increasing, stimulating, enhancing, promoting, inducing or activating in the subject insulin sensitivity. Statins include, but are not limited to atorvastatin, cerivastatin, fluvastatin, lovastatin, mevastatin, pitavastatin, pravastatin, rousuvastatin and simvastatin.

In some embodiments, a patient is treated with (e.g., administered) a compound of the present disclosure in an amount sufficient to treat or ameliorate one or more of the diseases and conditions recited above (e.g., a therapeutically effective amount). The compounds of the disclosure may also be useful in the prevention of one or more of the diseases recited therein.

In some embodiments, a compound of the present disclosure or a pharmaceutically acceptable salt thereof, or a pharmaceutical composition of the present disclosure is used in treating or preventing a disease caused by, or associated with, menin expression, activity, and/or function.

In some embodiments, a compound of the present disclosure or a pharmaceutically acceptable salt thereof, or a pharmaceutical composition of the present disclosure is used in treating or preventing cancer.

In some embodiments, a compound of the present disclosure or a pharmaceutically acceptable salt thereof, or a pharmaceutical composition of the present disclosure is used for treating or preventing a disease caused by, or associated with, menin expression, activity, and/or function.

In some embodiments, a compound of the present disclosure or a pharmaceutically acceptable salt thereof, or a pharmaceutical composition of the present disclosure is used in the manufacture of a medicament for treating or preventing a disease caused by, or associated with, menin expression, activity, and/or function.

In some embodiments, the present disclosure relates to a kit comprising a compound of the present disclosure or a pharmaceutically acceptable salt thereof, or a pharmaceutical composition of the present disclosure and instructions for its use.

In some embodiments, a compound of the present disclosure or a pharmaceutically acceptable salt thereof, or a pharmaceutical composition of the present disclosure is used for treating hematological malignancies. In some embodiments, a compound of the present disclosure or a pharmaceutically acceptable salt thereof, or a pharmaceutical composition of the present disclosure is used for treating hematological malignancies as described in WO2023150635A1, which disclosure is incorporated by reference herein.

In some embodiments, a compound of the present disclosure or a pharmaceutically acceptable salt thereof, or a pharmaceutical composition of the present disclosure is used for treating hematologic cancer with high interferon regulatory factor 8 (IRF8) expression. In some embodiments, a compound of the present disclosure or a pharmaceutically acceptable salt thereof, or a pharmaceutical composition of the present disclosure is used for treating hematologic cancer with high interferon regulatory factor 8 (IRF8) expression as described in WO2025015152A1, which disclosure is incorporated by reference herein.

In some embodiments, a compound of the present disclosure or a pharmaceutically acceptable salt thereof, or a pharmaceutical composition of the present disclosure is used for treating PIK3CA mutation colon cancer. In some embodiments, a compound of the present disclosure or a pharmaceutically acceptable salt thereof, or a pharmaceutical composition of the present disclosure is used for treating PIK3CA mutation colon cancer as described in CN Patent No. 118021815B, which disclosure is incorporated by reference herein.

In some embodiments, a compound of the present disclosure or a pharmaceutically acceptable salt thereof, or a pharmaceutical composition of the present disclosure is used for treating sickle cell disease. In some embodiments, a compound of the present disclosure or a pharmaceutically acceptable salt thereof, or a pharmaceutical composition of the present disclosure is used for treating sickle cell disease as described in U.S. Patent Publication No. 20240100049A1, which disclosure is incorporated by reference herein.

### Combination Therapy

The disclosure further relates to a combination therapy for treating a disease or a disorder described herein. In some embodiments, the combination therapy comprises administering at least one compound of the present disclosure in combination with one or more other pharmaceutically active agents for treating cancer or other disorders mediated by menin/MLL. In some embodiments, the combination therapy comprises administering at least one compound of the present disclosure in combination with one or more other pharmaceutically active agents, such as for the treatment of cancer. The pharmaceutically active agents can be combined with a compound of the disclosure in a single dosage form, or the therapeutics can be administered simultaneously or sequentially as separate dosage forms.

In some embodiments, the invention provides a combination therapy comprising a menin inhibitor of the present disclosure (e.g., a compound of Formula I, Formula II, Formula III, Formula III-a, Formula III-b, Formula III-c, Formula III-b-1, Formula III-b-2, Formula III-b-3, Formula III-c-1, Formula III-c-2, Formula III-c-3, etc.) and a CYP3A4 inhibitor. In certain embodiments, the invention provides for a pharmaceutical composition comprising: (a) a menin inhibitor of the present disclosure (e.g., a compound of Formula I, Formula II, Formula III, Formula III-a, Formula III-b, Formula III-c, Formula III-b-1, Formula III-b-2, Formula III-b-3, Formula III-c-1, Formula III-c-2, Formula III-c-3, etc.), and (b) a CYP3A4 inhibitor. In some embodiments, the invention is directed to a method for treating a patient comprising (a) administering a menin inhibitor of the present disclosure (e.g., a compound of Formula I, Formula II, Formula III, Formula III-a, Formula III-b, Formula III-c, Formula III-b-1, Formula III-b-2, Formula III-b-3, Formula III-c-1, Formula III-c-2, Formula III-c-3, etc.), and (b) administering a CYP3A4 inhibitor.

Some embodiments of this invention are directed to combination therapies designed to treat or manage cancer in a subject, wherein the combination therapies comprise administering a menin inhibitor of the present disclosure (e.g., a compound of Formula I, Formula II, Formula III, Formula III-a, Formula III-b, Formula III-c, Formula III-b-1, Formula III-b-2, Formula III-b-3, Formula III-c-1, Formula III-c-2, Formula III-c-3, etc.) in combination with a CYP3A4 inhibitor. In particular, some embodiments of this invention are directed to methods of treating or managing cancer in a subject, comprising administering a menin inhibitor in combination with a therapeutically effective amount of a CYP3A4 inhibitor administered simultaneously, separately or sequentially.

In some embodiments, the CYP3A inhibitor is: an antiarrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof. In some embodiments, the CYP3A inhibitor is: posaconazole, alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3—OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3—OH; ritonavir; saquinavir; sildenafil; simvastatin; starfruit; tacrolimus (FK506); tamoxifen; telaprevir; telithromycin; trazodone; triazolam; verapamil; telaprevir; vincristine; voriconazole; or any combinations thereof.

In some embodiments, the CYP3A4 inhibitor is posaconazole, cobicistat (GS-9350) or analogs or derivatives of cobicistat (GS-9350). In some embodiments, the CYP3A4 inhibitor is ketoconazole. In some embodiments, the CYP3A4 inhibitor is ritonavir. In some embodiments, the menin inhibitor and the CYP3A4 inhibitor are in separate dosage forms. In some embodiments, the pharmaceutical composition is in a combined dosage form. In some embodiments, the CYP3A4 inhibitor is posaconazole.

In some embodiments, the pharmaceutical composition comprises an amount of the CYP3A4 inhibitor that is effective to increase the oral bioavailability of the menin inhibitor. The compounds according to the disclosure may also be used in combination with immunotherapies, including but not limited to cell-based therapies, antibody therapies and cytokine therapies, for the treatment of a disease or disorder disclosed herein.

In certain embodiments, compounds according to the disclosure are used in combination with one or more passive immunotherapies, including but not limited to naked monoclonal antibody drugs and conjugated monoclonal antibody drugs. Examples of naked monoclonal antibody drugs that can be used include, but are not limited to, rituximab (Rituxan®), an antibody against the CD20 antigen; trastuzumab (Herceptin®), an antibody against the HER2 protein; alemtuzumab (Lemtrada®, Campath®), an antibody against the CD52 antigen; cetuximab (Erbitux®), an antibody against the EGFR protein; and bevacizumab (Avastin®) which is an anti-angiogenesis inhibitor of VEGF protein.

Examples of conjugated monoclonal antibodies that can be used include, but are not limited to, radiolabeled antibody ibritumomab tiuxetan (Zevalin®); radiolabeled antibody tositumomab (Bexxar®); and immunotoxin gemtuzumab ozogamicin (Mylotarg®) which contains calicheamicin; BL22, an anti-CD22 monoclonal antibody-immunotoxin conjugate; radiolabeled antibodies such as OncoScint® and ProstaScint®; brentuximab vedotin (Adcetris®); ado-trastuzumab emtansine (Kadcyla®, also called TDM-1).

Further examples of therapeutic antibodies that can be used include, but are not limited to, REOPRO® (abciximab), an antibody against the glycoprotein IIb/IIIa receptor on platelets; ZENAPAX® (daclizumab) an immunosuppressive, humanized anti-CD25 monoclonal antibody; PANOREX™, a murine anti-17-IA cell surface antigen IgG2a antibody; BEC2, a murine anti-idiotype (GD3 epitope) IgG antibody; IMC-C225, a chimeric anti-EGFR IgG antibody; VITAXIN™ a humanized anti-αVβ3 integrin antibody; Campath 1H/LDP-03, a humanized anti CD52 IgG1 antibody; Smart M195, a humanized anti-CD33 IgG antibody; LYMPHOCIDE™, a humanized anti-CD22 IgG antibody; LYMPHOCIDE™ Y-90; Lymphoscan; Nuvion® (against CD3; CM3, a humanized anti-ICAM3 antibody; IDEC-114 a primatized anti-CD80 antibody; IDEC-131 a humanized anti-CD40L antibody; IDEC-151 a primatized anti-CD4 antibody; IDEC-152 a primatized anti-CD23 antibody; SMART anti-CD3, a humanized anti-CD3 IgG; 5G1.1, a humanized anti-complement factor 5 (C5) antibody; D2E7, a humanized anti-TNF-α antibody; CDP870, a humanized anti-TNF-α Fab fragment; IDEC-151, a primatized anti-CD4 IgG1 antibody; MDX-CD4, a human anti-CD4 IgG antibody; CD20-streptdavidin (+biotin-yttrium 90); CDP571, a humanized anti-TNF-α IgG4 antibody; LDP-02, a humanized anti-α4ß7 antibody; OrthoClone OKT4A, a humanized anti-CD4 IgG antibody; ANTOVA™, a humanized anti-CD40L IgG antibody; ANTEGREN™, a humanized anti-VLA-4 IgG antibody; and CAT-152, a human anti-TGF-β2 antibody.

In certain embodiments, compounds according to the disclosure are used in combination with one or more targeted immunotherapies containing toxins but not an antibody, including but not limited to denileukin diftitox (Ontak®), IL-2 linked to diphtheria toxin.

The compounds according to the disclosure may also be used in combination with adjuvant immunotherapies for the treatment of a disease or disorder disclosed herein. Such adjuvant immunotherapies include, but are not limited to, cytokines, such as granulocyte-macrophage colony-stimulating factor (GM-CSF), granulocyte-colony stimulating factor (G-CSF), macrophage inflammatory protein (MIP)-1-alpha, interleukins (including IL-1, IL-2, IL-4, IL-6, IL-7, IL-12, IL-15, IL-18, IL-21, and IL-27), tumor necrosis factors (including TNF-alpha), and interferons (including IFN-alpha, IFN-beta, and IFN-gamma); aluminum hydroxide (alum); Bacille Calmette-Guérin (BCG); Keyhole limpet hemocyanin (KLH); Incomplete Freund's adjuvant (IFA); QS-21; DETOX; Levamisole; and Dinitrophenyl (DNP), and combinations thereof, such as, for example, combinations of interleukins, for example IL-2, with other cytokines, such as IFN-alpha.

In certain embodiments, compounds according to the disclosure are used in combination with vaccine therapy, including but not limited to autologous and allogeneic tumor cell vaccines, antigen vaccines (including polyvalent antigen vaccines), dendritic cell vaccines, and viral vaccines.

In another embodiment, the present disclosure comprises administering to a subject with cancer an effective amount of a compound of the disclosure and one or more additional anti-cancer therapies selected from: surgery, anti-cancer agents/drugs, biological therapy, radiation therapy, anti-angiogenesis therapy, immunotherapy, adoptive transfer of effector cells, gene therapy or hormonal therapy. Examples of anti-cancer agents/drugs are described below.

In some embodiments, the anti-cancer agents/drug is, for example, adriamycin, aactinomycin, bleomycin, vinblastine, cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; palbociclib; Yervoy® (ipilimumab); Mekinist™ (trametinib); peginterferon alfa-2b, recombinant interferon alfa-2b; Sylatron™ (peginterferon alfa-2b); Tafinlar® (dabrafenib); Zelboraf® (vemurafenib); or nivolumab.

The compounds according to the present disclosure can be administered in combination with existing methods of treating cancers, for example by chemotherapy, irradiation, or surgery. Thus, there is further provided a method of treating cancer comprising administering an effective amount of a compound of the disclosure, or a pharmaceutically acceptable salt form thereof, to a subject in need of such treatment, wherein an effective amount of at least one additional cancer chemotherapeutic agent is administered to the subject. Examples of suitable cancer chemotherapeutic agents include any of: abarelix, ado-trastuzumab emtansine, aldesleukin, alemtuzumab, alitretinoin, allopurinol, altretamine, anastrozole, arsenic trioxide, asparaginase, azacitidine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, emtansine, epirubicin, eribulin, erlotinib, estramustine, etoposide phosphate, etoposide, everolimus, exemestane, fentanyl citrate, filgrastim, floxuridine, fludarabine, fluorouracil, fruquintinib, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, ixabepilone, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, paclitaxel albumin-stabilized nanoparticle formulation, pamidronate, panitumumab, pegaspargase, pegfilgrastim, pemetrexed disodium, pentostatin, pertuzuma, pipobroman, plicamycin, procarbazine, quinacrine, rasburicase, rituximab, sorafenib, streptozocin, sulfatinib, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, volitinib, vorinostat, and zoledronate.

In particular embodiments, compounds according to the disclosure are used in combination with one or more anti-cancer agent selected from methotrexate, paclitaxel albumin-stabilized nanoparticle formulation, ado-trastuzumab emtansine, eribulin, doxorubicin, fluorouracil, everolimus, anastrozole, pamidronate disodium, exemestane, capecitabine, cyclophosphamide, docetaxel, epirubicin, toremifene, fulvestrant, letrozole, gemcitabine, gemcitabine hydrochloride, goserelin acetate, trastuzumab, ixabepilone, lapatinib ditosylate, megestrol acetate, tamoxifen citrate, pamidronate disodium, palbociclib, and pertuzumab for the treatment of breast cancer.

Other anti-cancer agents/drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; andrographolide; angiogenesis inhibitors; antagonist D; antagonist G; antarelix; anti-dorsalizing morphogenetic protein-1; antiandrogen; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors; castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclin-dependent kinase inhibitors; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate;fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor -1 receptor inhibitor; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor -saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitors; microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; zanoterone; zilascorb; zinostatin stimalamer; 5-fluorouracil; and leucovorin.

In some embodiments, the anti-cancer agent/drug is an agent that stabilizes microtubules. As used herein, a “microtubulin stabilizer” means an anti-cancer agent/drug which acts by arresting cells in the G2-M phases due to stabilization of microtubules. Examples of microtubulin stabilizers include ACLITAXEL® and Taxol® analogues. Additional examples of microtubulin stabilizers include without limitation the following marketed drugs and drugs in development: Discodermolide (also known as NVP—XX-A-296); Epothilones (such as Epothilone A, Epothilone B, Epothilone C(also known as desoxyepothilone A or dEpoA); Epothilone D (also referred to as KOS-862, dEpoB, and desoxyepothilone B); Epothilone E; Epothilone F; Epothilone B N-oxide; Epothilone A N-oxide; 16-aza-epothilone B; 21-aminoepothilone B (also known as BMS-310705); 21-hydroxyepothilone D (also known as Desoxyepothilone F and dEpoF), 26-fluoroepothilone); FR-182877 (Fujisawa, also known as WS-9885B), BSF-223651 (BASF, also known as ILX-651 and LU-223651); AC-7739 (Ajinomoto, also known as AVE-8063A and CS-39.HCl); AC-7700 (Ajinomoto, also known as AVE-8062, AVE-8062A, CS-39-L-Ser.HCl, and RPR-258062A); Fijianolide B; Laulimalide; Caribaeoside; Caribaeolin; Taccalonolide; Eleutherobin; Sarcodictyin; Laulimalide; Dictyostatin-1; Jatrophane esters; and analogs and derivatives thereof.

In another embodiment, the anti-cancer agent/drug is an agent that inhibits microtubules. As used herein, a “microtubulin inhibitor” means an anti-cancer agent which acts by inhibiting tubulin polymerization or microtubule assembly. Examples of microtubulin inhibitors include without limitation the following marketed drugs and drugs in development: Erbulozole (also known as R-55104); Dolastatin 10 (also known as DLS-10 and NSC-376128); Mivobulin isethionate (also known as CI-980); Vincristine; NSC-639829; ABT-751 (Abbott, also known as E-7010); Altorhyrtins (such as Altorhyrtin A and Altorhyrtin C); Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9); Cemadotin hydrochloride (also known as LU-103793 and NSC-D-669356); Auristatin PE (also known as NSC-654663); Soblidotin (also known as TZT-1027), LS-4559-P (Pharmacia, also known as LS-4577); LS-4578 (Pharmacia, also known as LS-477-P); LS-4477 (Pharmacia), LS-4559 (Pharmacia); RPR-112378 (Aventis); Vincristine sulfate; DZ-3358 (Daiichi); GS-164 (Takeda); GS-198 (Takeda); KAR-2 (Hungarian Academy of Sciences); SAH-49960 (Lilly/Novartis); SDZ-268970 (Lilly/Novartis); AM-97 (Armad/Kyowa Hakko); AM-132 (Armad); AM-138 (Armad/Kyowa Hakko); IDN-5005 (Indena); Cryptophycin 52 (also known as LY-355703); Vitilevuamide; Tubulysin A; Canadensol; Centaureidin (also known as NSC-106969); T-138067 (Tularik, also known as T-67, TL-138067 and TI-138067); COBRA-1 (Parker Hughes Institute, also known as DDE-261 and WHI-261); H10 (Kansas State University); H16 (Kansas State University); Oncocidin A1 (also known as BTO-956 and DIME); DDE-313 (Parker Hughes Institute); SPA-2 (Parker Hughes Institute); SPA-1 (Parker Hughes Institute, also known as SPIKET-P); 3-IAABU (Cytoskeleton/Mt. Sinai School of Medicine, also known as MF-569); Narcosine (also known as NSC-5366); Nascapine, D-24851 (Asta Medica), A-105972 (Abbott); Hemiasterlin; 3-BAABU (Cytoskeleton/Mt. Sinai School of Medicine, also known as MF-191); TMPN (Arizona State University); Vanadocene acetylacetonate; T-138026 (Tularik); Monsatrol; Inanocine (also known as NSC-698666); 3-IAABE (Cytoskeleton/Mt. Sinai School of Medicine); A-204197 (Abbott); T-607 (Tularik, also known as T-900607); RPR-115781 (Aventis); Eleutherobins (such as Desmethyleleutherobin, Desaetyleleutherobin, Isoeleutherobin A, and Z-Eleutherobin); Halichondrin B; D-64131 (Asta Medica); D-68144 (Asta Medica); Diazonamide A; A-293620 (Abbott); NPI-2350 (Nereus); TUB-245 (Aventis); A-259754 (Abbott); Diozostatin; (−)-Phenylahistin (also known as NSCL-96F037); D-68838 (Asta Medica); D-68836 (Asta Medica); Myoseverin B; D-43411 (Zentaris, also known as D-81862); A-289099 (Abbott); A-318315 (Abbott); HTI-286 (also known as SPA-110, trifluoroacetate salt) (Wyeth); D-82317 (Zentaris); D-82318 (Zentaris); SC-12983 (NCI); Resverastatin phosphate sodium; BPR—OY-007 (National Health Research Institutes); SSR-250411 (Sanofi); Combretastatin A4; eribulin (Halaven®), and analogs and derivatives thereof.

In further embodiments, compounds according to the disclosure are used in combination with one or more alkylating agents, antimetabolites, natural products, or hormones.

Examples of alkylating agents useful in the methods of the disclosure include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.).

Examples of antimetabolites useful in the methods of the disclosure include but are not limited to folic acid analog (e.g., methotrexate), or pyrimidine analogs (e.g., fluorouracil, floxouridine, cytarabine), and purine analogs (e.g., mercaptopurine, thioguanine, pentostatin). Examples of natural products useful in the methods of the disclosure include but are not limited to vinca alkaloids (e.g., vinblastin, vincristine), epipodophyllotoxins (e.g., etoposide, teniposide), antibiotics (e.g., actinomycin D, daunorubicin, doxorubicin, bleomycin, plicamycin, mitomycin) or enzymes (e.g., L-asparaginase).

Examples of hormones and antagonists useful for the treatment of cancer include but are not limited to adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), and gonadotropin releasing hormone analog (e.g., leuprolide).

Other agents that can be used in combination with the compounds of the disclosure for the treatment of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), and adrenocortical suppressant (e.g., mitotane, aminoglutethimide). Other anti-cancer agents/drugs that can be used in combination with the compounds of the disclosure include, but are not limited to, liver X receptor (LXR) modulators, including LXR agonists and LXR beta-selective agonists; aryl hydrocarbon receptor (AhR) inhibitors; inhibitors of the enzyme poly ADP ribose polymerase (PARP), including olaparib, iniparib, rucaparib, veliparib; inhibitors of vascular endothelial growth factor (VEGF) receptor tyrosine kinases, including cediranib; programmed cell death protein 1 (PD-1) inhibitors, including nivolumab (Bristol-Myers Squibb Co.) and pembrolizumab (Merck & Co., Inc.; MK-3475); MEK inhibitors, including cobimetinib; B-Raf enzyme inhibitors, including vemurafenib; cytotoxic T lymphocyte antigen (CTLA-4) inhibitors, including tremelimumab; programmed death-ligand 1 (PD-L1) inhibitors, including MEDI4736 (AstraZeneca); inhibitors of the Wnt pathway; inhibitors of epidermal growth factor receptor (EGFR) including AZD9291 (AstraZeneca), erlotinib, gefitinib, panitumumab, and cetuximab; adenosine A2A receptor inhibitors; adenosine A2B receptor inhibitors; colony-stimulating factor -1 receptor (CSF1R) inhibitors, including PLX3397 (Plexxikon), and inhibitors of CD73.

The compounds of the disclosure can be used in combination with one or more therapeutic strategies including immune checkpoint inhibitors, including inhibitors of PD-1, PD-L1, and CTLA-4.

The compounds of the disclosure can be used in combination with one or more anti-cancer agents selected from MCL-1 inhibitors, e.g., homoharringtonin (HHT) and omacetaxine; BCL-2 inhibitors, e.g., venetoclax (ABT-199), navitoclax (ABT-263), ABT-737, gossypol (AT-101), apogossypolone (ApoG2) and obatoclax; selective inhibitors of nuclear export (SINEs), e.g., selinexor (KPT-330).

In particular embodiments, the compounds of the disclosure are used in combination with one or more anti-cancer agents selected from methotrexate (Abitrexate®; Folex®; Folex PFS®, Mexate®; Mexate-AQ®); nelarabine (Arranon®); blinatumomab (Blincyto®); rubidomycin hydrochloride or daunorubicin hydrochloride (Cerubidine®); cyclophosphamide (Clafen®; Cytoxan®; Neosar®); clofarabine (Clofarex®; Clolar®); cytarabine (Cytosar-U®; Tarabine PFS®); dasatinib (Sprycel®); doxorubicin hydrochloride; asparaginase Erwinia chrysanthemi (Erwinaze); imatinib mesylate (Gleevec®); ponatinib hydrochloride (Iclusig®); mercaptopurine (Purinethol; Purixan); pegaspargase (Oncaspar®); prednisone; vincristine sulfate (Oncovin®, Vincasar PFS®, Vincrex®); vincristine sulfate liposome (Marqibo®); hyper-CVAD (fractionated cyclophosphamide, vincristine, adriamycin, and dexamethasone); arsenic trioxide (Trisenox®); idarubicin hydrochloride (Idamycin®); mitoxantrone hydrochloride; thioguanine (Tabloid®); ADE (cytarabine, daunorubicin, and etoposide); alemtuzumab (Lemtrada®, Campath®); chlorambucil (Ambochlorin®, Amboclorin®, Leukeran®, Linfolizin®); ofatumumab (Arzerra®); bendamustine hydrochloride (Treanda®); fludarabine phosphate (Fludara®); obinutuzumab (Gazyva®); ibrutinib (Imbruvica®); idelalisib (Zydelig®); mechlorethamine hydrochloride (Mustargen®); rituximab (Rituxan®); chlorambucil-prednisone; CVP (cyclophosphamide, vincristine, and prednisone); bosutinib (Bosulif®); busulfan (Busulfex®; Myleran®); omacetaxine mepesuccinate (Synribo®); nilotinib (Tasigna®); Intron® A (recombinant interferon Alfa-2b); DOTIL inhibitors, including EPZ-5676 (Epizyme, Inc.); and inhibitors of bromodomain and extra-terminal motif (BET) proteins (BET inhibitors), including MS417, JQ1, I-BET 762, and I-BET 151 for the treatment of leukemia.

Compounds of the disclosure can be used in combination with one or more other agents or therapies for the treatment of insulin resistance, pre-diabetes, diabetes (e.g., Type 2 diabetes or Type 1 diabetes), and risk of diabetes, including but not limited to insulins and insulin analogues, such as Humulin® (Eli Lilly), Lantus® (Sanofi Aventis), Novolin® (Novo Nordisk), and Exubera® (Pfizer); Avandamet® (metformin HCl and rosiglitazone maleate, GSK); Avandaryl® (glimepiride and rosiglitazone maleate, GSK); Metaglip® (glipizide and metformin HCl, Bristol Myers Squibb); Glucovance® (glyburide and metformin HCl, Bristol Myers Squibb); PPAR gamma agonists, such as Avandia® (rosiglitizone maleate, GSK) and Actos® (pioglitazone hydrochloride, Takeda/Eli Lilly); sulfonylureas, such as Amaryl® (glimepiride, Sanofi Aventis), Diabeta® (glyburide, Sanofi Aventis), Micronase®/Glynase® (glyburide, Pfizer), and Glucotrol®/Glucotrol XL® (glipizide, Pfizer); meglitinides, such as Prandin®/NovoNorm® (repaglinide, Novo Nordisk), Starlix® (nateglinide, Novartis), and Glufast® (mitiglinide, Takeda); biguanides, such as Glucophase®/Glucophase XR® (metformin HCl, Bristol Myers Squibb) and Glumetza® (metformin HCl, Depomed); thiazolidinediones; amylin analogs; GLP-1 analogs; DPP-IV inhibitors such as Januvia® (sitagliptin, Merck) and Galvus® (vildagliptin, Novartis); PTB-1 B inhibitors; protein kinase inhibitors (including AMP-activated protein kinase inhibitors); glucagon antagonists, glycogen synthase kinase-3 beta inhibitors; glucose-6-phoshatase inhibitors; glycogen phosphorylase inhibitors; sodium glucose co-transporter inhibitors; and alpha-glucosidase inhibitors, such as Glycet® (miglitol, Pfizer); statins, fibrates, and Zetia® (ezetimibe); alpha-blockers; beta-blockers; calcium channel blockers; diuretics; angiotensin converting enzyme (ACE) inhibitors; dual ACE and neutral endopeptidase (NEP) inhibitors; angiotensin-receptor blockers (ARBs); aldosterone synthase inhibitors; aldosterone-receptor antagonists; endothelin receptor antagonists; orlistat; phentermine; sibutramine; Acomplia® (rimonabant); thiazolidinediones (e.g., rosiglitazone, pioglitazone); SGLT 2 inhibitors (e.g., dapagliflozin, remogliflozin etabonate, sergliflozin, canagliflozin, and 1-chloro-4-(β-D-glucopyranos-1-yl)-2-[4-(('S)-tetrahydrofuran-3-yloxy)-benzyl]-benzene); PPAR-gamma-agonists (e.g., Gl 262570) and antagonists; PPAR-gamma/alpha modulators (e.g., KRP 297); alpha-glucosidase inhibitors (e.g., acarbose, voglibose); DPPIV inhibitors (e.g., Januvia® (sitagliptin), Galvus®/Zomelis® (vildagliptin), Onglyza® (saxagliptin), Nesina®/Vipidia® (alogliptin), and Tradjenta®/Trajenta (linagliptin)); alpha2-antagonists; glucagon-like protein-1 (GLP-1) receptor agonists and analogues (e.g., exendin-4); amylin; inhibitors of protein tyrosinephosphatase 1; substances that affect deregulated glucose production in the liver, e.g., inhibitors of glucose-6-phosphatase, or fructose-1,6-bisphosphatase, glycogen phosphorylase; glucagon receptor antagonists; inhibitors of phosphoenol pyruvate carboxykinase; glycogen synthase kinase and glucokinase activators; lipid lowering agents such as HMG-COA-reductase inhibitors (e.g., simvastatin, atorvastatin); fibrates (e.g., bezafibrate, fenofibrate), nicotinic acid and the derivatives thereof, PPAR-alpha agonists, PPAR-delta agonists; ACAT inhibitors (e.g., avasimibe); cholesterol absorption inhibitors such as ezetimibe; bile acid-binding substances such as cholestyramine; inhibitors of ileac bile acid transport; HDL-raising compounds such as CETP inhibitors and ABC1 regulators; active substances for treating obesity such as sibutramine and tetrahydrolipostatin; SDRIs; axokine; leptin; leptin mimetics; antagonists of the cannabinoid 1 receptor; and MCH-1 receptor antagonists; MC4 receptor agonists; NPY5 and NPY2 antagonists; beta3 adrenergic agonists such as SB-418790 and AD-9677; agonists of the 5HT2c receptor; GABA-receptor antagonists; Na-channel blockers; topiramate; protein-kinase C inhibitors; advanced glycation end product inhibitors; and aldose reductase inhibitors.

### Pharmaceutical Formulations, Administration, and Dosage Forms

When employed as pharmaceuticals, the compounds of the disclosure can be administered in the form of a pharmaceutical composition which refers to a combination of a compound of the disclosure, or its pharmaceutically acceptable salt, and at least one pharmaceutically acceptable carrier. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal, or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, one or more of the compounds of the disclosure above in combination with one or more pharmaceutically acceptable carriers. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

Compounds or compositions described herein may be administered to a patient using any amount and any route of administration effective for treating or lessening the severity of one or more of the diseases and conditions described herein. The exact amount required will vary from subject to subject, depending on the species, age, and general condition of the subject, the severity of the infection, disease or disorder, the particular agent, its mode of administration, and the like. Provided compounds are preferably formulated in a particular unit dosage form for ease of administration and uniformity of dosage. The expression “unit dosage form” as used herein refers to a physically discrete unit of agent appropriate for the patient to be treated.

The therapeutic dosage of the compounds of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. The dosage is likely to depend on such variables as the type and extent of progression of the disease or disorder, the overall health status of the particular patient, the relative biological efficacy of the compound selected, formulation of the excipient, and its route of administration.

### Numbered Embodiments

1. A compound of Formula I:

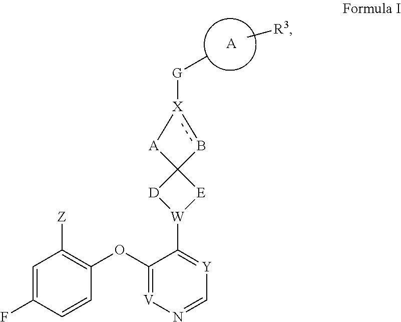

a stereoisomer, or a pharmaceutically acceptable salt thereof, wherein:
[symbol] denotes a single bond or a double bond, as valency permits;
A, B, D, and E are each independently selected from —C(R_{A1})(R_{A2})—, —C(R_{A1})(R_{A2})—C(R_{A1})(R_{A2})—, —C(R_{A1})(R_{A2})—O—, —C(R_{A1})(R_{A2})—NR^{A3}—, —C(═O)—, —C(R_{A1})(R_{A2})—C(═O)—, and —N═C(NH₂)—, wherein no more than one of A, B, D, and E is —C(R_{A1})(R_{A2})—O—, —C(R_{A1})(R_{A2})—NR^{A3}—, —C(R_{A1})(R_{A2})—C(═O)—, —C(═O)—, or—N═C(NH₂)—;
V is N or CH;
Y is N or CH;
G is O, CH₂, or NH;
Ring A is cyclohexyl or 6-membered heterocycloalkyl which has one heteroatom ring member being an oxygen;
W is N or CR^{W}, wherein R^{W} is H, halo, CN, OH, C₁₋₆ alkyl, C₁-6 alkoxy, amino, C₁₋₆ alkyl amino, or C₂₋₈ dialkylamino;
X is N or CRX, wherein R^{X} is absent, H, halo, CN, OH, C₁₋₆ alkyl, C₁-6 alkoxy, amino, C₁-6 alkyl amino, or C₂₋₈ dialkylamino;
Z is halo, C₁₋₆ alkyl, C₁-4 haloalkyl, C₁-4 cyanoalkyl, C₂-6 alkenyl, C₂-6 alkynyl, CN, NO₂, SR^{a3}, C(O)R^{b3}, C(O)NR¹R², C(O)OR^{a3}, OC(O)R^{b3}, OC(O)NR^{c3}R^{d3}, C(═NR^{e3}) NR^{c3}R^{d3}, NR^{c3}C(═NR^{c3}) NR^{c3}R^{d3}, NR^{c3}R^{d3}, NR^{c3}C(O)OR^{a3}, NR^{c3}C(O)NR^{c3}R^{d3}, NR^{c3}S(O)R^{b3}, NR^{c3}S(O)₂NR^{c3}R^{d3}, S(O)R^{b3}, S(O)NR^{c3}R^{d3}, S(O)₂R^{b3}, and P(O)R¹³R^{d3}, wherein said C₁₋₆ alkyl, C₁₋₄ haloalkyl, C₁-4 cyanoalkyl, C₂-6 alkenyl, and C₂₋₆ alkynyl are each optionally substituted with 1, 2, 3, or 4 substituents independently selected from Cy2, halo, CN, NO₂, CN, NO₂, OR^{a3}, SR^{a3}, C(O)R^{b3}, C(O)NR³R^{d3}, C(O)OR^{a3}, OC(O)R^{b3}, OC(O)NR³R^{d3}, C(═NR^{e3}) NR^{c3}R^{d3}, NR^{c3}C(═NR^{c3}) NR^{c3}R^{d3}, NR^{c3}R^{d3}, NR^{c3}C(O)R^{b3}, NR^{c3}C(O)OR^{a3}, NR³C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{A1} is independently selected from absent, H, halo, C₁₋₄ alkyl, C₁-4 alkoxy, C₁₋₄ haloalkyl, C₁-4 haloalkoxy, amino, C₁₋₆ alkylamino, C₂₋₈ dialkylamino, CN, NO₂, and OH;
each R^{A2} is independently selected from H, halo, C₁₋₄ alkyl, C₁-4 alkoxy, C₁₋₄ haloalkyl, C₁₋₄ haloalkoxy, amino, C₁₋₆ alkylamino, C₂₋₈ dialkylamino, CN, NO₂, and OH;
each R^{A3} is independently selected from H, C₁₋₄ alkyl, C₁-4 alkoxy, C₁₋₄ haloalkyl, C(O)R^{z}, and C(O)OR^{z}, wherein said C₁₋₆ alkyl is optionally substituted with phenyl, C₁₋₄ alkoxy, C₁₋₄ haloalkoxy, CN, NO₂, or OH;
R^{z} is H, C₁-6 alkyl, or phenyl;
each Cy2 is independently selected from C₆₋₁₄ aryl, C₃₋₁₈ cycloalkyl, 5-16 membered heteroaryl, and 4-18 membered heterocycloalkyl, each of which is optionally substituted with 1, 2, 3, or 4 substituents independently selected from R^{Cy2};
each R^{Cy2} is independently selected from halo, C₁₋₆ alkyl, C₁-4 haloalkyl, C₁-4 cyanoalkyl, C₂₋₆ alkenyl, C₂-6 alkynyl, phenyl, C₃₋₇ cycloalkyl, 5-6 membered heteroaryl, 4-7 membered heterocycloalkyl, CN, NO₂, OR^{a5}, SR^{a5}, C(O)R^{b5}, C(O)NR⁵R^{d5}, C(O)OR^{a5}, OC(O)R^{b5}, OC(O)NR^{c5}R^{d5}, C(═NR^{e5}) NR^{c5}R^{d5}, NR^{c5}C(═NR^{e5}) NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein said C₁₋₆ alkyl, C₁-4 haloalkyl, C₁-4 cyanoalkyl, C₂-6 alkenyl, C₂-6 alkynyl, phenyl, C₃₋₇ cycloalkyl, 5-6 membered heteroaryl, and 4-7 membered heterocycloalkyl are each optionally substituted with 1, 2, 3, or 4 substituents independently selected from CN, NO2, OR^{a5}, SR^{a5}, C(O)R^{b5}, C(O)NR^{c5}R⁴⁵, C(O)OR^{a5}, OC(O)R^{b5}, OC(O)NR^{c5}R^{d5}, C(═NR^{e5}) NR⁶⁵R⁴⁵, NR^{c5}C(═NR^{e5}) NR^{c 5}R^{d5}, NR^{c5}R^{d5}, NR^{c5}C(O)R^{b5}, NR^{c5}° C. (O)OR^{a5}, NR^{c5}° C. (O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
R¹ is
   (a) H, C₁₋₆ haloalkyl, C₁-6 alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁-6 alkyl, —C₃₋₁₂ cycloalkyl-C₁-6 alkyl, -(5-10 membered heteroaryl)-C₁₋₆ alkyl, or -(4-10 membered heterocycloalkyl)-C₁₋₆ alkyl, wherein said C₁₋₆ haloalkyl, C₁-6 alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁-6 alkyl, —C₃₋₁₂ cycloalkyl-C₁-6 alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or -C₃₋₁₂ cycloalkylene-N(R^{3a}′)(R^{3b}′), wherein the C₁₋₆ alkylene or C₃₋₁₂ cycloalkylene is optionally substituted with one or more R^{1as}, each R^{1as} is independently halo, oxo, C₁₋₆ alkyl, C₁-6 haloalkyl, C(═O)(R^{3a}′), N(R^{3a}′)(R^{3b}′), or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁-6 haloalkyl, C₃-12 cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es};
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR¹¹R^{d1}, C(═NR^{c1})NR^{c1}R^{d1}, NR^{c1}C(═NR^{c1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, N^{Rc1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO2, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{c1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{e1}S(O)R^{b1}, NR^{c1}S(O)₂R^{b1} NR^{c1}S(O)₂NR¹¹R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, R^{d1}, R^{a3}, R^{b3}, R¹³, R^{d3}, R^{a5}, R^{b5}, R^{e5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1}, R^{e3}, and R^{e5} is independently selected from H, C₁₋₆ alkyl, and CN;
each R^{g} is independently selected from OH, NO2, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a} is independently H, CN, or C₁₋₆ alkyl optionally substituted with one or more halo, C₆₋₁₀ aryl, or N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.
2. A compound of Formula II:

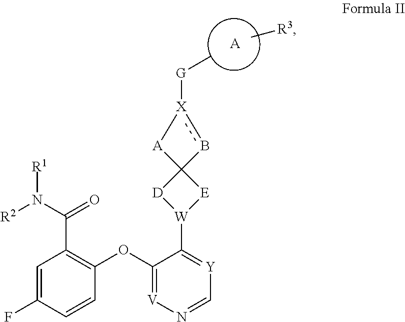

a stereoisomer, or a pharmaceutically acceptable salt thereof, wherein:
[symbol] denotes a single bond or a double bond, as valency permits;
A, B, D, and E are each independently selected from —C(R^{A1})(R^{A2})—, —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂)—;
V is N or CH;
Y is N or CH;
G is O, CH₂, or NH;
Ring A is cyclohexyl or 6-membered heterocycloalkyl which has one heteroatom ring member being an oxygen;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CRX, wherein R^{X} is absent, H, halo, CN, OH, C₁₋₆ alkyl, C₁₋₆ alkoxy, amino, C₁-6 alkyl amino, or C₂₋₈ dialkylamino;
each R^{A1} is independently selected from absent, H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A2} is independently selected from H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A3} is independently selected from H, C₁₋₄ alkyl, C₁₋₄ alkoxy, C₁₋₄ haloalkyl, C(O)R^{z}, and C(O)OR^{z}, wherein said C₁₋₆ alkyl is optionally substituted with phenyl, C₁₋₄ alkoxy, C₁₋₄ haloalkoxy, CN, NO₂, or OH;
R^{z} is H, C₁₋₆ alkyl, or phenyl;
R¹ is
   (a) H, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s},
   each R^{1s} is independently oxo or ═NR^{4a},
   (c) —C₁₋₆ alkylene-N(R^{3a}′)(R^{3b}′) or -C₃₋₁₂ cycloalkylene-N(R^{3a}′)(R^{3b}′), wherein the C₁₋₆ alkylene or C₃₋₁₂ cycloalkylene is optionally substituted with one or more R^{1as};
      each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es};
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{c1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1} NR^{c1}S(O)R^{b1}, NR^{c1}S(O)₂R^{b1}, NR^{c1}S(O)₂NR¹¹R^{d1}, S(O)R^{b1}, S(O)NR¹R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{c1})NR^{c1}R^{d1}, NR^{c1}C(═NR^{e1})NR¹¹R^{d1}, NR^{e1}R^{d1} NR^{c1}C(O)R^{b1}, NR^{c1}C(O)OR^{a1}, NR^{c1}C(O)NR^{c1}R^{d1}, NR^{e1}S(O)R^{b1}, NR^{c1}S(O)₂R^{b1} NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN) 2, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a} is independently H, CN, or C₁₋₆ alkyl optionally substituted with one or more halo, C₆₋₁₀ aryl, or N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.
3. The compound of any one of the preceding embodiments, a stereoisomer, or a pharmaceutically acceptable salt thereof, wherein when [symbol] is a double bond, X is C.
4. The compound of any one of the preceding embodiments, a stereoisomer, or a pharmaceutically acceptable salt thereof, wherein the spiro moiety represented by the below formula:

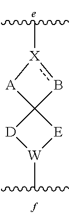

wherein e and f indicate points of attachment to the remainder of the molecule, is selected from:

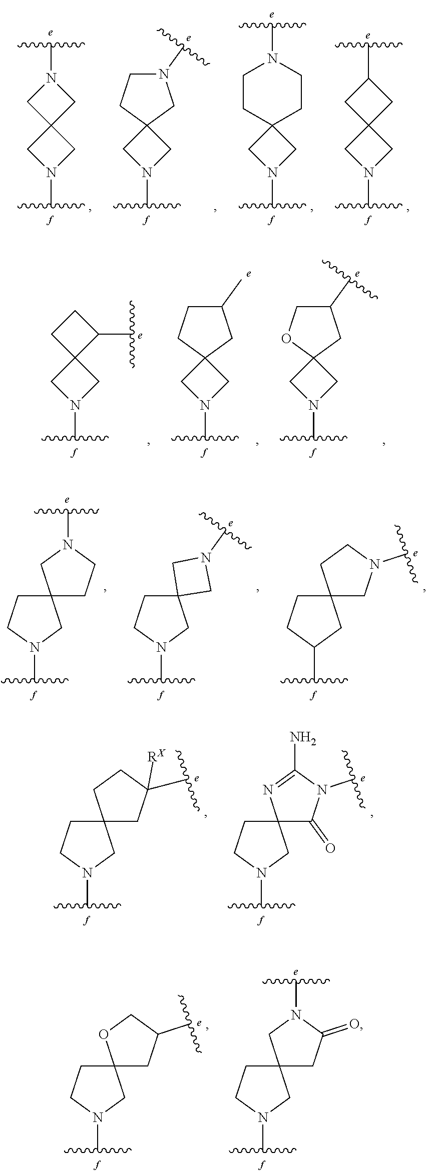

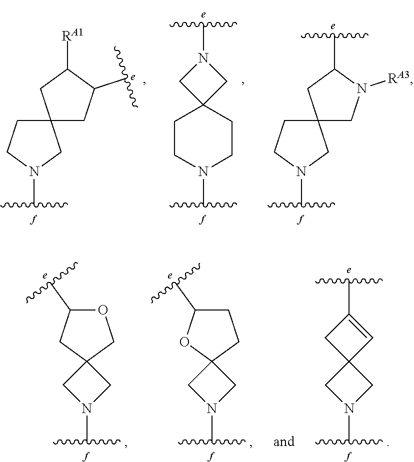

5. A compound of Formula III:

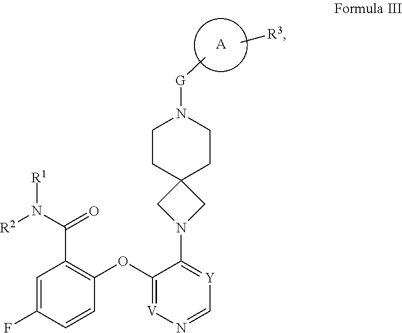

a stereoisomer, or a pharmaceutically acceptable salt thereof, wherein:
V is N or CH;
Y is N or CH;
G is O, CH₂, or NH;
Ring A is cyclohexyl or 6-membered heterocycloalkyl which has one heteroatom ring member being an oxygen;
R¹ is
   (a) —C₁₋₆ alkylene-N(R^{3a}′)(R^{3b}′) or -C₃₋₁₂ cycloalkylene-N(R^{3a}′)(R^{3b}′), wherein the C₁₋₆ alkylene or C₃₋₁₂ cycloalkylene is optionally substituted with one or more R^{1as},
      each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
   (b) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs},
      each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{c1}) NR¹¹R^{d1}, NR^{c1}C(═NR^{e1}) NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, N^{Rc1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{e1}R^{d1}, C(═NR^{c1}) NR^{c1}R^{d1}, NR^{c1}C(═NR^{c1}) NR¹¹R^{d1}, NR^{c1}R^{d1}, NR^{c1}C(O)R^{b1}, NR^{c1}C(O)OR^{a1}, NR^{c1}C(O)NR^{c1}R^{d1}, N^{Rc1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN) 2, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.
6. A compound of Formula III-a:

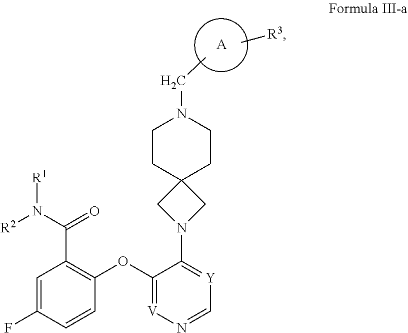

a stereoisomer, or a pharmaceutically acceptable salt thereof, wherein:
V is N or CH;
Y is N or CH;
Ring A is cyclohexyl or 6-membered heterocycloalkyl which has one heteroatom ring member being an oxygen;
R¹ is
   (a) —C₁₋₆ alkylene-N(R^{3a}′)(R^{3b}′) or -C₃₋₁₂ cycloalkylene-N(R^{3a}′)(R^{3b}′), wherein the C₁₋₆ alkylene or C₃₋₁₂ cycloalkylene is optionally substituted with one or more R^{1as};
      each R^{1as} is independently halo, oxo, C₁₋₆ alkyl, C₁₋₆ haloalkyl, C(═O)(R^{3a}′), N(R³a>)(R^{3b}′), or S(═O)₂R^{3a}′;
   (b) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(—O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es};
      each R^{1es} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1}) NR^{c1}R^{d1}, NR^{c1}C(═NR^{c1}) NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, N^{Rc1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{c1}) NR^{c1}R^{d1} NR^{c1}C(═NR^{c1}) NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, N^{Rc1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN) 2, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.
7. A compound of Formula III-b:

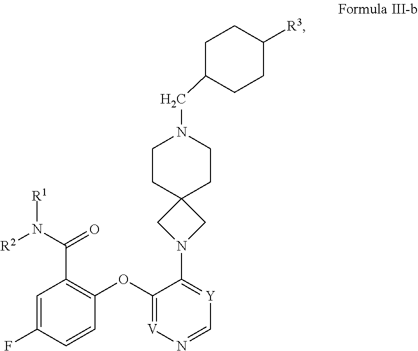

### or any one of Formulae III-b-1, III-b-2, III-b-3,

a stereoisomer, or a pharmaceutically acceptable salt thereof, wherein:
V is N or CH;
Y is N or CH;
R¹ is
   (a) —C₁₋₆ alkylene-N(R^{3a}′)(R^{3b}′) or -C₃₋₁₂ cycloalkylene-N(R^{3a}′)(R^{3b}′), wherein the C₁₋₆ alkylene or C₃₋₁₂ cycloalkylene is optionally substituted with one or more R^{1as}; each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
   (b) 3- to 12-membered heterocycloalkyl which has at least one nitrogen and optionally one or more other heteroatom ring members, wherein the 3- to 12-membered heterocycloalkyl is optionally substituted with one or more R^{1bs},
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es}; each R^{1es} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
      each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1}) NR^{c1}R^{d1} NR^{c1}C(═NR^{e1}) NR^{c1}R^{d1}, NR¹¹R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{e1}S(O)₂R^{b1}, NR^{e1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1}) NR^{c1}R^{d1}, NR^{c1}C(═NR^{c1}) NR^{c1}R^{d1}, NR¹¹R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
   each R^{a1}, R^{b1}, R^{e1}, and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   each R^{e1} is independently selected from H, C₁₋₆ alkyl, and CN;
   each R^{g} is independently selected from OH, NO₂, CN, halo, oxo, OBn, N(RN) 2, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
   each R^{4a}′ and R^{4b}′ is independently H, C₁₋₆ alkyl, C₃₋₁₂ cycloalkyl, or 3- to 12-membered heterocycloalkyl; and
   each R_{N} is independently H, C₁₋₆ alkyl, or C₁₋₆ haloalkyl;
   wherein any cycloalkyl or heterocycloalkyl group is optionally further substituted with 1 or 2 oxo groups.
8. A compound of Formula III-c:

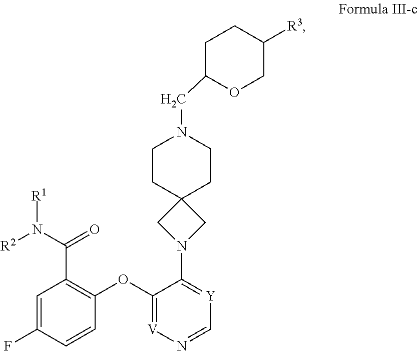

### or any one of Formulae III-c-1, III-c-2, or III-c-3

a stereoisomer, or a pharmaceutically acceptable salt thereof, wherein:
V is N or CH;
Y is N or CH;
R¹ is
   (a) —C₁₋₆ alkylene-N(R^{3a}′)(R^{3b}′) or -C₃₋₁₂ cycloalkylene-N(R^{3a}′)(R^{3b}′), wherein the C₁₋₆ alkylene or C₃₋₁₂ cycloalkylene is optionally substituted with one or more R^{1as};
      each R^{1as} is independently halo, oxo, C₁₋₆ alkyl, C₁₋₆ haloalkyl, C(═O)(R^{3a}′), N(R³a>)(R^{3b}′), or S(═O)₂R^{3a}′;
   (b) 3- to 12-membered heterocycloalkyl which has at least one nitrogen and optionally one or more other heteroatom ring members, wherein the 3- to 12-membered heterocycloalkyl is optionally substituted with one or more R^{1bs},
      each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(—O)(R^{3a}′), N(R³a>)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
      each R^{1cs} is independently halo, oxo, C₁₋₆ alkyl, C₁₋₆ haloalkyl, C(═O)(R^{3a}′), N(R³a>)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{c1}) NR^{c1}R^{d1}, NR^{c1}C(═NR^{e1}) NR¹¹R^{d1}, NR¹¹R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR¹¹R^{d1}, S(O)R^{b1}, S(O)NR^{c1}R^{d1}, S(O)₂R^{b1}, and S(O)₂NR¹¹R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR¹¹R^{d1}, C(═NR^{c1}) NR^{c1}R^{d1}, NR^{c1}C(═NR^{c1}) NR¹¹R^{d1}, NR^{e1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{e1}S(O)₂NR^{c1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN) 2, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.
9. The compound of any one of the preceding embodiments, wherein V is N.
10. The compound of any one of the preceding embodiments, wherein V is CH.
11. The compound of any one of the preceding embodiments, wherein Y is N.
12. The compound of any one of the preceding embodiments, wherein Y is CH.
13. The compound of any one of the preceding embodiments, wherein G is O.
14. The compound of any one of the preceding embodiments, wherein G is CH₂.
15. The compound of any one of the preceding embodiments, wherein G is NH.
16. The compound of any one of the preceding embodiments, wherein R² is C₁₋₆ alkyl optionally substituted with 1, 2, 3, 4, or 5 substituents independently selected from R^{g}, wherein:
each R^{g} is independently selected from OH, NO₂, CN, halo, oxo, OBn, N(RN) 2, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.
17. The compound of any one of the preceding embodiments, wherein R² is C₁₋₆ alkyl.
18. The compound of any one of the preceding embodiments, wherein R² is isopropyl.
19. The compound of any one of the preceding embodiments, wherein R¹ is-C₁₋₆ alkylene-N(R^{3a}′)(R^{3b}′) or -C₃₋₁₂ cycloalkylene-N(R^{3a}′)(R^{3b}′), wherein the C₁₋₆ alkylene or C₃₋₁₂ cycloalkylene is optionally substituted with one or more R^{1as},
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.
20. The compound of any one of the preceding embodiments, wherein R¹ is 3- to 12-membered heterocycloalkyl which has at least one nitrogen and optionally one or more other heteroatom ring members, wherein the 3- to 12-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.
21. The compound of any one of the preceding embodiments, wherein R¹ is

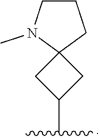 [SMILES: *C1CC2(CCCN2C)C1]

22. The compound of any one of the preceding embodiments, wherein R¹ is

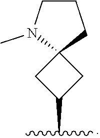

23. The compound of any one of the preceding embodiments, wherein R¹ is C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1es} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.
24. The compound of any one of the preceding embodiments, wherein R¹ is

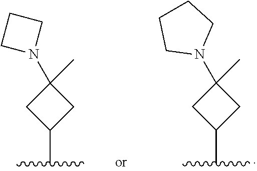

25. The compound of any one of the preceding embodiments, wherein R¹ is

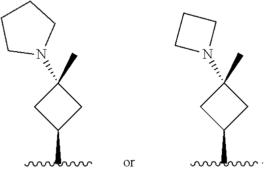

26. The compound of any one of the preceding embodiments, wherein Ring A is cyclohexyl.
27. The compound of any one of the preceding embodiments, wherein Ring A is 6-membered heterocycloalkyl which has one heteroatom ring member being an oxygen.
28. The compound of any one of the preceding embodiments, wherein Ring A is

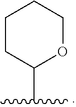 [SMILES: *C1CCCCO1]

29. The compound of any one of the preceding embodiments, wherein R³ is NR^{c1}S(O)₂R^{b1}, and wherein:
each R^{b1} and R^{c1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl.
30. The compound of any one of the preceding embodiments, wherein R³ is NR^{c1}S(O)₂R^{b1}, and each R^{b1} and R^{c1} is independently selected from H and C₁₋₆ alkyl.
31. The compound of any one of the preceding embodiments, wherein R³ is NHS(O)₂ (C₂H5).
32. The compound of any one of the preceding embodiments, wherein R³ is NR^{c1}S(O)₂NR^{c1}R^{d1}, and wherein:
each R^{e1} and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl.
33. The compound of any one of the preceding embodiments, wherein R³ is NR^{c1}S(O)₂NR^{c1}R^{d1}, and each R^{e1} and R^{d1} is independently selected from H and C₁₋₆ alkyl.
34. The compound of any one of the preceding embodiments, wherein R³ is NHS(O)₂NH(C₂H5).
35. The compound of any one of the preceding embodiments, wherein R³ is

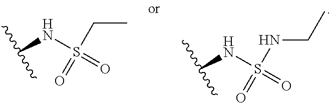

36. A compound as shown in Table 1 or a stereoisomer thereof, or a pharmaceutically acceptable salt thereof.
37. A compound as shown in Table 1 or a pharmaceutically acceptable salt thereof.
38. A compound as shown in Table 1.
39. A compound as shown in Table 1 or a pharmaceutically acceptable salt thereof, wherein the salt is hydrochloride.
40. The compound according to any one of the preceding embodiments, wherein the compound is useful for the treatment of cancer and wherein the compound minimizes hERG binding.
41. A pharmaceutical composition comprising a compound of any one of the preceding embodiments, or a pharmaceutically acceptable salt thereof, and at least one pharmaceutically acceptable carrier.
42. A pharmaceutical composition comprising a salt or crystalline form of the compound of any one of embodiments 1-40, and at least one pharmaceutically acceptable carrier.
43. A method of inhibiting the interaction between menin and MLL comprising contacting the menin and MLL with a compound of any one of embodiments 1-40 or a pharmaceutical composition of embodiment 41 or embodiment 42.
44. A method of treating cancer in a patient comprising administering to the patient a compound of any one of embodiments 1-40 or a pharmaceutical composition of embodiment 41 or embodiment 42.
45. The method of embodiment 44, wherein the cancer is a hematological cancer.
46. The method of embodiment 44, wherein the cancer is a leukemia.
47. The method of embodiment 44, wherein the cancer is a lymphoma.
48. The method of embodiment 44, wherein the cancer is mixed lineage leukemia (MLL), MLL-related leukemia, MLL-associated leukemia, MLL-positive leukemia, MLL-induced leukemia, rearranged mixed lineage (KMT2A-rearranged) leukemia (MLL-r), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CML), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sézary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), or Waldenstrom's macroglobulinemia.
49. The method of embodiment 44, wherein the cancer is an abstract nucleophosmin (NPM1)-mutated acute myeloid leukemia (i.e., NPM1mut acute myloid leukemia).
50. The method of embodiment 44, wherein the cancer is a rearranged mixed lineage (KMT2A-rearranged) leukemia (MLL-r).
51. The compound of any one of embodiments 1-40 or a pharmaceutically acceptable salt thereof, or the pharmaceutical composition of embodiment 41 or embodiment 42, for use in treating or preventing a disease caused by, or associated with, menin expression, activity, and/or function.
52. The compound of any one of embodiments 1-40 or a pharmaceutically acceptable salt thereof, or the pharmaceutical composition of embodiment 41 or embodiment 42, for use in treating or preventing cancer.
53. Use of the compound of any one of embodiments 1-40 or a pharmaceutically acceptable salt thereof, or the pharmaceutical composition of embodiment 41 or embodiment 42, for treating or preventing a disease caused by, or associated with, menin expression, activity, and/or function.
54. Use of the compound of any one of embodiments 1-40 or a pharmaceutically acceptable salt thereof, or the pharmaceutical composition of embodiment 41 or embodiment 42, in the manufacture of a medicament for treating or preventing a disease caused by, or associated with, menin expression, activity, and/or function.
55. Use of a compound of any one of embodiments 1-40 or a pharmaceutically acceptable salt thereof, or the pharmaceutical composition of embodiment 41 or embodiment 42, for treating or preventing cancer.
56. Use of the compound of any one of embodiments 1-40 or a pharmaceutically acceptable salt thereof, or the pharmaceutical composition of embodiment 41 or embodiment 42, in the manufacture of a medicament for treating or preventing cancer.
57. A kit comprising the compound of any one of 1-40 or a pharmaceutically acceptable salt thereof, or the pharmaceutical composition of embodiment 41 or embodiment 42 and instructions for its use.
58. A method of preparing a compound herein according to a scheme of the present disclosure or synthetic description in the Examples.
59. An intermediate useful in the preparation of any one of the compounds herein.
60. The compound of any one of embodiments 1-40, wherein the compound is Compound No. 1, a stereoisomer, or a pharmaceutically acceptable salt thereof.
61. The compound of any one of embodiments 1-40, wherein the compound is Compound No. 2, a stereoisomer, or a pharmaceutically acceptable salt thereof.
62. The compound of any one of embodiments 1-40, wherein the compound is Compound No. 3, a stereoisomer, or a pharmaceutically acceptable salt thereof.
63. The compound of any one of embodiments 1-40, wherein the compound is Compound No.
4, a stereoisomer, or a pharmaceutically acceptable salt thereof.
64. The compound of any one of embodiments 1-40, wherein the compound is Compound No. 5, a stereoisomer, or a pharmaceutically acceptable salt thereof.
65. The compound of any one of embodiments 1-40, wherein the compound is Compound No. 6, a stereoisomer, or a pharmaceutically acceptable salt thereof.
66. The compound of any one of embodiments 1-40, wherein the compound is Compound No. 7, a stereoisomer, or a pharmaceutically acceptable salt thereof.
67. The compound of any one of embodiments 1-40, wherein the compound is Compound No. 8, a stereoisomer, or a pharmaceutically acceptable salt thereof.
68. The compound of any one of embodiments 1-40, wherein the compound is Compound No.
9, a stereoisomer, or a pharmaceutically acceptable salt thereof.
69. A pharmaceutically acceptable salt of the compound of any one of embodiments 60-68.
70. The compound of any one of embodiments 60-69, or a pharmaceutically acceptable salt thereof, wherein the salt is a hydrochloride salt.
71. The compound according to any one of embodiments 60-70, wherein the compound is useful for the treatment of cancer and wherein the compound minimizes hERG binding.
72. A pharmaceutical composition comprising a compound of any one of embodiments 1-40 or 60-71, or a pharmaceutically acceptable salt thereof, and at least one pharmaceutically acceptable carrier.
73. A pharmaceutical composition comprising a salt or crystalline form of the compound of any one of embodiments 1-40 or 60-71, and at least one pharmaceutically acceptable carrier.
74. A method of inhibiting the interaction between menin and MLL comprising contacting the menin and MLL with a compound of any one of embodiments 1-40 or 60-71 or a pharmaceutical composition of embodiment 72 or embodiment 73.
75. A method of treating cancer in a patient comprising administering to the patient a compound of any one of embodiments 1-40 or 60-71 or a pharmaceutical composition of embodiment 72 or embodiment 73.
76. The method of embodiment 75, wherein the cancer is a hematological cancer.
77. The method of embodiment 75, wherein the cancer is a leukemia.
78. The method of embodiment 75, wherein the cancer is a lymphoma.
79. The method of embodiment 75, wherein the cancer is mixed lineage leukemia (MLL), MLL-related leukemia, MLL-associated leukemia, MLL-positive leukemia, MLL-induced leukemia, rearranged mixed lineage (KMT2A-rearranged) leukemia (MLL-r), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CML), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sézary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), or Waldenstrom's macroglobulinemia.
80. The method of embodiment 75, wherein the cancer is an abstract nucleophosmin (NPM1)-mutated acute myeloid leukemia (i.e., NPM1mut acute myloid leukemia).
81. The method of embodiment 75, wherein the cancer is a rearranged mixed lineage (KMT2A-rearranged) leukemia (MLL-r).
82. The compound of any one of embodiments 1-40 or 60-71 or a pharmaceutically acceptable salt thereof, or the pharmaceutical composition of embodiment 72 or embodiment 73, for use in treating or preventing a disease caused by, or associated with, menin expression, activity, and/or function.
83. The compound of any one of embodiments 1-40 or 60-71 or a pharmaceutically acceptable salt thereof, or the pharmaceutical composition of embodiment 72 or embodiment 73, for use in treating or preventing cancer.
84. Use of the compound of any one of embodiments 1-40 or 60-71 or a pharmaceutically acceptable salt thereof, or the pharmaceutical composition of embodiment 72 or embodiment 73, for treating or preventing a disease caused by, or associated with, menin expression, activity, and/or function.
85. Use of the compound of any one of embodiments 1-40 or 60-71 or a pharmaceutically acceptable salt thereof, or the pharmaceutical composition of embodiment 72 or embodiment 73, in the manufacture of a medicament for treating or preventing a disease caused by, or associated with, menin expression, activity, and/or function.
86. Use of a compound of any one of embodiments 1-40 or 60-71 or a pharmaceutically acceptable salt thereof, or the pharmaceutical composition of embodiment 72 or embodiment 73, for treating or preventing cancer.
87. Use of the compound of any one of embodiments 1-40 or 60-71 or a pharmaceutically acceptable salt thereof, or the pharmaceutical composition of embodiment 72 or embodiment 73, in the manufacture of a medicament for treating or preventing cancer.
88. A kit comprising the compound of any one of 1-40 or 60-71 or a pharmaceutically acceptable salt thereof, or the pharmaceutical composition of embodiment 72 or embodiment 73 and instructions for its use.

### EXAMPLES

As depicted in the Examples below, the compounds of the present disclosure were prepared by the methods described below. Although the Examples illustrate specific methods, those of skill in the art will recognize that other reasonable approaches might also be utilized to prepare these and related compounds.

| Abbreviation | Definition |
|---|---|
| ACN | Acetonitrile |
| AcOH | Acetic acid |
| (Boc)₂O | Di-tert-butyl dicarbonate |
| Cbz-Cl | Benzyl chloroformate; benzyl chloridocarbonate |
| CPME | Cyclopentyl methyl ether |
| DBU | 1,8-Diazabicyclo[5.4.0]undec-7-ene |
| DCE | 1,2-Dichloroethane |
| DCM | Dichloromethane (methylene chloride) |
| DIEA (DIPEA), | Diisopropylethylamine |
| iPr₂NEt | |
| DMA (DMAc) | Dimethylacetamide |
| DMAP | 4-Dimethylaminopyridine |
| DMF | Dimethylformamide |
| DMSO | Dimethylsulfoxide |
| dppf | 1,1-bis(Diphenylphosphino)ferrocene |
| Et₃N (TEA) | Triethylamine |
| EtOAc | Ethyl acetate |
| EtOH | Ethanol |
| h | Hour(s) |
| HATU | 1-[bis(Dimethylamino)methylene]-1H-1,2,3-triazolo[4,5- |
| | b]pyridinium 3-oxide hexafluorophosphate |
| HBTU | 2-(1H-Benzotriazole-1-yl)-1,1,3,3-tetramethyluronium |
| | hexafluorophosphate |
| HOBt | 1-Hydroxybenzonotrazole |
| HPLC | High performance liquid chromatography |
| IPA, iPrOH | Isopropyl alcohol |
| LCMS | Liquid chromatography-mass spectrometry |
| mCPBA | meta-Chloroperoxybenzoic acid |
| Me | Methyl |
| MeI | Methyl iodide |
| MeOH | Methanol |
| mg | Milligram |
| min | Minute(s) |
| mL | Milliliters |
| mmol | Millimoles |
| MTBE | tert-Butyl methyl ether |
| NMP | N-Methyl-2-pyrrolidone |
| NOE | Nuclear Overhauser effect |
| PdCl₂(dppf)•CH₂Cl₂ | [1,1′-bis(Diphenylphosphino)ferrocene]dichloropalladium(II), |
| | complex with dichloromethane |
| Pd(dppf)Cl₂ | (1,1′-Bis(diphenylphosphino)ferrocene)palladium(II) dichloride |
| PhMe | Toluene |
| PG | Protecting group |
| ROE | Rotating-frame Overhauser effect |
| RP | Reverse phase |
| RT | Room temperature |
| Rt | Retention time |
| SFC | Supercritical fluid chromatography |
| STAB | Sodium triacetoxyborohydride |
| T3P | Propanephosphonic acid anhydride |
| TBAI | Tetra-n-butylammonium iodide |
| Tf₂O | Trifluoromethanesulfonic anhydride |
| TFA | Trifluoroacetic acid |
| TFAA | Trifluoroacetic anhydride |
| TFE | Trifluoroethanol |
| THF | Tetrahydrofuran |
| TLC | Thin layer chromatography |
| TMSCl | Trimethylsilyl chloride |
| TMG | 1,1,3,3-Tetramethylguanidine |
| pTsCl (TsCl) | 4-Toluenesulfonyl chloride |
| UHP | Urea hydrogen peroxide |
| UPLC | Ultra-performance liquid chromatography |

### LCMS

Instrument names: Shimadzu LC2020 Nexera Series; Shimadzu MS2020 N-Series; Agilent 1290 Infinity II series with 1260 MSD.

Method A: Mobile Phase A: 5 mM NH₄HCO₃ in water, mobile phase B: ACN; Column: Phenomenex Kinetex EVO C18 (50×3.0) mm, 2.6 μm.

Method B: Mobile Phase A: 0.1% FA in water, mobile phase B: 0.05% FA in ACN; Column: ZORBAX ECLIPSE PLUS C18 (50×2.1) mm, 1.8 μm.

Method C: Mobile Phase: A: 0.1% TFA in water, mobile phase B: ACN; Column: ZORBAX ECLIPSE PLUS C18 (50×2.1) mm, 1.8 μm.

Method D: Mobile Phase A: 10 mM NH₄HCO₃ in water, mobile phase B: ACN; Column: Phenomenex Kinetex EVO C18 (50×3.0) mm, 2.6 μm.

Method E: Mobile Phase A: 0.1% FA in water, mobile phase B: 0.1% FA in ACN; Column: ZORBAX ECLIPSE PLUS C18 (50×2.1) mm, 1.8 μm.

### HPLC

Instrument names: Shimadzu LC; Prominence-I series instruments as followed using % with UV detection (Maxplot).

Method A: Mobile Phase A: 0.1% TFA in water, mobile phase B: ACN; Flow Rate: 1.2 mL/min; Column: Atlantis T3 C18 (150×4.6) mm, 3 μm.

Method B: Mobile Phase A: 0.1% FA in water, mobile phase B: ACN; Flow Rate: 1.2 mL/min; Column: Atlantis T3 C18 (150×4.6) mm, 3 μm.

Method C: Mobile Phase A: 10 mM NH₄HCO₃ in water, mobile phase B: ACN; Flow Rate: 1.2 mL/min; Column: X-Bridge C18 (150×4.6) mm, 3.5 μm.

### Prep HPLC

Method A: Mobile Phase A: 0.1% TFA in water; mobile phase B: ACN; Flow Rate: 15.0 mL/min; Column: X-Select CSH C18 (250×19) mm, 5 μm.

Method B: Mobile Phase A: 0.1% TFA in water; mobile phase B: ACN; Flow Rate: 18.0 mL/min; Column: X-Timate C18 (250×21.2) mm, 5 μm.

Method C: Mobile Phase A: 0.1% TFA in water; mobile phase B: ACN; Flow Rate: 15.0 mL/min; Column: X-Bridge C8 (150×19) mm, 5 μm.

Chiral SFC Instrument names: Shimadzu Nexera UC SFC Analytical; PIC-SFC Analytical-10

Method A: Mobile Phase A: CO₂; Co-solvent-0.5% Isopropylamine in MeOH (50:50); Flow Rate: 4.0 mL/min; % Co-Solvent: 50%; Column: CHIRALPAK-IK (250×4.6) mm, 5 μm.

Method B: Mobile Phase A: CO₂; Co-solvent-0.5% Isopropylamine in [ACN: MeOH (1:1)] (50:50); Flow Rate: 4.0 mL/min; % Co-Solvent: 50%; Column: LUX—I-Amylose 3 (250×4.6) mm, 5 μm.

Method C: Mobile Phase A: CO₂; Co-solvent-0.5% Isopropylamine in [ACN: MeOH (1:1)] (55:45); Flow Rate: 4.0 mL/min; % Co-Solvent: 45%; Column: LUX—I-Amylose 3 (250×4.6) mm, 5 μm.

Method D: Mobile Phase A: CO₂; Co-solvent-0.5% Isopropylamine in [ACN: MeOH (1:1)] (55:45); Flow Rate: 4.0 mL/min; % Co-Solvent: 45%; Column: I-Cellulose Z (250×4.6) mm, 5 μm.

Method E: Mobile Phase A: CO₂; Co-solvent-0.5% Isopropylamine in [ACN: MeOH (1:1)] (55:45); Flow Rate: 4.0 mL/min; % Co-Solvent: 45%; Column: Whelk-(R,R) (250×4.6) mm, 5 μm.

### Chiral Prep SFC

Method A: Mobile Phase A: CO₂; Co-solvent-0.5% Isopropylamine in MeOH (50:50); Flow Rate: 100 mL/min; % Co-Solvent: 50%; Column: CHIRALPAK-IK (250×30) mm, 5 μm.

### Intermediates

### Intermediate 1. Methyl 5-fluoro-2-(pyrimidin-5-yloxy)benzoate

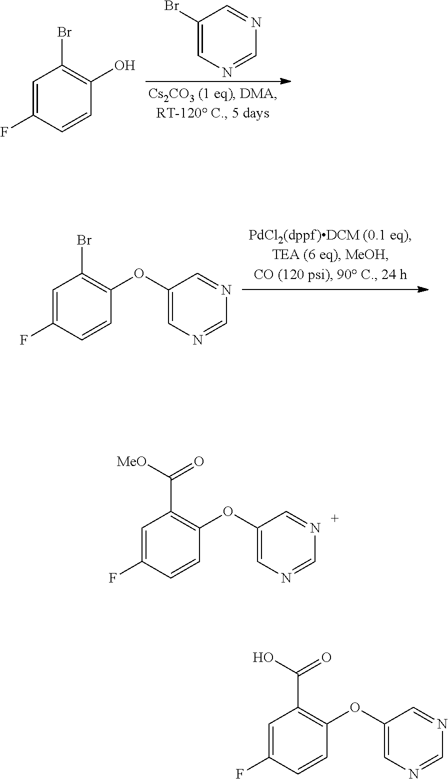 [SMILES: Oc1ccc(F)cc1Br ; Brc1cncnc1 ; Fc1ccc(Oc2cncnc2)c(Br)c1 ; COC(=O)c1cc(F)ccc1Oc1cncnc1 ; O=C(O)c1cc(F)ccc1Oc1cncnc1]

### Step 1. 5-(2-Bromo-4-fluorophenoxy)pyrimidine

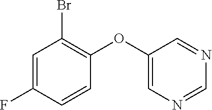 [SMILES: Fc1ccc(Oc2cncnc2)c(Br)c1]

Step 1 was performed following the same procedure in two batches, a 450 g batch and a 200 g batch.

In a dried, 10 L four neck round bottom flask under a nitrogen atmosphere, 2-bromo-4-fluorophenol (450 g, 2356 mmol) was dissolved in DMA (2.5 L) at RT. Cs2CO₃ (768 g, 2356 mmol) and 5-bromopyrimidine (375 g, 2356 mmol) were then added at RT, and the reaction was stirred at 120° C. for 5 days. The reaction progress was monitored by LCMS and TLC(10% EtOAc in hexane). The reaction mixtures from both batches (450 g and 200 g) were combined, quenched with water (3 L), and extracted with MTBE (2×5 L). The combined organic layer was washed with water (2 L) and brine solution (2 L), dried over anhydrous sodium sulfate and filtered, and the filtrate was concentrated under reduced pressure. The crude product was purified by silica-gel column chromatography using EtOAc in hexane (product eluted at 16% EtOAc in hexane) to obtain 5-(2-bromo-4-fluorophenoxy)pyrimidine (380 g, 38.9% combined yield) as an oil. ¹H NMR (400 MHz, DMSO-d₆): δ 9.00 (s, 1H), 8.57 (s, 2H), 7.81 (dd, J=2.9, 8.2 Hz, 1H), 7.46-7.33 (m, 2H); LCMS (Method E): Rt=1.79 min, m/z=269.2 [M+H]⁺, 78.45%.

### Step 2. Methyl 5-fluoro-2-(pyrimidin-5-yloxy)benzoate and 5-fluoro-2-(pyrimidin-5-yloxy)benzoic acid

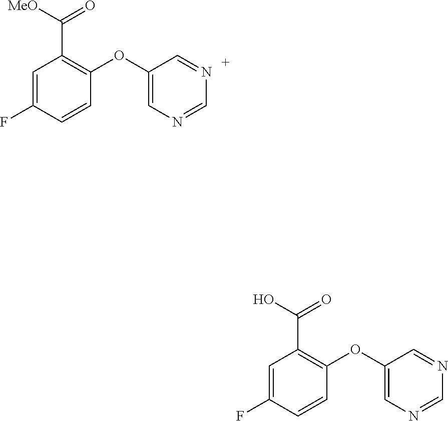 [SMILES: COC(=O)c1cc(F)ccc1Oc1cncnc1 ; O=C(O)c1cc(F)ccc1Oc1cncnc1]

Step 2 was performed following the same procedure in sixteen batches, a 21.5 g batch, and 15×20 g batches.

In a 300 mL stainless steel mini clave, 5-(2-bromo-4-fluorophenoxy)pyrimidine (20 g, 74.3 mmol) was dissolved in MeOH (150 mL), and the solution was degassed with argon for 30 min. TEA (61.0 mL, 446 mmol) was added, and the reaction was degassed for another 5 min. To the above solution, PdCl₂(dppf)-CH₂Cl₂ adduct (6.07 g, 7.43 mmol) was added, and the reaction was stirred under 120 psi carbon monoxide gas atmosphere (Caution: toxic gas) at 90° C. for 24 h. The reaction was monitored by TLC(40% EtOAc in hexane). After completion, the reaction was filtered through a Celite® pad, and the pad was washed with methanol (150 mL). The filtrate was concentrated under reduced pressure to obtain crude product (22 g). Similarly, all sixteen batches (21.5 g batch and 15×20 g batches) were filtered, and the filtrate was concentrated under reduced pressure to obtain crude product separately. The combined crude material (320 g) from sixteen batches was collected and dissolved in EtOAc (2 L). To this solution, 10% sodium bicarbonate (2 L) solution was added, and the mixture was extracted with EtOAc (2×2 L). The combined organic layer was dried over sodium sulfate and filtered, and the filtrate was concentrated under reduced pressure. The crude product was purified by silica gel flash column chromatography using EtOAc in hexane as the eluent (product eluted at 15-20% EtOAc in hexane) to obtain methyl 5-fluoro-2-(pyrimidin-5-yloxy)benzoate (198 g, 62.2% combined yield) as a solid. Further, the aqueous layer was acidified with conc. HCl (pH~2), and the precipitate was filtered and dried under vacuum to obtain crude 5-fluoro-2-(pyrimidin-5-yloxy)benzoic acid (25 g, 7.99% yield) as a solid. This material was used without further purification.

Methyl 5-fluoro-2-(pyrimidin-5-yloxy)benzoate: ¹H NMR (400 MHz, DMSO-d₆): δ 8.95 (s, 1H), 8.50 (s, 2H), 7.74 (dd, J=3.1, 8.8 Hz, 1H), 7.60 (ddd, J=3.3, 7.9, 9.0 Hz, 1H), 7.44 (dd, J=4.6, 9.0 Hz, 1H), 3.74 (s, 3H); LCMS (Method D): Rt=1.65 min, m/z=249.1 [M+H]⁺, 92.80%.

5-Fluoro-2-(pyrimidin-5-yloxy)benzoic acid: ¹H NMR (400 MHz, DMSO-d₆): δ 13.24 (br s, 1H), 8.92 (s, 1H), 8.47 (s, 2H), 7.70 (dd, J=3.3, 8.9 Hz, 1H), 7.55 (ddd, J=3.3, 7.9, 8.9 Hz, 1H), 7.40 (dd, J=4.6, 9.0 Hz, 1H); Rt=1.26 min, m/z=235.2 [M+H]⁺, 74.16%.

### Intermediate 2. Methyl 2-((4-chloropyrimidin-5-yl)oxy)-5-fluorobenzoate

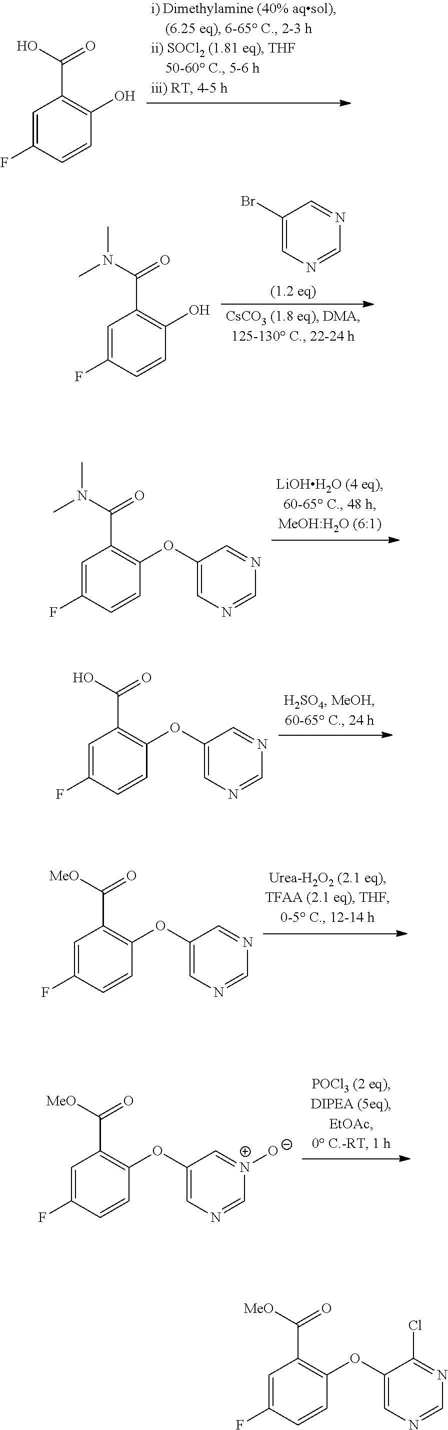

### Step 1. 5-Fluoro-2-hydroxy-N,N-dimethylbenzamide

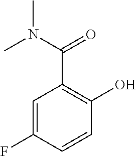

A 40% aqueous dimethylamine solution (140.0 L, 1200.0 mol) was heated at 60-65° C. for 2-3 h to release dimethylamine gas, which was purged into a cooled solution (0-5° C.) of THE (250.0 L).

In another reactor, thionyl chloride (41.0 kg, 347.4 moles) was added to a solution of 5-fluorosalicylic acid (30.0 kg, 192.0 moles) in THF (100.0 L) over a period of 1-2 h. The reaction mixture was heated to 50-60° C. for 5-6 h. After completion of the reaction, as judged by TLC, the reaction mixture was concentrated completely. THF was added and distilled out to remove the remaining thionyl chloride. The acid chloride thus obtained was dissolved in THF (30.0 L) and was added to the dimethylamine-THE solution at 0-5° C. The reaction mixture was stirred at RT for 4-5 h. After completion of the reaction, the reaction mixture was concentrated under a vacuum below 40° C. Ice water was added, and the mixture was stirred for 1-2 h at room temperature to precipitate a solid. The solid was filtered and washed with water. The solid was triturated with cyclohexane and centrifuged to give 5-fluoro-2-hydroxy-N,N-dimethylbenzamide (28.5 kg, 81% yield) as a solid. ¹H NMR (400 MHz, DMSO-d₆): δ 2.81 (s, 3H), 2.94 (s, 3H), 6.83-6.87 (m, 1H), 6.92-6.95 (m, 1H), 7.02-7.07 (m, 1H), 9.76 (br, 1H); HPLC(% purity): 99.63%.

### Step 2. 5-Fluoro-N,N-dimethyl-2-(pyrimidin-5-yloxy)benzamide

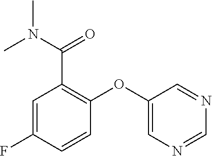 [SMILES: CN(C)C(=O)c1cc(F)ccc1Oc1cncnc1]

To a solution of 5-fluoro-2-hydroxy-N,N-dimethylbenzamide (28.0 kg, 153.0 moles) and 5-bromopyrimidine (29.16 kg, 183.0 moles) in dimethyl acetamide (180.0 L) was added cesium carbonate (89.73, 270.0 moles). The reaction mixture was heated to 125-130° C. for 22-24 h. After complete consumption of the starting material, as judged by TLC, the reaction mixture was cooled to 35-40° C. and filtered to remove the cesium carbonate. The reaction mixture was concentrated under vacuum. The residue thus obtained was dissolved in water (84.0 L) and extracted with DCM (2×56.0 L). The DCM layer was washed with 10% aqueous sodium hydroxide solution, followed by dilute HCl solution, dried over sodium sulfate, filtered, and concentrated to give 5-fluoro-N,N-dimethyl-2-(pyrimidin-5-yloxy)benzamide (24.0 kg, 60.1% yield) as a semisolid. ¹H NMR (400 MHz, DMSO-d₆): δ 8.94 (s, 1H), 8.54 (s, 2H), 7.25-7.34 (m, 3H), 2.85 (s, 3H), 2.84 (s, 3H); HPLC (% purity): 86.75%.

### Step 3. 5-Fluoro-2-(pyrimidin-5-yloxy)benzoic acid

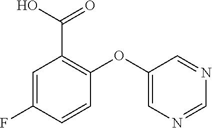 [SMILES: O=C(O)c1cc(F)ccc1Oc1cncnc1]

To a solution of 5-fluoro-N,N-dimethyl-2-(pyrimidin-5-yloxy)benzamide (23.5 kg, 90.03 moles) in 6:1 MeOH/H₂O (140.0 L) was added LiOH·H₂O (15.5 kg, 360.0 moles), and the reaction was heated at 60-65° C. for 48 h. The reaction mixture was cooled to 40° C., and the methanol was distilled out in vacuum below 40° C. The residue thus obtained was dissolved in water. The aqueous layer was washed with ethyl acetate. The aqueous layer was treated with activated carbon and filtered on Hyflo. The aqueous layer was acidified with concentrated HCl (1.5 L) to PH~1.0-2.0 to precipitate a solid. This solid was filtered, washed with water, and dried at 40-45° C. to give 5-fluoro-2-(pyrimidin-5-yloxy)benzoic acid (13.5 kg, 64.1% yield) as a solid. ¹H NMR (400 MHz, DMSO-d₆): δ 13.40 (br s, 1H), 8.92 (s, 1H), 8.46 (s, 2H), 7.69-7.71 (m, 1H), 7.52-7.57 (m, 1H), 7.38-7.42 (m, 1H); HPLC(% purity): 96.94%.

### Step 4. Methyl 5-fluoro-2-(pyrimidin-5-yloxy)benzoate

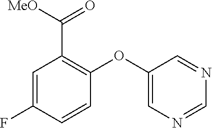 [SMILES: COC(=O)c1cc(F)ccc1Oc1cncnc1]

To a cooled solution of 5-fluoro-2-(pyrimidin-5-yloxy)benzoic acid (13.5 kg, 57.69 moles) in methanol (90.0 L) was added sulfuric acid (2.16 L), and the reaction was heated at 60-65° C. for 24 h. The reaction mixture was cooled to room temperature, and the methanol was distilled out completely under reduced pressure. The residue thus obtained was dissolved in DCM (54.0 L). The DCM layer was washed with 5% sodium bicarbonate solution and saturated sodium chloride solution. The organic layer was dried over anhydrous sodium sulfate, filtered, and concentrated to give methyl 5-fluoro-2-(pyrimidin-5-yloxy)benzoate (9.6 kg, 67.1% yield) as a solid. ¹H NMR (400 MHz, DMSO-d₆): δ 8.94 (s, 1H), 8.50 (s, 2H), 7.72-7.75 (m, 1H), 7.57-7.62 (m, 1H), 7.41-7.45 (m, 1H), 3.73 (s, 3H); HPLC(% purity): 98.37%; assay by Q-NMR (%): 99.21%.

Step 5. 5-(4-Fluoro-2-(methoxycarbonyl)phenoxy)pyrimidine 1-oxide

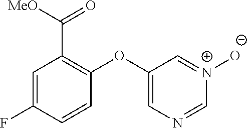 [SMILES: COC(=O)c1cc(F)ccc1Oc1cnc[n+]([O-])c1]

To a cooled solution of methyl 5-fluoro-2-(pyrimidin-5-yloxy)benzoate (9.6 kg, 38.7 moles) in THF (150 L) was added UHP (7.64 kg, 81.2 moles) at 0-5° C. (over a period of 30 min), and the reaction was stirred for 10 min at 0-5° C. TFAA (17.0 kg, 81.0 moles) was then added dropwise over 30-40 min at 0-5° C. The reaction was stirred at 0-5° C. for 12-14 h. After complete consumption of starting material, as judged by TLC, the reaction mixture was quenched by the addition of 5% NaHCO₃ solution at 0-5° C. DCM (60.0 L) was then added at 5-10° C., and the mixture was stirred for 20 min. The aqueous and organic layers were separated. The organic layer was washed with 5% NaHCO₃. Aqueous sodium thiosulfate solution was then added to the organic layer (DCM layer), and the mixture was stirred for 12 hours at 10° C. The organic layer was separated, dried over anhydrous Na₂SO₄, filtered, and concentrated under reduced pressure to give 5-(4-fluoro-2-(methoxycarbonyl)phenoxy)pyrimidine-1-oxide (5.12 kg, 50.0% yield) as solid. ¹HNMR (400 MHz, DMSO-d₆): δ 8.86 (s, 1H), 8.44 (s, 1H), 8.01 (s, 1H), 7.72-7.75 (m, 1H), 7.59-7.64 (m, 1H), 7.49-7.52 (m, 1H), 3.76 (s, 3H); HPLC(% purity): 99.60%.

### Step 6. Methyl 2-((4-chloropyrimidin-5-yl)oxy)-5-fluorobenzoate

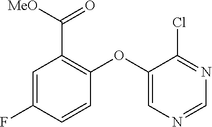 [SMILES: COC(=O)c1cc(F)ccc1Oc1cncnc1Cl]

In a 100 mL two neck round bottom flask under a nitrogen atmosphere, 5-(4-fluoro-2-(methoxycarbonyl)phenoxy)pyrimidine 1-oxide (20 g, 76 mmol) was dissolved in EtOAc (200 mL). To this solution, DIPEA (65.3 mL, 378 mmol) was added at 0° C., and the reaction was stirred at the same temperature for 15 min. To this reaction mixture, POCI3 (14.15 mL, 151 mmol) was added dropwise at 0° C. The reaction was warmed to RT and stirred for 1 h. The reaction was monitored by TLC(50% EtOAc in hexane). The reaction was concentrated under reduced pressure to afford crude compound. The crude compound was purified by silica gel flash column chromatography using EtOAc in hexane (product eluted at 25% EtOAc in hexane) to obtain methyl 2-((4-chloropyrimidin-5-yl)oxy)-5-fluorobenzoate (16.5 g, 72.2% yield) as a solid. ¹HNMR (400 MHz, DMSO-d₆): δ 8.81 (s, 1H), 8.30 (s, 1H), 7.76 (dd, J=3.1, 8.8 Hz, 1H), 7.61 (ddd, J=3.3, 7.9, 9.0 Hz, 1H), 7.46 (dd, J=4.6, 9.0 Hz, 1H), 3.75 (s, 3H); LCMS (Method A): Rt=1.74 min, m/z=283.1 [M+H]⁺; 93.65%.

### Intermediate 3. Methyl 2-((4-(2,7-diazaspiro[3.5]nonan-2-yl)pyrimidin-5-yl)oxy)-5-fluorobenzoate hydrochloride

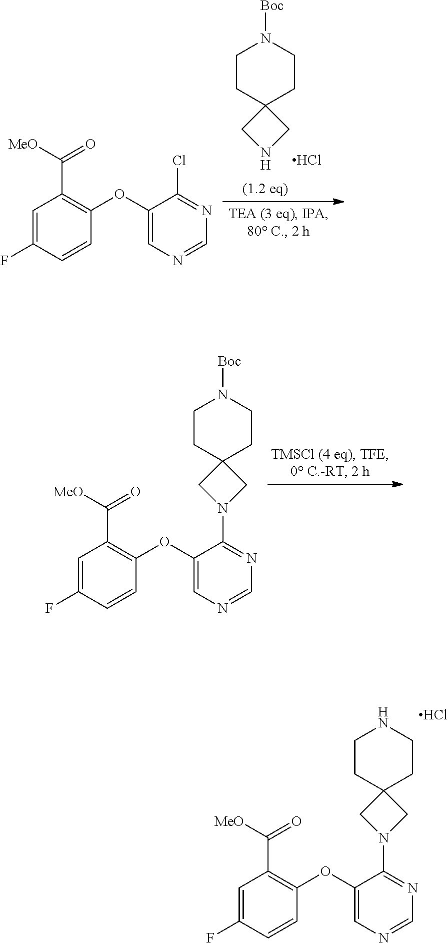 [SMILES: COC(=O)c1cc(F)ccc1Oc1cncnc1Cl ; CC(C)(C)OC(=O)N1CCC2(CC1)CNC2 ; Cl ; COC(=O)c1cc(F)ccc1Oc1cncnc1N1CC2(CCN(C(=O)OC(C)(C)C)CC2)C1 ; Cl ; COC(=O)c1cc(F)ccc1Oc1cncnc1N1CC2(CCNCC2)C1]

Step 1. tert-Butyl 2-(5-(4-fluoro-2-(methoxycarbonyl)phenoxy)pyrimidin-4-yl)-2,7-diazaspiro[3.5]nonane-7-carboxylate

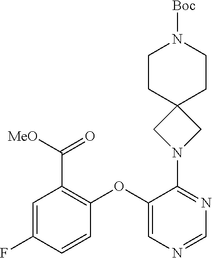 [SMILES: COC(=O)c1cc(F)ccc1Oc1cncnc1N1CC2(CCN(C(=O)OC(C)(C)C)CC2)C1]

To a stirred solution of methyl 2-((4-chloropyrimidin-5-yl)oxy)-5-fluorobenzoate (25 g, 88 mmol) in IPA (250 mL), tert-butyl 2,7-diazaspiro[3.5]nonane-7-carboxylate hydrochloride (27.9 g, 106 mmol) and TEA (37 mL, 265 mmol) were added at RT under a nitrogen atmosphere. The reaction was stirred at 80° C. for 2 h, and monitored by LCMS and TLC(10% MeOH in DCM). After completion, the reaction was concentrated under reduced pressure. The crude was purified by silica gel flash column chromatography using EtOAc in hexane (product eluted at 30% EtOAc in hexane) to obtain tert-butyl 2-(5-(4-fluoro-2-(methoxycarbonyl)phenoxy)pyrimidin-4-yl)-2,7-diazaspiro[3.5]nonane-7-carboxylate (41 g, 97% yield) as a solid. ¹H NMR (400 MHz, DMSO-d₆): δ 8.29-8.26 (m, 1H), 7.68-7.64 (m, 2H), 7.49 (ddd, J=3.3, 7.9, 9.0 Hz, 1H), 7.14 (dd, J=4.5, 9.1 Hz, 1H), 3.92 (s, 4H), 3.80 (s, 3H), 3.31-3.21 (m, 4H), 1.72-1.62 (m, 4H), 1.39 (s, 9H); LCMS (Method A): Rt 2.06 min, m/z: 473.6 [M+H]⁺, 98.45%.

### Step 2. Methyl 2-((4-(2,7-diazaspiro[3.5]nonan-2-yl)pyrimidin-5-yl)oxy)-5-fluorobenzoate hydrochloride

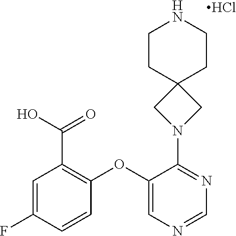 [SMILES: Cl ; O=C(O)c1cc(F)ccc1Oc1cncnc1N1CC2(CCNCC2)C1]

To a stirred solution of tert-butyl 2-(5-(4-fluoro-2-(methoxycarbonyl)phenoxy)pyrimidin-4-yl)-2,7-diazaspiro[3.5]nonane-7-carboxylate (20 g, 42.3 mmol) in TFE (200 mL), TMSCI (21.64 mL, 169 mmol) was added at 0° C. under a nitrogen atmosphere. The reaction was stirred RT for 2 h, and monitored by TLC(10% MeOH in DCM) and LCMS. After completion, the reaction was concentrated under reduced pressure and reconcentrated from EtOAc (2×50 mL) to obtain crude methyl 2-((4-(2,7-diazaspiro[3.5]nonan-2-yl)pyrimidin-5-yl)oxy)-5-fluorobenzoate hydrochloride (21 g, quantitative yield) as a solid. This material was used without further purification. ¹H NMR (400 MHz, DMSO-d₆): δ 9.07 (br s, 2H), 8.63-8.58 (m, 1H), 7.77 (dd, J=3.2, 8.7 Hz, 1H), 7.71 (s, 1H), 7.67-7.61 (m, 1H), 7.47 (dd, J=4.6, 9.1 Hz, 1H), 4.49-4.15 (m, 4H), 3.81 (s, 3H), 3.11-2.98 (m, 4H), 2.06-1.97 (m, 4H); LCMS (Method A): Rt 1.30 min, m/z: 373.4 [M+H]⁺, 97.61%.

### Intermediate 4. ((2S,5R)-5-Aminotetrahydro-2/I-pyran-2-yl)methyl 4-methylbenzenesulfonate hydrochloride

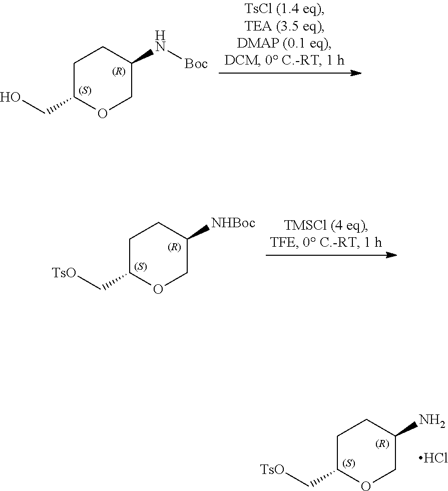

### Step 1. ((2S,5R)-5-((tert-Butoxycarbonyl)amino)tetrahydro-2H-pyran-2-yl)methyl 4-methylbenzenesulfonate

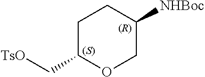

To a stirred solution of tert-butyl((3R,6S)-6-(hydroxymethyl)tetrahydro-2H-pyran-3-yl)carbamate (20 g, 86 mmol) in DCM (200 mL), TEA (42.2 mL, 303 mmol), DMAP (1.056 g, 8.65 mmol), and TsC1 (23.08 g, 121 mmol) were added at 0° C. The reaction was stirred at RT for 16 h, and monitored by LCMS and TLC(10% MeOH in DCM). After completion, the reaction was quenched with water (150 mL) and the mixture was extracted with DCM (2×200 mL). The combined organic layer was washed with brine solution (100 mL), dried over sodium sulfate and filtered, and the filtrate was concentrated under reduced pressure. The crude was purified by silica gel flash column chromatography using MeOH in DCM (product eluted at 5% MeOH in DCM). The pure fractions were concentrated under reduced pressure to obtain ((25,5R)-5-((tert-butoxycarbonyl)amino)tetrahydro-2H-pyran-2-yl)methyl 4-methylbenzenesulfonate (34 g, 97% yield) as a solid. ¹H NMR (400 MHz, DMSO-d₆): δ 7.78 (d, J=8.3 Hz, 2H), 7.49 (d, J=8.1 Hz, 2H), 6.74 (br d, J=7.9 Hz, 1H), 4.01-3.96 (m, 1H), 3.94-3.86 (m, 1H), 3.77-3.68 (m, 1H), 3.41-3.34 (m, 1H), 3.28-3.18 (m, 1H), 2.88 (t, J=10.7 Hz, 1H), 2.43 (s, 3H), 1.86-1.76 (m, 1H), 1.58-1.51 (m, 1H), 1.37 (s, 9H), 1.35-1.17 (m, 2H); LCMS (Method C): Rt=1.98 min, m/z=330.0 [M+H−56]^{+, 95.54}%.

### Step 2. ((2S,5R)-5-Aminotetrahydro-2H-pyran-2-yl)methyl 4-methylbenzenesulfonate hydrochloride

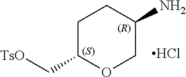

To a stirred solution of ((2S,5R)-5-((tert-butoxycarbonyl)amino)tetrahydro-2H-pyran-2-yl)methyl 4-methylbenzenesulfonate (5.0 g, 12.97 mmol) in TFE (50 mL), TMSCI (6.63 mL, 51.9 mmol) was added drop wise at 0° C. The reaction was stirred at RT for 1 h, and monitored with TLC(20% EtOAc in hexane). After completion, the reaction was concentrated under reduced pressure. The residue was reconcentrated from EtOAc (2×50 mL) to obtain crude ((2S,5R)-5-aminotetrahydro-2H-pyran-2-yl)methyl 4-methylbenzenesulfonate hydrochloride (4.6 g, 97% yield) as a solid. This material was used without further purification. ¹H NMR (400 MHz, DMSO-d₆): δ 8.01 (br s, 3H), 7.78 (d, J=8.3 Hz, 2H), 7.49 (d, J=8.1 Hz, 2H), 4.06-3.99 (m, 1H), 3.98-3.90 (m, 2H), 3.53-3.44 (m, 1H), 3.20 (t, J=10.8 Hz, 1H), 3.08-2.98 (m, 1H), 2.43 (s, 3H), 2.08-1.98 (m, 1H), 1.69-1.58 (m, 1H), 1.56-1.44 (m, 1H), 1.37-1.24 (m, 1H); LCMS (Method A): Rt=1.47 min, m/z=286.0 [M+H]⁺, 88.04%.

### Intermediate 5. ((2S,5R)-5-((N-Ethylsulfamoyl)amino)tetrahydro-2H-pyran-2-yl)methyl 4-methylbenzenesulfonate

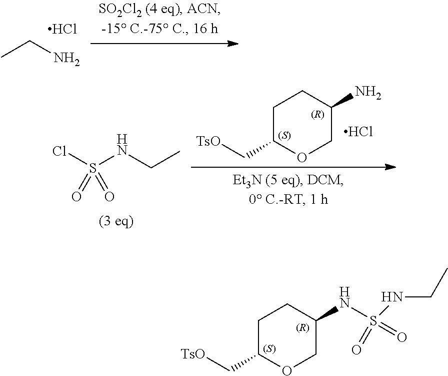

### Step 1. Ethylsulfamoyl chloride

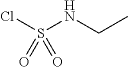

In a dried, 250 mL two neck round bottom flask under a nitrogen atmosphere, ethanamine hydrochloride (5 g, 61.3 mmol) was taken into ACN (50 mL). To this solution, sulfuryl dichloride (19.82 mL, 245 mmol) was added at −15° C. The reaction was stirred at 75° C. for 16 h. After completion, the reaction was concentrated under reduced pressure, and the residue was triturated with MTBE (250 mL). The solid was filtered, and the filtrate was concentrated under reduced pressure to obtain crude ethylsulfamoyl chloride (8.86 g, quantitative yield) as a liquid. This material was used without further purification.

### Step 2. ((2S,5R)-5-((N-Ethylsulfamoyl)amino)tetrahydro-2H-pyran-2-yl)methyl 4 methylbenzenesulfonate

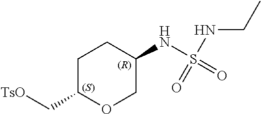

Step 2 was performed following the same procedure in two batches (3.6 g and 0.5 g).

In a dried, 250 mL two neck round bottom flask under a nitrogen atmosphere, ((2S,5R)-5-aminotetrahydro-2H-pyran-2-yl)methyl 4-methylbenzenesulfonate hydrochloride (3.6 g, 11.19 mmol) was dissolved in DCM (72 mL). To this solution, TEA (7.80 mL, 55.9 mmol) was added, then a solution of ethylsulfamoyl chloride (3.47 mL, 33.6 mmol) in DCM (3.5 mL) was added at RT. The reaction was stirred at RT for 1 h, and monitored by TLC(50% EtOAc in hexane). After completion, the reaction mixtures from both batches were combined and quenched with water (100 mL), and the mixture was extracted with EtOAc (2×10⁰ mL). The combined organic layer was dried over sodium sulfate and filtered, and the filtrate was concentrated under reduced pressure. The crude was purified by silica gel flash column chromatography using EtO Ac in hexane (product eluted at 50% EtOAc in hexane). The pure fractions were concentrated under reduced pressure to obtain ((2S,5R)-5-((N-ethylsulfamoyl)amino)tetrahydro-2H-pyran-2-yl)methyl 4-methylbenzenesulfonate (3.2 g, 59.79% combined yield) as a gummy solid. ¹H NMR (400 MHz, DMSO-d₆): δ 7.78 (d, J=8.4 Hz, 2H), 7.49 (d, J=8.0 Hz, 2H), 6.87 (d, J=7.0 Hz, 1H), 6.79 (t, J=5.8 Hz, 1H), 4.07-3.96 (m, 1H), 3.93-3.81 (m, 2H), 3.44-3.36 (m, 1H), 3.01-2.94 (m, 2H), 2.85-2.75 (m, 2H), 2.43 (s, 3H), 1.97-1.89 (m, 1H), 1.60-1.54 (m, 1H), 1.40-1.22 (m, 2H), 1.05 (t, J=7.2 Hz, 3H); LCMS (Method A): Rt 1.74 min, m/z: 393.0 [M+H]⁺, 93.43%.

### Intermediate 6. ((2S,5R)-5-(Ethylsulfonamido)tetrahydro-2H-pyran-2-yl)methyl 4-methylbenzenesulfonate

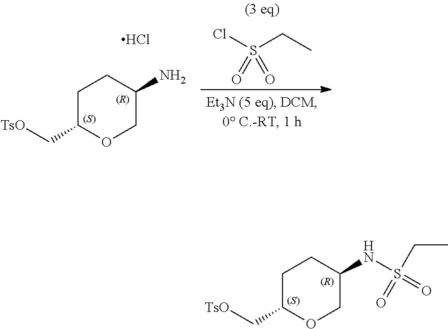

To a stirred solution of ((2S,5R)-5-aminotetrahydro-2H-pyran-2-yl)methyl 4-methylbenzenesulfonate hydrochloride (4.17 g, 12.96 mmol) in DCM (80 mL), TEA (9.11 mL, 64.8 mmol) was added at 0° C. under a nitrogen atmosphere. To this reaction mixture, a solution of ethanesulfonyl chloride (5.00 g, 38.9 mmol) in DCM (3.5 mL) was added at 0° C. The reaction was stirred at RT for 1 h, and monitored by TLC(50% EtOAc in hexane). After completion, the reaction was quenched with water (50 mL) and the mixture was extracted with DCM (2×10⁰ mL). The combined organic layer was dried over anhydrous sodium sulfate and filtered, and the filtrate was concentrated under reduced pressure. The crude was purified by silica gel flash column chromatography using EtOAc in hexane (product eluted with 60% EtOAc in hexane) to obtain ((2S,5R)-5-(ethylsulfonamido)tetrahydro-2H-pyran-2-yl)methyl 4-methylbenzenesulfonate (4.2 g, 85% yield) as a liquid. ¹H NMR (400 MHz, DMSO-d₆): δ 7.78 (d, J=8.3 Hz, 2H), 7.49 (d, J=8.0 Hz, 2H), 7.09 (d, J=7.6 Hz, 1H), 4.03-3.97 (m, 1H), 3.93-3.87 (m, 1H), 3.83-3.77 (m, 1H), 3.44-3.38 (m, 1H), 3.13-2.94 (m, 4H), 2.43 (s, 3H), 1.98-1.88 (m, 1H), 1.63-1.53 (m, 1H), 1.45-1.21 (m, 2H), 1.17 (t, J=7.3 Hz, 3H); LCMS (Method A): Rt=1.73 min, m/z=376.0 [M−H]⁻, 99.30%.

### Intermediate 7. ((1r,4r)-4-(Ethylsulfonamido)cyclohexyl)methyl 4-methylbenzenesulfonate

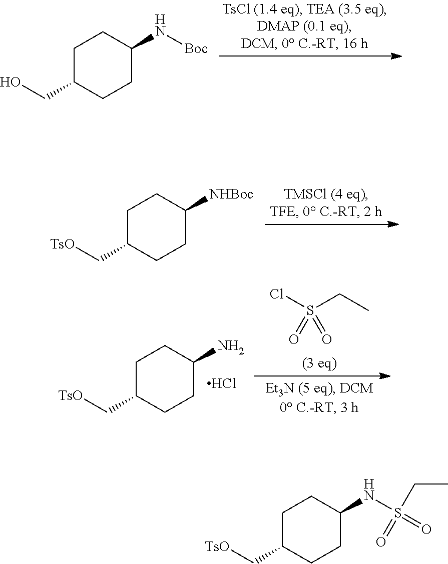

### Step 1. ((1r,4r)-4-((tert-Butoxycarbonyl)amino)cyclohexyl)methyl 4-methylbenzenesulfonate

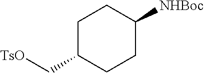 [SMILES: Cc1ccc(S(=O)(=O)OC[C@H]2CC[C@H](NC(=O)OC(C)(C)C)CC2)cc1]

This compound was synthesized following the procedure described for the synthesis of Intermediate 4, step 1, starting with 1 equivalent of tert-butyl((1r,4r)-4-(hydroxymethyl)cyclohexyl)carbamate and 1.4 equivalents of TsCl. After work-up, the crude product was purified by silica gel flash column chromatography using MeOH in DCM (product eluted at 5% MeOH in DCM) to obtain ((1r,4r)-4-((tert-butoxycarbonyl)amino)cyclohexyl)methyl 4-methylbenzenesulfonate as a solid.

Yield: 80%; ¹H NMR (400 MHz, DMSO-d₆): δ 7.78 (br d, J=8.1 Hz, 2H), 7.49 (br d, J=8.0 Hz, 2H), 6.69 (br d, J=7.4 Hz, 1H), 3.82 (br d, J=6.3 Hz, 2H), 3.16-3.00 (m, 1H), 2.43 (s, 3H), 1.73 (br d, J=10.9 Hz, 2H), 1.60 (br d, J=12.0 Hz, 2H), 1.54-1.43 (m, 1H), 1.36 (s, 9H), 1.14-1.00 (m, 2H), 0.97-0.83 (m, 2H); LCMS (Method D): Rt=2.20 min, m/z=328.1 [M+H−56]⁺, 92.91%.

### Step 2. ((1r,4r)-4-Aminocyclohexyl)methyl 4-methylbenzenesulfonate hydrochloride

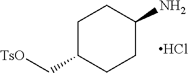 [SMILES: Cc1ccc(S(=O)(=O)OC[C@H]2CC[C@H](N)CC2)cc1 ; Cl]

This compound was synthesized following the procedure described for the synthesis of Intermediate 4, step 2, starting with 1 equivalent of ((1r,4r)-4-((tert-butoxycarbonyl)amino)cyclohexyl)methyl 4-methylbenzenesulfonate. After completion, the reaction was concentrated under reduced pressure. The residue was reconcentrated from EtOAc to obtain crude ((1r,4r)-4-aminocyclohexyl)methyl 4-methylbenzenesulfonate hydrochloride as a solid. This material was used without further purification.

Yield: 79%; ¹H NMR (400 MHz, DMSO-d₆): δ 8.04 (br s, 3H), 7.78 (d, J=8.3 Hz, 2H), 7.49 (d, J=8.0 Hz, 2H), 3.84 (d, J=6.1 Hz, 2H), 2.97-2.78 (m, 1H), 2.43 (s, 3H), 1.92 (br d, J=10.5 Hz, 2H), 1.67 (br d, J=12.1 Hz, 2H), 1.60-1.47 (m, 1H), 1.35-1.19 (m, 2H), 1.04-0.89 (m, 2H); LCMS (Method A): Rt=1.58 min, m/z=284.1 [M+H]⁺, 82.51%.

### Step 3. ((1r,4r)-4-(Ethylsulfonamido)cyclohexyl)methyl 4-methylbenzenesulfonate

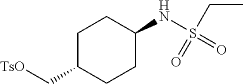 [SMILES: CCS(=O)(=O)N[C@H]1CC[C@H](COS(=O)(=O)c2ccc(C)cc2)CC1]

This compound was synthesized following the procedure described for the synthesis of Intermediate 6, starting with 1 equivalent of ((1r,4r)-4-aminocyclohexyl)methyl 4-methylbenzenesulfonate hydrochloride and 3 equivalents of ethanesulfonyl chloride. After work-up, the crude product was purified by silica gel flash column chromatography using EtOAc in hexane (product eluted t 60% EtOAc in hexane) to obtain ((1r,4r)-4-(ethylsulfonamido)cyclohexyl)methyl 4-methylbenzenesulfonate as a solid.

Yield: 46.4%; ¹H NMR (400 MHz, DMSO-d₆): δ 7.78 (d, J=8.1 Hz, 2H), 7.49 (br d, J=8.0 Hz, 2H), 6.99 (br d, J=7.6 Hz, 1H), 3.82 (d, J=6.3 Hz, 2H), 3.01-2.87 (m, 3H), 2.43 (s, 3H), 1.83 (br d, J=10.6 Hz, 2H), 1.61 (br d, J=12.4 Hz, 2H), 1.55-1.43 (m, 1H), 1.23-1.07 (m, 5H), 1.01-0.88 (m, 2H); LCMS (Method A): Rt=1.87 min, m/z=374.1 [M−H]⁻, 99.08%.

### Intermediate 8. 2-((4-(7-(((25,5R)-5-((N-Ethylsulfamoyl)amino)tetrahydro-2H-pyran-2-yl)methyl)-2,7-diazaspiro[3.5]nonan-2-yl)pyrimidin-5-yl)oxy)-5-fluorobenzoic acid

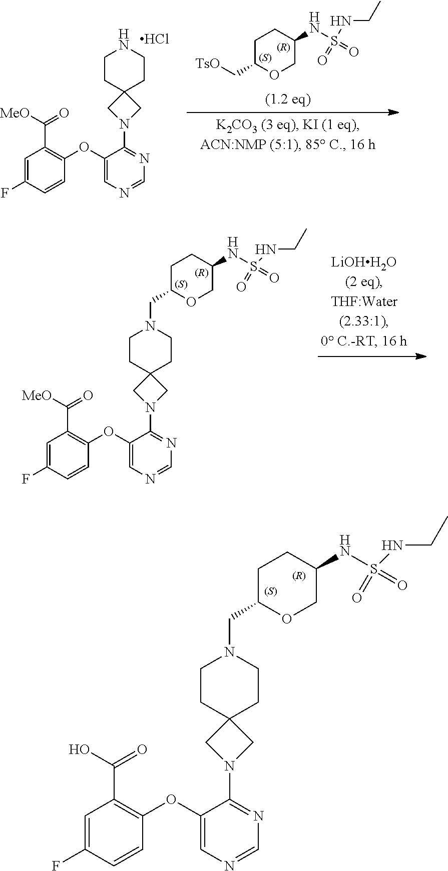

### Step 1. Methyl 2-((4-(7-(((2S,5R)-5-((N-ethylsulfamoyl)amino)tetrahydro-2H-pyran-2-yl)methyl)-2, 7-diazaspiro[3.5]nonan-2-yl)pyrimidin-5-yl)oxy)-5-fluorobenzoate

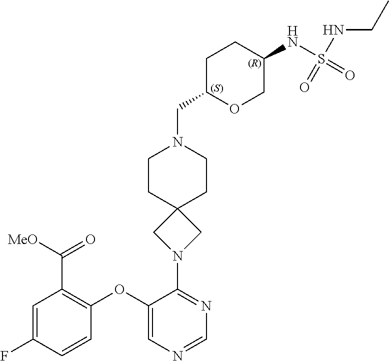

In a dried, 100 mL two neck round bottom flask under a nitrogen atmosphere, methyl 2-((4-(2,7-diazaspiro[3.5]nonan-2-yl)pyrimidin-5-yl)oxy)-5-fluorobenzoate hydrochloride (1.4 g, 3.42 mmol) was dissolved in 5:1 ACN: NMP (30 mL: 6 mL). To this solution, K₂CO₃ (1.420 g, 10.27 mmol), KI (0.568 g, 3.42 mmol) and ((2S,5R)-5-((N-ethylsulfamoyl)amino)tetrahydro-2H-pyran-2-yl)methyl 4-methylbenzenesulfonate (1.613 g, 4.11 mmol) were added at RT. The reaction was stirred at 85° C. for 16 h, and monitored by TLC(10% MeOH in DCM). After completion, the reaction was quenched with water (50 mL), and the mixture was extracted with 10% MeOH in DCM (2×50 mL). The combined organic layer was dried over sodium sulfate and filtered, and the filtrate was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN) to obtain methyl 2-((4-(7-(((2S,5R)-5-((N-ethylsulfamoyl)amino)tetrahydro-2H-pyran-2-yl)methyl)-2,7-diazaspiro[3.5]nonan-2-yl)pyrimidin-5-yl)oxy)-5-fluorobenzoate (0.750 g, 34.9% yield)) as a solid. ¹H NMR (400 MHz, DMSO-d₆): δ 8.28 (s, 1H), 7.68-7.64 (m, 2H), 7.48 (ddd, J=3.3, 8.0, 8.9 Hz, 1H), 7.10 (dd, J=4.4, 9.1 Hz, 1H), 6.86 (br d, J=7.0 Hz, 1H), 6.78 (t, J=5.8 Hz, 1H), 3.86 (br s, 4H), 3.80 (s, 3H), 3.31-3.29 (m, 1H), 3.06-2.95 (m, 2H), 2.86-2.78 (m, 2H), 2.36-2.17 (m, 6H), 1.99-1.87 (m, 1H), 1.74-1.63 (m, 5H), 1.43-1.30 (m, 1H), 1.27-1.17 (m, 1H), 1.06 (t, J=7.2 Hz, 3H), one proton merged with solvent peaks; LCMS (Method A): Rt 1.64 min, m/z: 593.2 [M+H]⁺, 94.39%.

### Step 2. 2-((4-(7-(((2S,5R)-5-((N-Ethylsulfamoyl)amino)tetrahydro-2H-pyran-2-yl)methyl)-2, 7-diazaspiro[3.5]nonan-2-yl)pyrimidin-5-yl)oxy)-5-fluorobenzoic acid

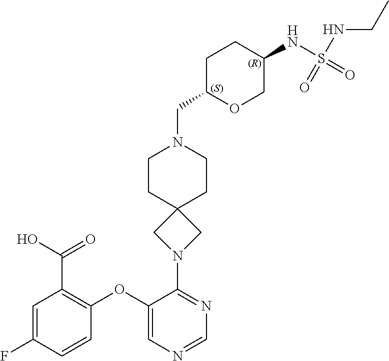

To a stirred solution of methyl 2-((4-(7-(((2S,5R)-5-((N-ethylsulfamoyl)amino)tetrahydro-2H-pyran-2-yl)methyl)-2,7-diazaspiro[3.5]nonan-2-yl)pyrimidin-5-yl)oxy)-5-fluorobenzoate (750 mg, 1.265 mmol) in 2.3:1 THF: H₂O (7 mL: 3 mL), LiOH·H₂O (106 mg, 2.53 mmol) was added at 0° C. The reaction was stirred at RT for 16 h, and monitored by LCMS and TLC(10% MeOH in DCM). After completion, the reaction was concentrated under reduced pressure. The residue was dissolved in water (10 mL), acidified with 1.5 N HCl (~5 pH), and the mixture was concentrated under reduced pressure. The residue was purified by reverse phase chromatography using a SiliCycle SiliaSep C18 column (80 g) on Biotage isolera one (Mobile phase A: 10 mM NH₄HCO₃ in water, B: ACN). The fractions were concentrated under reduced pressure and lyophilized to afford 2-((4-(7-(((2S,5R)-5-((N-ethylsulfamoyl)amino)tetrahydro-2H-pyran-2-yl)methyl)-2,7-diazaspiro[3.5]nonan-2-yl)pyrimidin-5-yl)oxy)-5-fluorobenzoic acid (600 mg, 81% yield) as a solid. ¹H NMR (400 MHz, DMSO-d₆): δ 8.12 (s, 1H), 7.35 (s, 1H), 7.20 (dd, J=2.9, 8.9 Hz, 1H), 7.02 (dt, J=3.3, 8.4 Hz, 1H), 6.94-6.89 (m, 1H), 6.87 (br d, J=6.9 Hz, 1H), 6.79 (t, J=5.8 Hz, 1H), 4.01-3.84 (m, 4H), 3.05-2.95 (m, 2H), 2.88-2.77 (m, 2H), 2.39-2.16 (m, 6H), 1.99-1.86 (m, 1H), 1.77-1.60 (m, 5H), 1.45-1.30 (m, 1H), 1.30-1.16 (m, 1H), 1.05 (t, J=7.2 Hz, 3H), three protons merged with solvent peaks; LCMS (Method A): Rt 1.15 min, m/z: 579.2 [M+H]⁺, 99.47%.

### Intermediate 9. 2-((4-(7-(((2S,5R)-5-(Ethylsulfonamido)tetrahydro-2H-pyran-2-yl)methyl)-2,7-diazaspiro[3.5]nonan-2-yl)pyrimidin-5-yl)oxy)-5-fluorobenzoic acid

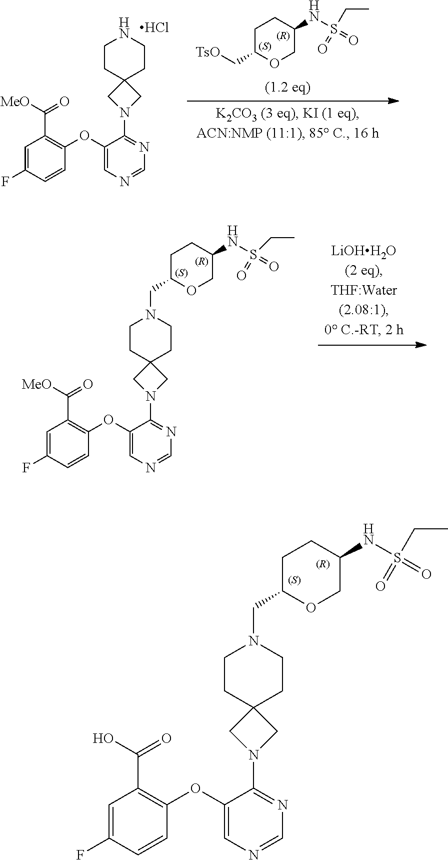

### Step 1. Methyl 2-((4-(7-(((2S,5R)-5-(ethylsulfonamido)tetrahydro-2H-pyran-2-yl)methyl)-2,7-diazaspiro[3.5]nonan-2-yl)pyrimidin-5-yl)oxy)-5-fluorobenzoate

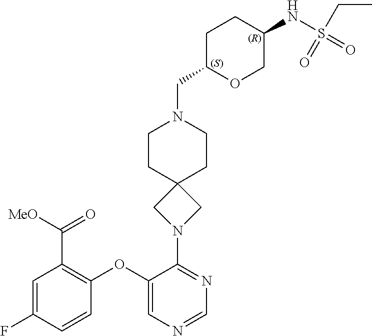

This compound was synthesized following the procedure described for the synthesis of Intermediate 8, step 1, starting with 1 equivalent of methyl 2-((4-(2,7-diazaspiro[3.5]nonan-2-yl)pyrimidin-5-yl)oxy)-5-fluorobenzoate hydrochloride and 1.2 equivalents of ((2S,5R)-5-(ethylsulfonamido)tetrahydro-2H-pyran-2-yl)methyl 4-methylbenzenesulfonate, except that solvent 11:1 ACN: NMP (33 mL: 3 mL) was used. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain methyl 2-((4-(7-(((2S,5R)-5-(ethylsulfonamido)tetrahydro-2H-pyran-2-yl)methyl)-2,7-diazaspiro[3.5]nonan-2-yl)pyrimidin-5-yl)oxy)-5-fluorobenzoate as a solid.

Yield: 60.5%; ¹H NMR (400 MHz, DMSO-d₆): δ 8.28 (s, 1H), 7.68-7.64 (m, 2H), 7.48 (ddd, J=3.3, 7.9, 9.0 Hz, 1H), 7.13-7.08 (m, 2H), 3.89-3.82 (m, 5H), 3.80 (s, 3H), 3.32-3.27 (m, 1H), 3.14-2.96 (m, 4H), 2.36-2.15 (m, 6H), 2.00-1.87 (m, 1H), 1.73-1.65 (m, 5H), 1.47-1.34 (m, 1H), 1.29-1.21 (m, 1H), 1.18 (t, J=7.3 Hz, 3H); LCMS (Method C): Rt=1.22 min, m/z=578.2 [M−H]⁻, 97.27%.

### Step 2. 2-((4-(7-(((2S,5R)-5-(Ethylsulfonamido)tetrahydro-2H-pyran-2-yl)methyl)-2,7-diazaspiro[3.5]nonan-2-yl)pyrimidin-5-yl)oxy)-5-fluorobenzoic acid

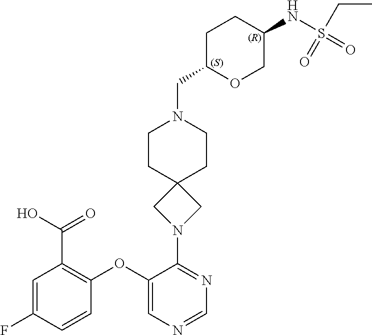

This compound was synthesized following the procedure described for the synthesis of Intermediate 8, step 2, starting with 1 equivalent of methyl 2-((4-(7-(((2S,5R)-5-(ethylsulfonamido)tetrahydro-2H-pyran-2-yl)methyl)-2,7-diazaspiro[3.5]nonan-2-yl)pyrimidin-5-yl)oxy)-5-fluorobenzoate, except that solvent 2.08:1 THF: H₂O (25 mL: 12 mL) were used. After work-up, the crude was purified by reverse phase chromatography using a SiliCycle SiliaSep C18 column (120 g) on Biotage isolera one (Mobile phase A: 10 mM NH₄HCO₃ in water, B: ACN). The fractions were concentrated under reduced pressure to afford 2-((4-(7-(((2S,5R)-5-(ethylsulfonamido)tetrahydro-2H-pyran-2-yl)methyl)-2,7-diazaspiro[3.5]nonan-2-yl)pyrimidin-5-yl)oxy)-5-fluorobenzoic acid as a solid.

Yield: 91%; ¹H NMR (400 MHz, DMSO-d₆): δ 8.21 (s, 1H), 7.53-7.47 (m, 2H), 7.36-7.29 (m, 1H), 7.16 (d, J=7.5 Hz, 1H), 7.07 (dd, J=4.6, 9.0 Hz, 1H), 3.93 (br s, 4H), 3.86-3.77 (m, 1H), 3.53-3.41 (m, 1H), 3.14-2.97 (m, 4H), 2.71-2.57 (m, 3H), 2.01-1.89 (m, 1H), 1.87-1.75 (m, 4H), 1.70-1.60 (m, 1H), 1.49-1.33 (m, 1H), 1.33-1.22 (m, 1H), 1.18 (t, J=7.3 Hz, 3H), four protons merged with solvent peaks; LCMS (Method A): Rt=1.15 min, m/z=564.2 [M+H]⁺, 99.48%.

### Intermediate 10. 2-((4-(7-(((1r,4r)-4-(Ethylsulfonamido)cyclohexyl)methyl)-2,7-diazaspiro[3.5]nonan-2-yl)pyrimidin-5-yl)oxy)-5-fluorobenzoic acid

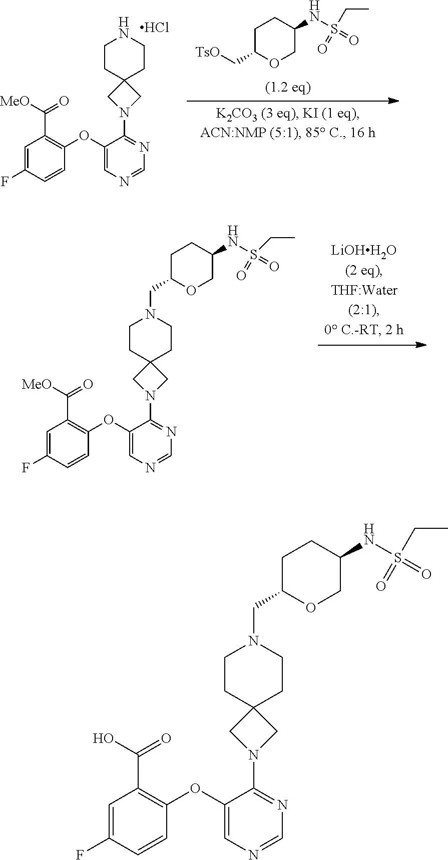 [SMILES: COC(=O)c1cc(F)ccc1Oc1cncnc1N1CC2(CCNCC2)C1 ; Cl ; CCS(=O)(=O)N[C@@H]1CC[C@@H](COS(=O)(=O)c2ccc(C)cc2)OC1 ; CCS(=O)(=O)N[C@@H]1CC[C@@H](CN2CCC3(CC2)CN(c2ncncc2Oc2ccc(F)cc2C(=O)OC)C3)OC1 ; CCS(=O)(=O)N[C@@H]1CC[C@@H](CN2CCC3(CC2)CN(c2ncncc2Oc2ccc(F)cc2C(=O)O)C3)OC1]

### Step 1. Methyl 2-((4-(7-(((1r,4r)-4-(ethylsulfonamido)cyclohexyl)methyl)-2,7-diazaspiro[3.5]nonan-2-yl)pyrimidin-5-yl)oxy)-5-fluorobenzoate

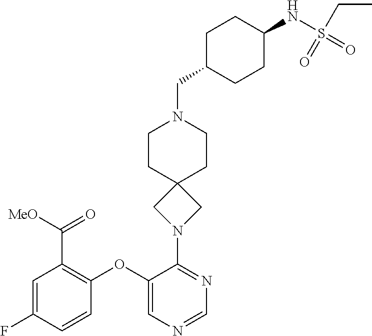 [SMILES: CCS(=O)(=O)N[C@H]1CC[C@H](CN2CCC3(CC2)CN(c2ncncc2Oc2ccc(F)cc2C(=O)OC)C3)CC1]

This compound was synthesized following the procedure described for the synthesis of Intermediate 8, step 1, starting with 1 equivalent of methyl 2-((4-(2,7-diazaspiro[3.5]nonan-2-yl)pyrimidin-5-yl)oxy)-5-fluorobenzoate hydrochloride and 1.2 equivalents of ((1r,4r)-4-(ethylsulfonamido)cyclohexyl)methyl 4-methylbenzenesulfonate. After completion, the reaction was quenched with water, and the mixture was extracted with EtOAc. The combined organic layer was dried over sodium sulfate and filtered, and the filtrate was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN) to obtain methyl 2-((4-(7-(((1r,4r)-4-(ethylsulfonamido)cyclohexyl)methyl)-2,7-diazaspiro[3.5]nonan-2-yl)pyrimidin-5-yl)oxy)-5-fluorobenzoate as a solid.

Yield: 71.5%; LCMS (Method A): Rt 1.85 min, m/z: 576.3 [M+H]⁺, 97.18%.

### Step 2. 2-(4-(7-(((1r,4r)-4-(Ethylsulfonamido)cyclohexyl)methyl)-2,7-diazaspiro[3.5]nonan-2-yl)pyrimidin-5-yl)oxy)-5-fluorobenzoic acid

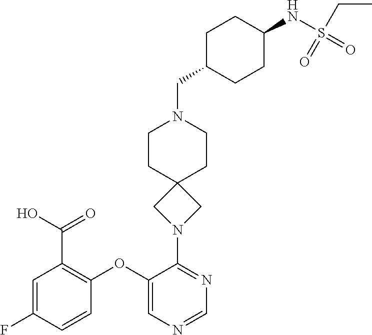 [SMILES: CCS(=O)(=O)N[C@H]1CC[C@H](CN2CCC3(CC2)CN(c2ncncc2Oc2ccc(F)cc2C(=O)O)C3)CC1]

This compound was synthesized following the procedure described for the synthesis of Intermediate 8, step 2, starting with 1 equivalent of methyl 2-((4-(7-(((1r,4r)-4-(ethylsulfonamido)cyclohexyl)methyl)-2,7-diazaspiro[3.5]nonan-2-yl)pyrimidin-5-yl)oxy)-5-fluorobenzoate. After work-up, the crude was purified by reverse phase chromatography using a SiliCycle SiliaSep C18 column (120 g) on Biotage isolera one (Mobile phase A: 10 mM NH₄HCO₃ in water, B: ACN). The fractions were concentrated under reduced pressure to afford 2-((4-(7-(((1r,4r)-4-(ethylsulfonamido)cyclohexyl)methyl)-2,7-diazaspiro[3.5]nonan-2-yl)pyrimidin-5-yl)oxy)-5-fluorobenzoic acid as a solid.

Yield: 66.2%; ¹H NMR (400 MHz, DMSO-d₆): δ 8.22 (s, 1H), 7.56-7.48 (m, 2H), 7.35 (dt, J=3.3, 8.4 Hz, 1H), 7.07 (dd, J=4.5, 9.0 Hz, 1H), 7.00 (d, J=7.8 Hz, 1H), 3.91 (br s, 4H), 3.02-2.91 (m, 3H), 2.46-2.35 (m, 3H), 2.16 (br d, J=5.6 Hz, 2H), 1.84 (br d, J=10.3 Hz, 2H), 1.89-1.70 (m, 6H), 1.49-1.36 (m, 1H), 1.28-1.14 (m, 5H), 0.96-0.79 (m, 2H), two protons merged with solvent peaks; LCMS (Method A): Rt 1.27 min, m/z: 562.0 [M+H]⁺, 98.61%.

### Intermediate 11. Benzyl((1r,3r)-3-amino-3-methylcyclobutyl)(isopronyl)carbamate

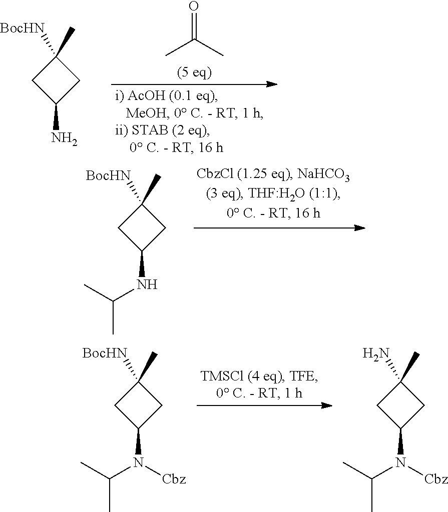

### Step 1. tert-Butyl((1r,3r)-3-(isopropylamino)-1-methylcyclobutyl)carbamate

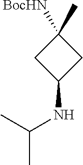 [SMILES: CC(C)N[C@H]1C[C@](C)(NC(=O)OC(C)(C)C)C1]

In a 10 L two neck dried round bottom flask under a nitrogen atmosphere, tert-butyl((1r,3r)-3-amino-1-methylcyclobutyl)carbamate (25 g, 125 mmol) was dissolved in MeOH (400 mL). To this solution, acetone (45.8 mL, 624 mmol) and AcOH (0.715 mL, 12.48 mmol) were added at 0° C., and the reaction was stirred at RT for 1 h. Sodium triacetoxyborohydride (52.9 g, 250 mmol) was then added at 0° C., and the reaction was stirred at RT for 16 h. The reaction was monitored by TLC(10% MeOH in DCM). After completion, the reaction was concentrated under reduced pressure. The crude was dissolved in saturated sodium bicarbonate solution (500 mL) and the mixture was extracted with DCM (3×500 mL). The combined organic layer was dried over anhydrous sodium sulfate and filtered, and the filtrate was concentrated under reduced pressure to afford crude tert-butyl (3-(isopropylamino)-1-methylcyclobutyl)carbamate (30 g, 99% yield) as a semi-solid. This material was used without further purification. ¹H NMR (400 MHz, DMSO-d₆): δ 6.87 (br s, 1H), 3.28 (quin, J=7.7 Hz, 1H), 2.76 (spt, J=6.2 Hz, 1H), 2.48-2.35 (m, 2H), 1.69-1.60 (m, 2H), 1.38 (s, 9H), 1.29 (s, 3H), 0.96 (d, J=6.3 Hz, 6H), one proton merged with solvent peaks.

### Step 2. Benzyl((1r,3r)-3-((tert-butoxycarbonyl)amino)-3-methylcyclobutyl)(isopropyl)carbamate

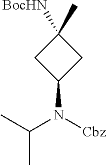 [SMILES: CC(C)N(C(=O)OCc1ccccc1)[C@H]1C[C@](C)(NC(=O)OC(C)(C)C)C1]

The reaction was performed following the same procedure in four individual batches (20 g, 20 g, 25 g, and 27 g).

To a stirred solution of tert-butyl((1r,3r)-3-(isopropylamino)-1-methylcyclobutyl)carbamate (25 g, 103 mmol) in THF (250 mL) and water (250 mL), solid sodium bicarbonate (26.0 g, 309 mmol) and Cbz-C1 (22.00 g, 129 mmol) were added at 0° C. The reaction was stirred at RT for 16 h. The reaction progress was monitored by TLC(30% EtOAc in hexane). After completion, the reaction was cooled to 0° C. and quenched with saturated sodium bicarbonate solution (100 mL), and the mixture was extracted with EtOAc (3×60 mL). The combined organic layer was dried over anhydrous sodium sulfate and filtered, and the filtrate was concentrated under reduced pressure. The crude was purified by silica gel flash column chromatography using EtOAc in hexane (product was eluted at 12% EtOAc in hexane) to obtain benzyl((1r,3r)-3-((tert-butoxycarbonyl)amino)-3-methylcyclobutyl)(isopropyl)carbamate (28 g, 72% yield) as a solid. LCMS (Method A): Rt=2.34 min, m/z=377.2 [M+H]⁺, 90.38%.

### Step 3. Benzyl((1r,3r)-3-amino-3-methylcyclobutyl)(isopropyl)carbamate

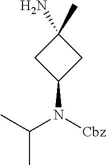 [SMILES: CC(C)N(C(=O)OCc1ccccc1)[C@H]1C[C@](C)(N)C1]

In a dried, 2 L two neck round bottom flask under a nitrogen atmosphere, benzyl((1r,3r)-3-((tert-butoxycarbonyl)amino)-3-methylcyclobutyl)(isopropyl)carbamate (100 g, 266 mmol) was dissolved in TFE (1000 mL). To this solution, TMSCI (135 mL, 1062 mmol) was added at 0° C. and the reaction was stirred at RT for 1 h. The reaction progress was monitored by TLC(10% MeOH in DCM). After completion, the reaction was concentrated under reduced pressure to obtain benzyl((1r,3r)-3-amino-3-methylcyclobutyl)(isopropyl)carbamate hydrochloride. The residue was basified using 10% NaHCO₃ solution (600 mL) and the mixture was extracted with 10% MeOH in DCM (3×700 mL). The combined organic layer was washed with brine solution, dried over anhydrous sodium sulfate, and filtered. The filtrate was concentrated under reduced pressure to obtain crude benzyl((1r,3r)-3-amino-3-methylcyclobutyl)(isopropyl)carbamate (73.2 g, 99% yield) as a liquid. This material was used without further purification. LCMS (Method C): Rt=1.44 min, m/z=277.1 [M+H]⁺, 99.75%.

### Intermediate 12. (1r,3r)-3-(Azetidin-1-yl)-N-isopropyl-3-methylcyclobutan-1-amine

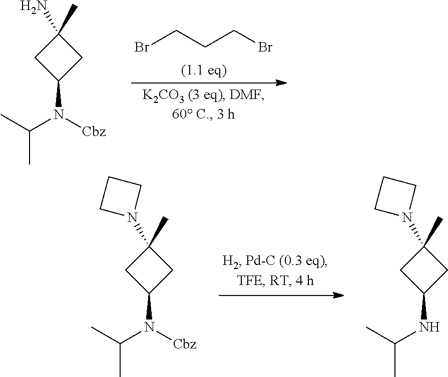 [SMILES: CC(C)N(C(=O)OCc1ccccc1)[C@H]1C[C@](C)(N)C1 ; BrCCCBr ; CC(C)N(C(=O)OCc1ccccc1)[C@H]1C[C@](C)(N2CCC2)C1 ; CC(C)N[C@H]1C[C@](C)(N2CCC2)C1]

### Step 1. Benzyl((1r, 3r)-3-(azetidin-1-vl)-3-methylcyclobutyl)(isopropyl)carbamate

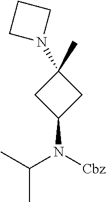 [SMILES: CC(C)N(C(=O)OCc1ccccc1)[C@H]1C[C@](C)(N2CCC2)C1]

In a 250 mL round bottom flask, benzyl((1r,3r)-3-amino-3-methylcyclobutyl)(isopropyl)carbamate (1 g, 3.62 mmol) was dissolved in DMF (100 mL). To this solution, K₂CO₃ (1.5 g, 10.85 mmol) and 1,3-dibromopropane (0.406 mL, 3.98 mmol) were added at RT. The reaction was stirred at 60° C. for 3 h, and monitored by TLC(10% MeOH in DCM). After completion, the reaction was quenched with ice-cold water (50 mL) and the mixture was extracted with EtOAc (3×50 mL). The combined organic layer was washed with ice-cold brine (3×50 mL), dried over anhydrous sodium sulfate, and filtered, and the filtrate was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure to afford benzyl((1r,3r)-3-(azetidin-1-yl)-3-methylcyclobutyl)(isopropyl)carbamate (800 mg, 69.5% yield) as a gummy solid. ¹H NMR (400 MHz, DMSO-d₆): δ 7.42-7.29 (m, 5H), 5.08 (s, 2H), 4.09-3.92 (m, 2H), 3.08 (t, J=6.9 Hz, 3H), 2.11 (br d, J=9.0 Hz, 3H), 1.86 (quin, J=6.9 Hz, 2H), 1.11 (d, J=6.8 Hz, 6H), 0.97 (br s, 3H), two protons merged with solvent peaks; LCMS (Method C): Rt=1.58 min, m/z=317.2 [M+H]⁺, 99.43%.

### Step 2. (1r,3r)-3-(Azetidin-1-yl)-N-isopropyl-3-methylcyclobutan-1-amine

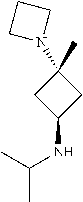

In a round bottom flask, ((1r,3r)-3-(azetidin-1-yl)-3-methylcyclobutyl)(isopropyl)carbamate (800 mg, 2.53 mmol) was dissolved in TFE (8 mL) and the solution was purged with nitrogen gas for 5 min. To this solution, Pd—C(807 mg, 0.758 mmol) was added under a nitrogen atmosphere. The reaction was stirred at RT for 4 h under a hydrogen atmosphere (balloon pressure), and monitored by TLC(10% MeOH in DCM). After completion, the reaction was filtered through a Celite® pad, and the pad was washed with MeOH (3×50 mL). The filtrate was concentrated under reduced pressure to afford crude (1r,3r)-3-(azetidin-1-yl)-N-isopropyl-3-methylcyclobutan-1-amine (527 mg, 56.5% yield) as semi-solid. This material was used without further purification. ¹H NMR (400 MHz, DMSO-d₆): δ 3.20-3.13 (m, 1H), 3.06 (t, J=6.9 Hz, 4H), 2.75-2.68 (m, 1H), 2.29-2.20 (m, 2H), 1.85 (quin, J=6.9 Hz, 2H), 1.43-1.36 (m, 2H), 1.04 (s, 3H), 0.94 (d, J=6.3 Hz, 6H), one proton merged with solvent peaks; LCMS (Method A): Rt=1.03 min, m/z=183.1 [M+H] *, 49.43%.

### Intermediate 13. (1r,3r)-N-Isopropyl-3-methyl-3-(pyrrolidin-1-yl)cyclobutan-1-amine

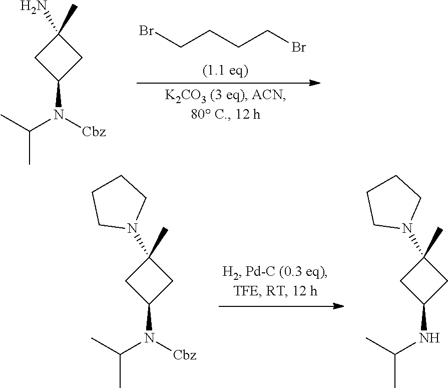 [SMILES: CC(C)N(C(=O)OCc1ccccc1)[C@H]1C[C@](C)(N)C1 ; BrCCCCBr ; CC(C)N(C(=O)OCc1ccccc1)[C@H]1C[C@](C)(N2CCCC2)C1 ; CC(C)N[C@H]1C[C@](C)(N2CCCC2)C1]

### Step 1. Benzyl isopropyl((1r,3r)-3-methyl-3-(pyrrolidin-1-yl)cyclobutyl)carbamate

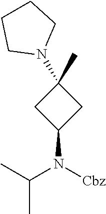 [SMILES: CC(C)N(C(=O)OCc1ccccc1)[C@H]1C[C@](C)(N2CCCC2)C1]

To a stirred solution of benzyl((1r,3r)-3-amino-3-methylcyclobutyl)(isopropyl)carbamate (30 g, 109 mmol) in ACN (150 mL), K₂CO₃ (45.0 g, 326 mmol) and 1,4-dibromobutane (14.26 mL, 119 mmol) were added at RT. The reaction was stirred at 80° C. for 12 h. The reaction progress was monitored by TLC(10% MeOH in DCM). After completion, the reaction was quenched with 10% NaHCO₃ solution (500 mL) and the mixture was extracted with EtOAc (2×500 mL). The combined organic layer was washed with brine solution (200 mL), dried over anhydrous sodium sulfate, and filtered. The filtrate was concentrated under reduced pressure to obtain crude benzyl isopropyl((1r,3r)-3-methyl-3-(pyrrolidin-1-yl)cyclobutyl)carbamate (31 g, 86% yield) as an oily liquid. This material was used without further purification. LCMS (Method B): Rt=1.40 min, m/z=331.2 [M+H]⁺, 99.76%.

### Step 2. (1r,3r)-N-Isopropyl-3-methyl-3-(pyrrolidin-1-yl)cyclobutan-1-amine

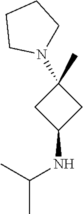

In a 500 mL round bottom flask under a nitrogen atmosphere, benzyl isopropyl((1r,3r)-3-methyl-3-(pyrrolidin-1-yl)cyclobutyl)carbamate (25 g, 76 mmol) was dissolved in TFE (300 mL). To this solution, 10% Pd—C(24.15 g, 22.69 mmol) was added at RT, and the reaction was stirred for 12 h at RT under a hydrogen atmosphere (balloon pressure). The reaction progress was monitored by TLC(10% MeOH in DCM). After completion, the reaction was filtered through a Celite® pad, and the pad was washed with THF (600 mL), then with EtOAc (600 mL). The filtrate was concentrated under reduced pressure to obtain crude (1r,3r)-N-isopropyl-3-methyl-3-(pyrrolidin-1-yl)cyclobutan-1-amine (15 g, 86% yield) as a semi-solid. This material was used without further purification. This material was used without further purification. LCMS (Method B): Rt=0.18 min, m/z=197.3 [M+H]⁺, 85.46%.

### Intermediate 14. 5-Fluoro-2-hydroxy-N-isopropyl-N-((1r,3r)-3-methyl-3-(pyrrolidin-1-yl)cyclobutyl)benzamide

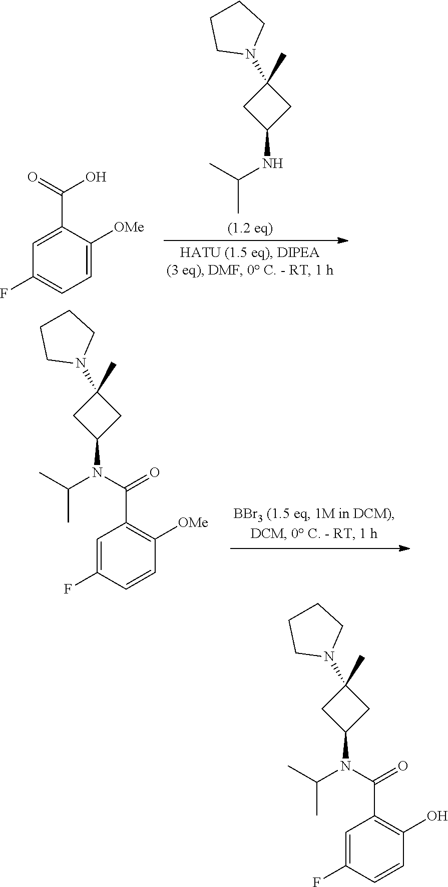

### Step 1. 5-Fluoro-N-isopropyl-2-methoxy-N-((1r,3r)-3-methyl-3-(pyrrolidin-1-yl)cyclobutyl)benzamide

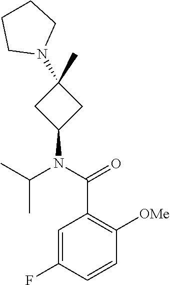 [SMILES: COc1ccc(F)cc1C(=O)N(C(C)C)[C@H]1C[C@](C)(N2CCCC2)C1]

To a stirred solution of 5-fluoro-2-methoxybenzoic acid (370 mg, 2.175 mmol) and (1r,3r)-N-isopropyl-3-methyl-3-(pyrrolidin-1-yl)cyclobutan-1-amine (512 mg, 2.61 mmol) in DMF (7 mL), DIPEA (1.139 mL, 6.52 mmol) and HATU (1240 mg, 3.26 mmol) were added at 0° C. The reaction was stirred at RT for 1 h, and monitored by TLC(10% MeOH in DCM). After completion, the reaction was quenched with cold water (50 mL) and the mixture was extracted with EtOAc (2×50 mL). The combined organic layer was washed with cold brine solution (2×30 mL), dried over anhydrous sodium sulfate and filtered, and the filtrate was concentrated under reduced pressure. The crude (800 mg) was purified by reverse phase chromatography using a SiliCycle SiliaSep C18 column (80 g cartridge) on Biotage isolera one (Mobile phase A: 10 mM NH₄HCO₃ in water, B: MeOH) to obtain 5-fluoro-N-isopropyl-2-methoxy-N-((1r,3r)-3-methyl-3-(pyrrolidin-1-yl)cyclobutyl)benzamide (400 mg, 47.7% yield) as a gummy liquid. LCMS (Method B): Rt=1.13 min, m/z=349.2 [M+H]⁺, 90.40%.

### Step 2. 5-Fluoro-2-hydroxy-N-isopropyl-N-((1r,3r)-3-methyl-3-(pyrrolidin-1-yl)cyclobutyl)benzamide

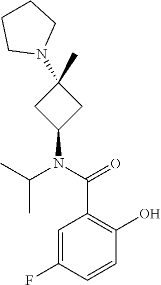 [SMILES: CC(C)N(C(=O)c1cc(F)ccc1O)[C@H]1C[C@](C)(N2CCCC2)C1]

To a stirred solution of 5-fluoro-N-isopropyl-2-methoxy-N-((1r,3r)-3-methyl-3-(pyrrolidin-1-yl)cyclobutyl)benzamide (700 mg, 2.009 mmol) in DCM (10 mL), BBr3 (3.01 mL, 3.01 mmol, 1M in DCM) was added at 0° C. The reaction was stirred at RT for 1 h, and monitored by TLC(10% MeOH in DCM). After completion, the reaction was concentrated under reduced pressure. The residue was diluted with 10% NaHCO₃ solution (70 mL) at 0° C., and the mixture was extracted with DCM (2×30 mL). The combined organic layer was dried over anhydrous sodium sulfate and filtered, and the filtrate was concentrated under reduced pressure to afford crude 5-fluoro-2-hydroxy-N-isopropyl-N-((1r,3r)-3-methyl-3-(pyrrolidin-1-yl)cyclobutyl)benzamide (600 mg, 78% yield) as a solid. This material was used without further purification. LCMS (Method A): Rt=1.46 min, m/z=335.1 [M+H]⁺, 87.75%.

### Intermediate 15. tert-Butyl(2s,4r)-2-(isopropylamino)-5-azaspiro[3.4]octane-5-carboxylate

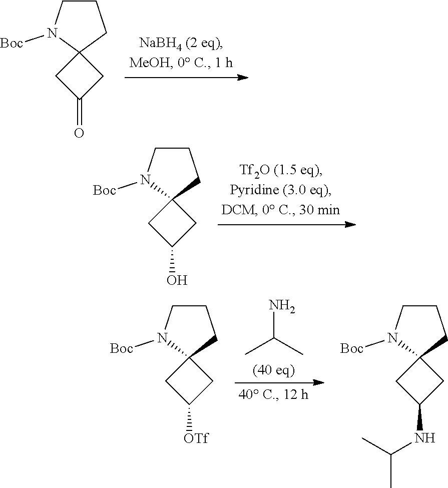

### Step 1. tert-Butyl(2r,4s)-2-hydroxy-5-azaspiro[3.4]octane-5-carboxylate

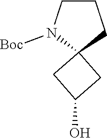 [SMILES: CC(C)(C)OC(=O)N1CCC[C@]12C[C@H](O)C2]

To a stirred solution of tert-butyl 2-oxo-5-azaspiro[3.4]octane-5-carboxylate (10 g, 44.4 mmol) in MeOH (100 mL), NaBH₄ (3.36 g, 89 mmol) was added over 20 min at 0° C., and the reaction was stirred at the same temperature for 1 h. The reaction progress was monitored by TLC (30% EtOAc in hexane). After completion, the reaction was quenched with ice-cold water (150 mL), and the mixture was extracted with EtOAc (2×200 mL). The combined organic layer was washed with brine solution (100 mL), dried over anhydrous sodium sulfate and filtered, and the filtrate was concentrated under reduced pressure to obtain crude tert-butyl(2r,4s)-2-hydroxy-5-azaspiro[3.4]octane-5-carboxylate (14.2 g, quantitative yield) as a solid. This material was used without further purification. LCMS (Method A): Rt=1.68 min, m/z=172.1 [M+H−56]⁺, 98.69%.

### Step 2. tert-Butyl(2r,4s)-2-(((trifluoromethyl) sulfonyl)oxy)-5-azaspiro[3.4]octane-5-carboxylate

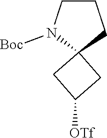 [SMILES: CC(C)(C)OC(=O)N1CCC[C@]12C[C@H](OS(=O)(=O)C(F)(F)F)C2]

To a stirred solution of tert-butyl(2r,4s)-2-hydroxy-5-azaspiro[3.4]octane-5-carboxylate (7 g, 30.8 mmol) in DCM (70 mL) and pyridine (7.46 mL, 92 mmol), Tf₂O (7.76 mL, 46.2 mmol) was added over 15 min at 0° C. The reaction was stirred at 0° C. for 30 min. The reaction progress was monitored by TLC(30% EtOAc in hexane). After completion, the reaction was quenched with ice-cold water (70 mL), and the mixture was extracted with DCM (2×10⁰ mL). The combined organic layer was washed with aqueous NaHCO₃ solution (70 mL), dried over anhydrous sodium sulfate and filtered, and the filtrate was concentrated under reduced pressure to afford crude tert-butyl(2r,4s)-2-(((trifluoromethyl) sulfonyl)oxy)-5-azaspiro[3.4]octane-5-carboxylate (10 g, 14.39 mmol, 46.7% yield). This compound was used without further purification. LCMS (Method A): Rt=2.36 min, m/z=303.9 [M+H−56]⁺, 51.70%.

### Step 3. tert-Butyl(2s, 4r)-2-(isopropylamino)-5-azaspiro[3.4]octane-5-carboxylate

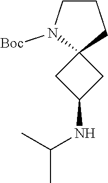 [SMILES: CC(C)N[C@H]1C[C@@]2(CCCN2C(=O)OC(C)(C)C)C1]

In 250 mL sealed tube, tert-butyl(2r,4s)-2-(((trifluoromethyl) sulfonyl)oxy)-5-azaspiro[3.4]octane-5-carboxylate (10 g, 27.8 mmol) was dissolved in isopropylamine (65.8 g, 1113 mmol) at RT, and the reaction was stirred at 40° C. for 12 h. The reaction progress was monitored by TLC(10% MeOH in DCM). After completion, the reaction was quenched with 10% NaHCO₃ solution (100 mL) and the mixture was extracted with 10% MeOH in DCM (3×100 mL). The combined organic layer was washed with brine solution (100 mL), dried over anhydrous sodium sulfate and filtered, and the filtrate was concentrated under reduced pressure to afford crude (tert-butyl(2s,4r)-2-(isopropylamino)-5-azaspiro[3.4]octane-5-carboxylate (10.5 g, quantitative yield) as a gummy liquid. This material was used without further purification. ¹H NMR (400 MHz, DMSO-d₆): δ 3.59-3.53 (m, 1H), 3.45-3.35 (m, 1H), 3.23 (t, J=6.6 Hz, 2H), 2.95-2.66 (m, 3H), 2.09-1.98 (m, 2H), 1.84-1.73 (m, 2H), 1.60 (quin, J=6.6 Hz, 2H), 1.53-1.34 (m, 9H), 1.05-0.86 (m, 6H); LCMS (Method B): Rt=1.25 min, m/z=269.6 [M+H]⁺, 83.12%.

### Intermediate 16. 2-((5-(2,7-Diazaspiro[3.5]nonan-2-yl)-1,2,4-triazin-6-yl)oxy)-5-fluoro-N-isopropyl-N-((1r,3r)-3-methyl-3-(pyrrolidin-1-yl)cyclobutyl)benzamide xHCI

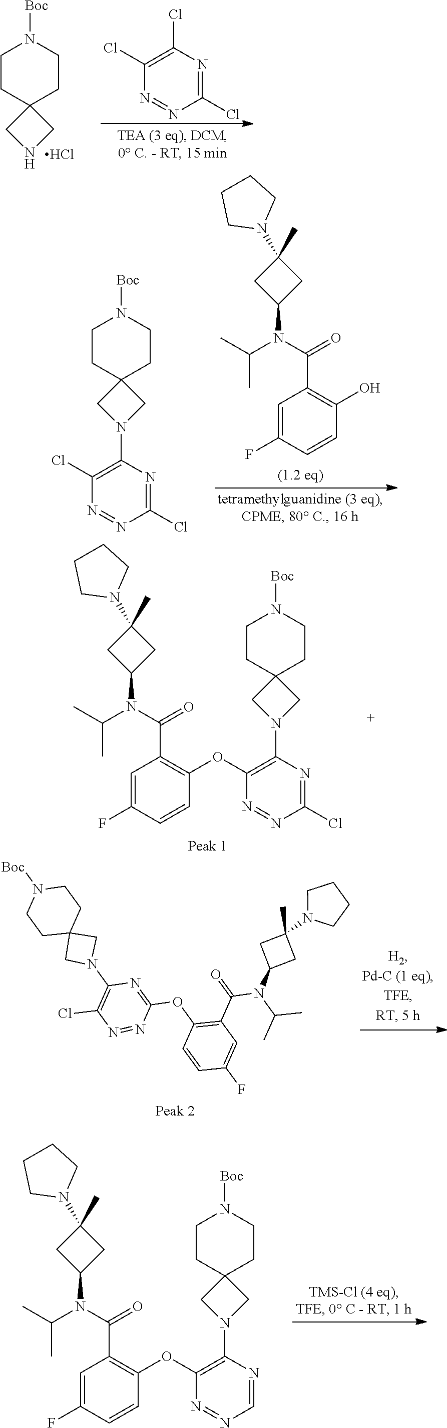

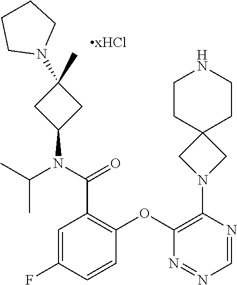 [SMILES: CC(C)N(C(=O)c1cc(F)ccc1Oc1nncnc1N1CC2(CCNCC2)C1)[C@H]1C[C@](C)(N2CCCC2)C1]

### Step 1. tert-Butyl 2-(3,6-dichloro-1,2,4-triazin-5-yl)-2,7-diazaspiro[3.5]nonane-7-carboxylate

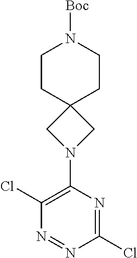 [SMILES: CC(C)(C)OC(=O)N1CCC2(CC1)CN(c1nc(Cl)nnc1Cl)C2]

To a stirred solution of tert-butyl 2,7-diazaspiro[3.5]nonane-7-carboxylate hydrochloride (10 g, 38.1 mmol) in DCM (200 mL), TEA (15.91 mL, 114 mmol) was added 0° C. under a nitrogen atmosphere. To this reaction mixture, 3,5,6-trichloro-1,2,4-triazine (7.02 g, 38.1 mmol) was added at 0° C. The reaction was stirred at 0° C. for 15 min, and monitored by TLC(10% MeOH in DCM).

After completion, the reaction was quenched with water (60 mL) and the mixture was extracted with DCM (3×60 mL). The combined organic layer was dried over anhydrous sodium sulfate and filtered, and the filtrate was concentrated under reduced pressure to afford crude tert-butyl 2-(3,6-dichloro-1,2,4-triazin-5-yl)-2,7-diazaspiro[3.5]nonane-7-carboxylate (13 g, 90% yield) as a solid. This material was used without further purification. ¹H NMR (400 MHz, DMSO-d₆): δ 4.44 (s, 2H), 3.89 (s, 2H), 3.39-3.21 (m, 4H), 1.76-1.64 (m, 4H), 1.40 (s, 9H); LCMS (Method B): Rt=2.08 min, m/z=374.2 [M+H]⁺, 98.56%.

### Step 2. tert-Butyl 2-(3-chloro-6-(4-fluoro-2-(isopropyl((1r,3r)-3-methyl-3-(pyrrolidin-1-yl)cyclobutyl)carbamoyl)phenoxy)-1,2,4-triazin-5-yl)-2,7-diazaspiro[3.5]nonane-7-carboxylate 2-(6-chloro-3-(4-fluoro-2-(isopropyl((1r,3r)-3-methyl-3-(pyrrolidin-1- and tert-butyl yl)cyclobutyl)carbamoyl)phenoxy)-1,2,4-triazin-5-yl)-2,7-diazaspiro[3.5]nonane-7-carboxylate

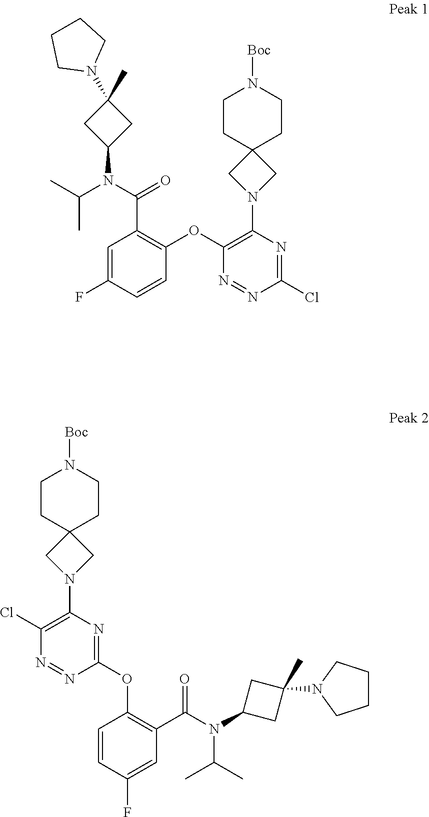 [SMILES: CC(C)N(C(=O)c1cc(F)ccc1Oc1nnc(Cl)nc1N1CC2(CCN(C(=O)OC(C)(C)C)CC2)C1)[C@H]1C[C@](C)(N2CCCC2)C1 ; CC(C)N(C(=O)c1cc(F)ccc1Oc1nnc(Cl)c(N2CC3(CCN(C(=O)OC(C)(C)C)CC3)C2)n1)[C@H]1C[C@](C)(N2CCCC2)C1]

In a dried, 100 mL round bottom flask under a nitrogen atmosphere, tert-butyl 2-(3,6-dichloro-1,2,4-triazin-5-yl)-2,7-diazaspiro[3.5]nonane-7-carboxylate (2 g, 5.34 mmol) and 5-fluoro-2-hydroxy-N-isopropyl-N-((1r,3r)-3-methyl-3-(pyrrolidin-1-yl)cyclobutyl)benzamide (2.145 g, 6.41 mmol) were dissolved in CPME (20 mL). To this reaction mixture, 1,1,3,3-tetramethylguanidine (2.007 mL, 16.03 mmol) was added at RT. The reaction was stirred at 80° C. for 16 h, and monitored by TLC (10% MeOH in DCM). After completion, the reaction was quenched with water (15 mL) and the mixture was extracted with 10% MeOH in DCM (3×50 mL). The combined organic layer was dried over anhydrous sodium sulfate and filtered, and the filtrate was concentrated under reduced pressure. The crude compound was purified by reverse phase chromatography using a SiliCycle SiliaSep C18 column (120 g) on Biotage isolera one (Mobile phase A: 10 mM NH₄HCO₃ in water, B: ACN) to afford a mixture of regioisomers, tert-butyl 2-(3-chloro-6-(4-fluoro-2-(isopropyl((1r,3r)-3-methyl-3-(pyrrolidin-1-yl)cyclobutyl)carbamoyl)phenoxy)-1,2,4-triazin-5-yl)-2,7-diazaspiro[3.5]nonane-7-carboxylate and tert-butyl 2-(6-chloro-3-(4-fluoro-2-(isopropyl((1r,3r)-3-methyl-3-(pyrrolidin-1-yl)cyclobutyl)carbamoyl)phenoxy)-1,2,4-triazin-5-yl)-2,7-diazaspiro[3.5]nonane-7-carboxylate (2.7 g, 73.2% yield) as a solid. LCMS (Method C): Rt=1.86 min, m/z=672.3 [M+H]⁺, 97.41%; Chiral SFC(Method A): Peak 1: Rt=1.59 min, 81.36%; Peak 2: Rt=2.41 min, 16.32%.

The mixture was separated by chiral Prep SFC(Method A) to obtain both regioisomers.

Peak 1. tert-Butyl 2-(3-chloro-6-(4-fluoro-2-(isopropyl((1r,3r)-3-methyl-3-(pyrrolidin-1-yl)cyclobutyl)carbamoyl)phenoxy)-1,2,4-triazin-5-yl)-2,7-diazaspiro[3.5]nonane-7-carboxylate (1.9 g, 70.0% yield) as a solid. LCMS (Method C): Rt=1.77 min, m/z=672.4 [M+H]⁺; HPLC (Method C): Rt=11.58 min, 99.44%; Chiral SFC(Method A): Rt=1.59 min, 100%.

Peak 2. tert-Butyl 2-(6-chloro-3-(4-fluoro-2-(isopropyl((1r,3r)-3-methyl-3-(pyrrolidin-1-yl)cyclobutyl)carbamoyl)phenoxy)-1,2,4-triazin-5-yl)-2,7-diazaspiro[3.5]nonane-7-carboxylate (0.37 g, 13.41% yield) as off white solid. LCMS (Method C): Rt=1.89 min, m/z=672.3 [M+H]⁺; HPLC(Method A): Rt=10.41 min, 95.80%; Chiral SFC(Method A): Rt=2.40 min, 98.88%.

### Step 3. tert-Butyl 2-(6-(4-fluoro-2-(isopropyl((1r,3r)-3-methyl-3-(pyrrolidin-1-yl)cyclobutyl)carbamoyl)phenoxy)-1,2,4-triazin-5-yl)-2,7-diazaspiro[3.5]nonane-7-carboxylate

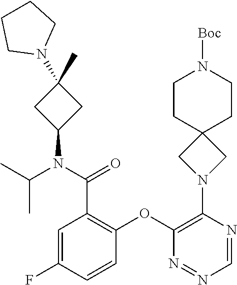 [SMILES: CC(C)N(C(=O)c1cc(F)ccc1Oc1nncnc1N1CC2(CCN(C(=O)OC(C)(C)C)CC2)C1)[C@H]1C[C@](C)(N2CCCC2)C1]

To a stirred solution of tert-butyl 2-(3-chloro-6-(4-fluoro-2-(isopropyl((1r,3r)-3-methyl-3-(pyrrolidin-1-yl)cyclobutyl)carbamoyl)phenoxy)-1,2,4-triazin-5-yl)-2,7-diazaspiro[3.5]nonane-7-carboxylate (1.9 g, 2.83 mmol) in TFE (25 mL), Pd—C(3.01 g, 2.83 mmol) was added at RT under a nitrogen atmosphere. The reaction was stirred at RT for 5 h under a hydrogen atmosphere (balloon pressure), and monitored by TLC(10% MeOH in DCM). After completion, the reaction was filtered through a Celite® pad, the pad was washed with MeOH (2×40 mL), and the filtrate was concentrated under reduced pressure. The crude product was purified by reverse phase chromatography using a SiliCycle SiliaSep C18 column (120 g) on Biotage isolera one (Mobile phase A: 10 mM NH₄HCO₃ in water, B: ACN) to obtain tert-butyl 2-(6-(4-fluoro-2-(isopropyl((1r,3r)-3-methyl-3-(pyrrolidin-1-yl)cyclobutyl)carbamoyl)phenoxy)-1,2,4-triazin-5-yl)-2,7-diazaspiro[3.5]nonane-7-carboxylate (1.1 g, 59% yield) as a solid. ¹H NMR (400 MHz, DMSO-d₆): δ 8.46 (s, 1H), 7.48-7.41 (m, 1H), 7.39-7.10 (m, 2H), 4.42-4.21 (m, 2H), 4.07-3.72 (m, 3H), 3.66-3.53 (m, 1H), 3.29-3.18 (m, 2H), 2.89-2.71 (m, 1H), 2.60-2.53 (m, 2H), 2.40-2.28 (m, 2H), 2.24-2.10 (m, 2H), 1.88-1.55 (m, 10H), 1.40 (s, 9H), 1.29-1.17 (m, 3H), 1.12-0.58 (m, 4H), three protons merged with solvent peaks; LCMS (Method A): Rt=2.11 min, m/z=638.5 [M+H]⁺; HPLC(Method C): Rt=10.24 min, 97.63%.

### Step 4. 2-((5-(2,7-Diazaspiro[3.5]nonan-2-yl)-1,2,4-triazin-6-yl)oxy)-5-fluoro-N-isopropyl-N-((1r,3r)-3-methyl-3-(pyrrolidin-1-yl)cyclobutyl)benzamide xHCl

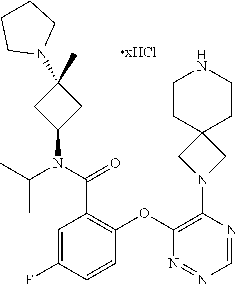 [SMILES: CC(C)N(C(=O)c1cc(F)ccc1Oc1nncnc1N1CC2(CCNCC2)C1)[C@H]1C[C@](C)(N2CCCC2)C1]

To a stirred solution of tert-butyl 2-(6-(4-fluoro-2-(isopropyl((1r,3r)-3-methyl-3-(pyrrolidin-1-yl)cyclobutyl)carbamoyl)phenoxy)-1,2,4-triazin-5-yl)-2,7-diazaspiro[3.5]nonane-7-carboxylate (1.1 g, 1.725 mmol) in TFE (15 mL), TMSCI (0.882 mL, 6.90 mmol) was added dropwise at 0° C. The reaction was stirred at RT for 1 h, and monitored with TLC(10% MeOH in DCM). After completion, the reaction was concentrated under reduced pressure. The residue was reconcentrated from EtOAc (2×50 mL) then was dried under vacuum to obtain crude 2-((5-(2,7-diazaspiro[3.5]nonan-2-yl)-1,2,4-triazin-6-yl)oxy)-5-fluoro-N-isopropyl-N-((1r,3r)-3-methyl-3-(pyrrolidin-1-yl)cyclobutyl)benzamide xHCI (1.2 g, quantitative yield) as a solid. This material was used without further purification. LCMS (Method C): Rt=1.04 min, m/z=538.3 [M+H]⁺, 98.26%.

### Example 1. 2-((4-(7-(((2S,5R)-5-((N-Ethylsulfamoyl)amino)tetrahydro-2H-pyran-2-yl)methyl)-2,7-diazaspiro[3.5]nonan-2-yl)pyrimidin-5-yl)oxy)-5-fluoro-N-isopropyl-N-((1r,3r)-3-methyl-3-(pyrrolidin-1-yl)cyclobutyl)benzamide (Compound No. 1)

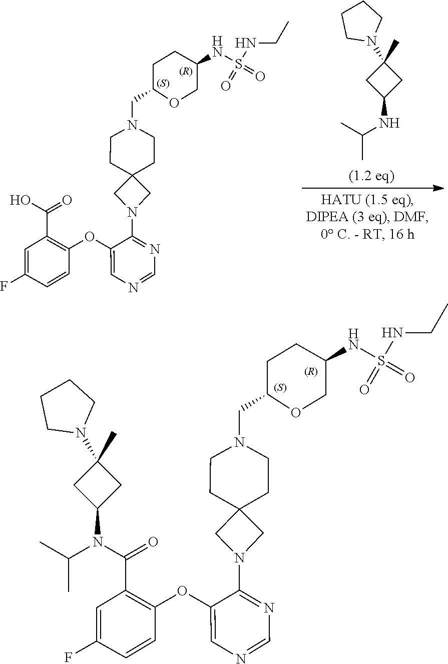

To a stirred solution of 2-((4-(7-(((2S,5R)-5-((N-ethylsulfamoyl)amino)tetrahydro-2H-pyran-2-yl)methyl)-2,7-diazaspiro[3.5]nonan-2-yl)pyrimidin-5-yl)oxy)-5-fluorobenzoic acid (300 mg, 0.518 mmol) and (1r,3r)-N-isopropyl-3-methyl-3-(pyrrolidin-1-yl)cyclobutan-1-amine (122 mg, 0.622 mmol) in DMF (3 mL), DIPEA (0.271 mL, 1.555 mmol) and HATU (296 mg, 0.778 mmol) were added at 0° C. The reaction was stirred at RT for 16 h, and monitored by TLC(10% MeOH in DCM). After completion, the reaction was quenched with water (50 mL) and the mixture was extracted with EtOAc (2×50 mL). The combined organic layer was washed with brine (50 mL), dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN), then by Prep HPLC(Method A). The fractions were concentrated under reduced pressure to obtain 2-((4-(7-(((2S,5R)-5-((N-ethylsulfamoyl)amino)tetrahydro-2H-pyran-2-yl)methyl)-2,7-diazaspiro[3.5]nonan-2-yl)pyrimidin-5-yl)oxy)-5-fluoro-N-isopropyl-N-((1r,3r)-3-methyl-3-(pyrrolidin-1-yl)cyclobutyl)benzamide as a TFA salt. The product was dissolved in 10% MeOH in DCM (50 mL), and washed with 10% NaOH solution (3×20 mL). The combined organic layer was dried over anhydrous sodium sulfate and filtered, and the filtrate was concentrated under reduced pressure. This product was repurified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford 2-((4-(7-(((2S,5R)-5-((N-ethylsulfamoyl)amino)tetrahydro-2H-pyran-2-yl)methyl)-2,7-diazaspiro[3.5]nonan-2-yl)pyrimidin-5-yl)oxy)-5-fluoro-N-isopropyl-N-((1r,3r)-3-methyl-3-(pyrrolidin-1-yl)cyclobutyl)benzamide (55 mg, 13.99% yield) as a solid.

Yield: 13.99%; ¹H NMR (400 MHz, DMSO-d₆): δ 8.30-8.23 (m, 1H), 7.81-7.55 (m, 1H), 7.32-6.99 (m, 3H), 6.87 (br d, J=5.1 Hz, 1H), 6.78 (br t, J=5.6 Hz, 1H), 4.10-3.64 (m, 8H), 3.05-2.94 (m, 2H), 2.88-2.77 (m, 3H), 2.41-2.08 (m, 10H), 2.02-1.76 (m, 2H), 1.73-1.55 (m, 10H), 1.47-1.17 (m, 6H), 1.10-0.91 (m, 9H); LCMS (Method C): Rt=1.15 min, m/z=757.4 [M+H]⁺; HPLC(Method A): Rt=6.90 min, 99.83%; Chiral SFC(Method B): Rt=2.44 min, 100%.

### Example 2. N-((1r,3r)-3-(Azetidin-1-yl)-3-methylcyclobutyl)-2-((4-(7-(((25,5R)-5-((N-ethylsulfamoyl)amino)tetrahydro-2H-pyran-2-yl)methyl)-2,7-diazaspiro[3.5]nonan-2-yl)pyrimidin-5-yl)oxy)-5-fluoro-N-isopropylbenzamide (Compound No. 2)

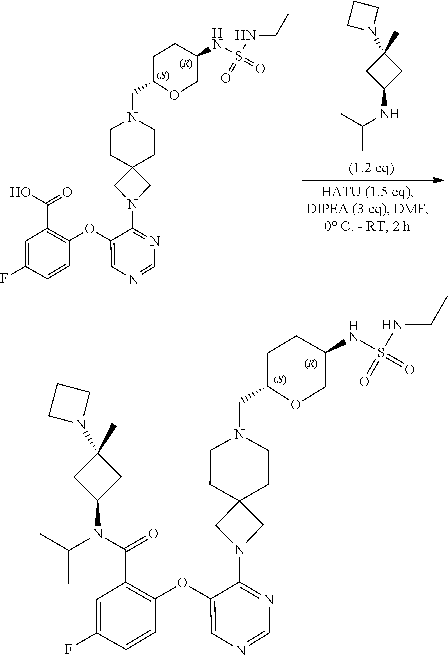

This compound was synthesized following the procedure described for the synthesis of Example 1, starting with 1 equivalent of 2-((4-(7-(((2S,5R)-5-((N-ethylsulfamoyl)amino)tetrahydro-2H-pyran-2-yl)methyl)-2,7-diazaspiro[3.5]nonan-2-yl)pyrimidin-5-yl)oxy)-5-fluorobenzoic acid and 1.2 equivalents of (1r,3r)-3-(azetidin-1-yl)-N-isopropyl-3-methylcyclobutan-1-amine. After work-up, the crude was purified by reverse phase chromatography using a SiliCycle SiliaSep C18 column (40 g) on Biotage isolera one (Mobile phase A: 10 mM NH₄HCO₃ in water, B: ACN), then by Prep HPLC(Method A). The fractions were concentrated I under reduced pressure to obtain N-((1r,3r)-3-(azetidin-1-yl)-3-methylcyclobutyl)-2-((4-(7-(((2S,5R)-5-((N-ethylsulfamoyl)amino)tetrahydro-2H-pyran-2-yl)methyl)-2,7-diazaspiro[3.5]nonan-2-yl)pyrimidin-5-yl)oxy)-5-fluoro-N-isopropylbenzamide as a TFA salt. The product was dissolved in 10% MeOH in DCM, and washed with 10% NaOH solution. The combined organic layer was dried over anhydrous sodium sulfate and filtered, and the filtrate was concentrated under reduced pressure. This product was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford N-((1r,3r)-3-(azetidin-1-yl)-3-methylcyclobutyl)-2-((4-(7-(((2S,5R)-5-((N-ethylsulfamoyl)amino)tetrahydro-2H-pyran-2-yl)methyl)-2,7-diazaspiro[3.5]nonan-2-yl)pyrimidin-5-yl)oxy)-5-fluoro-N-isopropylbenzamide as a solid.

Yield: 13.17%; ¹H NMR (400 MHz, DMSO-d₆): δ 8.31-8.21 (m, 1H), 7.80-7.57 (m, 1H), 7.33-6.99 (m, 3H), 6.87 (br d, J=3.4 Hz, 1H), 6.78 (br t, J=5.5 Hz, 1H), 4.01-3.64 (m, 7H), 3.12 (br t, J=6.6 Hz, 3H), 3.05-2.98 (m, 2H), 2.92 (br t, J=6.4 Hz, 2H), 2.87-2.72 (m, 3H), 2.66-2.59 (m, 1H), 2.32-1.99 (m, 6H), 1.97-1.64 (m, 9H), 1.47-1.16 (m, 6H), 1.10-0.93 (m, 9H); LCMS (Method C): Rt=1.11 min, m/z=743.4 [M+H]⁺; HPLC(Method A): Rt=6.75 min, 99.38%; Chiral SFC(Method B): Rt=2.01 min, 97.43%.

### Example 3. 2-((4-(7-(((2S,5R)-5-(Ethylsulfonamido)tetrahydro-2H-pyran-2-yl)methyl)-2,7-diazaspiro[3.5]nonan-2-yl)pyrimidin-5-yl)oxy)-5-fluoro-N-isopropyl-N-((1r,3r)-3-methyl-3-(pyrrolidin-1-yl)cyclobutyl)benzamide (Compound No. 3)

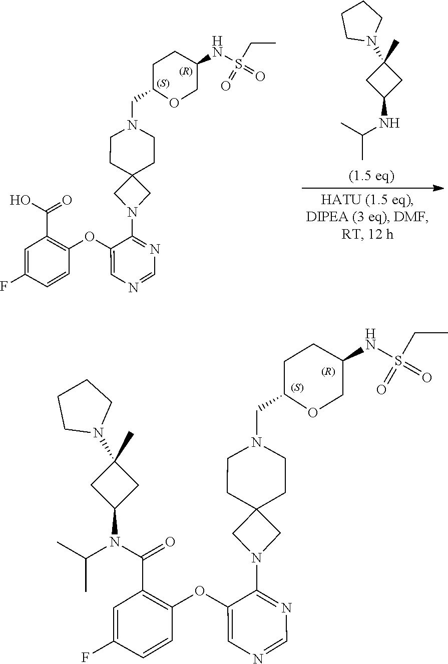

This compound was synthesized following the procedure described for the synthesis of Example 1, starting with 1 equivalent of 2-((4-(7-(((2S,5R)-5-(ethylsulfonamido)tetrahydro-2H-pyran-2-yl)methyl)-2,7-diazaspiro[3.5]nonan-2-yl)pyrimidin-5-yl)oxy)-5-fluorobenzoic acid and 1.5 equivalents of (1r,3r)-N-isopropyl-3-methyl-3-(pyrrolidin-1-yl)cyclobutan-1-amine. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain 2-((4-(7-(((2S,5R)-5-(ethylsulfonamido)tetrahydro-2H-pyran-2-yl)methyl)-2,7-diazaspiro[3.5]nonan-2-yl)pyrimidin-5-yl)oxy)-5-fluoro-N-isopropyl-N-((1r,3r)-3-methyl-3-(pyrrolidin-1-yl)cyclobutyl)benzamide as a solid.

Yield: 24.16%; ¹H NMR (400 MHz, DMSO-d₆): δ 8.26 (d, J=11.6 Hz, 1H), 7.82-7.54 (m, 1H), 7.33-6.98 (m, 4H), 4.13-3.65 (m, 7H), 3.15-2.95 (m, 5H), 2.87-2.70 (m, 2H), 2.42-2.35 (m, 2H), 2.32-2.07 (m, 7H), 2.03-1.76 (m, 3H), 1.73-1.57 (m, 9H), 1.46-0.93 (m, 15H); LCMS (Method C): Rt=1.10 min, m/z=742.4 [M+H]⁺; HPLC(Method A): Rt=6.79 min, 99.35%; Chiral SFC(Method C): Rt=3.64 min, 99.56%.

### Example 4. 2-((4-(7-(((1r,4r)-4-(Ethylsulfonamido)cyclohexyl)methyl)-2,7-diazaspiro[3.5]nonan-2-yl)pyrimidin-5-yl)oxy)-5-fluoro-N-isopropyl-N-((1r,3r)-3-methyl-3-(pyrrolidin-1-yl)cyclobutyl)benzamide (Compound No. 4)

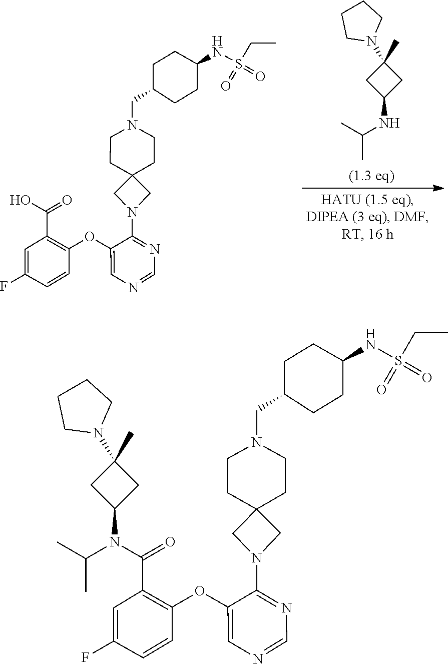

This compound was synthesized following the procedure described for the synthesis of Example 1, starting with 1 equivalent of 2-((4-(7-(((1r,4r)-4-(ethylsulfonamido)cyclohexyl)methyl)-2,7-diazaspiro[3.5]nonan-2-yl)pyrimidin-5-yl)oxy)-5-fluorobenzoic acid and 1.3 equivalents of (1r,3r)-N-isopropyl-3-methyl-3-(pyrrolidin-1-yl)cyclobutan-1-amine. After completion, the reaction was concentrated under reduced pressure. After work-up, the crude was purified by reverse phase chromatography using a SiliCycle SiliaSep C18 column (80 g) on Biotage isolera one (Mobile phase A: 10 mM NH₄HCO₃ in water, B: ACN), then by Prep HPLC(Method B). The fractions were concentrated under reduced pressure to obtain 2-((4-(7-(((1r,4r)-4-(ethylsulfonamido)cyclohexyl)methyl)-2,7-diazaspiro[3.5]nonan-2-yl)pyrimidin-5-yl)oxy)-5-fluoro-N-isopropyl-N-((1r,3r)-3-methyl-3-(pyrrolidin-1-yl)cyclobutyl)benzamide as a TFA salt. The product was dissolved in 10% MeOH in DCM, and washed with 10% NaOH solution. The combined organic layer was dried over anhydrous sodium sulfate and filtered, and the filtrate was concentrated under reduced pressure. This product was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford 2-((4-(7-(((1r,4r)-4-(ethylsulfonamido)cyclohexyl)methyl)-2,7-diazaspiro[3.5]nonan-2-yl)pyrimidin-5-yl)oxy)-5-fluoro-N-isopropyl-N-((1r,3r)-3-methyl-3-(pyrrolidin-1-yl)cyclobutyl)benzamide as a solid.

Yield: 36.3%; ¹H NMR (400 MHz, DMSO-d₆): δ 8.30-8.23 (m, 1H), 7.81-7.53 (m, 1H), 7.31-6.95 (m, 4H), 4.12-3.64 (m, 6H), 3.04-2.91 (m, 3H), 2.87-2.69 (m, 1H), 2.42-2.33 (m, 2H), 2.30-2.08 (m, 5H), 1.99 (br d, J=7.0 Hz, 2H), 1.90-1.78 (m, 3H), 1.77-1.57 (m, 10H), 1.48-1.28 (m, 4H), 1.27-1.14 (m, 7H), 1.11-0.94 (m, 5H), 0.92-0.80 (m, 2H), two protons merged with solvent peaks; LCMS (Method C): Rt=1.17 min, m/z=740.4 [M+H]⁺; HPLC (Method A): Rt=7.00 min, 97.75%; Chiral SFC(Method D): Rt=2.78 min, 98.80%.

### Example 5. 2-((5-(7-(((2S,5R)-5-((N-Ethylsulfamoyl)amino)tetrahydro-2H-pyran-2-yl)methyl)-2,7-diazaspiro[3.5]nonan-2-yl)-1,2,4-triazin-6-yl)oxy)-5-fluoro-N-isopropyl-N-((1r,3r)-3-methyl-3-(pyrrolidin-1-yl)cyclobutyl)benzamide xHCI (Compound No. 5)

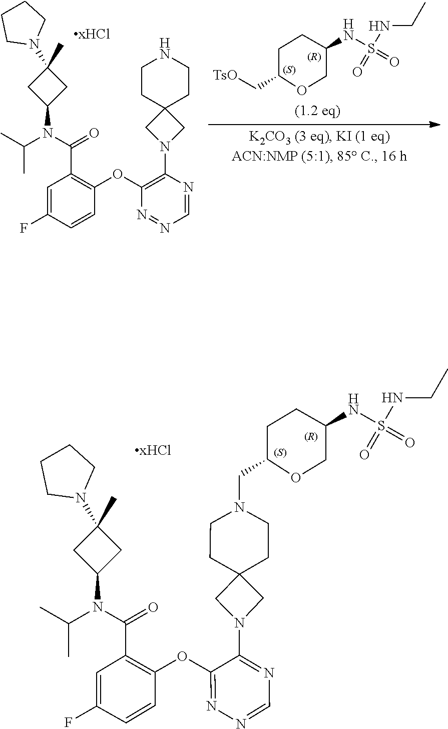

In a dried, 100 mL two neck round bottom flask under a nitrogen atmosphere, 2-((5-(2,7-diazaspiro[3.5]nonan-2-yl)-1,2,4-triazin-6-yl)oxy)-5-fluoro-N-isopropyl-N-((1r,3r)-3-methyl-3-(pyrrolidin-1-yl)cyclobutyl)benzamide xHCI (0.6 g, 1.045 mmol) was dissolved in 5:1 ACN: NMP (15 mL: 3 mL). K₂CO₃ (0.433 g, 3.14 mmol), KI (0.173 g, 1.045 mmol) and ((2S,5R)-5-((N-ethylsulfamoyl)amino)tetrahydro-2H-pyran-2-yl)methyl 4-methylbenzenesulfonate (0.492 g, 1.254 mmol) were then added at RT. The reaction was stirred at 85° C. for 16 h, and monitored by TLC(10% MeOH in DCM). After completion, the reaction was quenched with water (10 mL) and the mixture was extracted with 10% MeOH in DCM (3×20 mL). The combined organic layer was dried over anhydrous sodium sulfate and filtered, and the filtrate was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN). The fractions were concentrated under reduced pressure and lyophilized to afford 2-((5-(7-(((2S,5R)-5-((N-ethylsulfamoyl)amino)tetrahydro-2H-pyran-2-yl)methyl)-2,7-diazaspiro[3.5]nonan-2-yl)-1,2,4-triazin-6-yl)oxy)-5-fluoro-N-isopropyl-N-((1r,3r)-3-methyl-3-(pyrrolidin-1-yl)cyclobutyl)benzamide xHCI (317.67 mg, 38.6% yield) as a solid. ¹H NMR (400 MHz, DMSO-d₆): δ 8.45 (s, 1H), 7.48-7.11 (m, 3H), 6.93-6.75 (m, 3H), 4.32-4.14 (m, 2H), 4.05-3.72 (m, 5H), 3.69-3.53 (m, 1H), 3.24-2.95 (m, 5H), 2.87-2.77 (m, 4H), 2.31-2.11 (m, 5H), 2.00-1.91 (m, 2H), 1.86-1.55 (m, 10H), 1.48-1.18 (m, 8H), 1.10-1.04 (m, 7H), 0.74-0.59 (m, 1H); LCMS (Method C): Rt=1.16 min, m/z=758.4 [M+H]⁺; HPLC(Method B): Rt=5.91 min, 96.55%; Chiral SFC(Method D): Rt=4.40 min, 98.73%.

### Example 6. 2-((5-(7-(((2S,5R)-5-(Ethylsulfonamido)tetrahydro-2H-pyran-2-yl)methyl)-2,7-diazaspiro[3.5]nonan-2-yl)-1,2,4-triazin-6-yl)oxy)-5-fluoro-N-isopropyl-N-((1r,3r)-3-methyl-3-(pyrrolidin-1-yl)cyclobutyl)benzamide xHCI (Compound No. 6)

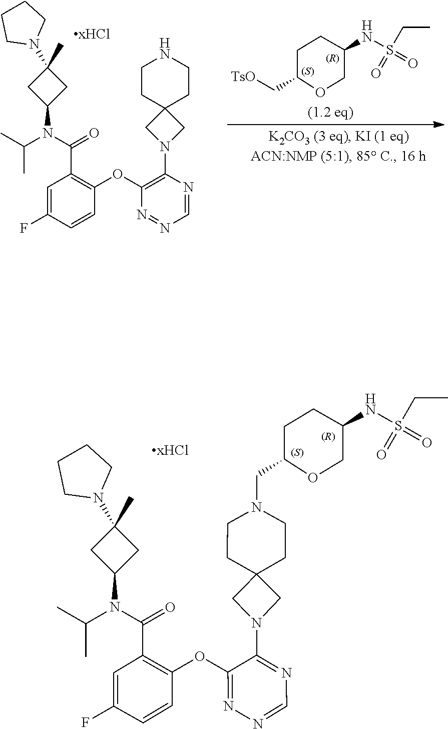

This compound was synthesized following the procedure described for the synthesis of Example 5, starting with 1 equivalent of 2-((5-(2,7-diazaspiro[3.5]nonan-2-yl)-1,2,4-triazin-6-yl)oxy)-5-fluoro-N-isopropyl-N-((1r,3r)-3-methyl-3-(pyrrolidin-1-yl)cyclobutyl)benzamide xHCI and 1.2 equivalents of ((2S,5R)-5-(ethylsulfonamido)tetrahydro-2H-pyran-2-yl)methyl 4-methylbenzenesulfonate. After work-up, the crude was purified by reverse phase chromatography using a SiliCycle SiliaSep C18 column (120 g) on Biotage isolera one (Mobile phase A: 10 mM NH₄HCO₃ in water, B: ACN). The fractions were concentrated under reduced pressure and lyophilized to afford 2-((5-(7-(((2S,5R)-5-(ethylsulfonamido)tetrahydro-2H-pyran-2-yl)methyl)-2,7-diazaspiro[3.5]nonan-2-yl)-1,2,4-triazin-6-yl)oxy)-5-fluoro-N-isopropyl-N-((1r,3r)-3-methyl-3-(pyrrolidin-1-yl)cyclobutyl)benzamide xHCl as a solid.

Yield: 29.6%; ¹H NMR (400 MHz, DMSO-d₆): δ 8.45 (s, 1H), 7.49-7.27 (m, 3H), 7.19-7.06 (m, 2H), 4.33-4.14 (m, 2H), 4.05-3.72 (m, 5H), 3.68-3.53 (m, 1H), 3.23-2.97 (m, 8H), 2.86-2.71 (m, 1H), 2.31-2.10 (m, 5H), 2.00-1.91 (m, 2H), 1.86-1.55 (m, 10H), 1.53-1.30 (m, 4H), 1.28-1.14 (m, 8H), 1.12-0.59 (m, 4H); LCMS (Method C): Rt=1.14 min, m/z=743.4 [M+H]⁺; HPLC(Method C): Rt=8.05 min, 97.11%; Chiral SFC(Method D): Rt=4.90 min, 100%.

### Example 7. 2-((5-(7-(((1r,4r)-4-(Ethylsulfonamido)cyclohexyl)methyl)-2,7-diazaspiro[3.5]nonan-2-yl)-1,2,4-triazin-6-yl)oxy)-5-fluoro-N-isopropyl-N-((1r,3r)-3-methyl-3-(pyrrolidin-1-yl)cyclobutyl)benzamide (Compound No. 7)

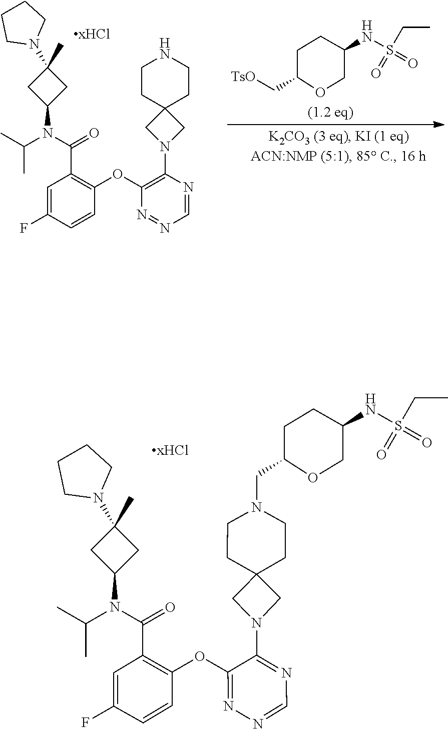 [SMILES: CC(C)N(C(=O)c1cc(F)ccc1Oc1nncnc1N1CC2(CCNCC2)C1)[C@H]1C[C@](C)(N2CCCC2)C1 ; CCS(=O)(=O)N[C@@H]1CC[C@@H](COS(=O)(=O)c2ccc(C)cc2)OC1 ; CCS(=O)(=O)N[C@@H]1CC[C@@H](CN2CCC3(CC2)CN(c2ncnnc2Oc2ccc(F)cc2C(=O)N(C(C)C)[C@H]2C[C@](C)(N4CCCC4)C2)C3)OC1]

This compound was synthesized following the procedure described for the synthesis of Example 5, starting with 1 equivalent of 2-((5-(2,7-diazaspiro[3.5]nonan-2-yl)-1,2,4-triazin-6-yl)oxy)-5-fluoro-N-isopropyl-N-((1r,3r)-3-methyl-3-(pyrrolidin-1-yl)cyclobutyl)benzamide xHCl and 1.2 equivalents of ((1r,4r)-4-(ethylsulfonamido)cyclohexyl)methyl 4-methylbenzenesulfonate. After work-up, the crude was purified by reverse phase chromatography using a SiliCycle SiliaSep C18 column (120 g) on Biotage isolera one (Mobile phase A: 10 mM NH₄HCO₃ in water, B: ACN), then by Prep HPLC(Method C). The fractions were concentrated under reduced pressure, washed with aqueous 10% NaOH solution, and the mixture was extracted with 10% MeOH in DCM. The combined organic layer was dried over anhydrous sodium sulfate and filtered, and the filtrate was concentrated under reduced pressure and lyophilized to afford 2-((5-(7-(((1r,4r)-4-(ethylsulfonamido)cyclohexyl)methyl)-2,7-diazaspiro[3.5]nonan-2-yl)-1,2,4-triazin-6-yl)oxy)-5-fluoro-N-isopropyl-N-((1r,3r)-3-methyl-3-(pyrrolidin-1-yl)cyclobutyl)benzamide as a solid.

Yield: 17.96%; ¹H NMR (400 MHz, DMSO-d₆): δ 8.45 (s, 1H), 7.48-7.12 (m, 3H), 7.00 (br d, J=7.6 Hz, 1H), 4.35-4.12 (m, 2H), 4.06-3.95 (m, 1H), 3.89-3.71 (m, 3H), 3.69-3.54 (m, 1H), 3.05-2.90 (m, 4H), 2.26-2.07 (m, 3H), 2.04-1.95 (m, 2H), 1.90-1.54 (m, 16H), 1.46-1.31 (m, 3H), 1.30-1.13 (m, 10H), 1.11-0.60 (m, 7H); LCMS (Method C): Rt=1.15 min, m/z=741.5 [M+H]⁺; HPLC(Method C): Rt=8.79 min, 99.33%; Chiral SFC(Method C): Rt=5.13 min, 95.19%.

### Example 8. 2-((4-(7-(((2S,5R)-5-(Ethylsulfonamido)tetrahydro-2H-pyran-2-yl)methyl)-2,7-diazaspiro[3.5]nonan-2-yl)pyrimidin-5-yl)oxy)-5-fluoro-N-isopropyl-N-((2s,4r)-5-methyl-5-azaspiro[3.4]octan-2-yl)benzamide (Compound No. 8)

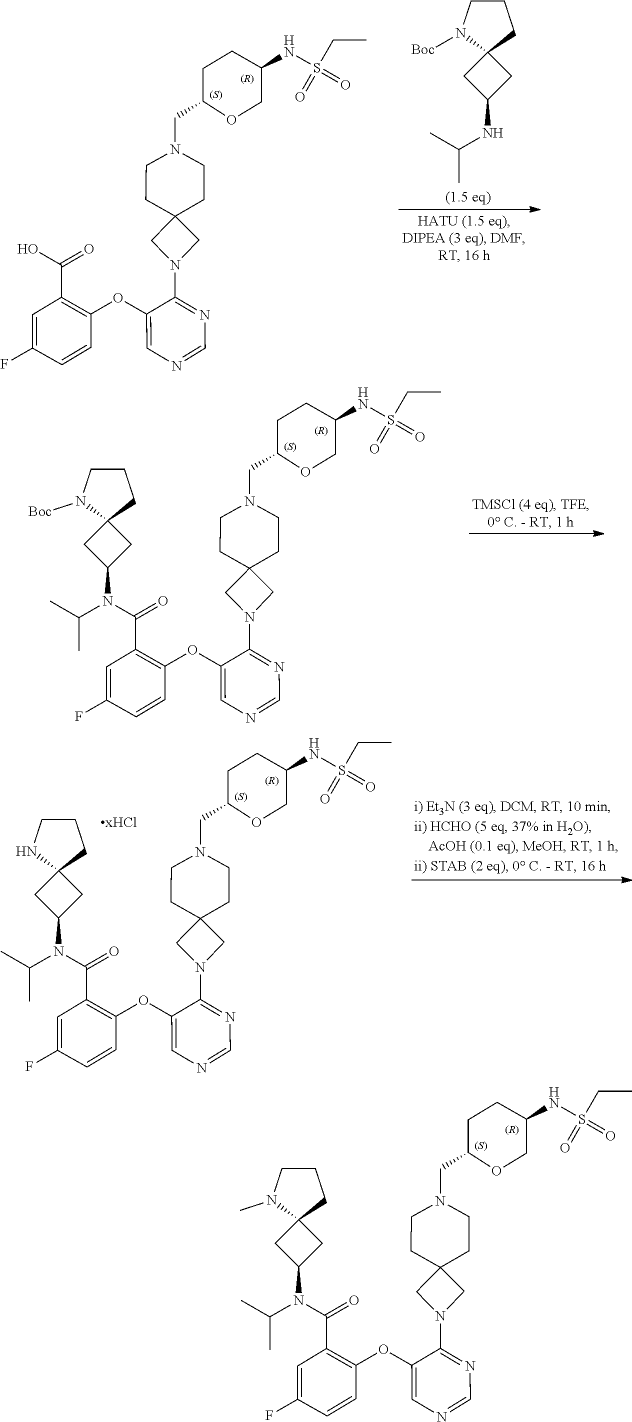

### Step 1. tert-Butyl(2s, 4r)-2-(2-((4-(7-(((2S,5R)-5-(ethylsulfonamido)tetrahydro-2H-pyran-2-yl)methyl)-2, 7-diazaspiro[3.5]nonan-2-yl)pyrimidin-5-yl)oxy)-5-fluoro-N-isopropylbenzamido)-5-azaspiro[3.4]octane-5-carboxylate

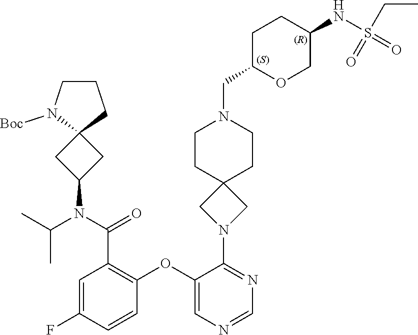

To a stirred solution of 2-((4-(7-(((2S,5R)-5-(ethylsulfonamido)tetrahydro-2H-pyran-2-yl)methyl)-2,7-diazaspiro[3.5]nonan-2-yl)pyrimidin-5-yl)oxy)-5-fluorobenzoic acid (0.5 g, 0.887 mmol) in DMF (5 mL), tert-butyl(2s,4r)-2-(isopropylamino)-5-azaspiro[3.4]octane-5-carboxylate (0.357 g, 1.331 mmol) was added at RT. To this reaction mixture, DIPEA (0.464 mL, 2.66 mmol) and HATU (0.506 g, 1.331 mmol) were added at 0° C., and the reaction was stirred at RT for 16 h. The reaction progress was monitored by TLC(10% MeOH in DCM). After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl(2s,4r)-2-(2-((4-(7-(((25,5R)-5-(ethylsulfonamido)tetrahydro-2HI-pyran-2-yl)methyl)-2,7-diazaspiro[3.5]nonan-2-yl)pyrimidin-5-yl)oxy)-5-fluoro-N-isopropylbenzamido)-5-azaspiro[3.4]octane-5-carboxylate (512 mg, 70.7% yield) as a solid. LCMS (Method C): Rt=1.53 min, m/z=814.4 [M+H]⁺, 99.78%.

### Step 2. 2-((4-(7-(((2S,5R)-5-(Ethylsulfonamido)tetrahydro-2H-pyran-2-yl)methyl)-2,7-diazaspiro[3.5]nonan-2-yl)pyrimidin-5-yl)oxy)-5-fluoro-N-isopropyl-N-((2s,4r)-5-azaspiro[3.4]octan-2-yl)benzamide xHCl

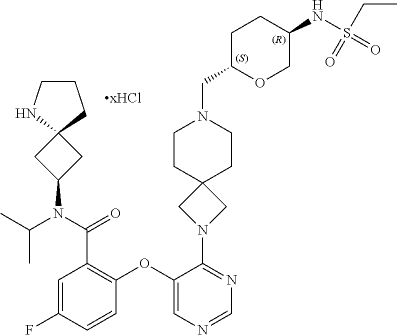

To a stirred solution of tert-butyl(2s,4r)-2-(2-((4-(7-(((2S,5R)-5-(ethylsulfonamido)tetrahydro-2H-pyran-2-yl)methyl)-2,7-diazaspiro[3.5]nonan-2-yl)pyrimidin-5-yl)oxy)-5-fluoro-N-isopropylbenzamido)-5-azaspiro[3.4]octane-5-carboxylate (512 mg, 0.629 mmol) in TFE (5 mL), TMSCI (0.322 mL, 2.52 mmol) was added at 0° C. The reaction was stirred at RT for 1 h, and monitored by TLC(10% MEOH in DCM). After completion, the reaction was concentrated under reduced pressure to afford crude 2-((4-(7-(((2S,5R)-5-(ethylsulfonamido)tetrahydro-2H-pyran-2-yl)methyl)-2,7-diazaspiro[3.5]nonan-2-yl)pyrimidin-5-yl)oxy)-5-fluoro-N-isopropyl-N-((2s,4r)-5-azaspiro[3.4]octan-2-yl)benzamide xHCI (630 mg, quantitative yield) as a solid. This material was used without further purification. LCMS (Method C): Rt=1.08 min, m/z=714.3 [M+H]⁺, 97.52%.

### Step 3. 2-((4-(7-(((2S,5R)-5-(Ethylsulfonamido)tetrahydro-2H-pyran-2-yl)methyl)-2,7-diazaspiro/3.5]nonan-2-yl)pyrimidin-5-yl)oxy)-5-fluoro-N-isopropyl-N-((2s,4r)-5-methyl-5-azaspiro[3.4]octan-2-yl)benzamide

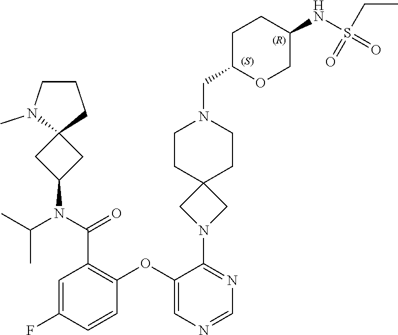

To a stirred solution of 2-((4-(7-(((2S,5R)-5-(ethylsulfonamido)tetrahydro-2H-pyran-2-yl)methyl)-2,7-diazaspiro[3.5]nonan-2-yl)pyrimidin-5-yl)oxy)-5-fluoro-N-isopropyl-N-((2s,4r)-5-azaspiro[3.4]octan-2-yl)benzamide xHCI (0.63 g, 0.840 mmol) in DCM (5 mL), TEA (0.255 g, 2.52 mmol) was added at RT. The reaction was stirred at RT for 15 min, then was concentrated under reduced pressure. The residue was dissolved in MeOH (5 mL), and acetic acid (5.04 mg, 0.084 mmol) and formaldehyde (0.116 ml, 4.20 mmol, 37% in H₂O) were added at RT. The reaction was stirred at RT for 1 h. The reaction was cooled to 0° C., sodium triacetoxyborohydride (0.356 g, 1.679 mmol) was added, and the reaction was stirred at RT for 16 h. The reaction was monitored by TLC(10% MeOH in DCM). After completion, the reaction was concentrated under reduced pressure. The crude was dissolved in 10% MeOH in DCM (100 mL) and quenched with aqueous sodium bicarbonate (100 mL). The organic layer was dried over sodium sulfate and filtered, and the filtrate was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain 2-((4-(7-(((25,5R)-5-(ethylsulfonamido)tetrahydro-2H-pyran-2-yl)methyl)-2,7-diazaspiro[3.5]nonan-2-yl)pyrimidin-5-yl)oxy)-5-fluoro-N-isopropyl-N-((2s,4r)-5-methyl-5-azaspiro[3.4]octan-2-yl)benzamide (206 mg, 33.6% yield) as a solid. ¹H NMR (400 MHz, DMSO-d₆): δ 8.30-8.21 (m, 1H), 7.81-7.52 (m, 1H), 7.32-6.98 (m, 4H), 3.99-3.62 (m, 7H), 3.16-2.89 (m, 5H), 2.84-2.72 (m, 1H), 2.39-2.14 (m, 10H), 2.09-1.87 (m, 5H), 1.73-1.61 (m, 6H), 1.60-1.21 (m, 7H), 1.18 (t, J=7.3 Hz, 3H), 1.11-0.92 (m, 4H); LCMS (Method C): Rt=1.08 min, m/z=728.4 [M+H]⁺; HPLC(Method A): Rt=6.74 min, 99.63%; Chiral SFC(Method E): Rt=2.29 min, 96.29%.

### Example 9. 2-((4-(7-(((1r,4r)-4-(Ethylsulfonamido)cyclohexyl)methyl)-2,7-diazaspiro[3.5]nonan-2-yl)pyrimidin-5-yl)oxy)-5-fluoro-N-isopropyl-N-((2s,4r)-5-methyl-5-azaspiro[3.4]octan-2-yl)benzamide (Compound No. 9)

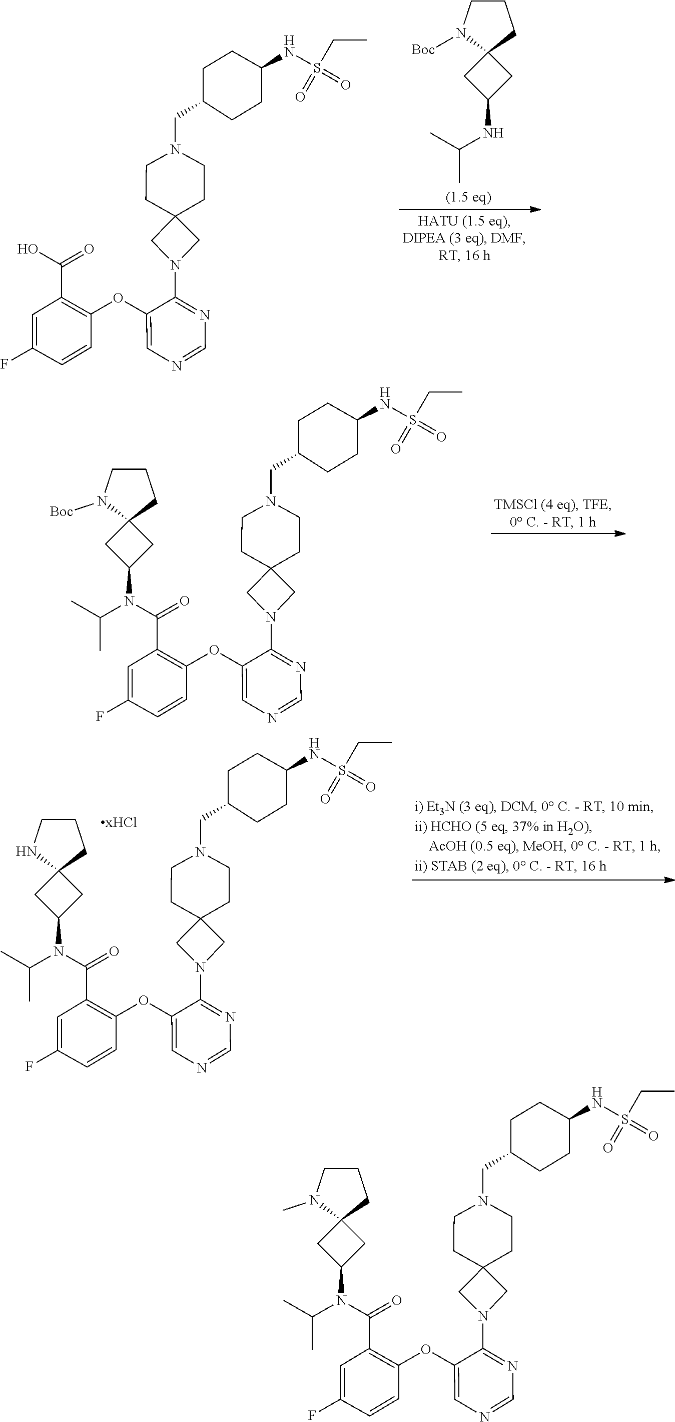

### Step 1. tert-Butyl(2s,4r)-2-(2-((4-(7-(((1r,4r)-4-(ethylsulfonamido)cyclohexyl)methyl)-2,7-diazaspiro[3.5]nonan-2-yl)pyrimidin-5-yl)oxy)-5-fluoro-N-isopropylbenzamido)-5-azaspiro[3.4]octane-5-carboxylate

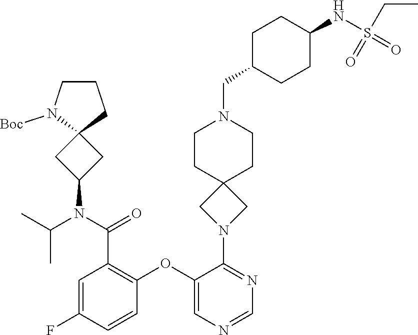 [SMILES: CCS(=O)(=O)N[C@H]1CC[C@H](CN2CCC3(CC2)CN(c2ncncc2Oc2ccc(F)cc2C(=O)N(C(C)C)[C@H]2C[C@@]4(CCCN4C(=O)OC(C)(C)C)C2)C3)CC1]

This compound was synthesized following the procedure described for the synthesis of Example 8, step 1, starting with 1 equivalent of 2-((4-(7-(((1r,4r)-4-(ethylsulfonamido)cyclohexyl)methyl)-2,7-diazaspiro[3.5]nonan-2-yl)pyrimidin-5-yl)oxy)-5-fluorobenzoic acid and 1 equivalent of tert-butyl(2s,4r)-2-(isopropylamino)-5-azaspiro[3.4]octane-5-carboxylate. After completion, the reaction was quenched with water and the mixture was extracted with EtOAc. The combined organic layer was washed with cold water and brine solution, dried over anhydrous sodium sulfate and filtered, the filtrate was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN) to obtain tert-butyl(2s,4r)-2-(2-((4-(7-(((1r,4r)-4-(ethylsulfonamido)cyclohexyl)methyl)-2,7-diazaspiro[3.5]nonan-2-yl)pyrimidin-5-yl)oxy)-5-fluoro-N-isopropylbenzamido)-5-azaspiro[3.4]octane-5-carboxylate as a solid. Yield: 58.2%; LCMS (Method A): Rt=2.39 min, m/z=812.2 [M+H]⁺, 97.85%.

### Step 2. 2-((4-(7-(((1r,4r)-4-(Ethylsulfonamido)cyclohexyl)methyl)-2,7-diazaspiro[3.5]nonan-2-yl)pyrimidin-5-yl)oxy)-5-fluoro-N-isopropyl-N-((2s,4r)-5-azaspiro[3.4]octan-2-yl)benzamide hydrochloride xHCl

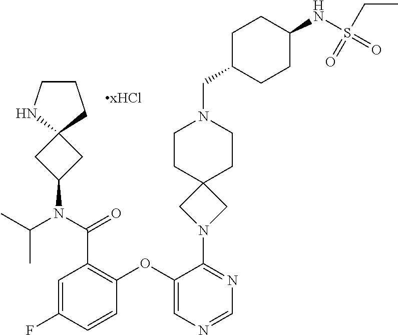 [SMILES: CCS(=O)(=O)N[C@H]1CC[C@H](CN2CCC3(CC2)CN(c2ncncc2Oc2ccc(F)cc2C(=O)N(C(C)C)[C@H]2C[C@@]4(CCCN4)C2)C3)CC1]

This compound was synthesized following the procedure described for the synthesis of Example 8, step 2, starting with 1 equivalent of tert-butyl(2s,4r)-2-(2-((4-(7-(((1r,4r)-4-(ethylsulfonamido)cyclohexyl)methyl)-2,7-diazaspiro[3.5]nonan-2-yl)pyrimidin-5-yl)oxy)-5-fluoro-N-isopropylbenzamido)-5-azaspiro[3.4]octane-5-carboxylate. After completion, the reaction was concentrated under reduced pressure. The residue was reconcentrated from EtOAc and the residue was dried under vacuum to obtain crude 2-((4-(7-(((1r,4r)-4-(ethylsulfonamido)cyclohexyl)methyl)-2,7-diazaspiro[3.5]nonan-2-yl)pyrimidin-5-yl)oxy)-5-fluoro-N-isopropyl-N-((2s,4r)-5-azaspiro[3.4]octan-2-yl)benzamide xHCl as a solid. This material was used without further purification.

Yield: 83%; LCMS (Method A): Rt=1.83 min, m/z=712.2 [M+H]⁺, 82.72%.

### Step 3. 2-(4-(7-(((1r,4r)-4-(Ethylsulfonamido)cyclohexyl)methyl)-2,7-diazaspiro[3.5]nonan-2-yl)pyrimidin-5-yl)oxy)-5-fluoro-N-isopropyl-N-((2s,4r)-5-methyl-5-azaspiro[3.4]octan-2-yl)benzamide

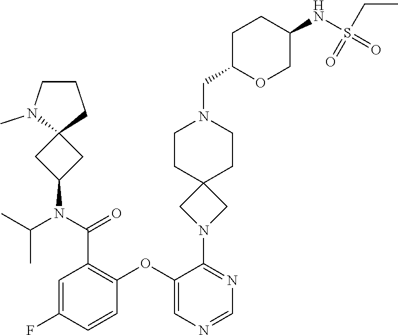 [SMILES: CCS(=O)(=O)N[C@@H]1CC[C@@H](CN2CCC3(CC2)CN(c2ncncc2Oc2ccc(F)cc2C(=O)N(C(C)C)[C@H]2C[C@@]4(CCCN4C)C2)C3)OC1]

This compound was synthesized following the procedure described for the synthesis of Example 8, 3, starting with step 1 equivalent of 2-((4-(7-(((1r,4r)-4-(ethylsulfonamido)cyclohexyl)methyl)-2,7-diazaspiro[3.5]nonan-2-yl)pyrimidin-5-yl)oxy)-5-fluoro-N-isopropyl-N-((2s,4r)-5-azaspiro[3.4]octan-2-yl)benzamide xHCI, except that 0.5 equivalent AcOH was used. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain 2-((4-(7-(((1r,4r)-4-(ethylsulfonamido)cyclohexyl)methyl)-2,7-diazaspiro[3.5]nonan-2-yl)pyrimidin-5-yl)oxy)-5-fluoro-N-isopropyl-N-((2s,4r)-5-methyl-5-azaspiro[3.4]octan-2-yl)benzamide as a solid.

Yield: 61.3%; ¹H NMR (400 MHz, DMSO-d₆): δ 8.33-8.19 (m, 1H), 7.84-7.50 (m, 1H), 7.34-7.14 (m, 2H), 7.04-6.95 (m, 2H), 4.00-3.63 (m, 6H), 3.02-2.90 (m, 4H), 2.84-2.72 (m, 1H), 2.47-2.41 (m, 1H), 2.34 (s, 3H), 2.30-2.14 (m, 4H), 2.10-1.81 (m, 8H), 1.78-1.62 (m, 7H), 1.60-1.49 (m, 2H), 1.45 (br d, J=6.4 Hz, 1H), 1.39-1.29 (m 2H), 1.26-1.14 (m, 5H), 1.09-0.94 (m, 4H), 0.92-0.79 (m, 2H); LCMS (Method C): Rt=1.06 min, m/z=726.3 [M+H]⁺; HPLC(Method A): Rt=6.85 min, 99.20%; Chiral SFC(Method D): Rt=3.02 min, 100%.

### Example 10. Menin-MLL Competition and MV4;11 Cell Proliferation Assays

Menin-MLL is a competition assay between human Menin and N-terminal portion of human MLL representing amino acids 4-43 of the protein. The interaction between Menin and MLL peptide was monitored by HTRF employing Terbium labeled anti-His6 antibody directed to the N-terminal His6-tag on recombinant Menin and FITC group covalently attached to the MILL peptide. The N-terminal fragment of MLL, retained in all MLL fusion proteins, is involved in the interactions with Menin, and this protein-protein interaction is critical for the MLL fusion proteins mediated leukemogenic transformations.

For IC50 determination test compounds were prepared as 10 mM DMSO stock solutions. Considering DMSO as the vehicle in the assay system. Lower sub-stocks of 50 μM were prepared from the 10 mM stock solution. To test the compounds in assay, 3.16-fold serial dilutions are made in 100% DMSO. Mid-stock of 50× compounds (50 μM) were serially diluted (3.16 fold) in 100% DMSO in Polypropylene plate. In assay plate 1 micro-litre of the previously prepared compound dilution was stamped. H-FL-Menin diluted to 4 nM in assay buffer (50 mM Tris-HCl, pH 7.4, 50 mM NaCl, freshly prepared 1 mM DTT, 0.01% BSA, 0.005% Triton X-100) was pre-incubated with 8 nM anti-His6-Tb for 30 min at room temperature. FITC-MLL-4-43 was diluted to 2 nM in assay buffer and 25 μL was dispensed into each well of the assay plate followed by addition of 25 μl of pre-incubated H-FL-Menin and anti-His6-Tb mixture. Final concentration H-FL-Menin diluted to InM in assay plate with 2 nM anti-His6-Tb and InM FITC-MLL-4-43. After 1 hr incubation at room temperature, the HTRF signal was measured on the Spark multi-label plate reader. Resulting data were captured as a ratio of RFU520/RFU485×1000. The max values were obtained from 0% inhibition in presence of 2% DMSO and the min. values were 100% inhibition in presence of 1 μM reference compound.

### Example 11. Cell Proliferation Assay on MV4;11 and MV4-11 [M327I-Heterozygous] Cells Using CTG Assay

### Test Compounds Preparation

Compounds were dissolved to obtain as 10 mM solution in DMSO. These stocks were diluted 1:5 to the top concentration to achieve 2 mM in 100% DMSO. For EC50 determination, compounds were serially diluted with semi-log in 100% DMSO. Each prepared DMSO solution were further diluted 1:500 in the cell culture media to obtain the 2×dosing solutions. The Final concentrations of tested compounds in the cell culture media ranged from 0.632 nM to 2000 nM which keeps % DMSO in assay as 0.1 across the wells.

### Experimental Procedure

MV4;11 [harboring the MLL-AF4; (ATCC Cat.No. CRL-9591)] & mutated cell line of MV4-11 [M327I-hetero; Source: DFCI, Boston, MA] cells were cultured in IMDM/RPMI with 10% HI FBS at 5% CO₂ and 37° C. The cell suspension containing 150,000 cells/mL in the culture medium was dispensed in 96-well cell culture plates 100 μL (15,000 cell/well) per well. Then 100 μL of 2× dosing media with test compounds were added to each well. Cells were cultured for 72 h at 37° C. and 5% CO₂ in a humidified incubator.

Post 72 h incubation, cultured cells were resuspended and 100 μL of the suspension from the assay plate was transferred into 96-well black OptiPlate (PerkinElmer Cat. No. 6005320). To this, 100 μL of Cell Titer Glo reagent was added, assay plates were shaken for 5 min at RT, and luminescence was read on Tecan Spark (Tecan) plate reader.

### Data Analysis

Percent inhibition values were calculated based on the high luminescence signal obtained with 0.1% DMSO control and the low signal observed with 5 μM Revumenib. The percent inhibition vs. concentration data were fit into a four-parameter model, and EC50 values were calculated from the fit as the concentrations corresponding to the inflection points on the dose-response curves. Data is shown in Table 2.

### Example 12. Determination of Binding Constants to Menin-MLL

For Ki determination, individual compounds were prepared as 10 mM DMSO stock solutions. Considering DMSO as the vehicle in the assay system. Lower sub-stocks of 16 μM were prepared from the 10 mM stock solution. To test the compounds in assay, 3.16-fold serial dilutions are made in 100% DMSO. Mid-stock of 50× compounds (16 μM) were serially diluted (3.16 fold) in 100% DMSO in Polypropylene plate. In assay plate 1 micro-litre of the previously prepared compound dilution was stamped. H-FL-Menin diluted to InM in assay buffer (50 mM Tris-HCl, pH 7.4, 50 mM NaCl, freshly prepared 1 mM DTT, 0.01% BSA, 0.005% Triton X-100) and pre-incubated with 2 nM anti-His6-Tb for 30 min at room temperature and then 25 μl was dispensed into each well. FITC-MLL-4-43 was diluted to 6.4 nM in assay buffer and 25 μL was dispensed into each well of the assay plate followed by addition of 25 μl of pre-incubated H-FL-Menin and anti-His6-Tb mixture. Final concentration H-FL-Menin diluted to 0.25 nM in assay plate with 0.5 nM anti-His6-Tb and 3.2 nM FITC-MLL-4-43. After 24 hr incubation at room temperature, the HTRF signal was measured on the Spark multi-label plate reader. Resulting data were captured as a ratio of RFU520/RFU485×1000. The max values were obtained from 0% inhibition in presence of 2% DMSO and the min. values were 100% inhibition in presence of 320 nM reference compound. Data is shown in Table 2.

TABLE 2
Shows (i) Menin-MLL binding data according to Homogeneous Time Resolved
Fluorescence (HTRF) assay with (i) individual compound Ki (nM) and (ii) the EC₅₀ (nM) against
MV4;11 and MV4;11 M327I leukemia cell lines. Ki data (nM) are provided below (″n/a″ refers
to data not available; ″+++″ means <1.0 nM; ″++″ means ≥1.0 nM and <1.5 nM; and ″+″
means ≥1.5 nM). EC50 (nM) against MV4;11 and MV4;11 M327I data are provided below (″n/a″ refers
to data not available; ″+++″ means <100 nM; ″++″ means ≥100 nM and <1000 nM; and ″+″
means ≥1000 nM).

| | | WT | | MV4;11 |
|---|---|---|---|---|
| Compound | | Menin | MV4;11 | M327I (het) |
| No. | Structure | Ki (nM) | EC₅₀ (nM) | EC₅₀ (nM) |
| 1 | 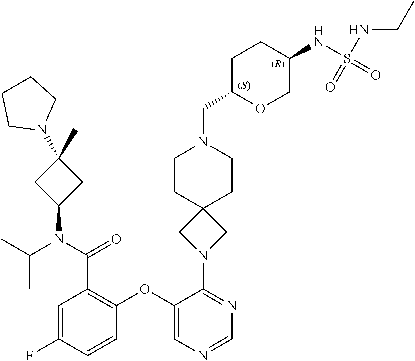 | +++ | +++ | + |
| 2 | 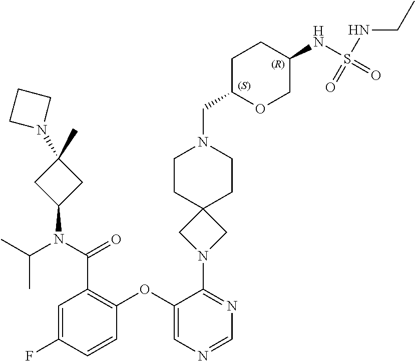 | +++ | +++ | + |
| 3 | 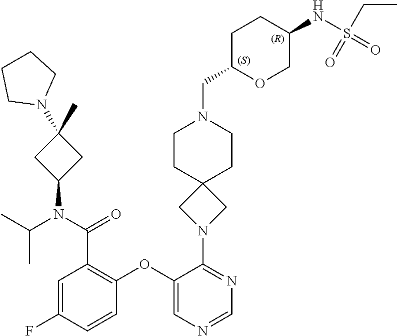 | +++ | +++ | + |
| 4 | 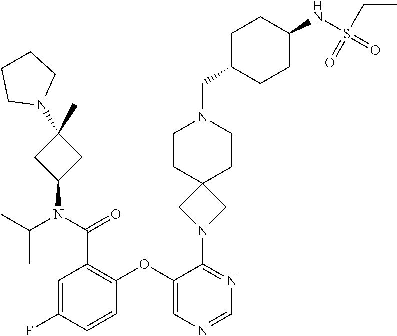 [SMILES: CCS(=O)(=O)N[C@H]1CC[C@H](CN2CCC3(CC2)CN(c2ncncc2Oc2ccc(F)cc2C(=O)N(C(C)C)[C@H]2C[C@](C)(N4CCCC4)C2)C3)CC1] | +++ | +++ | +++ |
| 5 | 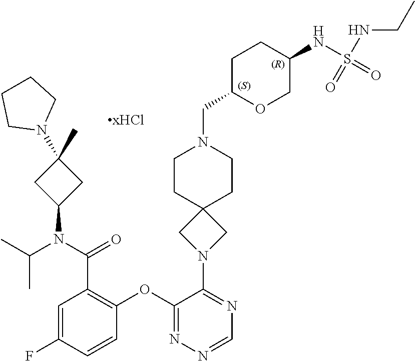 | +++ | +++ | + |
| 6 | 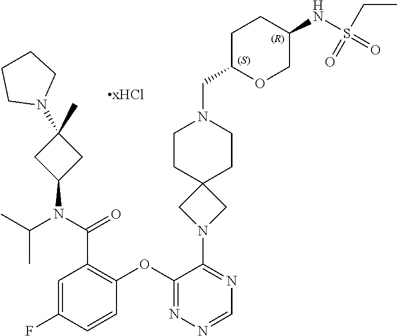 | +++ | +++ | + |
| 7 | 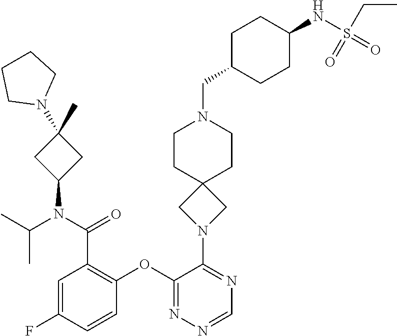 [SMILES: CCS(=O)(=O)N[C@H]1CC[C@H](CN2CCC3(CC2)CN(c2ncnnc2Oc2ccc(F)cc2C(=O)N(C(C)C)[C@H]2C[C@](C)(N4CCCC4)C2)C3)CC1] | +++ | +++ | + |
| 8 | 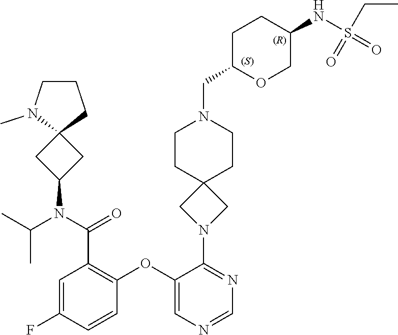 | +++ | +++ | + |
| 9 | 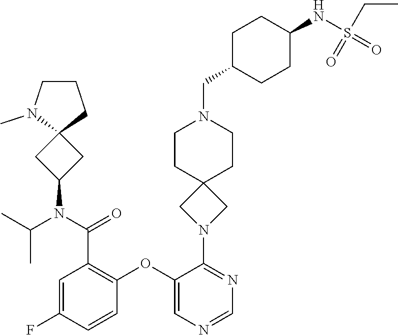 [SMILES: CCS(=O)(=O)N[C@H]1CC[C@H](CN2CCC3(CC2)CN(c2ncncc2Oc2ccc(F)cc2C(=O)N(C(C)C)[C@H]2C[C@@]4(CCCN4C)C2)C3)CC1] | +++ | +++ | + |

### Example 13. Patch Clamp Assay

The 35 mm culture dishes upon which cells were seeded at a density allowing single cells to be recorded were placed on the dish holder of the microscope and continuously perfused (at approximately 1 mL/min) with the bath solution. All solutions applied to cells including the pipette solution were maintained at room temperature (19° C. to 30° C.). After formation of a Gigaohm seal between the patch electrodes and individual hERG stably transfected HEK 293 cells (pipette resistance range: 2.0 MQ2 to 7.0 MQ; seal resistance range: >1G (2) the cell membrane across the pipette tip was ruptured to assure electrical access to the cell interior (whole-cell patch-configuration). In case the quality of the seal was poor, the process of seal formation was repeated with a different cell and a new pipette. As soon as a stable seal could be established, hERG outward tail currents were measured upon depolarization of the cell membrane to +20 mV for 2s (activation of channels) from a holding potential of −80 mV and upon subsequent repolarization to −40 m V for 3 s. This voltage protocol (as shown below) was run at least 10 times at intervals of 10 s. If current density was judged to be too low for measurement, another cell was recorded.

Once control recordings have been accomplished, cells were continuously perfused with a bath solution containing the test item 0.3% DMSO or 100 nM E-4031. During wash-in of the test item the voltage protocol indicated above was run continuously again at 10 s intervals until the steady-state level of block was reached.

### EQUIVALENTS

While we have described a number of embodiments of this disclosure, it is apparent that our basic examples may be altered to provide other embodiments that utilize the compounds and methods of this disclosure. The contents of all references (including literature references, issued patents, published patent applications, and co-pending patent applications)cited throughout this application are hereby expressly incorporated herein in their entireties by reference. Unless otherwise defined, all technical and scientific terms used herein are accorded the meaning commonly known to one with ordinary skill in the art. The foregoing description has been presented only for the purposes of illustration and is not intended to limit the disclosure to the precise form disclosed, but by the claims appended hereto. The references cited herein are not admitted to be prior art to the application.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

What is claimed is:

1. A compound of Formula I: 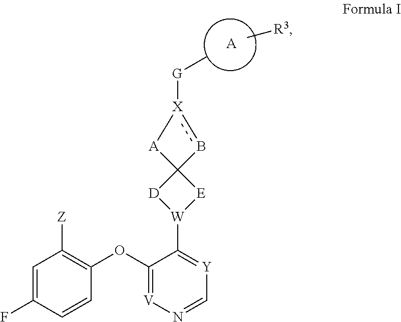
a stereoisomer, or a pharmaceutically acceptable salt thereof, wherein: [symbol]
denotes a single bond or a double bond, as valency permits;
A, B, D, and E are each independently selected from -C(R^{A1})(R^{A2})—, C(R^{A1})(R^{A2})-C(R^{A1})(R^{A2})—, -C(R^{A1})(R⁴²)—O—, -C(R^{A1})(R^{A2})-NR^{A3}-, -C(═O)—, —C(R^{A1})(R^{A2})—C(═O)—, and -N═C(NH₂)—, wherein no more than one of A, B, D, and E is —C(R^{A1})(R^{A2})—O—, -C(R^{A1})(R^{A2})-NR^{A3}-, -C(R^{A1})(R^{A2})—C(═O)—, -C(═O)—, or -N═C(NH₂)-;
V is N or CH;
Y is N or CH;
G is O, CH₂, or NH;
Ring A is cyclohexyl or 6-membered heterocycloalkyl which has one heteroatom ring member being an oxygen;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CRX, wherein R^{X} is absent, H, halo, CN, OH, C₁₋₆ alkyl, C₁₋₆ alkoxy, amino, C₁₋₆ alkyl amino, or C₂₋₈ dialkylamino;
Z is halo, C₁₋₆ alkyl, C₁₋₄ haloalkyl, C₁₋₄ cyanoalkyl, C₂₋₆ alkenyl, C₂₋₆ alkynyl, CN, NO₂, SR^{a3}, C(O)R^{b3}, C(O)NR¹R², C(O)OR^{a3}, OC(O)R^{b3}, OC(O)NR^{c3}R^{d3}, C(═NR^{e3})NR^{c3}R^{d3}, NR^{c3}C(═NR^{c3})NR^{c3}R^{d3}, NR^{c3}R^{d3}, NR^{c3}C(O)OR^{a3}, NR^{c3}C(O)NR^{c3}R^{d3}, NR^{c3}S(O)R^{b3}, NR^{c3}S(O)₂NR^{c3}R^{d3}, S(O)R^{b3}, S(O)NR^{c3}R^{d3}, S(O)₂R^{b3}, and P(O)R^{c3}R^{d3}, wherein said C₁₋₆ alkyl, C₁₋₄ haloalkyl, C₁₋₄ cyanoalkyl, C₂₋₆ alkenyl, and C₂₋₆ alkynyl are each optionally substituted with 1, 2, 3, or 4 substituents independently selected from Cy2, halo, CN, NO₂, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{c3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{A1} is independently selected from absent, H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A2} is independently selected from H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A3} is independently selected from H, C₁₋₄ alkyl, C₁₋₄ alkoxy, C₁₋₄ haloalkyl, C(O)Rz, and C(O)OR², wherein said C₁₋₆ alkyl is optionally substituted with phenyl, C₁₋₄ alkoxy, C₁₋₄ haloalkoxy, CN, NO₂, or OH;
R^{z} is H, C₁₋₆ alkyl, or phenyl;
each Cy2 is independently selected from C₆₋₁₄ aryl, C₃₋₁₈ cycloalkyl, 5-16 membered heteroaryl, and 4-18 membered heterocycloalkyl, each of which is optionally substituted with 1, 2, 3, or 4 substituents independently selected from R^{Cy2};
each R^{Cy2} is independently selected from halo, C₁₋₆ alkyl, C₁₋₄ haloalkyl, C₁₋₄ cyanoalkyl, C₂₋₆ alkenyl, C₂₋₆ alkynyl, phenyl, C₃₋₇ cycloalkyl, 5-6 membered heteroaryl, 4-7 membered heterocycloalkyl, CN, NO₂, OR^{a5}, SR^{a5}, C(O)R^{b5}, C(O)NR^{c5}R⁴⁵, C(O)OR^{a5}, OC(O)R^{b5}, OC(O)NRC5R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR⁶⁵R⁴⁵, NR^{c5}S(O)R^{b5}, NR^{c5}S(O)₂R^{b5}, NR^{c5}S(O)₂NR⁵R^{d5}, S(O)R^{b5}, S(O)NR^{c5}R^{d5}, S(O)₂R^{b5}, and S(O)₂NR^{c5}R^{d5}, wherein said C₁₋₆ alkyl, C₁₋₄ haloalkyl, C₁₋₄ cyanoalkyl, C₂₋₆ alkenyl, C₂₋₆ alkynyl, phenyl, C₃₋₇ cycloalkyl, 5-6 membered heteroaryl, and 4-7 membered heterocycloalkyl are each optionally substituted with 1, 2, 3, or 4 substituents independently selected from CN, NO2, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR⁶⁵R^{d5}, NR^{c5}C(═NR^{e5})NR^{c5}R^{d5}, NRCSR^{d5}, NR⁵° C. (O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5},
R¹ is
(a) H, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
(b) 3- to 12-membered heterocycloalkyl comprising a sulfur atom, wherein the sulfur atom of the 3- to 12-membered heterocycloalkyl is optionally substituted with one or two R^{1s};
each R^{1s} is independently oxo or =NR^{4a};
(c) -C₁₋₆ alkylene-N(R^{3a}′)(R^{3b}′) or -C₃₋₁₂ cycloalkylene-N(R^{3a}′)(R^{3b}′), wherein the C₁₋₆ alkylene or C₃₋₁₂ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
(d) 3- to 12-membered heterocycloalkyl which has at least one nitrogen and optionally one or more other heteroatom ring members, wherein the 3- to 12-membered heterocycloalkyl is optionally substituted with one or more R^{1bs},
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
(e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs},
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{e1}C(O)OR^{a1}, NR^{c1}C(O)NR^{c1}R^{d1}, NR^{c1}S(O)R^{b1}, NR^{c1}S(0)₂R^{b1}, NR^{e1}S(0)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO2, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1},
each R^{a1}, R^{b1}, R^{e1}, R^{d1}, R^{a3}, R^{b3}, R^{e3}, R^{d3}, R^{a5}, R^{b5}, R^{e5}, and R⁴⁵ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1}, R^{e3}, and R^{e5} is independently selected from H, C₁₋₆ alkyl, and CN;
each R^{g} is independently selected from OH, NO2, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO-C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a} is independently H, CN, or C₁₋₆ alkyl optionally substituted with one or more halo, C₆₋₁₀ aryl, or N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

2. A compound of Formula II: 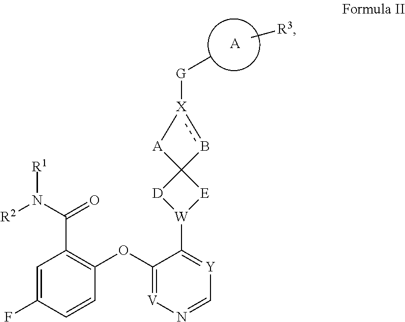
a stereoisomer, or a pharmaceutically acceptable salt thereof, wherein: [symbol]
denotes a single bond or a double bond, as valency permits;
A, B, D, and E are each independently selected from -C(R^{A1})(R^{A2})—, -C(R^{A1})(R^{A2})-C(R^{A1})(R^{A2})—, -C(R^{A1})(R^{A2})—O—, -C(R^{A1})(R^{A2})-NR^{A3}-, - C(═O)—, —C(R^{A1})(R^{A2})—C(═O)—, and -N═C(NH₂)—, wherein no more than one of A, B, D, and E is —C(R^{A1})(R^{A2})—O—, -C(R^{A1})(R^{A2})-NR^{A3}-, -C(R^{A1})(R^{A2})—C(═O)—, -C(═O)—, or -N═C(NH₂)-;
V is N or CH;
Y is N or CH;
G is O, CH₂, or NH;
Ring A is cyclohexyl or 6-membered heterocycloalkyl which has one heteroatom ring member being an oxygen;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CRX, wherein R^{X} is absent, H, halo, CN, OH, C₁₋₆ alkyl, C₁₋₆ alkoxy, amino, C₁₋₆ alkyl amino, or C₂₋₈ dialkylamino;
each R^{A1} is independently selected from absent, H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A2} is independently selected from H, halo, C₁₋₄ alkyl, C₁₋₄ alkoxy, C₁₋₄ haloalkyl, C₁₋₄ haloalkoxy, amino, C₁₋₆ alkylamino, C₂₋₈ dialkylamino, CN, NO₂, and OH;
each R^{A3} is independently selected from H, C₁₋₄ alkyl, C₁₋₄ alkoxy, C₁₋₄ haloalkyl, C(O)Rz, and C(O)OR², wherein said C₁₋₆ alkyl is optionally substituted with phenyl, C₁₋₄ alkoxy, C₁₋₄ haloalkoxy, CN, NO₂, or OH;
R^{z} is H, C₁₋₆ alkyl, or phenyl;
R¹ is
(a) H, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
(b) 3- to 12-membered heterocycloalkyl comprising a sulfur atom, wherein the sulfur atom of the 3- to 12-membered heterocycloalkyl is optionally substituted with one or two R^{1s};
each R^{1s} is independently oxo or =NR⁴a,
(c) -C₁₋₆ alkylene-N(R^{3a}′)(R^{3b}′) or -C₃₋₁₂ cycloalkylene-N(R^{3a}′)(R^{3b}′), wherein the C₁₋₆ alkylene or C₃₋₁₂ cycloalkylene is optionally substituted with one or more R^{1as};
each R¹as is independently halo, oxo, C₁₋₆ alkyl, C₁₋₆ haloalkyl, C(═O)(R^{3a}′), N(R^{3a}′)(R^{3b}′), or S(═O)₂R^{3a}′;
(d) 3- to 12-membered heterocycloalkyl which has at least one nitrogen and optionally one or more other heteroatom ring members, wherein the 3- to 12-membered heterocycloalkyl is optionally substituted with one or more Ribs,
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
(e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es},
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{e1}C(O)OR^{a1}, NR^{c1}C(O)NR^{c1}R^{d1}, NR^{c1}S(O)R^{b1}, NR^{c1}S(0)₂R^{b1}, NR^{e1}S(0)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO2, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO-C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a} is independently H, CN, or C₁₋₆ alkyl optionally substituted with one or more halo, C₆₋₁₀ aryl, or N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

3. The compound of any one of the preceding claims, a stereoisomer, or a pharmaceutically acceptable salt thereof, wherein when [symbol] is a double bond, X is C.

4. The compound of any one of the preceding claims, a stereoisomer, or a pharmaceutically acceptable salt thereof, wherein the spiro moiety represented by the below formula: 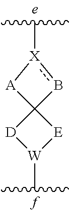
wherein e and f indicate points of attachment to the remainder of the molecule, is selected from:

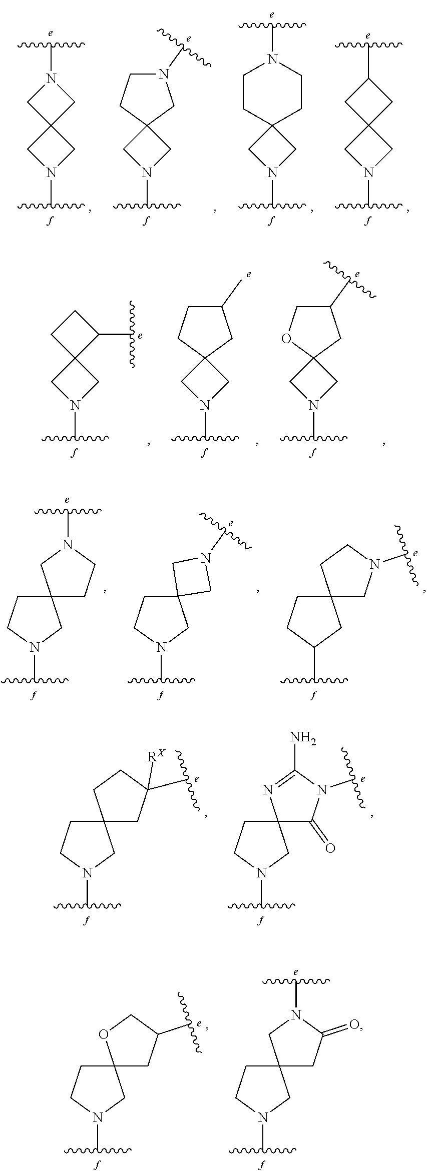

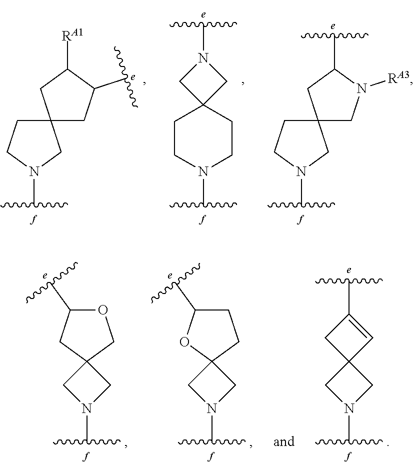

5. A compound of Formula III: 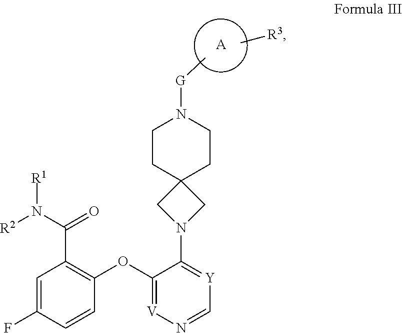
a stereoisomer, or a pharmaceutically acceptable salt thereof, wherein:
V is N or CH;
Y is N or CH;
G is O, CH₂, or NH;
Ring A is cyclohexyl or 6-membered heterocycloalkyl which has one heteroatom ring member being an oxygen;
R¹ is
(a) -C₁₋₆ alkylene-N(R³²′)(R^{3b}′) or -C₃₋₁₂ cycloalkylene-N(R^{3a}′)(R^{3b}′), wherein the C₁₋₆ alkylene or C₃₋₁₂ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
(b) 3- to 12-membered heterocycloalkyl which has at least one nitrogen and optionally one or more other heteroatom ring members, wherein the 3- to 12-membered heterocycloalkyl is optionally substituted with one or more R^{1bs},
each R¹bs is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
(c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es};
each R^{1es} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR¹R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{e1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{e1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₃₋₆ cycloalkyl, C₁₋₆ alkoxy, C₁₋₆ haloalkoxy, cyano-C₁₋₃ alkyl, HO-C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

6. A compound of Formula III-a: 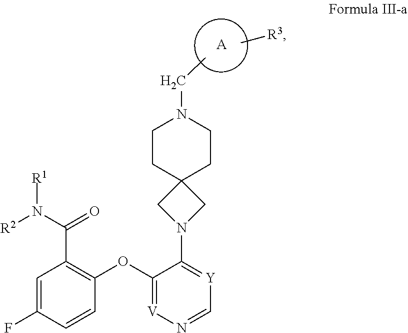
a stereoisomer, or a pharmaceutically acceptable salt thereof, wherein:
V is N or CH;
Y is N or CH;
Ring A is cyclohexyl or 6-membered heterocycloalkyl which has one heteroatom ring member being an oxygen;
R¹ is
(a) -C₁₋₆ alkylene-N(R^{3a}′)(R^{3b}′) or -C₃₋₁₂ cycloalkylene-N(R^{3a}′)(R^{3b}′), wherein the C₁₋₆ alkylene or C₃₋₁₂ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
(b) 3- to 12-membered heterocycloalkyl which has at least one nitrogen and optionally one or more other heteroatom ring members, wherein the 3- to 12-membered heterocycloalkyl is optionally substituted with one or more R^{1bs},
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
(c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es};
each R^{1es} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R³²′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1} NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{e1}S(O)R^{b1}, NR^{c1}S(O)₂R^{b1}, NR^{c1}S(0)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1} NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{e1}S(O)₂R^{b1}, NR^{e1}S(0)₂NR^{c1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, and Rd1 is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO-C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

7. A compound of Formula III-b: 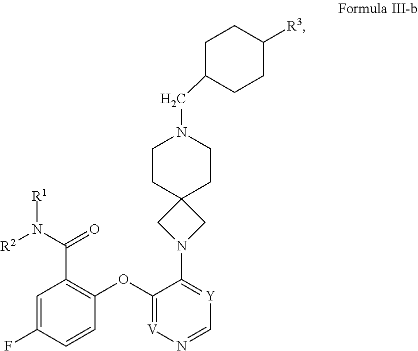
a stereoisomer, or a pharmaceutically acceptable salt thereof, wherein:
V is N or CH;
Y is N or CH;
R¹ is
(a) -C₁₋₆ alkylene-N(R^{3a}′)(R^{3b}′) or -C₃₋₁₂ cycloalkylene-N(R^{3a}′)(R^{3b}′), wherein the C₁₋₆ alkylene or C₃₋₁₂ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
(b) 3- to 12-membered heterocycloalkyl which has at least one nitrogen and optionally one or more other heteroatom ring members, wherein the 3- to 12-membered heterocycloalkyl is optionally substituted with one or more R^{1bs},
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
(c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R³²′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{e1}C(O)OR^{a1}, NR^{c1}C(O)NR^{c1}R^{d1}, NR^{c1}S(O)R^{b1}, NR^{c1}S(O)₂R^{b1}, NR^{e1}S(O)₂NRelR^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NRelR^{d1}, NR^{e1}C(O)R^{b1}, NR^{c1}C(O)OR^{a1}, NR^{e1}C(O)NR^{c1}R^{d1} NR^{e1}S(O)R^{b1}, NR^{c1}S(0)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{a1}, R^{b1}, R^{e1}, and Rd1 is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO-C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

8. A compound of Formula III-c: 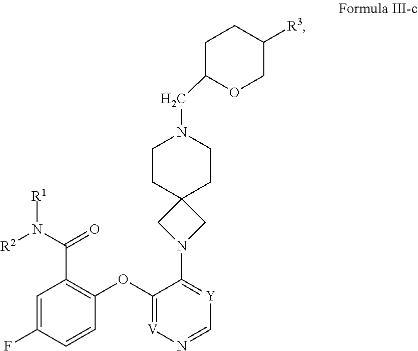
a stereoisomer, or a pharmaceutically acceptable salt thereof, wherein:
V is N or CH;
Y is N or CH;
R¹ is
(a) -C₁₋₆ alkylene-N(R^{3a}′)(R^{3b}′) or -C₃₋₁₂ cycloalkylene-N(R^{3a}′)(R^{3b}′), wherein the C₁₋₆ alkylene or C₃₋₁₂ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
(b) 3- to 12-membered heterocycloalkyl which has at least one nitrogen and optionally one or more other heteroatom ring members, wherein the 3- to 12-membered heterocycloalkyl is optionally substituted with one or more R^{1bs},
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
(c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1es},
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R³²′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, -C₆₋₁₀ aryl-C₁₋₆ alkyl, -C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
R³ is halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1} NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{e1}S(O)₂R^{b1}, NR^{c1}S(O)₂NR^{c1}R^{d1}, S(O)R^{b1}, S(O)NR^{c1}R^{d1}, S(O)₂R^{b1}, and
each R^{a1}, R^{b1}, R^{e1}, and Rd1 is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, -C₆₋₁₀ aryl-C₁₋₆ alkyl, -C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e1} is independently selected from H, C₁₋₆ alkyl, and CN;
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO-C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

9. The compound of any one of the preceding claims, wherein V is N.

10. The compound of any one of the preceding claims, wherein V is CH.

11. The compound of any one of the preceding claims, wherein Y is N.

12. The compound of any one of the preceding claims, wherein Y is CH.

13. The compound of any one of the preceding claims, wherein G is CH₂.

14. The compound of any one of the preceding claims, wherein R² is C₁₋₆ alkyl optionally substituted with 1, 2, 3, 4, or 5 substituents independently selected from R^{g}, wherein:
each R^{g} is independently selected from OH, NO₂, CN, halo, oxo, OBn, N(RN)₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO-C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

15. The compound of any one of the preceding claims, wherein R² is C₁₋₆ alkyl.

16. The compound of any one of the preceding claims, wherein R² is isopropyl.

17. The compound of any one of the preceding claims, wherein R¹ is-C₁₋₆ alkylene-N(R^{3a}′)(R^{3b}′) or -C₃₋₁₂ cycloalkylene-N(R^{3a}′)(R^{3b}′), wherein the C₁₋₆ alkylene or C₃₋₁₂ cycloalkylene is optionally substituted with one or more R^{1as};
each R^{1as} is independently halo, oxo, C₁₋₆ alkyl, C₁₋₆ haloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

18. The compound of any one of the preceding claims, wherein R¹ is 3- to 12-membered heterocycloalkyl which has at least one nitrogen and optionally one or more other heteroatom ring members, wherein the 3- to 12-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

19. The compound of any one of the preceding claims, wherein R¹ is

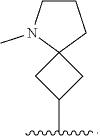 [SMILES: *C1CC2(CCCN2C)C1]

20. The compound of any one of the preceding claims, wherein R¹ is C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs}
each R^{1es} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

21. The compound of any one of the preceding claims, wherein R¹ is

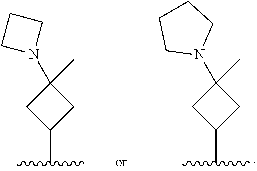

22. The compound of any one of the preceding claims, wherein Ring A is cyclohexyl.

23. The compound of any one of the preceding claims, wherein Ring A is 6-membered heterocycloalkyl which has one heteroatom ring member being an oxygen.

24. The compound of any one of the preceding claims, wherein Ring A is

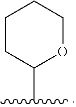 [SMILES: *C1CCCCO1]

25. The compound of any one of the preceding claims, wherein R³ is NR^{c1}S(O)₂R^{b1} or NR^{c1}S(O)₂NR^{c1}R^{d1}, and wherein:
each R^{b1}, R^{e1}, and Rd1 is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl.

26. The compound of any one of the preceding claims, wherein R³ is NR^{e1}S(O)₂R^{b1} or NR^{e1}S(O)₂NR^{e1}R^{d1}, and each R^{b1}, R^{e1}, and Rd1 is independently selected from H and C₁₋₆ alkyl.

27. The compound of any one of the preceding claims, wherein R³ is NHS(O)₂ (C₂H₅) or NHS(O)₂NH(C₂H₅)·28. The compound of any one of the preceding claims, wherein R³ is

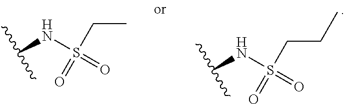

28. A compound as shown in Table 1 or a stereoisomer thereof, or a pharmaceutically acceptable salt thereof.

29. A compound as shown in Table 1 or a pharmaceutically acceptable salt thereof.

30. A compound as shown in Table 1.

31. A compound as shown in Table 1 or a pharmaceutically acceptable salt thereof, wherein the salt is hydrochloride.

32. The compound according to any one of the preceding claims, wherein the compound is useful for the treatment of cancer and wherein the compound minimizes hERG binding.

33. A pharmaceutical composition comprising a compound of any one of the preceding claims, or a pharmaceutically acceptable salt thereof, and at least one pharmaceutically acceptable carrier.

34. A pharmaceutical composition comprising a salt or crystalline form of the compound of any one of claims 1-32, and at least one pharmaceutically acceptable carrier.

35. A method of inhibiting the interaction between menin and MLL comprising contacting the menin and MLL with a compound of any one of claims 1-32 or a pharmaceutical composition of claim 33 or claim 34.

36. A method of treating cancer in a patient comprising administering to the patient a compound of any one of claims 1-32 or a pharmaceutical composition of claim 33 or claim 34.

37. The method of claim 36, wherein the cancer is a hematological cancer.

38. The method of claim 36, wherein the cancer is a leukemia or lymphoma.

39. The method of claim 36, wherein the cancer is mixed lineage leukemia (MLL), MLL-related leukemia, MLL-associated leukemia, MLL-positive leukemia, MLL-induced leukemia, rearranged mixed lineage (KMT2A-rearranged) leukemia (MLL-r), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CML), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sézary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), or Waldenstrom's macroglobulinemia.

40. The method of claim 36, wherein the cancer is an abstract nucleophosmin (NPM1)-mutated acute myeloid leukemia (i.e., NPM1mut acute myloid leukemia) or a rearranged mixed lineage (KMT2A-rearranged) leukemia (MLL-r).

41. The compound of any one of claims 1-32 or a pharmaceutically acceptable salt thereof, or the pharmaceutical composition of claim 33 or claim 34, for use in treating or preventing a disease caused by, or associated with, menin expression, activity, and/or function.

42. The compound of any one of claims 1-32 or a pharmaceutically acceptable salt thereof, or the pharmaceutical composition of claim 33 or claim 34, for use in treating or preventing cancer.

43. Use of the compound of any one of claims 1-32 or a pharmaceutically acceptable salt thereof, or the pharmaceutical composition of claim 33 or claim 34, for treating or preventing a disease caused by, or associated with, menin expression, activity, and/or function.

44. Use of the compound of any one of claims 1-32 or a pharmaceutically acceptable salt thereof, or the pharmaceutical composition of claim 33 or claim 34, in the manufacture of a medicament for treating or preventing a disease caused by, or associated with, menin expression, activity, and/or function.

45. Use of a compound of any of claims 1-32 or a pharmaceutically acceptable salt thereof, or the pharmaceutical composition of claim 33 or claim 34, for treating or preventing cancer.

46. Use of the compound of any one of claims 1-32 or a pharmaceutically acceptable salt thereof, or the pharmaceutical composition of claim 33 or claim 34, in the manufacture of a medicament for treating or preventing cancer.

47. A kit comprising the compound of any one of claims 1-32 or a pharmaceutically acceptable salt thereof, or the pharmaceutical composition of claim 33 or claim 34 and instructions for its use.

48. A method of preparing a compound herein according to a scheme of the present disclosure or synthetic description in the Examples.

49. An intermediate useful in the preparation of any one of the compounds herein.
